(19) United States
(12) Patent Application Publication
(10) Pub. No.: US 2026/0335474 A1
(43) Pub. Date: Oct. 29, 2026

(54) RADIOLABELED ANTI-LAG3 ANTIBODIES FOR IMMUNO-PET IMAGING

(71) Applicant: Regeneron Pharmaceuticals, Inc., Tarrytown, NY (US)

(72) Inventors: Marcus Kelly, New York, NY (US); Dangshe Ma, Pleasantville, NY (US); William Olson, Yorktown Heights, NY (US); Gavin Thurston, Millerton, NY (US); Richard Tavare, Salt Lake City, UT (US)

(21) Appl. No.: 19/730,681

(22) Filed: Jul. 2, 2026

Related U.S. Application Data
(63) Continuation of application No. 18/049,609, filed on Oct. 25, 2022, now Pat. No. 12,721,914.
Application No. 18/049,609 is a continuation of application No. 17/127,618, filed on Dec. 18, 2020, now Pat. No. 11,511,001.
Application No. 17/127,618 is a division of application No. 15/892,440, filed on Feb. 9, 2018, now Pat. No. 10,905,784.
(60) Provisional application No. 62/457,287, filed on Feb. 10, 2017.

(51) Int. Cl. A61K 51/10; A61K 39/00; C07C 259/00; C07K 16/28

(52) U.S. Cl. CPC A61K 51/1027; A61K 39/00; A61K 51/1039; C07C 259/00; C07K 16/2803; C07K 2317/21

(57) ABSTRACT

Radiolabeled anti-LAG3 antibodies and their use in immuno-PET imaging are provided herein. Included are methods of detecting the presence of LAG3 proteins in a patient or sample.

### CROSS-REFERENCE TO RELATED APPLICATIONS

This application is a continuation of U.S. patent application Ser. No. 18/049,609, filed Oct. 25, 2022, which is a continuation of U.S. patent application Ser. No. 17/127,618 filed Dec. 18, 2020, now U.S. Pat. No. 11,511,001, which is a divisional of U.S. patent application Ser. No. 15/892,440, filed Feb. 9, 2018, now U.S. Pat. No. 10,905,784 which claims the benefit under 34 U.S.C. § 119(e) of U.S. Provisional Application No. 62/457,287, filed Feb. 10, 2017, which is herein specifically incorporated by reference in its entirety.

### FIELD

This disclosure relates to radiolabeled anti-LAG3 antibodies and their use in immuno-PET imaging.

### SEQUENCE LISTING

An official copy of the sequence listing is submitted concurrently with the specification electronically via Patent Center. The contents of the electronic sequence listing (10329US04-US_Sequence_Listing_ST26.xml; Size: 708,608 bytes; and Date of Creation: Jul. 2, 2026) is herein incorporated by reference in its entirety.

### BACKGROUND

T cell co-stimulatory and co-inhibitory molecules (collectively named co-signaling molecules) play a crucial role in regulating T cell activation, subset differentiation, effector function and survival (Chen et al 2013, Nature Rev. Immunol. 13: 227-242). Following recognition of cognate peptide-MHC complexes on antigen-presenting cells by the T cell receptor (TCR), co-signaling receptors co-localize with T cell receptors at the immune synapse, where they synergize with TCR signaling to promote or inhibit T cell activation and function (Flies et al 2011, Yale J. Biol. Med. 84: 409-421). The ultimate immune response is regulated by a balance between co-stimulatory and co-inhibitory signals (“immune checkpoints”) (Pardoll 2012, Nature Reviews Cancer 12: 252-264). Lymphocyte activation gene-3 (LAG3) functions as one such ‘immune checkpoint’ in mediating peripheral T cell tolerance.

LAG3 (also called CD223) is a 503 amino acid transmembrane protein receptor expressed on activated CD4 and CD8 T cells, γδ T cells, natural killer T cells, B-cells, natural killer cells, plasmacytoid dendritic cells and regulatory T cells. LAG3 is a member of the immunoglobulin (Ig) superfamily. The primary function of LAG3 is to attenuate the immune response. LAG3 binding to MHC class II molecules results in delivery of a negative signal to LAG3-expressing cells and down-regulates antigen-dependent CD4 and CD8 T cell responses. LAG3 negatively regulates the ability of T cells to proliferate, produce cytokines and lyse target cells, termed as ‘exhaustion’ of T cells. LAG3 is also reported to play a role in enhancing T regulatory (Treg) cell function (Pardoll 2012, Nature Reviews Cancer 12: 252-264).

Immuno-positron emission tomography (PET) is a diagnostic imaging tool that utilizes monoclonal antibodies labeled with positron emitters, combining the targeting properties of an antibody with the sensitivity of positron emission tomography cameras. See, e.g., The Oncologist, 12: 1379 (2007); Journal of Nuclear Medicine, 52(8): 1171 (2011). Immuno-PET enables the visualization and quantification of antigen and antibody accumulation in vivo and, as such, can serve as an important tool for diagnostics and complementing therapy. For example, immuno-PET can aid in the selection of potential patient candidates for a particular therapy, as well as in the monitoring of treatment.

As LAG3 has emerged as a target for tumor immunotherapy and infectious immunotherapy, there is need for diagnostic tools for anti-LAG3 therapy, including, inter alia, diagnostic tools that enable the detection of suitable patient candidates for said therapy.

### BRIEF SUMMARY

Included in this disclosure are radiolabeled anti-LAG3 antibody conjugates for use in immuno-PET imaging.

In one aspect, the conjugate comprises an anti-LAG3 antibody or antigen-binding fragment thereof, a chelating moiety, and a positron emitter.

Provided herein are also processes for synthesizing said conjugates and synthetic intermediates useful for the same.

Provided herein are also methods of imaging a tissue that expresses LAG3, the methods comprising administering a radiolabeled anti-LAG3 antibody conjugate described herein to the tissue; and visualizing the LAG3 expression by positron emission tomography (PET) imaging.

Provided herein are also methods of imaging a tissue comprising LAG3-expressing cells, for example, LAG3-expressing intratumoral lymphocytes, the methods comprising administering a radiolabeled anti-LAG3 antibody conjugate described herein to the tissue, and visualizing the LAG3 expression by PET imaging.

Provided herein are also methods for detecting LAG3 in a tissue, the methods comprising administering a radiolabeled anti-LAG3 antibody conjugate described herein to the tissue; and visualizing the LAG3 expression by PET imaging. In one embodiment, the tissue is present in a human subject. In certain embodiments, the subject is a non-human mammal. In certain embodiments, the subject has a disease or disorder such as cancer, an inflammatory disease, or an infection.

Provided herein are also methods for identifying a patient to be suitable for anti-tumor therapy comprising an inhibitor of LAG3, the methods comprising selecting a patient with a solid tumor, administering a radiolabeled antibody conjugate described herein, and visualizing the administered radiolabeled antibody conjugate in the tumor by PET imaging wherein presence of the radiolabeled antibody conjugate in the tumor identifies the patient as suitable for anti-tumor therapy comprising an inhibitor of LAG3.

Provided herein are also methods of treating a tumor, the methods comprising selecting a subject with a solid tumor; determining that the solid tumor is LAG3-positive; and administering an anti-tumor therapy to the subject in need thereof. In certain embodiments, the anti-tumor therapy comprises an inhibitor of LAG3. In certain embodiments, the anti-tumor therapy comprises an inhibitor of the PD-1/PD-L1 signaling axis (e.g., an anti-PD-1 antibody or an anti-PD-L1 antibody). In certain embodiments, the anti-tumor therapy comprises an inhibitor of LAG3 and/or an inhibitor of the PD-1/PD-L1 signaling axis. In certain embodiments, the subject is administered a radiolabeled anti-LAG3 antibody conjugate described herein, and localization of the radiolabeled antibody conjugate is imaged via positron emission tomography (PET) imaging to determine if the tumor is LAG3-positive. In certain embodiments, the subject is further administered a radiolabeled anti-PD-1 antibody conjugate, and localization of the radiolabeled antibody conjugate is imaged via positron emission tomography (PET) imaging to determine if the tumor is PD-1-positive.

Provided herein are also methods for monitoring the efficacy of an anti-tumor therapy in a subject, wherein the methods comprise selecting a subject with a solid tumor wherein the subject is being treated with an anti-tumor therapy; administering a radiolabeled anti-LAG3 conjugate described herein to the subject; imaging the localization of the administered radiolabeled conjugate in the tumor by PET imaging; and determining tumor growth, wherein a decrease from the baseline in uptake of the conjugate or radiolabeled signal indicates efficacy of the anti-tumor therapy. In certain embodiments, the anti-tumor therapy comprises an inhibitor of LAG3 (e.g., an anti-LAG3 antibody). In certain embodiments, the anti-tumor therapy comprises an inhibitor of LAG3 and an inhibitor of the PD-1/PD-L1 signaling axis. In certain embodiments, the anti-tumor therapy comprises a PD-1 inhibitor (e.g., REGN2810, BGB-A317, nivolumab, pidilizumab, and pembrolizumab), a PD-L1 inhibitor (e.g., atezolizumab, avelumab, durvalumab, MDX-1105, and REGN3504, as well as those disclosed in Patent Publication No. US 2015-0203580), CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3xCD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, and an antibody-drug conjugate (ADC) (e.g., anti-CD19-DM4 ADC, and anti-DS6-DM4 ADC).

Provided herein are also methods for predicting response of a patient to an anti-tumor therapy, the methods comprising selecting a patient with a solid tumor; and determining if the tumor is LAG3-positive, wherein if the tumor is LAG3-positive it predicts a positive response of the patient to an anti-tumor therapy. In certain embodiments, the tumor is determined positive by administering a radiolabeled anti-LAG3 antibody conjugate of the present disclosure and localizing the radiolabeled antibody conjugate in the tumor by PET imaging wherein presence of the radiolabeled antibody conjugate in the tumor indicates that the tumor is LAG3-positive. In some embodiments, the anti-tumor therapy is selected from a PD-1 inhibitor (e.g., REGN2810, BGB-A317, nivolumab, pidilizumab, and pembrolizumab), a PD-L1 inhibitor (e.g., atezolizumab, avelumab, durvalumab, MDX-1105, and REGN3504), CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3xCD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, and an antibody-drug conjugate (ADC) (e.g., anti-CD19-DM4 ADC, and anti-DS6-DM4 ADC).

Provided herein are also methods for predicting response of a patient to an anti-tumor therapy comprising an inhibitor LAG3, the methods comprising selecting a patient with a solid tumor; and determining if the tumor is LAG3-positive, wherein if the tumor is LAG3-positive it indicates a positive response of the patient to an anti-tumor therapy comprising an inhibitor of LAG3. In certain embodiments, the tumor is determined positive by administering a radiolabeled anti-LAG3 antibody conjugate of the present disclosure and localizing the radiolabeled antibody conjugate in the tumor by PET imaging wherein presence of the radiolabeled antibody conjugate in the tumor indicates that the tumor is LAG3-positive.

### BRIEF DESCRIPTION OF THE FIGURES

FIG. 1 depicts UV/VIS spectrum of DFO modified anti-LAG3 antibody (mAb1-DFO).

FIG. 2 depicts HPLC-SEC of DFO modified anti-LAG3 antibody.

FIG. 3 depicts radio-SEC-HPLC of isotype-DFO-conjugate after ⁸⁹Zr radiolabeling for Study 1.

FIG. 4 depicts radio-SEC-HPLC of anti-LAG3-DFO-conjugate after ⁸⁹Zr radiolabeling for Study 1.

FIG. 5 depicts radio-SEC-HPLC of anti-LAG3-DFO-conjugate after ⁸⁹Zr radiolabeling for Study 2.

FIG. 6 depicts UV280-SEC-HPLC chromatogram and radio-iTLC trace of isotype-DFO-conjugate after ⁸⁹Zr radiolabeling for Study 1.

FIG. 7 depicts UV280-SEC-HPLC chromatogram and radio-iTLC trace of anti-LAG3-DFO-conjugate after ⁸⁹Zr radiolabeling for Study 1.

FIG. 8 depicts UV280-SEC-HPLC chromatogram and radio-iTLC trace of anti-LAG3-DFO-conjugate after ⁸⁹Zr radiolabeling for Study 2.

FIG. 9 provides representative images of ⁸⁹Zr-DFO-mAb1 injected at a protein dose of 5 mg/kg (Ms01) or 0.03 mg/kg (Ms14) demonstrating specific targeting of ⁸⁹Zr-DFO-mAb1 to Raji/hPBMC tumors using 0.03 mg/kg of ⁸⁹Zr-DFO-mAb1 and blocking at 5 mg/kg of ⁸⁹Zr-DFO-mAb1. Specific uptake in the spleen and lymph nodes is seen at the lower dose of 0.03 mg/kg ⁸⁹Zr-DFO-mAb1.

FIG. 10 shows LAG3 expression in tissue samples from PBMC/Raji xenografts (obtained at 27 days and 15 days after tumor implantation) and in melanoma clinical samples.

FIG. 11 provides data demonstrating REGN2810 anti-human PD-1 Ab and mAb1 anti-human LAG-3 respectively increase LAG-3+ T cells and PD-1+ T cells in tumor microenvironment.

FIG. 12 provides characteristics of the melanoma samples studied in Example 7.

FIG. 13 provides a schematic presentation of the therapeutic dosing regimen used in Example 8.

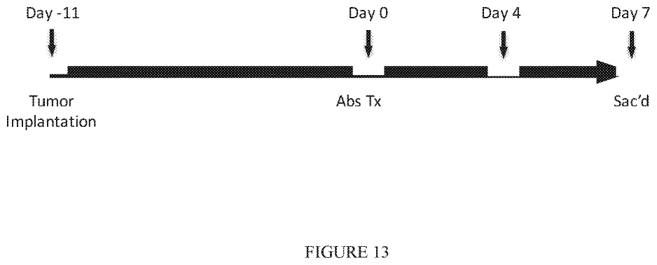

### DETAILED DESCRIPTION

### 1. Definitions

Unless defined otherwise herein, all technical and scientific terms used herein have the same meaning as commonly understood by one of ordinary skill in the art to which the disclosed subject matter belongs.

The term “LAG3” refers to the lymphocyte activation gene-3 protein, an immune checkpoint receptor or T cell co-inhibitor, also known as CD223. The amino acid sequence of full-length LAG3 is provided in GenBank as accession number NP_002277.4 and is also referred to herein as SEQ ID NO: 582. The term “LAG3” also includes protein variants of LAG3 having the amino acid sequence of SEQ ID NOs: 574, 575 or 576. The term “LAG3” includes recombinant LAG3 or a fragment thereof. The term also encompasses LAG3 or a fragment thereof coupled to, for example, histidine tag, mouse or human Fc, or a signal sequence such as the signal sequence of ROR1. For example, the term includes sequences exemplified by SEQ ID NO: 575, comprising a mouse Fc (mlgG2a) at the C-terminal, coupled to amino acid residues 29-450 of full-length ectodomain LAG3. Protein variants as exemplified by SEQ ID NO: 574 comprise a histidine tag at the C-terminal, coupled to amino acid residues 29-450 of full length ectodomain LAG3. Unless specified as being from a non-human species, the term “LAG3” means human LAG3.

LAG3 is a member of the immunoglobulin (Ig) superfamily. LAG3 is a type-1 transmembrane protein with four extracellular Ig-like domains D1 to D4 and is expressed on intratumoral lymphocytes including activated T cells, natural killer cells, B cells, plasmacytoid dendritic cells, and regulatory T cells. The LAG3 receptor binds to MHC class II molecules present on antigen presenting cells (APCs).

The term “B7-1” refers to the T-lymphocyte activation antigen, also known as costimulatory factor CD80. B7-1 is a 288 amino acid membrane receptor with an extracellular N-terminal domain which comprises IgV-like (aa 37-138) and IgC-like (aa 154-232) regions, a transmembrane domain (aa 243-263) and a C-terminal intracellular region (aa 263-288). The amino acid sequence of full-length B7-1 is provided in GenBank as accession number NP_005182.1.

As used herein, the term “T-cell co-inhibitor” refers to a ligand and/or receptor which modulates the immune response via T-cell activation or suppression. The term “T-cell co-inhibitor”, also known as T-cell co-signaling molecule, includes, but is not limited to, lymphocyte activation gene 3 protein (LAG-3, also known as CD223), programmed death-1 (PD-1), cytotoxic T-lymphocyte antigen-4 (CTLA-4), B and T lymphocyte attenuator (BTLA), CD-28, 2B4, LY108, T-cell immunoglobulin and mucin-3 (TIM3), T-cell immunoreceptor with immunoglobulin and ITIM domains (TIGIT; also known as VSIG9), leucocyte associated immunoglobulin-like receptor 1 (LAIR1; also known as CD305), inducible T-cell costimulator (ICOS; also known as CD278), B7-1 (CD80), and CD160.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains C_{H}1, C_{H}2 and C_{H}3). Each light chain is comprised of a light chain variable region (“LCVR or “VL”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The anti-LAG3 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-LAG3 monoclonal antibodies comprising variants of any of the HCVR, LCVR, and/or CDR amino acid sequences disclosed herein having one or more conservative substitutions. For example, the present disclosure includes anti-LAG3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

The term “multi-specific antigen-binding molecules”, as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multi-specific antigen-binding molecules” also includes bi-specific, tri-specific or multi-specific antibodies or antigen-binding fragments thereof. In certain embodiments, an antibody of the present disclosure is functionally linked to another antibody or antigen-binding fragment thereof to produce a bispecific antibody with a second binding specificity. Bispecific and multi-specific antibodies of the present disclosure are described elsewhere herein.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁸ M or less (e.g., a smaller K_{D} denotes a tighter binding). Methods for determining whether two molecules specifically bind are well known in the art and include, for example, equilibrium dialysis, surface plasmon resonance, and the like.

As described herein, antibodies have been identified by surface plasmon resonance, e.g., BIACORE™, which bind specifically to LAG3. Moreover, multi-specific antibodies that bind to one domain in LAG3 and one or more additional antigens or a bi-specific that binds to two different regions of LAG3 are nonetheless considered antibodies that “specifically bind”, as used herein.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to LAG3.

An “isolated antibody”, as used herein, is intended to refer to an antibody that is substantially free of other antibodies (Abs) having different antigenic specificities (e.g., an isolated antibody that specifically binds LAG3, or a fragment thereof, is substantially free of Abs that specifically bind antigens other than LAG3.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix. Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding). As used herein, the term “subject” refers to an animal, preferably a mammal, in need of amelioration, prevention and/or treatment of a disease or disorder such as chronic viral infection, cancer or autoimmune disease.

### II. Radiolabeled Immunoconjugates of LAG3 Antibodies for Immuno-PET Imaging

Provided herein are radiolabeled antigen-binding proteins that bind LAG3. In some embodiments, the radiolabeled antigen-binding proteins comprise an antigen-binding protein covalently linked to a positron emitter. In some embodiments, the radiolabeled antigen-binding proteins comprise an antigen-binding protein covalently linked to one or more chelating moieties, which are chemical moieties that are capable of chelating a positron emitter.

In some embodiments, antigen-binding proteins that bind LAG3, e.g., antibodies, are provided, wherein said antigen-binding proteins that bind LAG3 are covalently bonded to one or more moieties having the following structure:

-L-M_{Z}

wherein L is a chelating moiety; M is a positron emitter; and z, independently at each occurrence, is 0 or 1; and wherein at least one of z is 1.

In some embodiments, the radiolabeled antigen-binding protein is a compound of Formula (I):

M-L-A-[L-M_{Z}]ₖ (I)

A is a protein that binds LAG3; L is a chelating moiety; M is a positron emitter; z is 0 or 1; and k is an integer from 0-30. In some embodiments, k is 1.

In certain embodiments, the radiolabeled antigen-binding protein is a compound of Formula (II):

A-[L-M]ₖ (II)

wherein A is a protein that binds LAG3; L is a chelating moiety; M is a positron emitter; and k is an integer from 1-30.

In some embodiments, provided herein are compositions comprising a conjugate having the following structure:

A-Lₖ

wherein A is a protein that binds LAG3; L is a chelating moiety; and k is an integer from 1-30; wherein the conjugate is chelated with a positron emitter in an amount sufficient to provide a specific activity suitable for clinical PET imaging.

Suitable binding proteins, chelating moieties, and positron emitters are provided below.

### A. LAG3 Binding Proteins

Suitable LAG3 binding protein are proteins that specifically bind to LAG3, including those described in PCT/US16/56156, incorporated herein by reference in its entirety. Exemplary anti-LAG3 antibodies of the present disclosure are listed in Table 1 of PCT/US16/56156, also presented below.

TABLE 1
Amino Acid Sequence Identifiers

| | SEQ ID NOs: | | | | | | | |
|---|---|---|---|---|---|---|---|---|
| Antibody | | | | | | | | |
| Designation | HCVR | HCDR1 | HCDR2 | HCDR3 | LCVR | LCDR1 | LCDR2 | LCDR3 |
| H1M14985N | 2 | 4 | 6 | 8 | 10 | 12 | 14 | 16 |
| H1M14987N | 18 | 20 | 22 | 24 | 26 | 28 | 30 | 32 |
| H2M14811N | 34 | 36 | 38 | 40 | 42 | 44 | 46 | 48 |
| H2M14885N | 50 | 52 | 54 | 56 | 58 | 60 | 62 | 64 |
| H2M14926N | 66 | 68 | 70 | 72 | 74 | 76 | 78 | 80 |
| H2M14927N | 82 | 84 | 86 | 88 | 90 | 92 | 94 | 96 |
| H2M14931N | 98 | 100 | 102 | 104 | 106 | 108 | 110 | 112 |
| H2M18336N | 114 | 116 | 118 | 120 | 122 | 124 | 126 | 128 |
| H2M18337N | 130 | 132 | 134 | 136 | 138 | 140 | 142 | 144 |
| H4H15477P | 146 | 148 | 150 | 152 | 154 | 156 | 158 | 160 |
| H4H15483P | 162 | 164 | 166 | 168 | 170 | 172 | 174 | 176 |
| H4H15484P | 178 | 180 | 182 | 184 | 186 | 188 | 190 | 192 |
| H4H15491P | 194 | 196 | 198 | 200 | 202 | 204 | 206 | 208 |
| H4H17823P | 210 | 212 | 214 | 216 | 218 | 220 | 222 | 224 |
| H4H17826P2 | 226 | 228 | 230 | 232 | 234 | 236 | 238 | 240 |
| H4H17828P2 | 242 | 244 | 246 | 248 | 250 | 252 | 254 | 256 |
| H4sH15460P | 258 | 260 | 262 | 264 | 266 | 268 | 270 | 272 |
| H4sH15462P | 274 | 276 | 278 | 280 | 282 | 284 | 286 | 288 |
| H4sH15463P | 290 | 292 | 294 | 296 | 298 | 300 | 302 | 304 |
| H4sH15464P | 306 | 308 | 310 | 312 | 314 | 316 | 318 | 320 |
| H4sH15466P | 322 | 324 | 326 | 328 | 330 | 332 | 334 | 336 |
| H4sH15467P | 338 | 340 | 342 | 344 | 346 | 348 | 350 | 352 |
| H4sH15470P | 354 | 356 | 358 | 360 | 362 | 364 | 366 | 368 |
| H4sH15475P | 370 | 372 | 374 | 376 | 378 | 380 | 382 | 384 |
| H4sH15479P | 386 | 388 | 390 | 392 | 394 | 396 | 398 | 400 |
| H4sH15480P | 402 | 404 | 406 | 408 | 410 | 412 | 414 | 416 |
| H4sH15482P | 418 | 420 | 422 | 424 | 426 | 428 | 430 | 432 |
| H4sH15488P | 434 | 436 | 438 | 440 | 442 | 444 | 446 | 448 |
| H4sH15496P2 | 450 | 452 | 454 | 456 | 522 | 524 | 526 | 528 |
| H4sH15498P2 | 458 | 460 | 462 | 464 | 522 | 524 | 526 | 528 |
| H4sH15505P2 | 466 | 468 | 470 | 472 | 522 | 524 | 526 | 528 |
| H4sH15518P2 | 474 | 476 | 478 | 480 | 522 | 524 | 526 | 528 |
| H4sH15523P2 | 482 | 484 | 486 | 488 | 522 | 524 | 526 | 528 |
| H4sH15530P2 | 490 | 492 | 494 | 496 | 522 | 524 | 526 | 528 |
| H4sH15555P2 | 498 | 500 | 502 | 504 | 530 | 532 | 534 | 536 |
| H4sH15558P2 | 506 | 508 | 510 | 512 | 530 | 532 | 534 | 536 |
| H4sH15567P2 | 514 | 516 | 518 | 520 | 530 | 532 | 534 | 536 |
| H4H14813N | 538 | 540 | 542 | 544 | 546 | 548 | 550 | 552 |
| H4H17819P | 554 | 556 | 558 | 560 | 562 | 564 | 566 | 568 |

Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-LAG3 antibodies.

In some embodiments, the binding protein is an antibody or antigen binding fragment comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

In some embodiments, the binding protein is an antibody or antigen binding fragment comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

In some embodiments, the binding protein is an antibody or antigen binding fragment comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present disclosure provides antibodies, or antigen-binding fragments thereof, comprising an HCVR/LCVR amino acid sequence pair contained within any of the exemplary anti-LAG3 antibodies listed in Table 1. In certain embodiments, the HCVR/LCVR amino acid sequence pair is selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562. In certain embodiments, the HCVR/LCVR amino acid sequence pair is selected from one of SEQ ID NOs: 386/394 (e.g., H4sH15479P), 418/426 (e.g., H4sH15482P) or 538/546 (e.g., H4sH14813N). In certain other embodiments, the HCVR/LCVR amino acid sequence pair is selected from one of SEQ ID NOs: 458/464 (e.g., H4sH15498P2), 162/170 (e.g., H4H15483P), and 579/578 (e.g., H4H15482P).

In some embodiments, the binding protein is an antibody or antigen binding fragment comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

In some embodiments, the binding protein is an antibody or antigen binding fragment comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

In some embodiments, the binding protein is an antibody or antigen binding fragment comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present disclosure provides antibodies, or antigen-binding fragments thereof, comprising an HCDR3/LCDR3 amino acid sequence pair contained within any of the exemplary anti-LAG3 antibodies listed in Table 1. In certain embodiments, the HCDR3/LCDR3 amino acid sequence pair is selected from the group consisting of SEQ ID NOs: 392/400 (e.g., H4sH15479P), 424/432 (e.g., H4sH15482P), and 544/552 (e.g., H4sH14813N).

In some embodiments, the binding protein is an antibody or antigen binding fragment comprising a set of six CDRs (i.e., HCDR1-HCDR2-HCDR3-LCDR1-LCDR2-LCDR3) contained within any of the exemplary anti-LAG3 antibodies listed in Table 1. In certain embodiments, the HCDR1-HCDR2-HCDR3-LCDR1-LCDR2-LCDR3 amino acid sequence set is selected from the group consisting of SEQ ID NOs: 388-390-392-396-398-400 (e.g., H4sH15479P), 420-422-424-428-430-432 (e.g., H4sH15482P), and 540-542-544-548-550-552 (e.g., H4sH14813N).

In some embodiments, the binding protein is an antibody or antigen binding fragment comprising a set of six CDRs (i.e., HCDR1-HCDR2-HCDR3-LCDR1-LCDR2-LCDR3) contained within an HCVR/LCVR amino acid sequence pair as defined by any of the exemplary anti-LAG3 antibodies listed in Table 1. For example, in some embodiments, the binding protein is an antibody or antigen binding fragment comprising the HCDR1-HCDR2-HCDR3-LCDR1-LCDR2-LCDR3 amino acid sequences set contained within an HCVR/LCVR amino acid sequence pair selected from the group consisting of SEQ ID NOs: 386/394 (e.g., H4sH15479P), 418/426 (e.g., H4sH15482P) and 538/546 (e.g., H4sH14813N). Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In some embodiments, binding proteins are antibodies and antigen-binding fragments thereof that compete for specific binding to LAG3 with an antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

Additional exemplary anti-LAG3 antibodies useful herein include LAG525 (and other LAG3 antibodies disclosed in U.S. 20100233183), relatlimab (and other LAG3 antibodies disclosed in U.S. 20110150892), GSK2831781 (and other LAG3 antibodies disclosed in U.S. 20140286935), MGD013 (and other LAG3 antibodies disclosed in WO2015200119) and LAG3 antibodies disclosed in U.S. 20160222116, U.S. 20170022273, U.S. 20170097333, U.S. 20170137517, U.S. 20170267759, U.S. 20170290914, U.S. 20170334995, WO2016126858, WO2016200782, WO2017087589, WO2017087901, WO2017106129, WO2017149143, WO2017198741, WO2017219995, and WO2017220569.

Also provided herein are isolated antibodies and antigen-binding fragments thereof that block LAG3 binding to MHC class II. In some embodiments, the antibody or antigen-binding fragment thereof that blocks LAG3 binding may bind to the same epitope on LAG3 as MHC class II or may bind to a different epitope on LAG3 as MHC class II. In certain embodiments, the antibodies of the disclosure that block LAG3 binding to MHC class II comprise the CDRs of an HCVR having an amino acid sequence selected from the group consisting of HCVR sequences listed in Table 1; and the CDRs of a LCVR having an amino acid sequence selected from the group consisting of LCVR sequences listed in Table 1.

In alternate embodiments, the present disclosure provides antibodies and antigen-binding fragments thereof that do not block LAG3 binding to MHC class II.

In some embodiments, the binding proteins are antibodies and antigen-binding fragments thereof that bind specifically to LAG3 from human or other species. In certain embodiments, the antibodies may bind to human LAG3 and/or to cynomolgus LAG3.

In some embodiments, the binding proteins are antibodies and antigen-binding fragments thereof that cross-compete for binding to LAG3 with a reference antibody or antigen-binding fragment thereof comprising the CDRs of a HCVR and the CDRs of a LCVR, wherein the HCVR and LCVR each has an amino acid sequence selected from the HCVR and LCVR sequences listed in Table 1.

In one embodiment, the binding protein is an isolated antibody or antigen-binding fragment that has one or more of the following characteristics: (a) blocks the binding of LAG3 or to MHC class II; (b) binds specifically to human LAG3 and/or cynomolgus LAG3; (c) blocks LAG3-induced impairment of T cell activation and rescues T cell signaling; and (d) suppresses tumor growth and increases survival in a subject with cancer.

In some embodiments, the antibody or antigen binding fragment thereof may bind specifically to LAG3 in an agonist manner, i.e., it may enhance or stimulate LAG3 binding and/or activity; in other embodiments, the antibody may bind specifically to LAG3 in an antagonist manner, i.e., it may block LAG3 from binding to its ligand.

In some embodiments, the antibody or antigen binding fragment thereof may bind specifically to LAG3 in an neutral manner, i.e., it binds but does not block or enhance or stimulate LAG3 binding and/or activity.

In certain embodiments, the antibodies or antigen-binding fragments are bispecific comprising a first binding specificity to LAG3 and a second binding specificity for a second target epitope. The second target epitope may be another epitope on LAG3 or on a different protein. In certain embodiments, the second target epitope may be on a different cell including a different T cell, a B-cell, a tumor cell or a virally infected cell.

In certain embodiments, an isolated antibody or antigen-binding fragment thereof is provided that binds specifically to human lymphocyte activation gene 3 (LAG3) protein, wherein the antibody or antigen-binding fragment thereof has a property selected from the group consisting of: (a) binds monomeric human LAG3 with a binding dissociation equilibrium constant (K_{D}) of less than about 10 nM as measured in a surface plasmon resonance assay at 25 °C (using the assay format as defined in Example 3 of PCT/US16/56156, or a substantially similar assay); (b) binds monomeric human LAG3 with a K_{D} less than about 8 nM as measured in a surface plasmon resonance assay at 37 °C; (c) binds dimeric human LAG3 with a K_{D} less than about 1.1 nM as measured in a surface plasmon resonance assay at 25 °C; (d) binds dimeric human LAG3 with a K_{D} less than about 1 nM as measured in a surface plasmon resonance assay at 37 °C; (e) binds to a hLAG3-expressing cell with an EC₅₀ less than about 8 nM as measured in a flow cytometry assay; (f) binds to a mfLAG3-expressing cell with a EC₅₀ less than about 2.3 nM as measured in a flow cytometry assay; (g) blocks binding of hLAG3 to human MHC class II with IC₅₀ less than about 32 nM as determined by a cell adherence assay; (h) blocks binding of hLAG3 to mouse MHC class II with IC₅₀ less than about 30 nM as determined by a cell adherence assay; (i) blocks binding of hLAG3 to MHC class II by more than 90% as determined by a cell adherence assay; (j) rescues LAG3-mediated inhibition of T cell activity with EC₅₀ less than about 9 nM as determined in a luciferase reporter assay; and (k) binds to activated CD4+ and CD8+ T cells with EC₅₀ less than about 1.2 nM, as determined in a fluorescence assay.

In some embodiments, the antibodies and antigen-binding fragments thereof bind LAG3 with a dissociative half-life (t½) of greater than about 1.6 minutes as measured by surface plasmon resonance at 25 °C or 37 °C, e.g., using an assay format as defined in Example 3 of PCT/US16/56156, or a substantially similar assay. In certain embodiments, the antibodies or antigen-binding fragments bind LAG3 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1100 minutes, as measured by surface plasmon resonance at 25 °C or 37 °C, e.g., using an assay format as defined in Example 3 of PCT/US16/56156 (e.g., mAb-capture or antigen-capture format), or a substantially similar assay.

In some embodiments, antibodies or antigen-binding fragments thereof bind to a human LAG3-expressing cell with an EC₅₀ less than about 8 nM as measured by a flow cytometry assay as defined in Example 5 of PCT/US16/56156, or a substantially similar assay. In certain embodiments, the antibodies or antigen-binding fragments thereof bind to a hLAG3-expressing cell with an EC₅₀ less than about 5 nM, less than about 2 nM, less than about 1 nM, or less than about 0.5 nM, as measured by a flow cytometry assay, e.g., using the assay format in Example 5 of PCT/US16/56156, or a substantially similar assay.

In some embodiments, antibodies or antigen-binding fragments thereof bind to a cynomolgus monkey LAG3-expressing cell with an EC₅₀ less than about 2.5 nM as measured by a flow cytometry assay as defined in Example 5 of PCT/US16/56156, or a substantially similar assay. In certain embodiments, the antibodies or antigen-binding fragments thereof bind to a mfLAG3-expressing cell with an EC₅₀ less than about 2 nM, or less than about 1 nM, as measured by a flow cytometry assay, e.g., using the assay format as defined in Example 5 of PCT/US16/56156, or a substantially similar assay.

In some embodiments, antibodies or antigen-binding fragments thereof block LAG3 binding to MHC class II (e.g., human HLA-DR2) with an IC₅₀ of less than about 32 nM as determined using a cell adherence assay, e.g., as shown in Example 7 of PCT/US16/56156, or a substantially similar assay. In certain embodiments, the antibodies or antigen-binding fragments thereof block LAG3 binding to human MHC class II with an IC₅₀ less than about 25 nM, less than about 20 nM, less than about 10 nM, or less than about 5 nM, as measured by a cell adherence assay, e.g., using the assay format as defined in Example 7 of PCT/US16/56156, or a substantially similar assay.

In some embodiments, the antibodies or antigen-binding fragments thereof block LAG3 binding to MHC class II with an IC₅₀ of less than about 30 nM as determined using a cell adherence assay, e.g., as shown in Example 7 of PCT/US16/56156, or a substantially similar assay. In certain embodiments, the antibodies or antigen-binding fragments thereof block mouse LAG3 binding to human MHC class II with an IC₅₀ less than about 25 nM, less than about 20 nM, less than about 10 nM, or less than about 5 nM, as measured by a cell adherence assay, e.g., using the assay format as defined in Example 7 of PCT/US16/56156, or a substantially similar assay.

In some embodiments, the antibodies or antigen-binding fragments thereof block binding of LAG3 to human or mouse MHC class II by more than 90% as measured by a cell adherence assay as defined in Example 7 of PCT/US16/56156, or a substantially similar assay.

In some embodiments, the antibodies or antigen-binding fragments thereof block LAG-induced T cell down-regulation with an EC₅₀ less than 9 nM as measured by a T cell/APC luciferase reporter assay as defined in Example 8 of PCT/US16/56156, or a substantially similar assay. In certain embodiments, the antibodies or antigen-binding fragments thereof block LAG3-induced T cell down-regulation with an EC₅₀ less than about 5 nM, less than about 1 nM, less than about 0.5 nM, or less than about 0.1 nM, as measured by a T cell/APC luciferase reporter assay, e.g., using the assay format as defined in Example 8 of PCT/US16/56156, or a substantially similar assay.

In some embodiments, the antibodies or antigen-binding fragments thereof bind to cynomolgus activated CD4+ and CD8+ T cells with an EC₅₀ less than about 1.2 nM as measured by a fluorescence assay as defined in Example 9 of PCT/US16/56156, or a substantially similar assay. In certain embodiments, the antibodies or antigen-binding fragments thereof bind to cynomolgus activated CD4+ and CD8+ T cells with an EC₅₀ less than about 1.1 nM, less than about 1 nM, less than about 0.5 nM, less than about 0.2 nM, or less than about 0.1 nM, as measured by a fluorescence assay, e.g., using the assay format as defined in Example 9 of PCT/US16/56156, or a substantially similar assay.

In one embodiment, the antibody or fragment thereof is a monoclonal antibody or antigen-binding fragment thereof that binds to LAG3, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 194, 210, 226, 242, 258, 274, 290, 306, 322, 338, 354, 370, 386, 402, 418, 434, 450, 458, 466, 474, 482, 490, 498, 506, 514, 538, and 554, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 522, 530, 546, and 562, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 464, 472, 480, 488, 496, 504, 512, 520, 544, and 560, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 192, 208, 224, 240, 256, 272, 288, 304, 320, 336, 352, 368, 384, 400, 416, 432, 448, 528, 536, 552, and 568, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 196, 212, 228, 244, 260, 276, 292, 308, 324, 340, 356, 372, 388, 404, 420, 436, 452, 460, 468, 476, 484, 492, 500, 508, 516, 540, and 556, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118, 134, 150, 166, 182, 198, 214, 230, 246, 262, 278, 294, 310, 326, 342, 358, 374, 390, 406, 422, 438, 454, 462, 470, 478, 486, 494, 502, 510, 518, 542, and 558, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 524, 532, 548, and 564, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 110, 126, 142, 158, 174, 190, 206, 222, 238, 254, 270, 286, 302, 318, 334, 350, 366, 382, 398, 414, 430, 446, 526, 534, 550, and 566, or a substantially similar sequence thereof having at least 90%, at least 95%, at least 98% or at least 99% sequence identity; (v) binds monomeric human LAG3 with a binding dissociation equilibrium constant (K_{D}) of less than about 10 nM as measured in a surface plasmon resonance assay at 25 °C; (vi) binds monomeric human LAG3 with a K_{D} less than about 8 nM as measured in a surface plasmon resonance assay at 37 °C; (vii) binds dimeric human LAG3 with a K_{D} less than about 1.1 nM as measured in a surface plasmon resonance assay at 25 °C; (viii) binds dimeric human LAG3 with a K_{D} less than about 1 nM as measured in a surface plasmon resonance assay at 37 °C; (ix) binds to a hLAG3-expressing cell with an EC₅₀ less than about 8 nM as measured in a flow cytometry assay; (x) binds to a mfLAG3-expressing cell with a EC₅₀ less than about 2.3 nM as measured in a flow cytometry assay; (xi) blocks binding of hLAG3 to human MHC class II with IC₅₀ less than about 32 nM as determined by a cell adherence assay; (xii) blocks binding of hLAG3 to mouse MHC class II with IC₅₀ less than about 30 nM as determined by a cell adherence assay; (xiii) blocks binding of hLAG3 to MHC class II by more than 90% as determined by a cell adherence assay; (xiv) rescues LAG3-mediated inhibition of T cell activity with EC₅₀ less than about 9 nM as determined in a luciferase reporter assay; (xv) binds to activated CD4+ and CD8+ T cells with EC₅₀ less than about 1.2 nM, as determined in a fluorescence assay; and (xvi) suppresses tumor growth and increases survival in a subject with cancer.

In one embodiment, the antibody or fragment thereof is a monoclonal antibody or antigen-binding fragment thereof that blocks LAG3 binding to MHC class II, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 194, 210, 226, 242, 258, 274, 290, 306, 322, 338, 354, 370, 386, 402, 418, 434, 450, 458, 466, 474, 482, 490, 498, 506, 514, 538, and 554, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, 202, 218, 234, 250, 266, 282, 298, 314, 330, 346, 362, 378, 394, 410, 426, 442, 522, 530, 546, and 562, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 232, 248, 264, 280, 296, 312, 328, 344, 360, 376, 392, 408, 424, 440, 456, 464, 472, 480, 488, 496, 504, 512, 520, 544, and 560, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 192, 208, 224, 240, 256, 272, 288, 304, 320, 336, 352, 368, 384, 400, 416, 432, 448, 528, 536, 552, and 568, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 196, 212, 228, 244, 260, 276, 292, 308, 324, 340, 356, 372, 388, 404, 420, 436, 452, 460, 468, 476, 484, 492, 500, 508, 516, 540, and 556, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118, 134, 150, 166, 182, 198, 214, 230, 246, 262, 278, 294, 310, 326, 342, 358, 374, 390, 406, 422, 438, 454, 462, 470, 478, 486, 494, 502, 510, 518, 542, and 558, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, 204, 220, 236, 252, 268, 284, 300, 316, 332, 348, 364, 380, 396, 412, 428, 444, 524, 532, 548, and 564, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 110, 126, 142, 158, 174, 190, 206, 222, 238, 254, 270, 286, 302, 318, 334, 350, 366, 382, 398, 414, 430, 446, 526, 534, 550, and 566, or a substantially similar sequence thereof having at least 90%, at least 95%, at least 98% or at least 99% sequence identity; (v) binds monomeric human LAG3 with a binding dissociation equilibrium constant (K_{D}) of less than about 10 nM as measured in a surface plasmon resonance assay at 25 °C; (vi) binds monomeric human LAG3 with a K_{D} less than about 8 nM as measured in a surface plasmon resonance assay at 37 °C; (vii) binds dimeric human LAG3 with a K_{D} less than about 1.1 nM as measured in a surface plasmon resonance assay at 25 °C; (viii) binds dimeric human LAG3 with a K_{D} less than about 1 nM as measured in a surface plasmon resonance assay at 37 °C; (ix) binds to a hLAG3-expressing cell with an EC₅₀ less than about 8 nM as measured in a flow cytometry assay; (x) binds to a mfLAG3-expressing cell with a EC₅₀ less than about 2.3 nM as measured in a flow cytometry assay; (xi) blocks binding of hLAG3 to human MHC class II with IC₅₀ less than about 32 nM as determined by a cell adherence assay; (xii) blocks binding of hLAG3 to mouse MHC class II with IC₅₀ less than about 30 nM as determined by a cell adherence assay; (xiii) blocks binding of hLAG3 to MHC class II by more than 90% as determined by a cell adherence assay; (xiv) rescues LAG3-mediated inhibition of T cell activity with EC₅₀ less than about 9 nM as determined in a luciferase reporter assay; (xv) binds to activated CD4+ and CD8+ T cells with EC₅₀ less than about 1.2 nM, as determined in a fluorescence assay; and (xvi) suppresses tumor growth and increases survival in a subject with cancer.

In certain embodiments, the antibodies may function by blocking or inhibiting the MHC class II-binding activity associated with LAG3 by binding to any other region or fragment of the full length protein, the amino acid sequence of which is shown in SEQ ID NO: 582.

In certain embodiments, the antibodies are bi-specific antibodies. The bi-specific antibodies can bind one epitope in one domain and can also bind a second epitope in a different domain of LAG3. In certain embodiments, the bi-specific antibodies bind two different epitopes in the same domain. In one embodiment, the multi-specific antigen-binding molecule comprises a first antigen-binding specificity wherein the first binding specificity comprises the extracellular domain or fragment thereof of LAG3; and a second antigen-binding specificity to another epitope of LAG3.

In certain embodiments, the anti-LAG3 antibodies or antigen-binding fragments thereof bind an epitope within any one or more of the regions exemplified in LAG3, either in natural form, as exemplified in SEQ ID NO: 582, or recombinantly produced, as exemplified in SEQ ID NOS: 574-576, or to a fragment thereof. In some embodiments, the antibodies bind to an extracellular region comprising one or more amino acids selected from the group consisting of amino acid residues 29-450 of LAG3. In some embodiments, the antibodies bind to an extracellular region comprising one or more amino acids selected from the group consisting of amino acid residues 1-533 of cynomolgus LAG3, as exemplified by SEQ ID NO: 576.

In certain embodiments, anti-LAG3 antibodies and antigen-binding fragments thereof interact with one or more epitopes found within the extracellular region of LAG3 (SEQ ID NO: 588). The epitope(s) may consist of one or more contiguous sequences of 3 or more (e.g., 3, 4, 5, 6, 7, 8, 9, 10, 11, 12, 13, 14, 15, 16, 17, 18, 19, 20 or more) amino acids located within the extracellular region of LAG3. Alternatively, the epitope may consist of a plurality of non-contiguous amino acids (or amino acid sequences) located within the extracellular region of LAG3. The epitope of LAG3 with which the exemplary antibody H4sH15482P interacts is defined by the amino acid sequence LRRAGVTWQHQPDSGPPAAAPGHPLAPGPHPAAPSSWGPRPRRY (SEQ ID NO: 589), which corresponds to amino acids 28 to 71 of SEQ ID NO: 588. Accordingly, also included are anti-LAG3 antibodies that interact with one or more amino acids contained within the region consisting of amino acids 28 to 71 of SEQ ID NO: 588 (i.e., the sequence LRRAGVTWQHQPDSGPPAAAPGHPLAPGPHPAAPSSWGPRPRRY [SEQ ID NO: 589]).

The present disclosure includes anti-LAG3 antibodies that bind to the same epitope, or a portion of the epitope, as any of the specific exemplary antibodies described herein in Table 1, or an antibody having the CDR sequences of any of the exemplary antibodies described in Table 1. Likewise, also included are anti-LAG3 antibodies that compete for binding to LAG3 or a LAG3 fragment with any of the specific exemplary antibodies described herein in Table 1, or an antibody having the CDR sequences of any of the exemplary antibodies described in Table 1. For example, the present disclosure includes anti-LAG3 antibodies that cross-compete for binding to LAG3 with one or more antibodies provided herein (e.g., H4sH15482P, H4sH15479P, H4sH14813N, H4H14813N, H4H15479P, H4H15482P, H4H15483P, H4sH15498P, H4H15498P, H4H17828P2, H4H17819P, and H4H17823P).

The antibodies and antigen-binding fragments described herein specifically bind to LAG3 and modulate the interaction of LAG3 with MHC class II. The anti-LAG3 antibodies may bind to LAG3 with high affinity or with low affinity. In certain embodiments, the antibodies are blocking antibodies wherein the antibodies bind to LAG3 and block the interaction of LAG3 with MHC class II. In some embodiments, the blocking antibodies of the disclosure block the binding of LAG3 to MHC class II and/or stimulate or enhance T-cell activation. In some embodiments, the blocking antibodies are useful for stimulating or enhancing the immune response and/or for treating a subject suffering from cancer, or a chronic viral infection. The antibodies when administered to a subject in need thereof may reduce the chronic infection by a virus such as human immunodeficiency virus (HIV), hepatitis B virus (HBV), hepatitis C virus (HCV), human papilloma virus (HPV), lymphocytic choriomeningitis virus (LCMV), and simian immunodeficiency virus (SIV) in the subject. They may be used to inhibit the growth of tumor cells in a subject. They may be used alone or as adjunct therapy with other therapeutic moieties or modalities known in the art for treating cancer, or viral infection. In certain embodiments, the anti-LAG3 antibodies that bind to LAG3 with a low affinity are used as multi-specific antigen-binding molecules wherein the first binding specificity binds to LAG3 with a low affinity and the second binding specificity binds to an antigen selected from the group consisting of a different epitope of LAG3 and another T-cell co-inhibitor.

In some embodiments, the antibodies bind to LAG3 and reverse the anergic state of exhausted T cells. In certain embodiments, the antibodies bind to LAG3 and inhibit regulatory T cell activity. In some embodiments, the antibodies may be useful for stimulating or enhancing the immune response and/or for treating a subject suffering from cancer, a viral infection, a bacterial infection, a fungal infection, or a parasitic infection. The antibodies when administered to a subject in need thereof may reduce chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection.

In certain embodiments, the antibodies of the present disclosure are agonist antibodies, wherein the antibodies bind to LAG3 and enhance the interaction of LAG3 and MHC class II. In some embodiments, the activating antibodies enhance binding of LAG3 to MHC class II and/or inhibit or suppress T-cell activation. The activating antibodies of the present disclosure may be useful for inhibiting the immune response in a subject and/or for treating autoimmune disease.

Certain anti-LAG3 antibodies are able to bind to and neutralize the activity of LAG3, as determined by in vitro or in vivo assays. The ability of the antibodies to bind to and neutralize the activity of LAG3 may be measured using any standard method known to those skilled in the art, including binding assays, or activity assays, as described herein.

Non-limiting, exemplary in vitro assays for measuring binding activity are illustrated in Examples provided in PCT/US16/56156: in Example 3, the binding affinities and kinetic constants of human anti-LAG3 antibodies for human LAG3 were determined by surface plasmon resonance and the measurements were conducted on a Biacore 4000 or T200 instrument; in Example 4, blocking assays were used to determine cross-competition between anti-LAG3 antibodies; Examples 5 and 6 describe the binding of the antibodies to cells overexpressing LAG3; in Example 7, binding assays were used to determine the ability of the anti-LAG3 antibodies to block MHC class II-binding ability of LAG3 in vitro; in Example 8, a luciferase assay was used to determine the ability of anti-LAG3 antibodies to antagonize LAG3 signaling in T cells; and in Example 9, a fluorescence assay was used to determine the ability of anti-LAG3 antibodies to bind to activated monkey CD4+ and CD8+ T cells.

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to LAG3. An antibody fragment may include a Fab fragment, a F(ab′)₂ fragment, a Fv fragment, a dAb fragment, a fragment containing a CDR, or an isolated CDR. In certain embodiments, the term “antigen-binding fragment” refers to a polypeptide or fragment thereof of a multi-specific antigen-binding molecule. In such embodiments, the term “antigen-binding fragment” includes, e.g., MHC class II molecule which binds specifically to LAG3. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (ODR) such as a ODR3 peptide), or a constrained FR3-CDR3-FR4 peptide. Other engineered molecules, such as domain-specific antibodies, single domain antibodies, domain-deleted antibodies, chimeric antibodies, ODR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one ODR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers.

Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-OH1; (ii) V_{H}—C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (iX) V_{L}-C_{H}2; (X) V_{L}-C_{H}3; (Xi) V_{L}-C_{H}1-C_{H}2; (xii) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

The anti-LAG3 antibodies and antibody fragments of the present disclosure encompass proteins having amino acid sequences that vary from those of the described antibodies, but that retain the ability to bind LAG3. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

According to certain embodiments of the present disclosure, anti-LAG3 antibodies comprise an Fc domain comprising one or more mutations which enhance or diminish antibody binding to the FcRn receptor, e.g., at acidic pH as compared to neutral pH. For example, the present disclosure includes anti-LAG3 antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present disclosure includes anti-LAG3 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). In one embodiment, the present disclosure includes anti-LAG3 antibodies comprising an Fc domain comprising a S108P mutation in the hinge region of IgG4 to promote dimer stabilization. All possible combinations of the foregoing Fc domain mutations, and other mutations within the antibody variable domains disclosed herein, are contemplated within the scope of the present disclosure.

The present disclosure also includes anti-LAG3 antibodies comprising a chimeric heavy chain constant (C_{H}) region, wherein the chimeric C_{H} region comprises segments derived from the C_{H} regions of more than one immunoglobulin isotype. For example, the antibodies of the disclosure may comprise a chimeric C_{H} region comprising part or all of a C_{H}2 domain derived from a human IgG1, human IgG2 or human IgG4 molecule, combined with part or all of a C_{H}3 domain derived from a human IgG1, human IgG2 or human IgG4 molecule. According to certain embodiments, the antibodies of the disclosure comprise a chimeric C_{H} region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric C_{H} region as described herein may, in certain embodiments, exhibit modified Fc effector functions without adversely affecting the therapeutic or pharmacokinetic properties of the antibody. (See, e.g., US Patent Publication No. 20140243504, the disclosure of which is hereby incorporated by reference in its entirety). In certain embodiments, the Fc region comprises a sequence selected from the group consisting of SEQ ID NOs: 569, 570, 571, 572 and 573.

### B. Positron Emitters and Chelating Moieties

Suitable positron emitters include, but are not limited to, those that form stable complexes with the chelating moiety and have physical half-lives suitable for immuno-PET imaging purposes. Illustrative positron emitters include, but are not limited to, ⁸⁹Zr, ⁶⁸Ga, ⁶⁴Cu, ⁴⁴Sc, and ⁸⁶Y. Suitable positron emitters also include those that directly bond with the LAG3 binding protein, including, but not limited to, ⁷⁶Br and ¹²⁴I, and those that are introduced via prosthetic group, e.g., ¹⁸F.

The chelating moieties described herein are chemical moieties that are covalently linked to the LAG3 binding protein, e.g., anti-LAG3 antibody and comprise a portion capable of chelating a positron emitter, i.e., capable of reacting with a positron emitter to form a coordinated chelate complex. Suitable moieties include those that allow efficient loading of the particular metal and form metal-chelator complexes that are sufficiently stable in vivo for diagnostic uses, e.g., immuno-PET imaging. Illustrative chelating moieties include those that minimize dissociation of the positron emitter and accumulation in mineral bone, plasma proteins, and/or bone marrow depositing to an extent suitable for diagnostic uses.

Examples of chelating moieties include, but are not limited to, those that form stable complexes with positron emitters ⁸⁹Zr, ⁶⁸Ga, ⁶⁴Cu, 44Sc, and/or ⁸⁶Y. Illustrative chelating moieties include, but are not limited to, those described in Nature Protocols, 5(4): 739, 2010; Bioconjugate Chem., 26(12): 2579 (2015); Chem Commun (Camb), 51(12): 2301 (2015); Mol. Pharmaceutics, 12: 2142 (2015); Mol. Imaging Biol., 18: 344 (2015); Eur. J. Nucl. Med. Mol. Imaging, 37:250 (2010); Eur. J. Nucl. Med. Mol. Imaging (2016). doi:10.1007/s00259-016-3499-x; Bioconjugate Chem., 26(12): 2579 (2015); WO 2015/140212A1; and U.S. Pat. No. 5,639,879, incorporated by reference in their entireties.

Illustrative chelating moieties also include, but are not limited to, those that comprise desferrioxamine (DFO), 1,4,7,10-tetraacetic acid (DOTA), diethylenetriaminepentaacetic acid (DTPA), ethylenediaminetetraacetic acid (EDTA), (1,4,7,10-Tetraazacyclododecane-1,4,7,10-tetra(methylene phosphonic) acid (DOTP), 1R, 4R, 7R, 10R)-α′α″α′″-Tetramethyl-1,4,7,10-tetraazacyclododecane-1,4,7,10-tetraacetic acid (DOTMA), 1,4,8,11-Tetraazacyclotetradecane-1,4,8, 11-tetraacetic acid (TETA), H₄octapa, H₆phospa, H₂dedpa, H₅decapa, H₂azapa, HOPO, DO2A, 1,4,7,10-Tetrakis(carbamoylmethyl)-1,4,7,10-tetraazacyclododecane (DOTAM), 1,4,7-triazacyclononane-N,N′,N″-triacetic acid (NOTA), 1,4,7,10-Tetrakis(carbamoylmethyl)-1,4,7,10-tetraazacyclododecane (DOTAM), 1,4,8,11-tetraazabicyclo[6.6.2]hexadecane-4, 11-dicetic acid (CB-TE2A), 1,4,7,10-Tetraazacyclododecane (Cyclen), 1,4,8,11-Tetraazacyclotetradecane (Cyclam), octadentate chelators, e.g., DFO*, which can be conjugated to the antibody via DFO*-pPhe-NCS (See Vugt et al., Eur J Nuci Med Mol Imaging (2017) 44: 286-295), hexadentate chelators, phosphonate-based chelators, macrocyclic chelators, chelators comprising macrocyclic terephthalamide ligands, bifunctional chelators, fusarinine C and fusarinine C derivative chelators, triacetylfusarinine C (TAFC), ferrioxamine E (FOXE), ferrioxamine B (FOXB), ferrichrome A (FCHA), and the like.

In some embodiments, the chelating moieties are covalently bonded to the LAG3 binding protein, e.g., antibody or antigen binding fragment thereof, via a linker moiety, which covalently attaches the chelating portion of the chelating moiety to the binding protein. In some embodiments, these linker moieties are formed from a reaction between a reactive moiety of the LAG3 binding protein, e.g., cysteine or lysine of an antibody, and reactive moiety that is attached to a chelator, including, for example, a p-isothiocyanatobenyl group and the reactive moieties provided in the conjugation methods below. In addition, such linker moieties optionally comprise chemical groups used for purposes of adjusting polarity, solubility, steric interactions, rigidity, and/or the length between the chelating portion and the LAG3 binding protein.

### C. Preparation of Radiolabeled Anti-LAG3 Conjugates

The radiolabeled anti-LAG3 protein conjugates can be prepared by (1) reacting a LAG3 binding protein, e.g., antibody, with a molecule comprising a positron emitter chelator and a moiety reactive to the desirable conjugation site of the LAG3 binding protein and (2) loading the desirable positron emitter.

Suitable conjugation sites include, but are not limited to, lysine and cysteine, both of which can be, for example, native or engineered, and can be, for example, present on the heavy or light chain of an antibody. Cysteine conjugation sites include, but are not limited to, those obtained from mutation, insertion, or reduction of antibody disulfide bonds. Methods for making cysteine engineered antibodies include, but are not limited to, those disclosed in WO2011/056983. Site-specific conjugation methods can also be used to direct the conjugation reaction to specific sites of an antibody, achieve desirable stoichiometry, and/or achieve desirable chelator-to-antibody ratios. Such conjugation methods are known to those of ordinary skill in the art and include, but are not limited to cysteine engineering and enzymatic and chemo-enzymatic methods, including, but not limited to, glutamine conjugation, Q295 conjugation, and transglutaminase-mediated conjugation, as well as those described in J. Clin. Immunol., 36: 100 (2016), incorporated herein by reference in its entirety. Suitable moieties reactive to the desirable conjugation site generally enable efficient and facile coupling of the LAG3 binding protein, e.g., antibody and positron emitter chelator. Moieties reactive to lysine and cysteine sites include electrophilic groups, which are known to those of ordinary skill. In certain aspects, when the desired conjugation site is lysine, the reactive moiety is an isothiocyanate, e.g., p-isothiocyanatobenyl group or reactive ester. In certain aspects, when the desired conjugation site is cysteine, the reactive moiety is a maleimide.

When the chelator is desferrioxamine (DFO), suitable reactive moieties include, but are not limited to, an isothiocyantatobenzyl group, an n-hydroxysuccinimide ester,2,3,5,6 tetrafluorophenol ester, n-succinimidyl-S-acetylthioacetate, and those described in BioMed Research International, Vol 2014, Article ID 203601, incorporated herein by reference in its entirety. In certain embodiments, the LAG3 binding protein is an antibody and the molecule comprising a positron emitter chelator and moiety reactive to the conjugation site is p-isothiocyantatobenzyl-desferrioxamine (p-SCN-Bn-DFO):

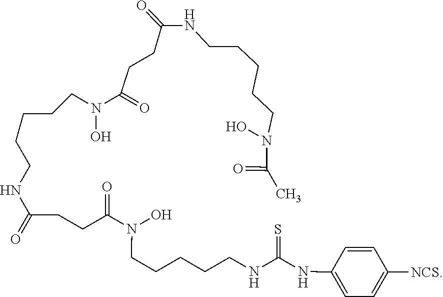

Positron emitter loading is accomplished by incubating the LAG3 binding protein chelator conjugate with the positron emitter for a time sufficient to allow coordination of said positron emitter to the chelator, e.g., by performing the methods described in the examples provided herein, or substantially similar method.

### D. Illustrative Embodiments of Conjugates

Included in the instant disclosure are radiolabeled antibody conjugates comprising an antibody or antigen binding fragment thereof that binds human LAG3 and a positron emitter.

Also included in the instant disclosure are radiolabeled antibody conjugates comprising an antibody or antigen binding fragment thereof that binds human LAG3, a chelating moiety, and a positron emitter.

In some embodiments, the chelating moiety comprises a chelator capable of forming a complex with ⁸⁹Zr. In certain embodiments, the chelating moiety comprises desferrioxamine. In certain embodiments, the chelating moiety is p-isothiocyanatobenzyl-desferrioxamine.

In some embodiments, the positron emitter is ⁸⁹Zr. In some embodiments, less than 1.0% of the anti-LAG3 antibody is conjugated with the positron emitter, less than 0.9% of the anti-LAG3 antibody is conjugated with the positron emitter, less than 0.8% of the anti-LAG3 antibody is conjugated with the positron emitter, less than 0.7% of the anti-LAG3 antibody is conjugated with the positron emitter, less than 0.6% of the anti-LAG3 antibody is conjugated with the positron emitter, less than 0.5% of the anti-LAG3 antibody is conjugated with the positron emitter, less than 0.4% of the anti-LAG3 antibody is conjugated with the positron emitter, less than 0.3% of the anti-LAG3 antibody is conjugated with the positron emitter, less than 0.2% of the anti-LAG3 antibody is conjugated with the positron emitter, or less than 0.1% of the anti-LAG3 antibody is conjugated with the positron emitter.

In some embodiments, the chelating moiety-to-antibody ratio of the conjugate is from 1 to 2.

In a particular embodiment, chelating moiety is p-isothiocyanatobenzyl-desferrioxamine and the positron emitter is ⁸⁹Zr. In another particular embodiment, the chelating moiety is p-isothiocyanatobenzyl-desferrioxamine and the positron emitter is ⁸⁹Zr, and the chelating moiety-to-antibody ratio of the conjugate is from 1 to 2.

In some embodiments, provided herein are antigen-binding proteins that bind LAG3, wherein said antigen-binding proteins that bind LAG3 are covalently bonded to one or more moieties having the following structure:

-L-M_{Z}

wherein L is a chelating moiety; M is a positron emitter; and z, independently at each occurrence, is 0 or 1; and wherein at least one of z is 1. In certain embodiments, the radiolabeled antigen-binding protein is a compound of Formula (I):

M-L-A-[L-M_{Z}]ₖ (I)

A is a protein that binds LAG3; L is a chelating moiety; M is a positron emitter; z is 0 or 1; and k is an integer from 0-30. In some embodiments, k is 1.

In some embodiments, L is:

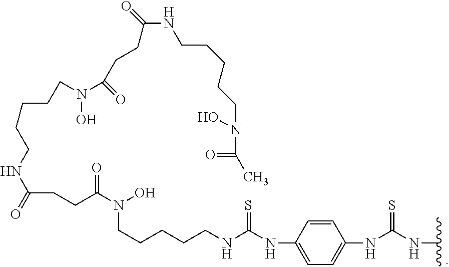 [SMILES: *NC(=S)Nc1ccc(NC(=S)NCCCCCN(O)C(=O)CCC(=O)NCCCCCN(O)C(=O)CCC(=O)NCCCCCN(O)C(C)=O)cc1]

In some embodiments, M is ⁸⁹Zr.

In some embodiments, k is an integer from 1 to 2. In some embodiments, k is 1.

In some embodiments, -L-M is

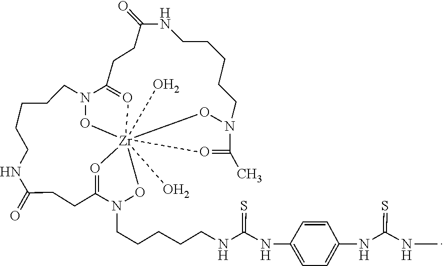

Included in the instant disclosure are also methods of synthesizing a radiolabeled antibody conjugates comprising contacting a compound of Formula (III):

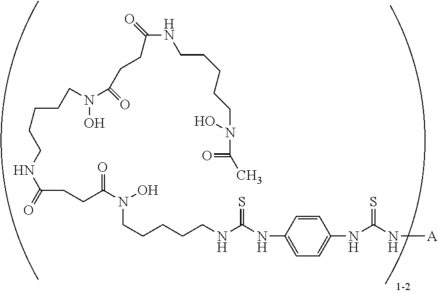

with ⁸⁹Zr, wherein A is an antibody or antigen-binding fragment thereof that binds LAG3. In certain embodiments, the compound of Formula (III) is synthesized by contacting an antibody, or antigen binding fragment thereof, that binds LAG3, with p-SCN-Bn-DFO.

Provided herein is also the product of the reaction between a compound of Formula (III) with ⁸⁹Zr.

Provided herein are compounds of Formula (III):

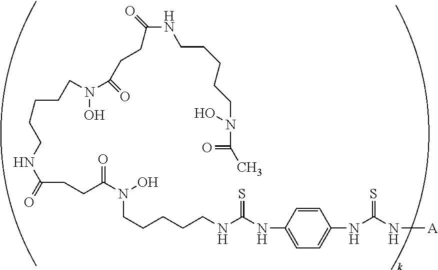

wherein A is an antibody or antigen binding fragment thereof that binds LAG3 and k is an integer from 1-30. In some embodiments, k is 1 or 2.

In some embodiments, provided herein are compositions comprising a conjugate having the following structure:

A-Lₖ

wherein A is a protein that binds LAG3; L is a chelating moiety; and k is an integer from 1-30; wherein the conjugate is chelated with a positron emitter in an amount sufficient to provide a specific activity suitable for clinical PET imaging. In some embodiments, the amount of chelated positron emitter is an amount sufficient to provide a specific activity of about 1 to about 20 mCi per 1-50 mg of the protein that binds LAG3. In some embodiments, the amount of chelated positron emitter is an amount sufficient to provide a specific activity of up to 20 mCi, up to 15 mCi, or up to 10 mCi per 1-50 mg of the protein that binds LAG3, for example, in a range of about 3 to about 20 mCi, about 5 to about 20 mCi, about 1 to about 15 mCi, about 3 to about 15 mCi, about 5 to about 15 mCi, about 1 to about 10 mCi, or about 3 to about 10 mCi.

In some embodiments, the antibody or antigen-binding fragment thereof binds monomeric human LAG3 with a binding dissociation equilibrium constant (K_{D}) of less than about 2 nM as measured in a surface plasmon resonance assay at 37 °C.

In some embodiments, the antibody or antigen-binding fragment thereof binds monomeric human LAG3 with a K_{D} less than about 1.5 nM in a surface plasmon resonance assay at 25 °C.

In some embodiments, the antibody or antigen-binding fragment thereof binds dimeric human LAG3 with a K_{D} of less than about 90 pM as measured in a surface plasmon resonance assay at 37 °C.

In some embodiments, the antibody or antigen-binding fragment thereof that binds dimeric human LAG3 with a K_{D} less than about 20 pM in a surface plasmon resonance assay at 25 °C.

In some embodiments, the antibody or antigen-binding fragment thereof competes for binding to human LAG3 with a reference antibody comprising the complementarity determining regions (CDRs) of a HCVR, wherein the HCVR has an amino acid sequence selected from the group consisting of HCVR sequences listed in Table 1; and the CDRs of a LCVR, wherein the LCVR has an amino acid sequence selected from the group consisting of LCVR sequences listed in Table 1. In some embodiments, the reference antibody or antigen-binding fragment thereof comprises an HCVR/LCVR amino acid sequence pair as set forth in Table 1. In some embodiments, the reference antibody comprises an HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562.

In some embodiments, the antibody or antigen-binding fragment thereof enhances LAG3 binding to MHC class II. In some embodiments, the antibody or antigen binding fragment thereof blocks LAG3 binding to MHC class II. In some embodiments, the antibody or antigen binding fragment thereof do not increase or decrease LAG3 binding to its ligands.

In some embodiments, the antibody or antigen-binding fragment thereof comprises the complementarity determining regions (CDRs) of a HCVR, wherein the HCVR has an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370, 386, 402, 418, 434, 450, 458, 466, 474, 482, 490, 498, 506, 514, 538, and 554; and the CDRs of a LCVR, wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 522, 530, 546, and 562. In certain embodiments, the isolated antibody comprises an HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562. In certain embodiments, the isolated antibody comprises an HCVR/LCVR amino acid sequence pair selected from the group consisting of SEQ ID NOs: 386/394, 418/426, 538/546, 577/578, 579/578, and 580/581.

In some embodiments, the antibody is a human monoclonal antibody or antigen-binding fragment thereof that binds specifically to human LAG3, wherein the antibody or antigen-binding fragment thereof comprises a heavy chain variable region (HCVR) having an amino acid sequence selected from the group consisting of HCVR sequences listed in Table 1.

In some embodiments, the antibody is a human monoclonal antibody or antigen-binding fragment thereof that binds specifically to human LAG3, wherein the antibody or antigen-binding fragment thereof comprises a light chain variable region (LCVR) having an amino acid sequence selected from the group consisting of LCVR sequences listed in Table 1.

In some embodiments, the antibody a human monoclonal antibody or antigen-binding fragment thereof that binds specifically to human LAG3, wherein the antibody or antigen-binding fragment thereof comprises (a) a HCVR having an amino acid sequence selected from the group consisting of HCVR sequences listed in Table 1; and (b) a LCVR having an amino acid sequence selected from the group consisting of LCVR sequences listed in Table 1.

In some embodiments, the antibody or antigen-binding fragment thereof comprises three heavy chain complementarity determining regions (CDRs) (HCDR1, HCDR2 and HCDR3) contained within any one of the heavy chain variable region (HCVR) sequences listed in Table 1; and three light chain CDRs (LCDR1, LCDR2 and LCDR3) contained within any one of the light chain variable region (LCVR) sequences listed in Table 1.

In some embodiments, the antibody or antigen-binding fragment thereof comprises:
(a) a HCDR1 domain having an amino acid sequence selected from the group consisting of SEQ ID NOs: 4, 20, 36, 52, 68, 84, 100, 116, 132, 148, 164, 180, 196, 212, 228, 244, 260, 276, 292, 308, 324, 340, 356, 372, 388, 404, 420, 436, 452, 460, 468, 476, 484, 492, 500, 508, 516, 540, and 556;
(b) a HCDR2 domain having an amino acid sequence selected from the group consisting of SEQ ID NOs: 6, 22, 38, 54, 70, 86, 102, 118, 134, 150, 166, 182, 198, 214, 230, 246, 262, 278, 294, 310, 326, 342, 358, 374, 390, 406, 422, 438, 454, 462, 470, 478, 486, 494, 502, 510, 518, 542, and 558;
(c) a HCDR3 domain having an amino acid sequence selected from the group consisting of SEQ ID NOs: 8, 24, 40, 56, 72, 88, 104, 120, 136, 152, 168, 184, 200, 216, 232, 248, 264, 280, 296, 312, 328, 344, 360, 376, 392, 408, 424, 440, 456, 464, 472, 480, 488, 496, 504, 512, 520, 544, and 560;
(d) a LCDR1 domain having an amino acid sequence selected from the group consisting of SEQ ID NOs: 12, 28, 44, 60, 76, 92, 108, 124, 140, 156, 172, 188, 204, 220, 236, 252, 268, 284, 300, 316, 332, 348, 364, 380, 396, 412, 428, 444, 524, 532, 548, and 564;
(e) a LCDR2 domain having an amino acid sequence selected from the group consisting of SEQ ID NOs: 14, 30, 46, 62, 78, 94, 110, 126, 142, 158, 174, 190, 206, 222, 238, 254, 270, 286, 302, 318, 334, 350, 366, 382, 398, 414, 430, 446, 526, 534, 550, and 566; and
(f) a LCDR3 domain having an amino acid sequence selected from the group consisting of SEQ ID NOs: 16, 32, 48, 64, 80, 96, 112, 128, 144, 160, 176, 192, 208, 224, 240, 256, 272, 288, 304, 320, 336, 352, 368, 384, 400, 416, 432, 448, 528, 536, 552, and 568.

In some embodiments, the antibody or antigen-binding fragment comprises a HCVR/LCVR amino acid sequence pair selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562. In some embodiments, the antibody or antigen-binding fragment comprises a HCVR/LCVR amino acid sequence pair selected from the group consisting of SEQ ID NOs: 386/394, 418/426, and 538/546.

### E. Scaled Manufacturing for Production of Anti-LAG3 Antibody-Chelator Conjugates

Included in the present disclosure are scaled-up manufacturing processes for producing anti-LAG3 antibodies conjugated to a chelator. The anti-LAG3 antibody-chelator conjugates are in a form suitable for radiolabeling.

Good manufacturing processes are adhered to in all aspects of production, including maintaining a sterile environment, practicing aseptic procedures, keeping records of all processes, and documenting product quality, purity, strength, and identity, and any deviations therefrom.

The scaled-up manufacturing process is, in some embodiments, much faster than the manufacturing process for research and development. In some embodiments, the scaled-up manufacturing process can take less than 12 hours, or less than 10 hours, or less than 8 hours, or less than 6 hours, or less than 4 hours, or less than or about 2 hours.

In some embodiments, a first step comprises ultrafiltration and diafiltration (UFDF), using a 30-50 kDa membrane, of the anti-LAG3 antibody to remove excipients, conjugation interfering species, and salts that inhibit the conjugation process. Exemplary membrane polymers include polyethersulfone (PES), cellulose acetate (CA), and regenerated cellulose (RC). In this step, the antibody is buffer exchanged in a low ionic strength and non-interfering buffer solution. The buffer pH can be between about 4.5 to about 6, or about 5 to about 6, or about 5.3 to about 5.7, or about 5.5. Buffer systems contemplated herein include any buffer system lacking a primary amine. Exemplary buffers include acetate, phosphate, or citrate buffers. The buffer provides protein stability during pre-conjugation processing. The process volume can be further reduced to concentrate the antibody, then sterile filtered.

Following the pre-conjugation UFDF, the concentrated and filtered antibody can be transferred into an amine free carbonate buffer system. The carbonate buffer system can have a pH in a range from about 8.5 to about 9.6, or from about 9.0 to about 9.6, or from about 9.2 to about 9.4, or from about 9.4 to about 9.6, or a pH of about 9.4.

A chelator, for example, DFO, in solvent is added to a target concentration into the buffer system containing the antibody, and additional solvent can be added to the solution to a desired percentage. The chelator can be added in molar excess of the antibody, for example, 3.5-5:1 chelator to antibody. The total reaction volume can be up to 5 L.

The reaction temperature and the reaction time are inversely related. For example, if the reaction temperature is higher, the reaction time is lower. If the reaction temperature is lower, the reaction time is higher. Illustratively, at a temperature above about 18 °C, the reaction may take less than 2 hours; at a temperature below 18 °C, the reaction may take more than 2 hours.

The conjugation reaction can be terminated by quenching, for example, by the addition of acetic acid.

In some embodiments, conjugation of the antibody with deferoxamine is performed to produce DFO-mAb conjugates. In some embodiments, conjugation of the antibody with p-SCN-Bn-deferoxamine is performed to produce DFO-mAb conjugates.

Exemplary solvents for the chelator include DMSO and DMA. Subsequent UFDF steps utilize membranes, and the membrane is chosen based on the solvent system used in the conjugation step. For example, DMA dissolves PES membranes, so the two could not be used in the same system.

Carbonate buffers are not preferred for stability of the conjugate during long term storage. Thus, once the antibody-chelator conjugates have been formed, they can be buffer exchanged into a buffer chosen specifically for long term storage and stability. Exemplary buffers include citrate, acetate, phosphate, arginine, and histidine buffers. A further UFDF step can be performed to remove residual salts and to provide a suitable concentration, excipient level, and pH of the conjugated monoclonal antibody. The resulting antibody-chelator conjugates can be sterile filtered and stored for subsequent formulation.

### III. Methods of Using Radiolabeled Immunoconjugates

In certain aspects, the present disclosure provides diagnostic and therapeutic methods of use of the radiolabeled antibody conjugates of the present disclosure.

According to one aspect, the present disclosure provides methods of detecting LAG3 in a tissue, the methods comprising administering a radiolabeled anti-LAG3 antibody conjugate of the provided herein to the tissue; and visualizing the LAG3 expression by positron emission tomography (PET) imaging. In certain embodiments, the tissue comprises cells or cell lines. In certain embodiments, the tissue is present in a subject, wherein the subject is a mammal. In certain embodiments, the subject is a human subject. In certain embodiments, the subject has a disease or disorder selected from the group consisting of cancer, infectious disease and inflammatory disease. In one embodiment, the subject has cancer. In certain embodiments, the infectious disease is a bacterial infection caused by, for example, rickettsial bacteria, bacilli, klebsiella, meningococci and gonococci, proteus, pneumonococci, pseudomonas, streptococci, staphylococci, serratia, Borriella, Bacillus anthricis, Chlamydia, Clostridium, Corynebacterium diphtheriae, Legionella, Mycobacterium leprae, Mycobacterium lepromatosis, Salmonella, Vibrio cholerae, and Yersinia pestis. In certain embodiments, the infectious disease is a viral infection caused by, for example, human immunodeficiency virus (HIV), hepatitis C virus (HCV), hepatitis B virus (HBV), herpes virus (e.g., VZV, HSV-1, HAV-6, HSV-II, CMV, and Epstein Barr virus), human papilloma virus (HPV), lymphocytic choriomeningitis virus (LCMV), and simian immunodeficiency virus (SIV). In certain embodiments, the infectious disease is a parasitic infection caused by, for example, Entamoeba spp., Enterobius vermicularis, Leishmania spp., Toxocara spp., Plasmodium spp., Schistosoma spp., Taenia solium, Toxoplasma gondii, and Trypanosoma cruzi. In certain embodiments, the infectious disease is a fungal infection caused by, for example, Aspergillus (fumigatus, niger, etc.), Blastomyces dermatitidis, Candida (albicans, krusei, glabrata, tropicalis, etc.), Coccidioides immitis, Cryptococcus neoformans, Genus Mucorales (mucor, absidia, rhizopus, etc.), Histoplasma capsulatum, Paracoccidioides brasiliensis, and Sporothrix schenkii.

According to one aspect, the present disclosure provides methods of imaging a tissue that expresses LAG3 comprising administering a radiolabeled anti-LAG3 antibody conjugate of the present disclosure to the tissue; and visualizing the LAG3 expression by positron emission tomography (PET) imaging. In one embodiment, the tissue is comprised in a tumor. In one embodiment, the tissue is comprised in a tumor cell culture or tumor cell line. In one embodiment, the tissue is comprised in a tumor lesion in a subject. In one embodiment, the tissue is intratumoral lymphocytes in a tissue. In one embodiment, the tissue comprises LAG3-expressing cells.

According to one aspect, the present disclosure provides methods for measuring response to a therapy, wherein the response to a therapy is measured by measuring inflammation. The methods, according to this aspect, comprise administering a radiolabeled antibody conjugate provided herein to a subject in need thereof and visualizing the LAG3 expression by positron emission tomography (PET) imaging. In certain embodiments, the inflammation is present in a tumor in the subject. In certain embodiments, an increase in LAG3 expression correlates to increase in inflammation in a tumor. In certain embodiments, the inflammation is present in an infected tissue in the subject. In certain embodiments, an decrease in LAG3 expression correlates to a decrease in inflammation in an infected tissue.

According to one aspect, the present disclosure provides methods for measuring response to a therapy, wherein the response to a therapy is measured by measuring inflammation. The methods, according to this aspect, comprise (i) administering a radiolabeled antibody conjugate provided herein to a subject in need thereof and visualizing the LAG3 expression by positron emission tomography (PET) imaging, and (ii) repeating step (i) one or more times after initiation of therapy. In certain embodiments, the inflammation is present in a tissue in the subject. In certain embodiments, an increase in LAG3 expression correlates to increase in inflammation in the tissue. In certain embodiments, a decrease in LAG3 expression correlates to a decrease in inflammation in the tissue. In certain embodiments, LAG3 expression visualized in step (i) is compared to LAG3 expression visualized in step (ii).

According to one aspect, the present disclosure provides methods for determining if a patient is suitable for anti-tumor therapy comprising an inhibitor of LAG3, the methods comprising selecting a patient with a solid tumor, administering a radiolabeled antibody conjugate of the present disclosure, and localizing the administered radiolabeled antibody conjugate in the tumor by PET imaging wherein presence of the radiolabeled antibody conjugate in the tumor identifies the patient as suitable for anti-tumor therapy comprising an inhibitor of LAG3.

According to one aspect, the present disclosure provides methods for identifying a candidate for anti-tumor therapy comprising an inhibitor of LAG3 and an inhibitor of the PD-1/PD-L1 signaling axis, the methods comprising selecting a patient with a solid tumor, administering a radiolabeled antibody conjugate of the present disclosure, and localizing the administered radiolabeled antibody conjugate in the tumor by PET imaging wherein presence of the radiolabeled antibody conjugate in the tumor identifies the patient as suitable for anti-tumor therapy comprising an inhibitor of LAG3. In some embodiments, the patient is further administered a radiolabeled anti-PD-1 conjugate and the administered radiolabeled anti-PD-1 conjugate is localized in the tumor by PET imaging, wherein presence of the radiolabeled antibody conjugate in the tumor identifies the patient as suitable for anti-tumor therapy comprising an inhibitor of the PD-1/PD-L1 signaling axis.

Provided herein are also methods for predicting response of a patient to an anti-tumor therapy, the methods comprising selecting a patient with a solid tumor; and determining if the tumor is LAG3-positive, wherein if the tumor is LAG3-positive it predicts a positive response of the patient to an anti-tumor therapy. In certain embodiments, the tumor is determined positive by administering a radiolabeled anti-LAG3 antibody conjugate of the present disclosure and localizing the radiolabeled antibody conjugate in the tumor by PET imaging wherein presence of the radiolabeled antibody conjugate in the tumor indicates that the tumor is LAG3-positive.

In some embodiments, the anti-tumor therapy is selected from a PD-1 inhibitor (e.g., REGN2810, BGB-A317, nivolumab, pidilizumab, and pembrolizumab), a PD-L1 inhibitor (e.g., atezolizumab, avelumab, durvalumab, MDX-1105, and REGN3504, as well as those disclosed in Patent Publication No. US 2015-0203580), CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3xCD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, and an antibody-drug conjugate (ADC) (e.g., anti-CD19-DM4 ADC, and anti-DS6-DM4 ADC).

In some embodiments, the anti-tumor therapy is selected from the following: nivolumab, ipilimumab, pembrolizumab, and combinations thereof.

According to one aspect, the present disclosure provides methods for predicting response of a patient to an anti-tumor therapy comprising an inhibitor of LAG3, the methods comprising selecting a patient with a solid tumor, determining if the tumor is LAG3-positive, wherein a positive response of the patient is predicted if the tumor is LAG3-positive. In certain embodiments, the tumor is determined positive by administering a radiolabeled antibody conjugate of the present disclosure and localizing the radiolabeled antibody conjugate in the tumor by PET imaging wherein presence of the radiolabeled antibody conjugate in the tumor indicates that the tumor is LAG3-positive.

According to one aspect, the present disclosure provides methods for predicting response of a patient to an anti-tumor therapy comprising an inhibitor of LAG3 in combination with an inhibitor of the PD-1/PD-L1 signaling axis, the methods comprising selecting a patient with a solid tumor, determining if the tumor is LAG3 positive and PD-1-positive, wherein a positive response of the patient is predicted if the tumor is LAG3 positive and PD-1-positive. In certain embodiments, the tumor is determined LAG3 positive by administering a radiolabeled anti-LAG3 conjugate and localizing the radiolabeled antibody conjugate in the tumor by PET imaging wherein presence of the radiolabeled antibody conjugate in the tumor indicates that the tumor is LAG3-positive. In certain embodiments, the tumor is determined PD-1 positive by further administering a radiolabeled anti-PD-1 conjugate and localizing the radiolabeled anti-PD-1 conjugate in the tumor by PET imaging wherein presence of the radiolabeled antibody conjugate in the tumor indicates that the tumor is PD-1-positive.

According to one aspect, the present disclosure provides methods for detecting a LAG3-positive tumor in a subject. The methods, according to this aspect, comprise selecting a subject with a solid tumor; administering a radiolabeled antibody conjugate of the present disclosure to the subject; and determining localization of the radiolabeled antibody conjugate by PET imaging, wherein presence of the radiolabeled antibody conjugate in a tumor indicates that the tumor is LAG3-positive.

In some aspects, the subject in need thereof is administered a dose of about 20 mg or less, a dose of about 15 mg or less, a dose of about 10 mg or less, for example, a dose of 2 mg, or 5 mg, or 10 mg, of a radiolabeled anti-LAG3 antibody conjugate.

As used herein, the expression “a subject in need thereof” means a human or non-human mammal that exhibits one or more symptoms or indications of cancer, and/or who has been diagnosed with cancer, including a solid tumor and who needs treatment for the same. In many embodiments, the term “subject” may be interchangeably used with the term “patient”.

For example, a human subject may be diagnosed with a primary or a metastatic tumor and/or with one or more symptoms or indications including, but not limited to, unexplained weight loss, general weakness, persistent fatigue, loss of appetite, fever, night sweats, bone pain, shortness of breath, swollen abdomen, chest pain/pressure, enlargement of spleen, and elevation in the level of a cancer-related biomarker (e.g., CA125). The expression includes subjects with primary or established tumors. In specific embodiments, the expression includes human subjects that have and/or need treatment for a solid tumor, e.g., colon cancer, breast cancer, lung cancer, prostate cancer, skin cancer, liver cancer, bone cancer, ovarian cancer, cervical cancer, pancreatic cancer, head and neck cancer, and brain cancer. The term includes subjects with primary or metastatic tumors (advanced malignancies). In certain embodiments, the expression “a subject in need thereof” includes patients with a solid tumor that is resistant to or refractory to or is inadequately controlled by prior therapy (e.g., treatment with an anti-cancer agent). For example, the expression includes subjects who have been treated with one or more lines of prior therapy such as treatment with chemotherapy (e.g., carboplatin or docetaxel). In certain embodiments, the expression “a subject in need thereof” includes patients with a solid tumor which has been treated with one or more lines of prior therapy but which has subsequently relapsed or metastasized. In certain embodiments, the term includes subjects having an inflammatory disease or disorder including, but not limited to, cancer, rheumatoid arthritis, atherosclerosis, periodontitis, hay fever, heart disease, coronary artery disease, infectious disease, bronchitis, dermatitis, meningitis, asthma, tuberculosis, ulcerative colitis, Crohn's disease, inflammatory bowel disease, hepatitis, sinusitis, psoriasis, allergy, fibrosis, lupus, vasiculitis, ankylosing spondylitis, Graves' disease, Celiac disease, fibromyalgia, and transplant rejection.

In certain embodiments, the methods of the present disclosure are used in a subject with a solid tumor. The terms “tumor”, “cancer” and “malignancy” are interchangeably used herein. As used herein, the term “solid tumor” refers to an abnormal mass of tissue that usually does not contain cysts or liquid areas. Solid tumors may be benign (not cancer) or malignant (cancer). In some embodiments, the tumor is metastatic. For the purposes of the present disclosure, the term “solid tumor” means malignant solid tumors. The term includes different types of solid tumors named for the cell types that form them, viz. sarcomas, carcinomas and lymphomas. In certain embodiments, the term “solid tumor” includes cancers including, but not limited to, colorectal cancer, ovarian cancer, prostate cancer, breast cancer, brain cancer, cervical cancer, bladder cancer, anal cancer, uterine cancer, colon cancer, liver cancer, melanoma, metastatic melanoma, pancreatic cancer, lung cancer, endometrial cancer, bone cancer, testicular cancer, skin cancer, kidney cancer, stomach cancer, esophageal cancer, head and neck cancer, salivary gland cancer, and myeloma.

In some embodiments, the methods disclosed herein can be used in a subject with cancer, for example, a subject having blood cancer, brain cancer, renal cell cancer, ovarian cancer, bladder cancer, prostate cancer, breast cancer, hepatic cell carcinoma, bone cancer, colon cancer, non-small-cell lung cancer, squamous cell carcinoma of head and neck, colorectal cancer, mesothelioma, B cell lymphoma, and melanoma. In some aspects, the cancer is metastatic, for example, metastatic melanoma.

According to one aspect, the present disclosure provides methods of treating a tumor in a subject. The methods, according to this aspect, comprise selecting a subject with a solid tumor; determining that the tumor is LAG3-positive; and administering one or more doses of an inhibitor of LAG3. In certain embodiments, the tumor is determined to be LAG3-positive by administering a radiolabeled antibody conjugate of the present disclosure to the subject; and visualizing the radiolabeled antibody conjugate in the tumor by PET imaging, wherein presence of the radiolabeled antibody conjugate in the tumor indicates that the tumor is LAG3-positive.

In a further aspect, the methods of treating comprise administering one or more doses of an inhibitor of LAG3 in combination with a CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3xCD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants or any other therapy care to treat cancer. In certain embodiments, an inhibitor of LAG3 may be used in combination with cancer vaccines including dendritic cell vaccines, oncolytic viruses, tumor cell vaccines, etc. to augment the anti-tumor response. Examples of cancer vaccines that can be used in combination with an inhibitor of LAG3 include MAGE3 vaccine for melanoma and bladder cancer, MUC1 vaccine for breast cancer, EGFRv3 (e.g., Rindopepimut) for brain cancer (including glioblastoma multiforme), or ALVAC-CEA (for CEA+ cancers).

In certain embodiments, an inhibitor of LAG3 may be used in combination with radiation therapy in methods to generate long-term durable anti-tumor responses and/or enhance survival of patients with cancer. In some embodiments, the inhibitor of LAG3, e.g. an anti-LAG3 antibody, may be administered prior to, concomitantly or after administering radiation therapy to a cancer patient. For example, radiation therapy may be administered in one or more doses to tumor lesions followed by administration of one or more doses of anti-LAG3 antibodies. In some embodiments, radiation therapy may be administered locally to a tumor lesion to enhance the local immunogenicity of a patient's tumor (adjuvinating radiation) and/or to kill tumor cells (ablative radiation) followed by systemic administration of an anti-LAG3 antibody. For example, intracranial radiation may be administered to a patient with brain cancer (e.g., glioblastoma multiforme) in combination with systemic administration of an anti-LAG3 antibody. In certain embodiments, the anti-LAG3 antibodies may be administered in combination with radiation therapy and a chemotherapeutic agent (e.g., temozolomide) or a VEGF antagonist (e.g., aflibercept).

In certain embodiments, an inhibitor of LAG3 may be administered in combination with one or more anti-viral drugs to treat viral infection caused by, for example, LCMV, HIV, HPV, HBV or HCV. Examples of anti-viral drugs include, but are not limited to, zidovudine, lamivudine, abacavir, ribavirin, lopinavir, efavirenz, cobicistat, tenofovir, rilpivirine and corticosteroids.

In certain embodiments, an inhibitor of LAG3 may be administered in combination with one or more anti-bacterial drugs to treat bacterial infection caused by, for example, rickettsial bacteria, bacilli, klebsiella, meningococci and gonococci, proteus, pneumonococci, pseudomonas, streptococci, staphylococci, serratia, Borriella, Bacillus anthricis, Chlamydia, Clostridium, Corynebacterium diphtheriae, Legionella, Mycobacterium leprae, Mycobacterium lepromatosis, Salmonella, Vibrio cholerae, and Yersinia pestis. Examples of anti-bacterial drugs include, but are not limited to, penicillins, tetracyclines, cephalosporins, quinolones, lincomycins, macrolides, ketolides, sulfonamides, glycopeptides, aminoglycosides, and carbapenems.

In certain embodiments, an inhibitor of LAG3 may be administered in combination with one or more anti-fungal drugs to treat fungal infection caused by, for example, Aspergillus (fumigatus, niger, etc.), Blastomyces dermatitidis, Candida (albicans, krusei, glabrata, tropicalis, etc.), Coccidioides immitis, Cryptococcus neoformans, Genus Mucorales (mucor, absidia, rhizopus, etc.), Histoplasma capsulatum, Paracoccidioides brasiliensis, and Sporothrix schenkii. Examples of anti-fungal drugs include, but are not limited to, amphotericin B, fluconazole, vorixonazole, posaconazole, itraconazole, voriconazole, anidulafungin, caspofungin, micafungin, and flucytosine.

In certain embodiments, an inhibitor of LAG3 may be administered in combination with one or more anti-parasitic drugs to treat parasitic infection caused by, for example, Entamoeba spp., Enterobius vermicularis, Leishmania spp., Toxocara spp., Plasmodium spp., Schistosoma spp., Taenia solium, Toxoplasma gondii, and Trypanosoma cruzi. Examples of anti-parasitic drugs include, but are not limited to, praziquantel, oxamniquine, metronidazole, tinidazole, nitazoxanide, dehydroemetine or chloroquine, diloxanide furoate, iodoquinoline, chloroquine, paromomycin, pyrantel pamoate, albendazole, nifurtimox, and benznidazole.

The additional therapeutically active agent(s)/component(s) may be administered prior to, concurrent with, or after the administration of the inhibitor of LAG3. For purposes of the present disclosure, such administration regimens are considered the administration of a LAG3 inhibitor “in combination with” a second therapeutically active component.

In some aspects, the methods of treating comprise selecting a subject with a bacterial infection, a viral infection, a fungal infection, or a parasitic infection; determining that an affected tissue in the subject is LAG3-positive; and administering one or more doses of a therapeutic agent appropriate to the infection. In certain embodiments, the affected tissue is determined to be LAG3-positive by administering a radiolabeled anti-LAG3 conjugate of the present disclosure to the subject; and visualizing the radiolabeled antibody conjugate in the subject by PET imaging, wherein presence of the radiolabeled antibody conjugate in a tissue indicates that the tissue is LAG3-positive. In certain embodiments, the steps of administering and visualizing are performed one or more times in order to monitor the effectiveness of the therapeutic agent in treating the infection.

In some aspects, the methods of treating comprise selecting a subject with a solid tumor; determining that the tumor is LAG3-positive and PD-1-positive; and administering one or more doses of an inhibitor of LAG3 and/or one or more doses of an inhibitor of the PD-1/PD-L1 signaling axis (e.g., an anti-PD-1 antibody or an anti-PD-L1 antibody). In certain embodiments, the tumor is determined to be LAG3-positive by administering a radiolabeled anti-LAG3 conjugate of the present disclosure to the subject; and visualizing the radiolabeled antibody conjugate in the tumor by PET imaging, wherein presence of the radiolabeled antibody conjugate in the tumor indicates that the tumor is LAG3-positive. In certain embodiments, the tumor is determined to be PD-1-positive by administering a radiolabeled anti-PD-1 conjugate of the present disclosure to the subject; and visualizing the radiolabeled anti-PD-1 conjugate in the tumor by PET imaging, wherein presence of the radiolabeled anti-PD-1 conjugate in the tumor indicates that the tumor is PD-1-positive.

Exemplary anti-PD-1 antibodies include REGN2810, BGB-A317, nivolumab, pidilizumab, and pembrolizumab.

Exemplary anti-PD-L1 antibodies include atezolizumab, avelumab, durvalumab, MDX-1105, and REGN3504, as well as those disclosed in Patent Publication No. US 2015-0203580.

The inhibitor of the PD-1/PD-L1 signaling axis may be administered prior to, concurrent with, or after the administration of the inhibitor of LAG3. For purposes of the present disclosure, such administration regimens are considered the administration of a LAG3 inhibitor “in combination with” an inhibitor of the PD-1/PD-L1 signaling axis.

As used herein, the terms “treat”, “treating”, or the like, mean to alleviate symptoms, eliminate the causation of symptoms either on a temporary or permanent basis, to delay or inhibit tumor growth, to reduce tumor cell load or tumor burden, to promote tumor regression, to cause tumor shrinkage, necrosis and/or disappearance, to prevent tumor recurrence, to prevent or inhibit metastasis, to inhibit metastatic tumor growth, and/or to increase duration of survival of the subject.

According to one aspect, the present disclosure provides methods for monitoring the efficacy of an anti-tumor therapy in a subject, wherein the methods comprise selecting a subject with a solid tumor wherein the subject is being treated with an anti-tumor therapy; administering a radiolabeled anti-LAG3 conjugate of the present disclosure to the subject; imaging the localization of the administered radiolabeled conjugate in the tumor by PET imaging; and determining tumor growth, wherein a decrease from the baseline in radiolabeled signal indicates efficacy of the anti-tumor therapy. In certain embodiments, the anti-tumor therapy comprises an inhibitor of LAG3. In certain embodiments, the anti-tumor therapy further comprises an inhibitor of the PD-1/PD-L1 signaling axis (e.g., an anti-PD-1 antibody or an anti-PD-L1 antibody).

In certain embodiments, the present disclosure provides methods to assess changes in the inflammatory state of a tumor, the methods comprising selecting a subject with a solid tumor wherein the subject is being treated with an anti-tumor therapy; administering a radiolabeled anti-LAG3 conjugate provided herein to the subject; and imaging the localization of the administered radiolabeled conjugate in the tumor by PET imaging, wherein an increase from the baseline in radiolabeled signal indicates increase in inflammation and efficacy of the anti-tumor therapy. In certain embodiments, the anti-tumor therapy comprises an inhibitor of LAG3 and/or an inhibitor of the PD-1/PD-L1 signaling axis (e.g., an anti-PD-1 antibody or an anti-PD-L1 antibody). In certain embodiments, the anti-tumor therapy comprises a PD-1 inhibitor (e.g., REGN2810, BGB-A317, nivolumab, pidilizumab, and pembrolizumab), a PD-L1 inhibitor (e.g., atezolizumab, avelumab, durvalumab, MDX-1105, and REGN3504), CTLA-4 inhibitor (e.g., ipilimumab), a TIM3 inhibitor, a BTLA inhibitor, a TIGIT inhibitor, a CD47 inhibitor, a GITR inhibitor, an antagonist of another T cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), a CD20 inhibitor (e.g., an anti-CD20 antibody such as rituximab), an antibody to a tumor-specific antigen [e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9], a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3xCD20 bispecific antibody, or PSMAxCD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, and an antibody-drug conjugate (ADC) (e.g., anti-CD19-DM4 ADC, and anti-DS6-DM4 ADC).

As used herein, the term “baseline,” with respect to LAG3 expression in the tumor, means the numerical value of uptake of the radiolabeled conjugate for a subject prior to or at the time of administration of a dose of anti-tumor therapy. The uptake of the radiolabeled conjugate is determined using methods known in the art (see, for example, Oosting et al 2015, J. Nucl. Med. 56: 63-69). In certain embodiments, the anti-tumor therapy comprises an inhibitor of LAG3.

In some embodiments, sequential iPET scanning and tumor biopsies are performed before and after treatment with standard of care immunotherapies. Such immunotherapies can be selected from the following: nivolumab, ipilimumab, pembrolizumab, and combinations thereof.

To determine whether there is efficacy in anti-tumor therapy, the uptake of the radiolabeled conjugate is quantified at baseline and at one or more time points after administration of the LAG3 inhibitor. For example, the uptake of the administered radiolabeled antibody conjugate (e.g., radiolabeled anti-LAG3 antibody conjugate) may be measured at day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the LAG3 inhibitor (e.g., an anti-LAG3 antibody). The difference between the value of the uptake at a particular time point following initiation of treatment and the value of the uptake at baseline is used to establish whether anti-tumor therapy is efficacious (tumor regression or progression).

In certain embodiments, the radiolabeled antibody conjugate is administered intravenously or subcutaneously to the subject. In certain embodiments, the radiolabeled antibody conjugate is administered intra-tumorally. Upon administration, the radiolabeled antibody conjugate is localized in the tumor. The localized radiolabeled antibody conjugate is imaged by PET imaging and the uptake of the radiolabeled antibody conjugate by the tumor is measured by methods known in the art. In certain embodiments, the imaging is carried out 1, 2, 3, 4, 5, 6 or 7 days after administration of the radiolabeled conjugate. In certain embodiments, the imaging is carried out on the same day upon administration of the radiolabeled antibody conjugate.

In certain embodiments, the antibody or antigen-binding fragment thereof that binds specifically to LAG3. In certain embodiments, the anti-LAG3 antibody comprises the CDRs of a HCVR, wherein the HCVR has an amino acid sequence selected from the group consisting of SEQ ID NOs: 2, 18, 34, 50, 66, 82, 98, 114, 130, 146, 162, 178, 194, 210, 226, 242, 258, 274, 290, 306, 322, 338, 354, 370, 386, 402, 418, 434, 450, 458, 466, 474, 482, 490, 498, 506, 514, 538, and 554; and the CDRs of a LCVR, wherein the LCVR has an amino acid sequence selected from the group consisting of SEQ ID NOs: 10, 26, 42, 58, 74, 90, 106, 122, 138, 154, 170, 186, 202, 218, 234, 250, 266, 282, 298, 314, 330, 346, 362, 378, 394, 410, 426, 442, 522, 530, 546, and 562.

In certain embodiments, the LAG3 inhibitor comprises an antibody or antigen-binding fragment thereof that binds specifically to LAG3. In certain embodiments, the anti-LAG3 antibody is BMS986016. In certain other embodiments, the LAG3 inhibitor comprises an antibody or antigen-binding fragment thereof that binds specifically to LAG3. In one embodiment, the anti-LAG3 antibody comprises an HCVR of SEQ ID NO: 418 and a LCVR of SEQ ID NO: 426.

### IV. EXAMPLES

Certain embodiments of the disclosure are illustrated by the following non-limiting examples.

### Example 1: Generation of Human Antibodies to LAG3

Human antibodies to LAG3 were generated using a fragment of LAG3 that ranges from about amino acids 29-450 of GenBank Accession NP_002277.4 (SEQ ID NO: 582) genetically fused to a mouse Fc region. The immunogen was administered directly, with an adjuvant to stimulate the immune response, to a VELOCIMMUNE® mouse (i.e., an engineered mouse comprising DNA encoding human Immunoglobulin heavy and kappa light chain variable regions), as described in U.S. Pat. No. 8,502,018 B2, or to a humanized Universal Light Chain (ULC) Velocimmune® mouse, as described in WO 2013022782. The antibody immune response was monitored by a LAG3-specific immunoassay. When a desired immune response was achieved splenocytes were harvested and fused with mouse myeloma cells to preserve their viability and form hybridoma cell lines. The hybridoma cell lines were screened and selected to identify cell lines that produce LAG3-specific antibodies. Using this technique, and the immunogen described above, several anti-LAG3 chimeric antibodies (i.e., antibodies possessing human variable domains and mouse constant domains) were obtained. Fully human versions of the antibodies can be made by replacing the mouse constant region with a human constant region. Exemplary antibodies generated in this manner from the VELOCIMMUNE® mice were designated as H1M14985N, H1M14987N, H2M14811N, H2M14885N, H2M14926N, H2M14927N, H2M14931N, H2M18336N, H2M18337N and H4H14813N.

Anti-LAG3 antibodies were also isolated directly from antigen-positive B cells (from either of the immunized mice) without fusion to myeloma cells, as described in U.S. Pat. No. 7,582,298, herein specifically incorporated by reference in its entirety. Using this method, several anti-LAG3 antibodies (i.e., antibodies possessing human variable domains and human constant domains) were obtained; exemplary antibodies generated in this manner were designated as follows: H4H15477P, H4H15483P, H4H15484P, H4H15491P, H4H17823P, H4H17826P2, H4H17828P2, H4sH15460P, H4sH15462P, H4sH15463P, H4sH15464P, H4sH15466P, H4sH15467P, H4sH15470P, H4sH15475P, H4sH15479P, H4sH15480P, H4sH15482P, H4sH15488P, H4sH15496P2, H4sH15498P2, H4sH15505P2, H4sH15518P2, H4sH15523P2, H4sH15530P2, H4sH15555P2, H4sH15558P2, H4sH15567P2, and H4H17819P.

Exemplary antibodies H4sH15496P2, H4sH15498P2, H4sH15505P2, H4sH15518P2, H4sH15523P2, H4sH15530P2, H4sH15555P2, H4sH15558P2, and H4sH15567P2 were generated from B-cells from the ULC VELOCIMMUNE® mice.

The biological properties of the exemplary antibodies generated in accordance with the methods of this Example are described in detail in the Examples set forth below.

### Example 2: Conjugation of anti-LAG3 antibody H4sH15482P with p-SCN-Bn-DFO

In order to modify the parental anti-LAG3 antibody, H4sH15482P (having an HCVR/LCVR sequence pair of SEQ ID NOs: 418/426; hereinafter referred to as mAb1), and an isotype control antibody to be suitable for ImmunoPET studies with radiolabeling, a chelator, p-SCN-bn-Deferoxamine (DFO; Macrocylics, Cat #: B-705), was attached to the antibodies.

For the modification, mAb1, was first buffer exchanged into PBS, pH 7.2 from histidine buffer by dialysis at 4 °C overnight (Slide-A-Lyzer Dialysis Cassette G2 10k MWCO; ThermoScientific) then buffer exchanged again using a PD-10 column (GE Healthcare, Cat. #: 17-0851-01) into a buffer composed of 50 mM carbonate buffer, 150 mM NaCl, pH 9.0 (conjugation buffer). To determine the concentration following the buffer exchanges, the samples were measured on a Nanodrop 2000 UV/VIS spectrometer (Thermo Scientific) using the MacVector sequence based extinction coefficient of 223400 M⁻¹ cm⁻¹ and molecular weight 145709 g/mol (see Table 2). In 15 a mL polypropylene tube, 1485.24 uL of mAb1 (70 mg) was added to 5374.8 uL of conjugation buffer. A 139 μL solution of DFO in DMSO was added in one-quarter increments to the mAb1 solution, each time gently being mixed by pipetting up-and-down. The final solution was 10 mg/mL mAb1 in conjugation buffer, 2% DMSO with 3-fold mole-to-mole excess of DFO. This solution was allowed to incubate in a 37 °C water bath with no additional stirring.

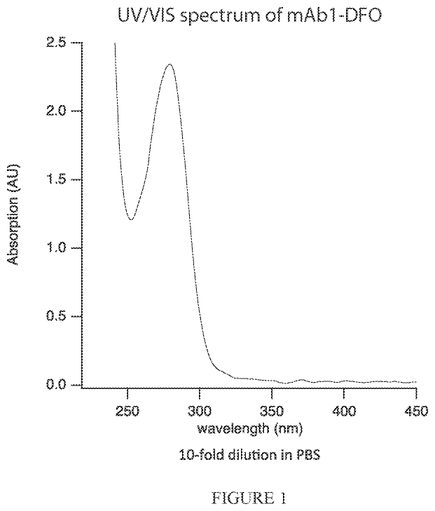

After 30 minutes at 37 °C, the solution was promptly passed through a PD-10 desalting column (GE Healthcare, Cat. #: 17-0851-01), pre-equilibrated with a buffer containing 250 mM NaAcO at pH 5.4 (formulation buffer). The volume of the solution was reduced by approximately 50% with a 10K MWCO concentrator (Amicon Ultra-15 Centrifugal Filter Unit, EMD Millipore, Cat #: UFC901024). The final solution was sterile-filtered via a syringe filter (Acrodisc 13 mm syringe filter, Pall Corporation, Cat #: 4602). The concentration and DFO-to-Antibody Ratio (DAR) was subsequently measured by UV/VIS spectroscopy. See FIG. 1. For the absorbance measurement, the DFO-conjugated antibody was measured against the formulation buffer at 252 nm (A252), 280 nm (A280) and 600 nm (A600). For the calculation, the background was corrected at each absorbance value using the equation:

$${A}_{λ}^{′} = {A}_{λ} - {A}_{600}$$

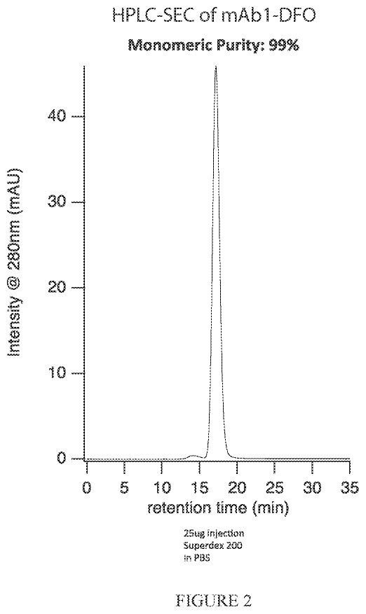

The antibody conjugate was tested for aggregation using SEC chromatography, with 25 μg of the sample injected onto a Superdex 200 column (GE Healthcare, Cat. No. 17-5175-01) monitored at 280 nm with a PBS mobile phase (0.75 mL/min). See FIG. 2. The antibody integrity was evaluated by SDS-PAGE 4-20% Tris/Gly pre-cast gel (Novex) with 2 μg of the sample loaded. The antibody concentration, conjugate concentration, and DAR were calculated using the equations below:

### Antibody Concentration Calculation

$$Conc mAb ( mg / mL ) = \frac{{A}_{280}^{′}}{{ϵ}_{280}} * MW$$

### Conjugate Concentration Calculation

$$Conc conjugate ( mg / mL ) = \frac{{A}_{252}^{′} - 1.53 {A}_{280}^{′}}{{ϵ}_{252} - 1.53 {ϵ}_{280}} * MW$$

### DAR Calculation

$$DAR = \frac{{ϵ}_{252} {A}_{280}^{′} - {ϵ}_{280} {A}_{252}^{′}}{18800 {A}_{252}^{′} - 28700 {A}_{280}^{′}}$$

TABLE 2
Molar extinction coefficients and molecular weight

| mAb | MW (gmo1⁻¹) | ε₂₈₀ (M⁻¹cm⁻¹) | ε₂₅₂ (M⁻¹cm⁻¹) |
|---|---|---|---|
| mAb1 | 145709 | 223400 | 87077 |

TABLE 3
UV DAR, percent aggregate and
concentration post DFO-attachment

| | | Concentration | |
|---|---|---|---|
| Antibody | UV DAR | (mg/mL) | % aggregate |
| mAb1 | 1.48 | 13.58 | 1.4% |

### Example 3: ⁸⁹Zr Chelation of DFO Conjugated Monoclonal Antibodies

For usage in ImmunoPET in vivo studies, the DFO-conjugated anti-LAG3 antibody, mAb1, and a DFO-conjugated isotype control antibody were radiolabeled with ⁸⁹Zr.

DFO-conjugated antibody was first brought to 1.25 mg/mL in 1 M HEPES, pH 7.2. The composition of the DFO-Ab conjugate solutions for each study is listed in Table 4. Separately, ⁸⁹Zr solution was prepared using the compositions for each corresponding study shown in Table 5. Stock ⁸⁹Zr-oxalic acid solution was obtained from 3D Imaging. The final radioactivity of the solution was first confirmed using a Capintec CRC-25R dose calibrator (Capintec #520), then immediately combined with the DFO-Ab conjugate solution, gently mixed (pipetting up-and-down) and subsequently incubated for 45 minutes at room temperature.

After the incubation, the mixtures were transferred to desalting columns, either PD-10 (GE Healthcare, Cat. #: 17-0851-01) for study 1 or NAP-5 (GE Healthcare, Cat. #17-0853-02) for study 2, pre-equilibrated with 250 mM sodium acetate at pH 5.4 for gravity-fed desalting. For study 1, the reaction mixture was added to a PD-10 column. After the contents of the reaction entered the column bed, the flow through was discarded. The product was eluted with 250 mM sodium acetate at pH 5.4 (formulation buffer) and eluate was collected as per manufacturer's instructions. For study 2, the mixture was transferred to a NAP-5 column, and the flow through was discarded. The product was eluted with 250 mM sodium acetate at pH 5.4 (formulation buffer) and eluate was collected per the manufacturer's instructions. The Ab concentration was subsequently measured by UV/VIS spectroscopy, calculated using the appropriate extinction coefficient and the absorption at 280 nm using the equation:

Concentration in mg/mL=Absorption at 280 nm+Extinction coefficient at 280 nm (found in Table 6)

The final mass measured in grams was recorded in Table 7. The radioactivity was then measured using the dose calibrator and reported in Table 7. The final material (5 ug) was analyzed using a SEC-HPLC with UV 280 and radioisotope detector connected in series (Agilent 1260 with Lablogic Radio-TLC/HPLC Detector, SCAN-RAM) using a Superdex 200 Increase column with PBS mobile phase at a flow rate of 0.75 mL/min. The radiotrace was used for determining radiochemical purity (100%−percent of unlabeled ⁸⁹Zr) by comparing the integration of the total protein peak (~10 to 16 min) and unlabeled ⁸⁹Zr peak (~25 min). The percent monomeric purity was determined by the UV 280 trace by comparing the integration of the high molecular weight (HMW) species peak (10 min to ~15 min) to the monomer (~16 min).

The specific activity and protein recovery (%) of each radiolabeled conjugate was determined using the following equations:

Mass of conjugate in mg=concentration in mg/mL×mass of solution in grams a.

Specific activity in mCi/mg=activity of vial in mCi÷mass of conjugate in mg b.

Protein recovery=starting conjugate mass (mg)÷Mass of conjugate in mg c.

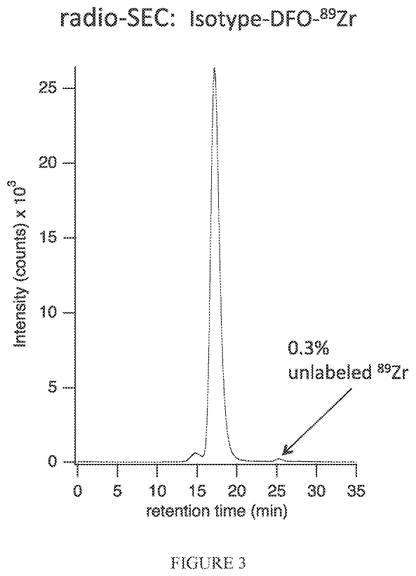

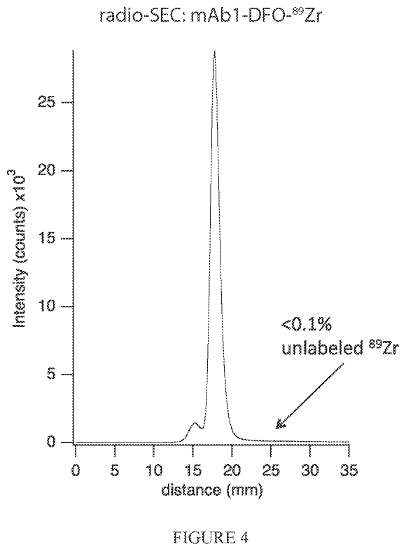

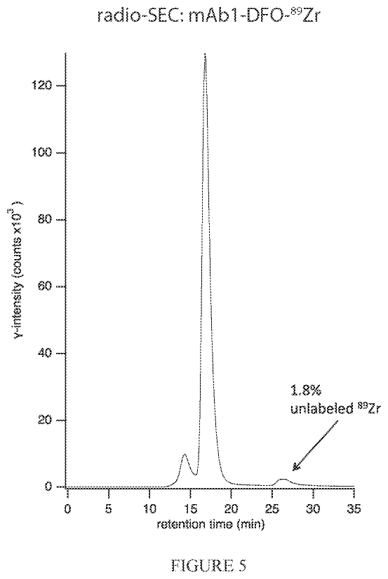

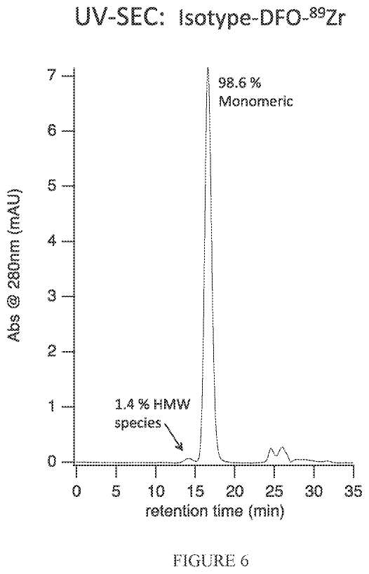

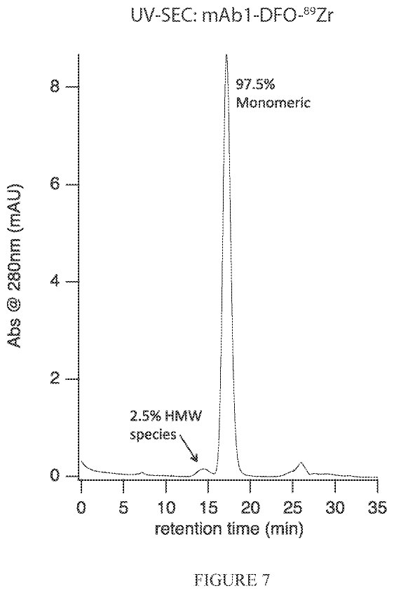

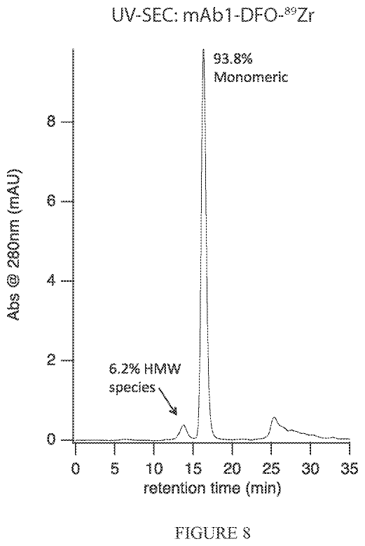

Finally the appearance was noted and recorded in Table 7. The results are consolidated in Table 7. The radio-SEC-HPLC chromatograms, shown in FIGS. 3-5, confirm at least 98% radiochemical purity. The UV280-HPLC SEC chromatograms shown in FIGS. 6-8 confirm the highly monomeric product (>90%).

TABLE 4
DFO-antibody conjugate preparation for radiolabeling

| Radio- | | Radio- | Concen- | | Conjugate | Total | Final |
|---|---|---|---|---|---|---|---|
| labeling | Study | labeling | tration | | mass | volume | Concentration |
| # | # | Lots | (mg/mL) | DAR* | (mg) | (uL) | (mg/mL) |
| 1 | 1 | Isotype-DFO-⁸⁹Zr | 15.4 | 1.53 | 250 | 200 | 1.25 |
| 2 | 1 | mAb1-DFO-⁸⁹Zr | 13.6 | 1.48 | 500 | 400 | 1.25 |
| 3 | 2 | mAb1-DFO-⁸⁹Zr | 13.6 | 1.48 | 100 | 80 | 1.25 |

*DAR is defined as the DFO to Antibody Ratio

TABLE 5
⁸⁹Zr reaction solution preparation for radiolabeling

| | | | | 1M | Final | Final | Specific |
|---|---|---|---|---|---|---|---|
| Radio- | Study | Radio-labeling | ⁸⁹Zr-oxalate | HEPES, pH | Vol | Activity | Activity |
| labeling | # | Lots | (uL) | 7.2 (uL) | (uL) | (uCi) | (uCi/uL) |
| 1 | 1 | Isotype- | ~3 | 500 | 1000 | 995 | 1.0 |
| | | DFO-⁸⁹Zr | | | | | |
| 2 | 1 | mAb1- | ~5 | 500 | 2000 | 2060 | 1.0 |
| | | DFO-⁸⁹Zr | | | | | |
| 3 | 2 | mAb1- | ~6 | 394 | 400 | 2010 | 5.0 |
| | | DFO-⁸⁹Zr | | | | | |

TABLE 6
Extinction coefficients for conjugate lots

| Radiolabeling Lot | ε₂₈₀ (AU ml mg⁻¹ cm⁻¹) |
|---|---|
| Isotype-DFO-⁸⁹Zr | 1.70 |
| mAb1-DFO-⁸⁹Zr | 1.72 |

TABLE 7
Summary of ⁸⁹Zr labeled DFO-Ab conjugates for in vivo imaging and
biodistribution studies

| | | | | Radio- | Mono- | | | Specific |
|---|---|---|---|---|---|---|---|---|
| Radio- | | | | chemical | meric | Protein | Conc. | Activity |
| label- | Study | Conjugate | Appear- | Purity* | Purity** | Recovery | (mg/ | (mCi/ |
| ing | # | Lots | ance | (%) | (%) | (%) | mL) | mg) |
| 1 | 1 | Isotype- | Clear | 99.7% | 98.6% | 70% | 0.108 | 3.41 |
| | | DFO-⁸⁹Zr | | | | | | |
| 2 | 1 | mAb1- | Clear | >99.9% | 97.5% | 70% | 0.133 | 3.58 |
| | | DFO-⁸⁹Zr | | | | | | |
| 3 | 2 | mAb1- | Clear | 98.2% | 93.8% | 57% | 0.121 | 14.7 |
| | | DFO-⁸⁹Zr | | | | | | |

*by radio-SEC-HPLC,
**by UV-SEC-HPLC

### Example 4: Immunoreactivity

The immunoreactivity (IR) of the radiolabeled anti-LAG3 antibody and isotype control antibody was measured as follows. In these assays, 20 ng of the respective ⁸⁹Zr labeled antibodies were added to 15×10⁶ MC38-cOVA/eGFP-mLAG3^{-/-}hLAG3Tg cells in a final volume of 1 mL. Samples were incubated for 45 minutes (at 37 °C, 5% CO₂) with continuous mixing before undergoing 2 washes with media to remove any unbound antibody. The radioactivity of the test cell pellets was then counted in an automatic gamma counter (2470 Wizard2, Perkin Elmer) against 2 reference standards containing the same 20 ng of ⁸⁹Zr labeled antibody. The percentage immunoreactivity was determined for the samples using the average of the standards as a measure of total activity.

As seen in Table 8, ⁸⁹Zr labeled anti-LAG3 antibody retained immunoreactivity following conjugation and radiolabeling, with 86% IR.

TABLE 8
Immunoreactivity of ⁸⁹Zr
chelated DFO-conjugates

| Samples | Zr89 CPM |
|---|---|
| Standard 1 | 39643 |
| Standard 2 | 40134 |
| Average of Standards | 39889 |
| Cells | 34261 |
| IR | 86% |

### Example 5: Selective Localization of Radiolabeled Anti-LAG3 Antibody to LAG3 Positive Tumors in Mice

### Implantation of Tumors and Allocation of Dosing Groups:

For in vivo imaging studies, a LAG3 positive tumor line was used. First, a murine colon carcinoma cell-line MC38-cOVA/eGFP-mLAG3^{-/-}hLAG3Tg was used. Here, cells over-express human LAG3 and full-length chicken ovalbumin fused with eGFP that was introduced by lentiviral transduction (pLVX EF1a and pLKO SSFV, respectively). For MC38-cOVA/eGFP-mLAG3^{-/-}hLAG3^{Tg} tumor allografts, 1×10⁶ cells were implanted subcutaneously into the left flank of male NCr nude (Taconic, Hudson NY). Once tumors had reached an average volume of 100-150 mm³ (~Day 7 post implantation), mice were randomized into groups of 5 and dosed with test or control ⁸⁹Zr radiolabeled antibodies.

### Dosing and Biodistribution of ⁸⁹Zr-DFO-mAb1:

For the initial study in nude mice bearing MC38/ova/LAG3 tumors, mice received 50±1 μCi of ⁸⁹Zr labeled antibody with a protein dose ~0.6 mg/kg. For the biodistribution studies, mice were euthanized 6 days post-dosing and blood was collected via cardiac puncture. Tumors and normal tissues were then excised and placed in counting tubes. Count data for ⁸⁹Zr in CPM was then collected by measuring samples on an automatic gamma counter (Wizard 2470, Perkin Elmer). All tissues were also weighed and the percent-injected dose per gram (% ID/g) was calculated for each sample using standards prepared from the injected material.

### Results, Summary, and Conclusion:

In this example, the NCr mice bearing MC38/ova/hLAG3 tumors received ⁸⁹Zr conjugated anti-LAG3 mAb1 or non-binding antibody at a final dose of 50ρCi/mouse. Mice were subsequently left for 6 days until blood, tumor and tissues were taken and the % ID/g for the samples was calculated for all samples. The average % ID/g for each antibody is presented in Table 9. From this, the clear high uptake in MC38/ova/hLAG3 tumors is apparent over other normal tissues, with tumor uptake of 43.1% being significantly higher than the next highest uptake of 6.6% ID/g observed in the thymus. The specificity of anti-LAG3 mAb1 uptake into tumor is apparent in the significantly reduced tumor uptake of 7.8% observed for the non-binding antibody.

TABLE 9

| | ⁸⁹Zr- mAb1 | | ⁸⁹Zr-non-binding Ab | |
|---|---|---|---|---|
| | AVERAGE | STDEV | AVERAGE | STDEV |
| SAMPLE | % ID/G | % ID/G | % ID/G | % ID/G |
| LIVER | 0.5 | 6.2 | 3.9 | 0.3 |
| SPLEEN | 4.2 | 0.8 | 6.7 | 0.8 |
| KIDNEY | 5.1 | 0.8 | 6.2 | 1.2 |
| BONE | 4.3 | 2.1 | 4.9 | 1.0 |
| LUNG | 3.1 | 2.3 | 9.3 | 2.1 |
| HEART | 2.6 | 0.9 | 6.5 | 2.4 |
| BLOOD | 5.9 | 3.1 | 15.7 | 2.6 |
| THYMUS | 6.7 | 1.7 | 12.1 | 1.8 |
| MC38/ova/LAG3 | 43.1 | 9.5 | 7.8 | 0.4 |
| S. BOWEL | 1.7 | 0.5 | 2.8 | 0.5 |

### Example 6: Selective Localization of Radiolabeled Anti-LAG3 Antibody to Raji/PBMC Tumors in Mice

This Example describes the in vivo imaging and ex vivo biodistribution of a Zirconium-89 labeled DFO-anti-LAG3 antibody conjugate in NSG mice co-implanted with Raji cells and human PBMC.

The exemplary antibody used in this Example was MAb1, comprising HCVR/LCVR of SEQ ID NOs: 418/426.

### Implantation of Tumors and Allocation of Dosing Groups:

To demonstrate specificity of the radiolabeled antibody for LAG3 targeting, 2×10⁶ Raji cells and 5×10⁵ human PBMC (Lot 0151029, ReachBio Research Labs) were co-implanted into the right flank of female NSG mice (8-10 weeks old, Jackson Labs). 14 days post-tumor implantation, mice were randomized into groups of 4 and injected intravenously with varying protein doses of ⁸⁹Zr-DFO-mAb1.

### Dosing and PET/CT Imaging of ⁸⁹Zr-DFO-mAb1:

Mice bearing Raji/hPBMC tumors were injected with 5, 0.3, 0.1, or 0.03 mg/kg ⁸⁹Zr-DFO-mAb1 at day 14 post-tumor implantation. Mice who received 0.1 and 0.03 mg/kg doses received ~30 or ~9 μCi of radiolabeled ⁸⁹Zr-DFO-mAb1, respectively. The mice who received 5 or 0.3 mg/kg protein doses received ~30 μCi of radiolabeled ⁸⁹Zr-DFO-mAb1 and additional non-DFO conjugated mAb1 (L5) as supplement to yield the final injected total protein dose.

PET imaging of antibody localization was assessed 6 days after administration of ⁸⁹Zr-DFO-mAb1. A Sofie Biosciences G8 PET/CT was used to acquire PET/CT images (Sofie Biosciences and Perkin Elmer). The instrument was pre-calibrated for detection of ⁸⁹Zr prior to image acquisition. The energy window ranged from 150 to 650 keV with a reconstructed resolution of 1.4 mm at the center of the field of view. Mice underwent induction anesthesia using isoflurane and were kept under continuous flow of isoflurane during imaging. Static 10-minute images were acquired using the G8 acquisition software and subsequently reconstructed using the pre-configured settings. Image data was corrected for decay and other parameters. CT images were acquired following PET acquisition and subsequently co-registered with the PET images. Images were prepared using VivoQuant post-processing software (inviCRO Imaging Services).

### Biodistribution of ⁸⁹Zr-DFO-mAb1:

For biodistribution studies, mice were euthanized at the final time-point (6 days post-⁸⁹Zr-DFO-mAb1 administration) and blood was collected via cardiac puncture. Raji/hPBMC tumors and normal tissues were then excised, placed in counting tubes, and weighed. Count data for ⁸⁹Zr in CPM was then collected by measuring samples on an automatic gamma counter (Wizard 2470, Perkin Elmer). The percent-injected dose per gram (% ID/g) was calculated for each sample using standards prepared from the injected material.

### Results, Summary, and Conclusions:

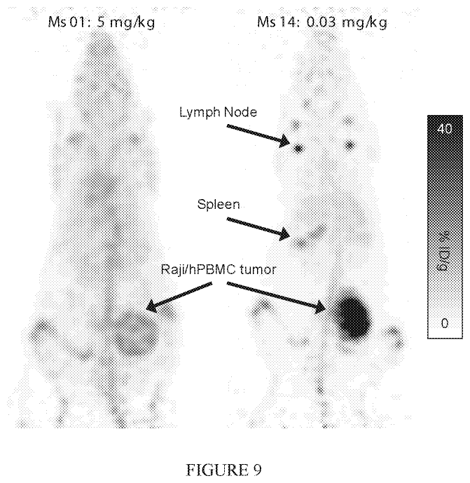

This study demonstrates antigen-specific targeting of ⁸⁹Zr-DFO-mAb1 to LAG3 expressed on human lymphocytes in subcutaneous Raji/hPBMC tumors grown in NSG mice. The blocking dose of 5 mg/kg ⁸⁹Zr-DFO-mAb1 showed increased blood uptake (% ID/g) and lower tumor uptake (% ID/g) in Raji/hPBMC tumors compared to the lower doses of 0.3, 0.1, and 0.03 mg/kg ⁸⁹Zr-DFO-mAb1 (Table 10). Furthermore, as the protein dose decreased, the average tumor-to-blood ratio increased demonstrating specificity to Lag-3 in vivo (Table 10). In addition to targeting Lag-3 expressed in the Raji/hPBMC tumors, the lower doses of 0.3, 0.1, and 0.03 mg/kg ⁸⁹Zr-DFO-mAb1 demonstrated targeting to the spleen and axillary lymph nodes of tumor bearing mice. Representative PET images (FIG. 9) at day 6 post ⁸⁹Zr-DFO-mAb1 administration demonstrate higher targeting of ⁸⁹Zr-DFO-mAb1 to the tumor, spleen, and axillary lymph nodes at 0.03 mg/kg compared 5 mg/kg.

TABLE 10
Ex vivo biodistribution at day 6 after administration of ⁸⁹Zr-DFO-mAb1 injected
at protein doses of 5. 0.3, 0.1, or 0.03 mg/kg in NSG mice bearing Raji/hPBMC tumors.
Values are shown as average and standard deviations of % ID/g and tumor-to-blood ratios

| | ⁸⁹Zr-DFO-mAb1 | | ⁸⁹Zr-DFO-mAb1 | | ⁸⁹Zr-DFO-mAb1 | | ⁸⁹Zr-DFO-mAb1 | |
|---|---|---|---|---|---|---|---|---|
| | 5 mg/kg | | 0.3 mg/kg | | 0.1 mg/kg | | 0.03 mg/kg | |
| | Average | STDEV | Average | STDEV | Average | STDEV | Average | STDEV |
| SAMPLE | % ID/g | % ID/g | % ID/g | % ID/g | % ID/g | % ID/g | % ID/g | % ID/g |
| Blood | 18.45 | 1.69 | 12.17 | 3.20 | 8.13 | 4.28 | 7.81 | 5.37 |
| Tumor | 20.52 | 5.34 | 40.43 | 8.09 | 33.26 | 10.81 | 48.92 | 28.53 |
| Thymus | 7.78 | 0.64 | 6.57 | 2.04 | 7.98 | 4.71 | 3.22 | 2.43 |
| Heart | 5.5 | 0.45 | 3.74 | 0.57 | 2.79 | 1.14 | 2.39 | 1.47 |
| Lungs | 10.14 | 0.54 | 8.30 | 2.40 | 9.72 | 1.63 | 8.14 | 1.08 |
| Spleen | 7.74 | 0.17 | 22.32 | 13.82 | 103.68 | 126.79 | 59.20 | 40.84 |
| Intestine | 1.82 | 0.23 | 1.43 | 0.20 | 0.80 | 0.44 | 1.19 | 0.23 |
| Liver | 4.51 | 0.26 | 5.56 | 1.16 | 9.75 | 3.87 | 10.75 | 5.58 |
| Kidney | 6.73 | 0.99 | 6.17 | 1.28 | 5.77 | 1.59 | 5.49 | 1.56 |
| Bone | 8.78 | 1.75 | 8.39 | 3.10 | 8.87 | 2.64 | 9.83 | 1.54 |
| Tumor-to-blood | 1.10 | 0.21 | 3.46 | 1.05 | 5.44 | 3.60 | 9.71 | 8.27 |
| ratio | | | | | | | | |

### Example 7: LC-PRM-MS Quantitation of LAG3 in Raji/PBMC Xenografts and Clinical Samples

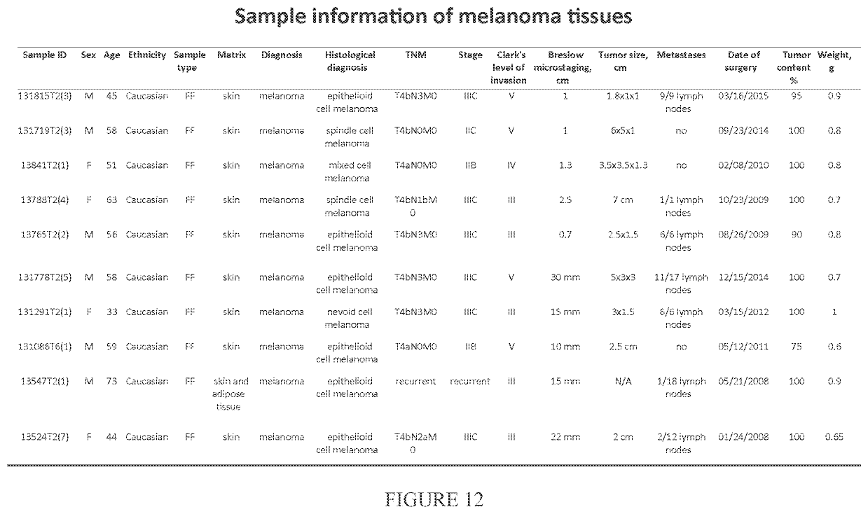

Frozen tissue samples (Raji/PBMC tumors, mouse spleens, and melanoma tissue; see FIG. 12 for source and characteristics of melanoma tissues) were lysed with lysis buffer (8 M urea in 50 mM NH₄HCO₃ with 1% RapiGest). Tissues were cut into small pieces and were homogenized with 1 mL lysis buffer in a tight fitting dounce homogenizer. The lysate was incubated on ice for 30 mins with sonication for 30 sec every 10 mins to achieve complete protein extraction. The lysate was centrifuged at 14,000 g for 10 mins. Protein concentration was measured by BOA assay. Each sample was diluted to 1 mg/mL then was centrifuged at 14,000 g for 10 mins and was stored in aliquots at −80 °C.

Unimplanted NSG mouse spleen lysate was used as the surrogate matrices to generate the standard curve for LAG3 quantitation. LAG3.Fc was spiked into each of 100 μg of mouse spleen lysate at a final concentration ranging from 0.39 to 50 ng/mg protein (1:2 serial dilution). Standards, xenografts and clinical melanoma lysates were precipitated in 900 μL of cold acetone overnight and then denatured in 90 μL of 8M Urea/TCEP buffer at 37 °C for 1 hr. Heavy labeled human LAG3 peptide (FVWSSLDTPSQR¹³C6¹⁵N4) was added to all samples as internal standard. The standards and test samples were alkylated with IAA at room temperature for 30 min and digested by lys-C(1:100 w/w) for 4 hrs then by trypsin (1:20 w/w) overnight at 37 °C. Samples were quenched with 10% FA to reach a final Vol. of 100 μL.

Each processed sample (2 L) was injected onto a pre-equilibrated nano C18 trap column and was separated by an easy nano C18 separation column. The flow rate was 250 nL/min (Mobile Phase A: water:formic acid/100:0.1 [V:V] and Mobile Phase B: acetonitrile:formic acid/100:0.1 [V:V]). Retention time and peak area were determined using Skyline software. The calibration curve was generated by plotting the peak area ratio of LAG3.Fc reference standard (unlabeled LAG3 peptide FVWSSLDTPSQR¹²C₆¹⁴N₄ generated by tryptic digest of hLAG3) to the internal standard (stable isotope-labeled LAG3 peptide). The concentration of LAG3 in each sample was calculated using linear regression. The lowest concentration of LAG3 reference standard (0.39 ng/mg protein) was within the dynamic range of the assay and was defined as the assay's lower limit of quantification.

### Results Summary and Conclusions:

LAG3 quantitation was performed on tissue samples from 4 of PBMC/Raji xenografts from 27 days, 5 xenografts from 15 days after tumor implantation and 10 melanoma clinical samples. The tissue weights, protein amounts, extraction yield and LAG3 expression were listed in Table 11. Bmax was calculated based on the following equation with an estimation of tumor density at 1 g/mL.

$$Bmax ( nM ) = \frac{\begin{matrix}LAG 3 ( ng / mg protein ) \times \\ Total Protein Amount ( mg ) \times 10 E 6\end{matrix}}{5 \text{.74} * 10 E 4 \times Tumor Weight ( mg )}$$

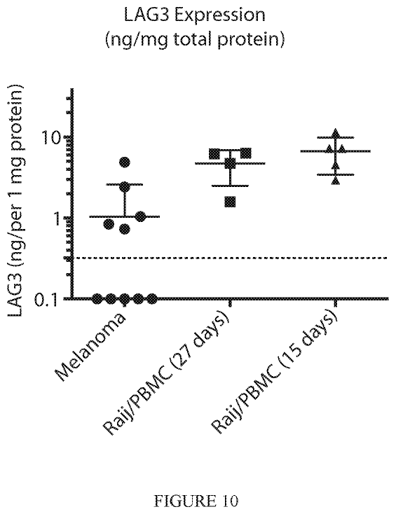

Five of 10 melanoma tissue samples were detected as LAG3 positive with an average expression level of 2.52±1.87 nM. This expression level is similar to Raji/PBMC model at 27 days (3.79±1.93 nM) and at 15 days (6.06±4.04 nM). See Table 11 and also FIG. 10.

TABLE 11

| | | | Total | | | |
|---|---|---|---|---|---|---|
| | | Tissue | Protein | | Lag3 | |
| | | Weight | Amount | % | (ng/mg | Bmax |
| | | (mg) | (mg) | protein | protein) | (nM) |
| Melanoma | 131815T2(3) | 290 | 9.1 | 3.14% | BLQ | BLQ |
| Tissue | 131719T2(3) | 230 | 17.6 | 7.65% | BLQ | BLQ |
| | 13841T2(1) | 220 | 20.1 | 9.14% | 0.73 | 1.16 |
| | 13788T2(4) | 250 | 24.1 | 9.64% | 1.04 | 1.75 |
| | 13765T2(2) | 250 | 19.4 | 7.76% | BLQ | BLQ |
| | 131778T2(5) | 180 | 9.2 | 5.11% | BLQ | BLQ |
| | 131291T2(1) | 240 | 17.4 | 7.25% | 0.84 | 1.06 |
| | 131086T6(1) | 180 | 9.32 | 5.18% | BLQ | BLQ |
| | 13547T2(1) | 220 | 16.1 | 7.32% | 2.42 | 3.08 |
| | 13524T2(7) | 200 | 13 | 6.50% | 4.90 | 5.53 |
| | Mean | 226 | 15.5 | 6.87% | 1.99 | 2.52 |
| | SD | 34 | 5.2 | 1.96% | 1.76 | 1.87 |
| Raji/PBMC | 85100_0 | 419.5 | 20.9 | 4.98% | 4.74 | 4.10 |
| Xenograft | 85101_8 | 248.9 | 10.3 | 4.14% | 1.58 | 1.14 |
| (27 Days) | 85104_23 | 256.5 | 9.74 | 3.80% | 6.24 | 4.12 |
| | 85103_19 | 112.5 | 5.92 | 5.26% | 6.32 | 5.78 |
| | Mean | 259 | 11.72 | 4.54% | 4.72 | 3.79 |
| | SD | 126 | 6.43 | 0.69% | 2.21 | 1.93 |
| Raji/PBMC | 213_1 | 140 | 8.8 | 6.29% | 11.46 | 12.5 |
| Xenograft | 213_2 | 260 | 10.14 | 3.90% | 4.54 | 3.08 |
| (15 Days) | 213_3 | 230 | 9.3 | 4.04% | 7.22 | 5.09 |
| | 213_4 | 160 | 7.9 | 4.94% | 2.95 | 2.54 |
| | 213_5 | 50 | 2.8 | 5.60% | 7.23 | 7.05 |
| | Mean | 168 | 7.8 | 4.95% | 6.68 | 6.06 |
| | SD | 82 | 6.43 | 0.69% | 2.21 | 1.93 |

### Example 8: Up-Regulation of Human LAG-3 and PD-1 Expression on T Cells in the Tumor Microenvironment by Therapy with REGN2810 (Anti-Human PD-1 Ab) and mAb1 (Anti-Human LAG-3 Ab)

This experiment was carried out to evaluate the modulation of expression levels of human LAG-3 and PD-1 on T cells in the tumor microenvironment upon treatment with REGN2810 and mAb1 using Regeneron's proprietary PD-1^{hu/hu}/LAG-3^{hu/hu} double humanized immune-competent mice. The tumor cell line used in this experiment is a murine colon carcinoma cell line MC38 (obtained from NCI at Frederick, MD, Laboratory of Tumor Immunology and Biology), which has been engineered in house to express full-length chicken ovalbumin fused with eGFP, thus referred here as MC38-cOVA/eGFP. The expression level of human LAG-3 was evaluated ex vivo on both CD4 and CD8 T cells from enzymatically disassociated tumors extracted from tumor bearing double humanized mice. All surface staining was performed with commercially available fluorochrome directly conjugated to antibodies (anti-human LAG-3 antibody: eBioscience, Clone 3DS223H; anti-human PD-1 antibody: BioLegend, Clone EH12.2H7), following standard protocol. Briefly, tumor cells were washed with PBS once, washed with ice cold staining buffer once, stained with commercial available fluorochrome directly conjugated anti-human PD-1 or anti-human LAG-3 antibody in staining buffer for 30 min on ice in the dark, washed with 2 ml of PBS once again. Fixable dye eFluor506 was also included following manufacturer's protocol (eBioscience). Samples were acquired on BD FACSCanto II™ IVD10 equipped with DIVA v8. Data were further analyzed with FlowJo v10.0.6 or the later version.

### Results Summary and Conclusions:

Table 12 provides a schematic presentation of the therapeutic dosing regimen in pre-clinical tumor setting. 1×10⁶ MC38-cOVA/eGFP cells were implanted s.c. into PD-1^{hu/hu}/LAG-3^{hu/hu} double humanized immune-competent mice. At about Day 11, mice were randomized into four groups with average tumor volumes of 100 mm³ and started treatment as indicated. Tumor samples were collected 3 days after the second dose.

TABLE 12
Therapeutic dosing regimen.

| Group | Treatment | # Mice |
|---|---|---|
| Isotype | 25 mg/kg, 2× week, 2 doses, IP | 10 |
| REGN2810(PD-1) | 10 mg/kg, 2× week, 3 doses, IP | 12 |
| mAb1 (anti-human | 25 mg/kg, 2× week, 2 doses, IP | 12 |
| LAG-3) | | |
| REGN2810 + mAb1 | 10 mg/kg + 25 mg/kg, 2× week, | 12 |
| | 2 doses, IP | |

As shown in Table 13, the combination of anti-human PD-1 (REGN2810) and anti-human LAG-3 (mAb1) significantly inhibited tumor growth in MC38-cOVA/eGFP syngeneic tumor model in double humanized mice. Tumor-bearing mice (tumor sizes of about 100 mm³) were treated with an hlgG4 isotype control antibody, REGN2810 (anti-human PD-1, hlgG4), mAb1 (anti-human LAG-3, hlgG4s), and combination of REGN2810 and mAb1, twice a week for two doses, and tumor sizes were measured by caliper. Tumor volume was calculated as V=L×W²/2. In the control group, tumor sizes ranged from 300 to 869 mm³ with median value of 548 mm³. REGN2810 treated group showed reduced tumor sizes (121 to 721 mm³ with median at 466 mm³), but the differences did not reach statistical significance. Whereas mAb1-treated group showed no difference from the isotype control group either (203 to 721 mm³ with median at 592 mm³), the combination treatment significantly delayed tumor growth (113 to 621 mm³ with median at 289 mm³, p<0.01).

TABLE 13
Anti-human PD-1 (REGN2810) and anti-Human LAG-3 (mAb1)
significantly inhibited tumor growth in MC38-cOVA/GFP
syngeneic tumor model in double humanized mice

| | Iso** | αhPD-1 | αhLAG-3** | Combo |
|---|---|---|---|---|
| Mice/group | 10 | 12 | 12 | 12 |
| Minimum | 299.9 | 120.9 | 202.6 | 113.4 |
| 25% Percentile | 437.6 | 321.3 | 426.9 | 192.6 |
| Median | 548.4 | 465.5 | 592.1 | 289.1 |
| 75% Percentile | 617.6 | 597.8 | 631.1 | 349.7 |
| Maximum | 868.7 | 710.6 | 760.7 | 631.4 |

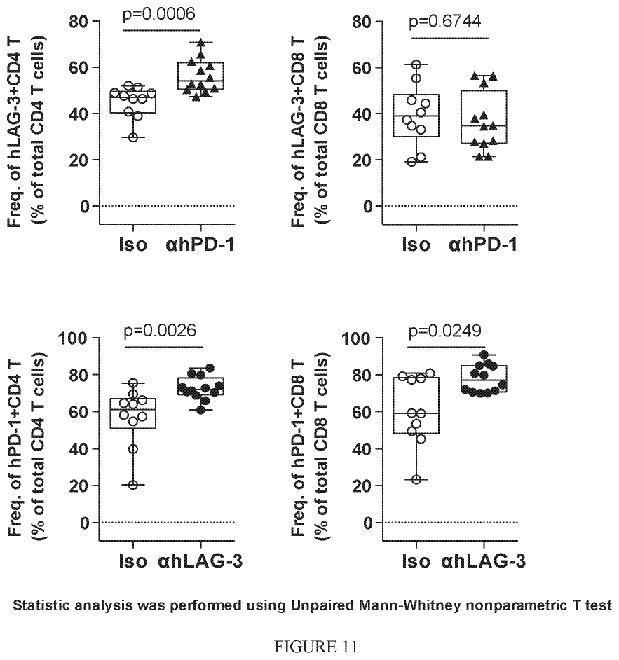

REGN2810 anti-human PD-1 Ab and mAb1 anti-human LAG-3 respectively increased LAG-3+ T cells and PD-1+ T cells in tumor microenvironment, as can be seen in FIG. 11. Tumors from individual mice were dissociated by GentalMACs (Miltenyi Biotech) according to the Manufacturer's protocol. Samples were stained with a panel of Abs and analyzed by flow cytometer. Data presented were pre-gated on FSC/SSC, viability, singlets, CD45+CD3+ cells, then further gated on CD4 or CD8 T cells. The expression of human LAG-3 and human PD-1 were evaluated between different groups. To eliminate the possible Ab cross-competition, REGN2810—and combination-treated groups were excluded from human PD-1 analysis. Similarly, mAb1—and combination-treated groups were also excluded from human LAG-3 analysis. After two therapeutic doses, REGN2810 significantly increased the frequency of human LAG-3+CD4 T cells in tumor microenvironment by ~24% (p=0.0006), though it did seem to have a direct modulatory role for LAG-3 expression on CD8 T cells with the dosing regimen tested. Interestingly, mAb1 also increased the frequency of human PD-1+CD4 (p=0.0026) and CD8 T cells (p=0.0249) in tumor microenvironment by ~28%, respectively. See FIG. 11.

The results from the studies performed here clearly demonstrate that anti-LAG3 antibody labeled with ⁸⁹Zr can significantly and specifically localize to tumors. One may envision a scenario where the anti-LAG3 antibody is used in the selection of patients with LAG3 positive tumors for subsequent treatment with LAG3 inhibitors, alone or in combination with other anti-cancer therapeutics including inhibitors of the PD-1/PD-L1 signaling axis.

### Example 9: Scaled-Up Manufacturing Process for Producing DFO-Anti-LAG3 Antibody Conjugates

This example details the scaled-up manufacturing process for preparing the anti-LAG3 antibody to be suitable for radiolabeling by attaching p-SCN-bn-Deferoxamine (DFO) to the anti-LAG3 antibody (mAb, H4sH15482P) described herein: (1) ultrafiltration and diafiltration (UFDF) processes prior to mAb conjugation removes excipients that inhibit the conjugation process; (2) following the pre-conjugation UFDF, conjugation of the mAb with p-SCN-Bn-deferoxamine is performed to produce DFO-mAb conjugates; and (3) a post-conjugation UFDF to remove residual salts provides a suitable concentration, excipient level, and pH of the conjugated monoclonal antibody. The resulting DFO-mAb conjugates are then provided in a buffered state with improved stability for subsequent formulation.

### (1) Pre-Conjugation Ultrafiltration and Diafiltration (UFDF)

100 g mAb was buffer exchanged into a 5 mM acetate buffer solution having a pH of 5.50 using a Sius Prostream (TangenX Technology Corporation) membrane (membrane capacity of ≤500 g/m²) to remove residual salts prior to conjugation. The process volume was reduced to further concentrate the antibody, then the antibody was sterile filtered using a Sartopore 2 (Sartorius) membrane having a 0.45/0.2 μm (heterogeneous PES double layer) or equivalent pore size. The acetate buffer temperature was kept at a target temperature of 20±5 °C. The solutions were well mixed.

### (2) Conjugation

The concentrated and filtered antibody (20 g) was transferred into a conjugation vessel containing an amine free carbonate buffer system (56 mM Carbonate, 167 mM Sodium Chloride, pH 9.40) resulting in negligible levels of residual acetate. DFO (25 mM p-SCN-Bn-Deferoxamine) was solubilized in DMSO and added to the conjugation vessel, along with additional DMSO such that the DMSO was present in a final amount of 5%. DFO was added in molar excess at a ratio of 4.5:1 DFO to mAb. The total reaction volume equaled 2.0 L. The buffer system was mixed throughout the addition of the reaction ingredients and throughout the reaction time.

The reaction temperature was controlled for specific time by using an equation which relates temperature to reaction time. In this instance, the reaction temperature was held at 20±2 °C for 180 minutes. The reaction was quenched by the addition of 2M acetic acid (23 mL/L), resulting in the solution having a pH of 6.

### (3) Post-Conjugation UFDF

After the conjugation step, the quenched DFO-mAb conjugation solution was buffer exchanged into histidine buffer (10 mM Histidine, pH 5.50 with 0.0005% (w/v) super refined polysorbate 80 added as a shear protectant) to remove residual process salts, DMSO, and unreacted DFO. Once diafiltered, the solution was then concentrated and subsequently formulated. The histidine buffer was selected for long term storage of protein at −80 °C. The same Sius Prostream membrane mentioned in step (1) was used in the final UFDF step. The resulting concentrated DFO-mAb conjugate solution was sterile filtered using the Sartopore 2 filter mentioned above.

UV-DAR (target of 1.5) and protein concentration determination was performed as described in Example 2.

TABLE 14
Molar Extinction Coefficients
and Molecular Weight

| | MW | □280 | □252 |
|---|---|---|---|
| Antibody | (g mo1⁻¹) | (L g⁻¹cm⁻¹) | (L g⁻¹cm⁻¹) |
| H4sH15482P | 145709 | 223400 | 87077 |

### Example 10: ImmunoPET Imaging of LAG3 in Tumors Using an ⁸⁹Zr-DFO-Anti-LAG3 Antibody Conjugate in Patients with Metastatic Melanoma

The primary objective of this study is to determine the safety and tolerability of ⁸⁹Zr-DFO-anti-LAG3 antibody conjugate, in which the anti-LAG3 antibody used in the radiolabeled conjugate is H4sH15482P. Outcome measures monitor adverse events and routine laboratory tests for safety.

The secondary objectives of the study are:
Study part A: To qualify ⁸⁹Zr-DFO-anti-LAG3 PET as a biomarker for the evaluation of LAG3 expression in tumors. This will be accomplished by evaluating the safety of the ⁸⁹Zr-DFO-anti-LAG3 PET tracer, determining optimal tracer mass dose and optimal post-injection imaging time, establishing the relationship of tumor PET signal with LAG3 tissue-based expression, and evaluating dosimetry in patients. Part A comprises a sequential tracer dose escalation design, with tumor biopsy. Imaging and blood draws at days 1, 4, and 7 post tracer injection permit blood poos SUV with subsequent calculation of tumor: blood ratios at the time of imaging; clinical dosimetry based on tissue radiation absorbed dose and effective dose calculated from PET image acquisition data and tracer activity concentration in blood; standardized uptake values (SUVs—decay-corrected activity concentration in target tissue divided by the mean activity concentration in the body at the time of injection) across the tumor regions of interest; maximal SUVs within tumor regions of interest (ROIs) (SUVₘₐₓ); and plasma tracer activity concentration, with calculation of area under the curve (AUC_{0-7 days}).
Study part B: To explore the construct and criterion validity of ⁸⁹Zr-DFO-anti-LAG3 PET by correlating the PET signal with tissue-based LAG3 expression and clinical outcome (objective response rate and progression-free survival) after I0 therapy. Sequential iPET scanning and tumor biopsies are performed before and after treatment with standard of care immunotherapies selected from the following: nivolumab, ipilimumab, pembrolizumab, and combinations as allowed by label.

The utility of the immune-PET (iPET) tracer can be initially assessed by testing for ability to detect the presence of LAG3 tumors, as well as changes in LAG3 signal induced by an established immunotherapy, and by exploring the correlation of the iPET signal with clinical outcomes (criterion validation: against biologically and clinically meaningful outcomes).

A safe, optimal mass dose of ⁸⁹Zr-DFO-anti-LAG3 can be identified that shows adequate tumor uptake by PET, tracer PK, and dosimetry. Selection of three tracer mass dose levels is based on preclinical mouse xenograft imaging and biodistribution studies, and on clinical and preclinical data using unlabeled anti-LAG3 therapeutic antibodies. The planned mass dose escalation is 2 mg, 5 mg, and 10 mg. The approach is to use doses that are sub-therapeutic or pharmacologically inert, so as not to interfere with prospective anti-tumor therapy.

The optimal mass dose will demonstrate tumor SUV, maximal SUV (SUVₘₐₓ) within the tumor lesion region of interest (ROI) and tumor:blood ratio all >1 (and ideally a tumor-blood ratio of 3-4) in at least one lesion (ideally in >1 lesion, in patients with several metastases).

Tracer activity in plasma (or serum) and/or blood pool SUV (the activity PK measures for this study) will be detectable throughout the 7-day imaging window, following dosing, suggesting adequate availability of tracer to compartmentalize into tumor lesions. Ratios of tumor and blood signal will be based on SUVs, although other activity concentration units may be used. The same applies to measurements of blood activity concentration, which could be reported in terms of absolute units or normalized units.

LAG3 PET signal intensity in a biopsied lesion will covary with degree of LAG3 expression in the tissue biopsy using semi-quantitative measures.

The autoradiographic LAG3 PET signal will correlate spatially with LAG3 expression in tissue biopsy samples.

LAG3 PET signal intensity will increase following treatment with an immunotherapy.

LAG3 PET signal intensity increase will correlate with response following treatment with an immunotherapy.

Additionally, exploratory objectives and outcome measures include determining expression of LAG3 in tissue biopsies in correlation with tumor ⁸⁹Zr-DFO-anti-LAG3 uptake using immunohistochemistry, RNAscope, liquid chromatography mass spectrometry (LC/MS), and autoradiography. For part B only, exploratory objectives include measuring changes in ⁸⁹Zr-DFO-anti-LAG3 signal after treatment and correlation of ⁸⁹Zr-DFO-anti-LAG3 signal with clinical outcome after treatment. The outcome measures include SUV, SUVmax, tumor:blood ratio, and clinical outcome following immunotherapy treatment (serial CT for the purpose of calculation of responder status using RECIST 1.1 and tumor volume), objective response rate, and progression-free survival.

### Patient Target Population

The target population will consist of patients 18 years of age or older with advanced metastatic melanoma, histologically or cytologically confirmed diagnosis, with at least one lesion amendable to biopsy. The patient must have an ECOG performance status of less than or equal to 2, an anticipated life expectancy of at least 3 months, and adequate organ and bone marrow function.

Inclusion of patients with an indication that has a high prevalence of the target will support assessment of LAG3 iPET tumor localization which is a key outcome of the study. Detection and correlation of post-immunotherapy LAG3 expression with clinical outcomes requires a patient population with well characterized clinical response rates to immunotherapies. Metastatic melanoma patients represent a patient population with established response rates to checkpoint inhibitors as well as the high levels of prevalence and expression of LAG3.

### Study Design

The study comprises part A (construct validation) and part B (criterion validation). Duration of the study is 9 weeks for Part A (4 weeks screening, 1 week tracer dosing, scans and biopsy, 4 weeks safety follow up), and 18 weeks for Part B (4 weeks screening, 1 week tracer dosing, scans and biopsy, up to 8 weeks on immunotherapy, 1 week second tracer dose and scan, 4 weeks safety follow up).

### Part A

Part A is a dose finding study in which patients receive a single tracer dose, followed by serial scans and a biopsy over a 7 day period. Once the scanning sequence and biopsy are completed, subjects can immediately be treated with a standard of care immunotherapy regime (anti-PD-1 alone or in combination with anti-CTLA4 according to labeled indication).

### Dose Cohorts in Part A

Part A comprises three sequential dose cohorts, consisting of 3 patients, with potential to expand the cohort to a total of 6 patients (3+3 design). Dose escalation decisions will be informed by a) safety and b) evaluation of iPET positivity. Dose limiting toxicity (DLT) is defined as a Grade 3 or higher (NTCAE) adverse event (AE) related to or possibly related to ⁸⁹Zr-DFO-anti-LAG3, one week following tracer administration. For hematologic lab AEs, DLT is defined as Grade 4 or higher. Tumor uptake positivity/tumor localization is defined by a tumor:blood ratio greater than 1. Adequate PK is defined by SUV in blood in the range of 1-5 at optimum imaging time (4 or 7 days post-injection).

Cohort expansion to 6 patients will occur if any of the following conditions are met: (a) exactly 1 patient experiences a DLT or (b) at least 1 patient out of 3 shows tumor localization and adequate PK and no more than 1 patient experiences a DLT.

At the completion of a cohort of either 3 or 6 subjects, dose escalation will occur to a higher available dose if fewer than 3 patients in an expanded cohort experience a DLT.

Part A of study will stop if any of the following conditions are met (Part A stopping rules): more than 1 patient in a cohort experiences a DLT; more than 3 patients show visual tumor localization and adequate PK in each of two consecutive expanded cohorts; or no higher doses are available for escalation.

Upon reaching a Part A stopping rule, Part B dose will be selected as follows: a) if two or three expanded cohorts show more than 3 patients with tumor localization and adequate PK, then the dose cohort with tumor localization in more patients, or the highest tumor: blood ratios, will be chosen. When these are similar between cohorts, the lower dose will be chosen. b) if one cohort shows more than 3 patients with tumor localization and adequate PK, this dose will be chosen. c) if no cohorts show more than 3 patients with tumor localization and adequate PK, the study will terminate without progression to Part B.

### Part B

Part B will measure LAG3 iPET signal at the defined tracer dose and post-injection time point (determined in part A), both pre- and post-immunotherapy to assess the hypotheses surrounding the role of LAG3 as an indicator of tumor inflammatory response (exploratory objectives). All patients in Part B will receive the optimal tracer mass dose and post-injection imaging timing as identified in Part A.

Part B patients will receive LAG3 iPET scanning at baseline as well as a biopsy prior to therapy. Patients will then receive a standard of care immunotherapy (currently these are monoclonal antibody-based PD-1 and CTLA-4 pathway blockers), according to the label. Four to eight weeks later an additional iPET scan will be undertaken followed by a second biopsy if feasible.

Patients in Part A who received the optimal tracer mass dose and achieved adequate scan quality may be eligible for Part B and receive a total of two iPET tracer injections. The total number of subjects in Part B (including those that enter from Part A) will not exceed 20.

### Biopsy Considerations

Lesions will be selected for biopsy on the basis of accessibility and size (typically at least 20 mm diameter). All patients will undergo a baseline biopsy on the last day of the first set of iPET scans, regardless of whether the iPET study is positive or not. In this way, tissues from patients with a wide range of LAG3 tissue expression will be collected for correlation with LAG3 signal, including negative patients. The biopsy will be scheduled no later than 7 days from date of injection in order to minimize delay of therapy to the patient.

A sequence of assessments that starts with a biopsy followed by the tracer dosing and scans, and then the initiation of therapy may be preferable for practical reasons.

For Part B, a second biopsy after the second scan may be undertaken if feasible and will be optional. Sequential biopsies will be taken from the same site if practicable.

Autoradiography studies will be performed in a subset of biopsied tumors that are positive on iPET scan, with adjacent slices stained against LAG3.

### Study Interventions

### Part A

Following screening, each subject will receive a dose of ⁸⁹Zr-DFO-anti-LAG3 followed by three sequential iPET scans over 6-7 days. Starting dose will be 2 mg, as determined from animal studies and modeling. No later than 1 day after the last iPET scan, the subject will undergo radiology-guided biopsy. If available, archived biopsy tumor tissue will also be analyzed by IHC for LAG3 expression.

For Part A, biopsy is optional, since not all subjects will receive the eventually identified optimal tracer dose.

Decision to progress to Part B will be made on the basis of Part A data and recruitment rate.

### Part B

Following screening, each melanoma patient will receive a ⁸⁹Zr-DFO-anti-LAG3 at the optimized mass dose (from Part A) followed by PET scanning at the optimal post-injection time point (from Part A). Then, no later than 1 day after iPET imaging, the subject will undergo radiology guided biopsy of a lesion. Subsequently, the patient will be treated, open-label, with available approved immunotherapy regimens (dosed as per label). Subjects will receive a second scan 4-8 weeks after commencement of immunotherapy. A second biopsy after the second scan may be undertaken if feasible and will be optional.

All patients will be screened by an ¹⁸F-FDG PET/CT scan. CT portion of the PET/CT scan must be of diagnostic quality or a diagnostic CT scan acquired during the screening period must be available to assess location and dimension of lesions. These scans will be used to evaluate the lesions for metabolic activity/viability and appropriate dimensions.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

| SEQ ID | | |
|---|---|---|
| NO. | Sequence | Description |
| 1. | gaggtgcagc tgttggagtc tgggggaggc ttggtacagc ctggggggtc | DNA |
| | cctgagactc | nucleotide |
| | tcctgtgtgg cctctggatt cacctttagc acctatgcca tgagttgggt | sequence |
| | ccgccaggct | |
| | ccagggatgg ggctggagtg ggtctcaagt attagtggta gtggtcgtaa | |
| | cacatactat | |
| | gcagactccg tgaagggccg gttcaccatc tccagagaca attccaagaa | |
| | cacgctgttt | |
| | cttcaaatga acagcctgag agccgaggac acggccgttt attactgtgc | |
| | gaaagagtcc | |
| | gtaactggaa cttcgtccta ctactacggt gtggacgtct ggggccaagg | |
| | gaccacggtc | |
| | accgtctcct cg | |
| 2. | EVQLLESGGG LVQPGGSLRL SCVASGFTFS | AA amino |
| | TYAMSWVRQA PGMGLEWVSS ISGSGRNTYY | acid |
| | ADSVKGRFTI SRDNSKNTLF LQMNSLRAED | sequence |
| | TAVYYCAKES VTGTSSYYYG VDVWGQGTTV | |
| | TVSS | |
| 3. | ggattcacct ttagcaccta tgcc | DNA |
| | | nucleotide |
| | | sequence |
| 4. | GFTFSTYA | AA amino |
| | | acid |
| | | sequence |
| 5. | attagtggta gtggtcgtaa caca | DNA |
| | | nucleotide |
| | | sequence |
| 6. | ISGSGRNT | AA amino |
| | | acid |
| | | sequence |
| 7. | gcgaaagagt ccgtaactgg aacttcgtcc tactactacg gtgtggacgt | DNA |
| | c | nucleotide |
| | | sequence |
| 8. | AKESVTGTSS YYYGVDV | AA amino |
| | | acid |
| | | sequence |
| 9. | gacatccaga tgacccagtc tccatcctcc ctgtctgcat ctgtaggaga | DNA |
| | cagagtcacc atcacttgcc gggcaagtca gagcattagc agttatttaa | nucleotide |
| | attggtatca tcagaaacca gggaaagccc caaagctcct gatctatgct | sequence |
| | gcatccagtt tgcaaaatgg ggtcccatca aggttcagtg gcagtggatc | |
| | tgggacagat ttcactctca ccatcagcag tctgcaacct gaagattttg | |
| | catcttacta ctgtcaacag agttacagaa ccccgctcac tttcggcgga | |
| | gggaccaagg tggagatcaa a | |
| 10. | DIQMTQSPSS LSASVGDRVT ITCRASQSIS SYLNWYHQKP | AA amino |
| | GKAPKLLIYA ASSLQNGVPS RFSGSGSGTD FTLTISSLQP | acid |
| | EDFASYYCQQ SYRTPLTFGG GTKVEIK | sequence |
| 11. | cagagcatta gcagttat | DNA |
| | | nucleotide |
| | | sequence |
| 12. | QSISSY | AA amino |
| | | acid |
| | | sequence |
| 13. | gctgcatcc | DNA |
| | | nucleotide |
| | | sequence |
| 14. | AAS | AA amino |
| | | acid |
| | | sequence |
| 15. | caacagagtt acagaacccc gctcact | DNA |
| | | nucleotide |
| | | sequence |
| 16. | QQSYRTPLT | AA amino |
| | | acid |
| | | sequence |
| 17. | caggtgcagc tggaggagtc tgggggaggc gtggtccagc ctgggaggtc | DNA |
| | cctgagactc | nucleotide |
| | tcctgtgcag cgtctggatt caccttcagt tggtatggca tgcactgggt | sequence |
| | ccgccaggct | |
| | ccaggcaagg ggctggagtg ggtggcactt atatggtatg atggaactaa | |
| | taaaaagtat | |
| | ggagactccg tgaagggccg attcaccatt tccagagaca attccaagaa | |
| | cacggtgtat | |
| | ctgcaaatga acagcctgag agccgaggac acggctgtgt attactgtgc | |
| | gagagattgt | |
| | ggacatagtg gcaacgatcg ggggacttac tattactact acggtatgga | |
| | cgtctggggc | |
| | caagggacca cggtcaccgt ctcctca | |
| 18. | QVQLEESGGG VVQPGRSLRL SCAASGFTFS | AA amino |
| | WYGMHWVRQA PGKGLEWVAL IWYDGTNKKY | acid |
| | GDSVKGRFTI SRDNSKNTVY LQMNSLRAED | sequence |
| | TAVYYCARDC GHSGNDRGTY YYYYGMDVWG | |
| | QGTTVTVSS | |
| 19. | ggattcacct tcagttggta tggc | DNA |
| | | nucleotide |
| | | sequence |
| 20. | GFTFSWYG | AA amino |
| | | acid |
| | | sequence |
| 21. | atatggtatg atggaactaa taaa | DNA |
| | | nucleotide |
| | | sequence |
| 22. | IWYDGTNK | AA amino |
| | | acid |
| | | sequence |
| 23. | gcgagagatt gtggacatag tggcaacgat cgggggactt actattacta | DNA |
| | ctacggtatg | nucleotide |
| | gacgtc | sequence |
| 24. | ARDCGHSGND RGTYYYYYGM DV | AA amino |
| | | acid |
| | | sequence |
| 25. | gacatccaga tgacccagtc tccatcctcc ctgtctgcat ctgtaggaga | DNA |
| | cagagtcacc | nucleotide |
| | atcacttgcc gggcaagtca gagcattagc agctatttaa attggtatca | sequence |
| | gcagaaacca | |
| | gggaaagccc ctaagctcct gatctatgct gcatccagtt tgcaaagtgg | |
| | ggtcccatca | |
| | aggttcagtg gcagtggatc tgggacagat ttcactctca ccatcagcag | |
| | tctgcaacct | |
| | gaagattttg caacttacta ctgtcaacag agttacagta cccctccgat | |
| | caccttcggc | |
| | caagggacac gactggagat taaa | |
| 26. | DIQMTQSPSS LSASVGDRVT ITCRASQSIS | AA amino |
| | SYLNWYQQKP GKAPKLLIYA ASSLQSGVPS | acid |
| | RFSGSGSGTD FTLTISSLQP EDFATYYCQQ | sequence |
| | SYSTPPITFG QGTRLEIK | |
| 27. | cagagcatta gcagctat | DNA |
| | | nucleotide |
| | | sequence |
| 28. | QSISSY | AA amino |
| | | acid |
| | | sequence |
| 29. | gctgcatcc | DNA |
| | | nucleotide |
| | | sequence |
| 30. | AAS | AA amino |
| | | acid |
| | | sequence |
| 31. | caacagagtt acagtacccc tccgatcacc | DNA |
| | | nucleotide |
| | | sequence |
| 32. | QQSYSTPPIT | AA amino |
| | | acid |
| | | sequence |
| 33. | caggtgcagc tacagcagtg gggcgcagga ctgttgaagc cttcggagac | DNA |
| | cctgtccctc | nucleotide |
| | acctgcgctg tctatggtgg gtccttcagt ggttactact ggaactggat | sequence |
| | ccgccagccc | |
| | ccagggaagg ggctggagtg ggttggggaa atcagtcata gaggaaccac | |
| | caactacaac | |
| | ccgtccctca agagtcgagt caccatatca ctggacacgt ccaagaacca | |
| | gttctccctg | |
| | aaactgacct ctgtgaccgc cgcggacacg gctgtgtatt actgttcgag | |
| | agacgaggaa | |
| | ctggaattcc gtttctttga ctactggggc cagggaaccc tggtcaccgt | |
| | ctcctca | |
| 34. | QVQLQQWGAG LLKPSETLSL TCAVYGGSFS | AA amino |
| | GYYWNWIRQP PGKGLEWVGE ISHRGTTNYN | acid |
| | PSLKSRVTIS LDTSKNQFSL KLTSVTAADT | sequence |
| | AVYYCSRDEE LEFRFFDYWG QGTLVTVSS | |
| 35. | ggtgggtcct tcagtggtta ctac | DNA |
| | | nucleotide |
| | | sequence |
| 36. | GGSFSGYY | AA amino |
| | | acid |
| | | sequence |
| 37. | atcagtcata gaggaaccac c | DNA |
| | | nucleotide |
| | | sequence |
| 38. | ISHRGTT | AA amino |
| | | acid |
| | | sequence |
| 39. | tcgagagacg aggaactgga attccgtttc tttgactac | DNA |
| | | nucleotide |
| | | sequence |
| 40. | SRDEELEFRF FDY | AA amino |
| | | acid |
| | | sequence |
| 41. | gaaattgtgt tgacacagtc tccagccacc ctgtctttgt ctccagggga | DNA |
| | aagagccacc | nucleotide |
| | ctctcctgca gggccagtca gagtgttagc agctatttag cctggtacca | sequence |
| | acaaaaacct | |
| | ggccaggctc ccaggctcct cgtctatggt gcatccaaca gggccactgg | |
| | catcccagcc | |
| | aggttcagtg gcagtgggtc tgggacagac ttcactctca ccatcagcag | |
| | cctagagcct | |
| | gaagattttg cattttatta ctgtcagcag cgtagcaact ggccgctcac | |
| | tttcggcgga | |
| | gggaccaagg tggagatcaa a | |
| 42. | EIVLTQSPAT LSLSPGERAT LSCRASQSVS | AA amino |
| | SYLAWYQQKP GQAPRLLVYG ASNRATGIPA | acid |
| | RFSGSGSGTD FTLTISSLEP EDFAFYYCQQ | sequence |
| | RSNWPLTFGG GTKVEIK | |
| 43. | cagagtgtta gcagctat | DNA |
| | | nucleotide |
| | | sequence |
| 44. | QSVSSY | AA amino |
| | | acid |
| | | sequence |
| 45. | ggtgcatcc | DNA |
| | | nucleotide |
| | | sequence |
| 46. | GAS | AA amino |
| | | acid |
| | | sequence |
| 47. | cagcagcgta gcaactggcc gctcact | DNA |
| | | nucleotide |
| | | sequence |
| 48. | QQRSNWPLT | AA amino |
| | | acid |
| | | sequence |
| 49. | cagctgcagc tgcaggagtc gggcccagga ctggtgaagc cttcggagac | DNA |
| | cctgtccctc | nucleotide |
| | acctgcactg tctctggtga ctccatcatc agtaatagtt attactgggg | sequence |
| | ctggatccgc | |
| | cagcccccag ggaaggggct ggagtggatt ggcaatttct tttatactgg | |
| | ggccacctac | |
| | tacaacccgt ccctcaagag tcgagtcacc atatccgctg acacgtccaa | |
| | gaatcagttc | |
| | tccctgaagc tgagctctgt gaccgccgca gacacggctc tgtattattg | |
| | tgcgagttat | |
| | aataggaatt accggttcga cccctggggc cagggaaccc tggtcaccgt | |
| | ctcctca | |
| 50. | QLQLQESGPG LVKPSETLSL TCTVSGDSII SNSYYWGWIR | AA amino |
| | QPPGKGLEWI GNFFYTGATY | acid |
| | YNPSLKSRVT ISADTSKNQF SLKLSSVTAA DTALYYCASY | sequence |
| | NRNYRFDPWG QGTLVTVSS | |
| 51. | ggtgactcca tcatcagtaa tagttattac | DNA |
| | | nucleotide |
| | | sequence |
| 52. | GDSIISNSYY | AA amino |
| | | acid |
| | | sequence |
| 53. | ttcttttata ctggggccac c | DNA |
| | | nucleotide |
| | | sequence |
| 54. | FFYTGAT | AA amino |
| | | acid |
| | | sequence |
| 55. | gcgagttata ataggaatta ccggttcgac ccc | DNA |
| | | nucleotide |
| | | sequence |
| 56. | ASYNRNYRFD P | AA amino |
| | | acid |
| | | sequence |
| 57. | gacatccaga tgacccagtc tccatcctcc ctgtctgcat ctgtaggaga | DNA |
| | cagagtcacc | nucleotide |
| | atcacttgcc gggcaagtca gagcattagc agctatttaa attggtatca | sequence |
| | gcagaaacca | |
| | gggaaagccc ctaagctcct gatctatgct gcatccagtt tgcaaagtgg | |
| | ggtcccatca | |
| | aggttcagtg gcagtggatc tgggacagat ttcactctca ccatcagcag | |
| | tctgcaacct | |
| | gaagattttg caacttactt ctgtcaacag agttacagta cccctccgat | |
| | caccttcggc | |
| | caagggacac gactggagat taaa | |
| 58. | DIQMTQSPSS LSASVGDRVT ITCRASQSIS | AA amino |
| | SYLNWYQQKP GKAPKLLIYA ASSLQSGVPS | acid |
| | RFSGSGSGTD FTLTISSLQP EDFATYFCQQ | sequence |
| | SYSTPPITFG QGTRLEIK | |
| 59. | cagagcatta gcagctat | DNA |
| | | nucleotide |
| | | sequence |
| 60. | QSISSY | AA amino |
| | | acid |
| | | sequence |
| 61. | gctgcatcc | DNA |
| | | nucleotide |
| | | sequence |
| 62. | AAS | AA amino |
| | | acid |
| | | sequence |
| 63. | caacagagtt acagtacccc tccgatcacc | DNA |
| | | nucleotide |
| | | sequence |
| 64. | QQSYSTPPIT | AA amino |
| | | acid |
| | | sequence |
| 65. | caggtgcagc tacagcagtg gggcgcagga ctgttgaagc cttcggagac | DNA |
| | cctgtccctc | nucleotide |
| | acctgcgctg tctatggtgg gtccttcagt acttactact ggagctggat | sequence |
| | ccgccagccc | |
| | ccagggaagg ggctggagtg gattggagag atcaatcata gtggaaacgc | |
| | cgactacaac | |
| | ccgtccctca agagtcgagt ctccatatca gtggacacgt ccaagaacca | |
| | gttctccctg | |
| | aggctgagct ctgtgaccgc cgcggacacg gctatttatt actgtgcgag | |
| | agcgggctat | |
| | tgtagtagtc ccacctgcta ttcctactac tacttcggta tggacgtctg | |
| | gggccaaggg | |
| | accacggtca ccgtctcctc a | |
| 66. | QVQLQQWGAG LLKPSETLSL TCAVYGGSFS | AA amino |
| | TYYWSWIRQP PGKGLEWIGE INHSGNADYN | acid |
| | PSLKSRVSIS VDTSKNQFSL RLSSVTAADT | sequence |
| | AIYYCARAGY CSSPTCYSYY YFGMDVWGQG | |
| | TTVTVSS | |
| 67. | ggtgggtcct tcagtactta ctac | DNA |
| | | nucleotide |
| | | sequence |
| 68. | GGSFSTYY | AA amino |
| | | acid |
| | | sequence |
| 69. | atcaatcata gtggaaacgc c | DNA |
| | | nucleotide |
| | | sequence |
| 70. | INHSGNA | AA amino |
| | | acid |
| | | sequence |
| 71. | gcgagagcgg gctattgtag tagtcccacc tgctattcct actactactt | DNA |
| | cggtatggac | nucleotide |
| | gtc | sequence |
| 72. | ARAGYCSSPT CYSYYYFGMD V | AA amino |
| | | acid |
| | | sequence |
| 73. | gaaattgtgt tgacgcagtc tccaggcacc ctgtctttgt ctctagggga | DNA |
| | aagagccacc | nucleotide |
| | ctctcctgca gggccagtca gagtgttatc agcagcttct tagcctggta | sequence |
| | ccagcagaaa | |
| | cctggccagg ctcccaggct cctcatctat ggtgcatcca gcagggccac | |
| | tggcttccca | |
| | gacaggttca gtggcagtgg gtctgggaca gacttcactc tcaccatccg | |
| | cagactggag | |
| | cctgaagatt ttgcagtgta ttactgtcag cagtatggta actcaccttg | |
| | gacgttcggc | |
| | caagggacca aggtggagat caaa | |
| 74. | EIVLTQSPGT LSLSLGERAT LSCRASQSVI SSFLAWYQQK | AA amino |
| | PGQAPRLLIY GASSRATGFP | acid |
| | DRFSGSGSGT DFTLTIRRLE PEDFAVYYCQ QYGNSPWTFG | sequence |
| | QGTKVEIK | |
| 75. | cagagtgtta tcagcagctt c | DNA |
| | | nucleotide |
| | | sequence |
| 76. | QSVISSF | AA amino |
| | | acid |
| | | sequence |
| 77. | ggtgcatcc | DNA |
| | | nucleotide |
| | | sequence |
| 78. | GAS | AA amino |
| | | acid |
| | | sequence |
| 79. | cagcagtatg gtaactcacc ttggacg | DNA |
| | | nucleotide |
| | | sequence |
| 80. | QQYGNSPWT | AA amino |
| | | acid |
| | | sequence |
| 81. | caggtcacct tgaaggagtc tggtcctgtg ctggtgaaac ccacagagac | DNA |
| | cctcacgctg | nucleotide |
| | acctgcaccg tctctgggtt ctcactcagc aatgctggga tgggtgtgag | sequence |
| | ctgggtccgt | |
| | cagccccctg ggaaggccct ggagtggctt gcacacattt tttcgaatga | |
| | cgagaagtcc | |
| | tacagcacat ctctgaggac cagactcacc atctccaagg acacctccaa | |
| | aagccaggtg | |
| | gtccttaccg tgaccaactt ggaccctgtg gacacagcca catatttctg | |
| | tgcacggata | |
| | ccagagttta ccagctcgtc gtgggctctc tactacttct acggtatgga | |
| | cgtctggggc | |
| | caagggacca cggtcaccgt ctcctca | |
| 82. | QVTLKESGPV LVKPTETLTL TCTVSGFSLS | AA amino |
| | NAGMGVSWVR QPPGKALEWL AHIFSNDEKS | acid |
| | YSTSLRTRLT ISKDTSKSQV VLTVTNLDPV | sequence |
| | DTATYFCARI PEFTSSSWAL YYFYGMDVWG | |
| | QGTTVTVSS | |
| 83. | gggttctcac tcagcaatgc tgggatgggt | DNA |
| | | nucleotide |
| | | sequence |
| 84. | GFSLSNAGMG | AA amino |
| | | acid |
| | | sequence |
| 85. | attttttcga atgacgagaa g | DNA |
| | | nucleotide |
| | | sequence |
| 86. | IFSNDEK | AA amino |
| | | acid |
| | | sequence |
| 87. | gcacggatac cagagtttac cagctcgtcg tgggctctct actacttcta | DNA |
| | cggtatggac | nucleotide |
| | gtc | sequence |
| 88. | ARIPEFTSSS WALYYFYGMD V | AA amino |
| | | acid |
| | | sequence |
| 89. | gaaattgtgt tgacgcagtc tccaggcacc ctgtctttgt ctccagggga | DNA |
| | aagcgccacc | nucleotide |
| | ctctcctgca gggccagtca gagtattacc agcacctact tcgcctggta | sequence |
| | ccagcagaaa | |
| | cctggccagg ctcccaggct cctcatctat gctacatcca gcagggccac | |
| | tggcgtccca | |
| | gacaggttca gtggcagtgg gtctgggacg gacttcactc tcaccatcag | |
| | cagactggag | |
| | cctgatgatt ttgcagtgta ttactgtcag caatatggta ggtcaccttg | |
| | gacgttcggc | |
| | caagggacca aggtggaagt caaa | |
| 90. | EIVLTQSPGT LSLSPGESAT LSCRASQSIT STYFAWYQQK | AA amino |
| | PGQAPRLLIY ATSSRATGVP | acid |
| | DRFSGSGSGT DFTLTISRLE PDDFAVYYCQ QYGRSPWTFG | sequence |
| | QGTKVEVK | |
| 91. | cagagtatta ccagcaccta c | DNA |
| | | nucleotide |
| | | sequence |
| 92. | QSITSTY | AA amino |
| | | acid |
| | | sequence |
| 93. | gctacatcc | DNA |
| | | nucleotide |
| | | sequence |
| 94. | ATS | AA amino |
| | | acid |
| | | sequence |
| 95. | cagcaatatg gtaggtcacc ttggacg | DNA |
| | | nucleotide |
| | | sequence |
| 96. | QQYGRSPWT | AA amino |
| | | acid |
| | | sequence |
| 97. | caggttcagc tggtgcagtc tggagctgag gtgaagaagc ctggggcctc | DNA |
| | agtgaaggtc | nucleotide |
| | tcctgcaagg cttctggtta cacctttacc agttatggta tcagctgggt | sequence |
| | gcgacaggcc | |
| | cctggacaag ggcttgagtg gatgggatgg atcagcgctt acaatgataa | |
| | cacaaactat | |
| | gcacagaagc tccagggcag agtcaccatg accgcagaca catccacgaa | |
| | tacagcctac | |
| | atggagctaa ggagcctgag atctgacgac acggccattt attactgtgt | |
| | gcgatggaat | |
| | tggggttccg tctactggta cttcgatctc tggggccgtg gcaccctggt | |
| | cactgtctcc | |
| | tca | |
| 98. | QVQLVQSGAE VKKPGASVKV SCKASGYTFT | AA amino |
| | SYGISWVRQA PGQGLEWMGW ISAYNDNTNY | acid |
| | AQKLQGRVTM TADTSTNTAY MELRSLRSDD | sequence |
| | TAIYYCVRWN WGSVYWYFDL WGRGTLVTVS | |
| | S | |
| 99. | ggttacacct ttaccagtta tggt | DNA |
| | | nucleotide |
| | | sequence |
| 100. | GYTFTSYG | AA amino |
| | | acid |
| | | sequence |
| 101. | atcagcgctt acaatgataa caca | DNA |
| | | nucleotide |
| | | sequence |
| 102. | ISAYNDNT | AA amino |
| | | acid |
| | | sequence |
| 103. | gtgcgatgga attggggttc cgtctactgg tacttcgatc tc | DNA |
| | | nucleotide |
| | | sequence |
| 104. | VRWNWGSVYW YFDL | AA amino |
| | | acid |
| | | sequence |
| 105. | gaaattgtgt tgacgcagtc tccaggcacc ctgtctttgt ctccagggga | DNA |
| | aagagccacc | nucleotide |
| | ctctcctgca gggccagtca gattattagc agcagctact ttgcctggta | sequence |
| | ccagcagaaa | |
| | cctggccagg ctcccaggct cctcatctat ggtgcgtcca gcagggccac | |
| | tggcatccca | |
| | gacaggttca gtggcagtgt gtctgggaca gacttcactc tcaccatcag | |
| | cagactggag | |
| | cctgaagatt ttgcaatgta tttctgtcag cagtatggta actcaccttg | |
| | gacgttcggc | |
| | caagggacca aggtggaaat caaa | |
| 106. | EIVLTQSPGT LSLSPGERAT LSCRASQIIS SSYFAWYQQK | AA amino |
| | PGQAPRLLIY GASSRATGIP | acid |
| | DRFSGSVSGT DFTLTISRLE PEDFAMYFCQ QYGNSPWTFG | sequence |
| | QGTKVEIK | |
| 107. | cagattatta gcagcagcta c | DNA |
| | | nucleotide |
| | | sequence |
| 108. | QIISSSY | AA amino |
| | | acid |
| | | sequence |
| 109. | ggtgcgtcc | DNA |
| | | nucleotide |
| | | sequence |
| 110. | GAS | AA amino |
| | | acid |
| | | sequence |
| 111. | cagcagtatg gtaactcacc ttggacg | DNA |
| | | nucleotide |
| | | sequence |
| 112. | QQYGNSPWT | AA amino |
| | | acid |
| | | sequence |
| 113. | cagatcacct tgaaggagtc tggtcctacg ctggtgaaac ccacacagac | DNA |
| | cctcacgctg | nucleotide |
| | acttgcacct tctctgggtt ctcactcaac actcatagag tgggtgtagg | sequence |
| | ctggatccgg | |
| | cagcccccag gaaaggccct ggagtggctt gcactcattt atgggaatga | |
| | tgttaagaac | |
| | tacagcccat ctctggagac caggctcacc atcgccaagg acacctccaa | |
| | aaaccaggtg | |
| | gtccttacaa tgaccaacat ggaccctgtg gacacagcca catatttctg | |
| | ttcgtacata | |
| | acgggggaag gaatgtactg gggccaggga accctggtca ccgtctcctc | |
| | a | |
| 114. | QITLKESGPT LVKPTQTLTL TCTFSGFSLN THRVGVGWIR | AA amino |
| | QPPGKALEWL ALIYGNDVKN | acid |
| | YSPSLETRLT IAKDTSKNQV VLTMTNMDPV DTATYFCSYI | sequence |
| | TGEGMYWGQG TLVTVSS | |
| 115. | gggttctcac tcaacactca tagagtgggt | DNA |
| | | nucleotide |
| | | sequence |
| 116. | GFSLNTHRVG | AA amino |
| | | acid |
| | | sequence |
| 117. | atttatggga atgatgttaa g | DNA |
| | | nucleotide |
| | | sequence |
| 118. | IYGNDVK | AA amino |
| | | acid |
| | | sequence |
| 119. | tcgtacataa cgggggaagg aatgtac | DNA |
| | | nucleotide |
| | | sequence |
| 120. | SYITGEGMY | AA amino |
| | | acid |
| | | sequence |
| 121. | gatgttgtga tgactcagtc tccactctcc ctgtccgtca cccttggaca | DNA |
| | gccggcctcc | nucleotide |
| | atttcctgta ggtctagtca aaacctcatg tacagtgatg gaaacaccta | sequence |
| | cttgaattgg | |
| | tttcaccaga ggccaggcca atctccaagg cgtctaattt ataaggtttc | |
| | taaccgggac | |
| | tctggggtcc cagacagatt cagcggcagt gggtcaggca ctgatttcac | |
| | actgaaaatc | |
| | agcagggtgg aggctgagga tgttggggtt tattactgca tgcaaggtac | |
| | acactggtac | |
| | acatttggcc aggggaccaa gctggagatc aaa | |
| 122. | DVVMTQSPLS LSVTLGQPAS ISCRSSQNLM | AA amino |
| | YSDGNTYLNW FHQRPGQSPR RLIYKVSNRD | acid |
| | SGVPDRFSGS GSGTDFTLKI SRVEAEDVGV | sequence |
| | YYCMQGTHWY TFGQGTKLEI K | |
| 123. | caaaacctca tgtacagtga tggaaacacc tac | DNA |
| | | nucleotide |
| | | sequence |
| 124. | QNLMYSDGNT Y | AA amino |
| | | acid |
| | | sequence |
| 125. | aaggtttct | DNA |
| | | nucleotide |
| | | sequence |
| 126. | KVS | AA amino |
| | | acid |
| | | sequence |
| 127. | atgcaaggta cacactggta caca | DNA |
| | | nucleotide |
| | | sequence |
| 128. | MQGTHWYT | AA amino |
| | | acid |
| | | sequence |
| 129. | caggtgcagc tgcagcagtg gggcgcagga ctattgaagc cttcggagac | DNA |
| | cctgtccctc | nucleotide |
| | acctgcgctg tctatggtgg gtctttcagt ggttattact ggagctggat | sequence |
| | ccgccagccc | |
| | ccagggaagg gtctggaatg gattggggaa atcaatcata gaggaaacac | |
| | caactacaac | |
| | ccgtccctca agagtcgagt caccatatca ctcgacacgt ccaagaaaca | |
| | gttctccctg | |
| | aacctgagtt ctgtgaccgc cgcggacacg gctatgtatt actgtacgag | |
| | agacgaagaa | |
| | caggaactac gtttccttga ctactggggc cagggaaccc tggtcaccgt | |
| | ctcctca | |
| 130. | QVQLQQWGAG LLKPSETLSL TCAVYGGSFS | AA amino |
| | GYYWSWIRQP PGKGLEWIGE INHRGNTNYN | acid |
| | PSLKSRVTIS LDTSKKQFSL NLSSVTAADT | sequence |
| | AMYYCTRDEE QELRFLDYWG QGTLVTVSS | |
| 131. | ggtgggtctt tcagtggtta ttac | DNA |
| | | nucleotide |
| | | sequence |
| 132. | GGSFSGYY | AA amino |
| | | acid |
| | | sequence |
| 133. | atcaatcata gaggaaacac c | DNA |
| | | nucleotide |
| | | sequence |
| 134. | INHRGNT | AA amino |
| | | acid |
| | | sequence |
| 135. | acgagagacg aagaacagga actacgtttc cttgactac | DNA |
| | | nucleotide |
| | | sequence |
| 136. | TRDEEQELRF LDY | AA amino |
| | | acid |
| | | sequence |
| 137. | gagattgtgt tgacacagtc tccagccacc ctgtctttgt ctccagggga | DNA |
| | aagagccacc | nucleotide |
| | ctctcctgca gggccagtca ggatattagc acctacttag cctggtacca | sequence |
| | acagagagct | |
| | ggccaggctc ccaggctcct catctatggt gcttccaaca gggccactgg | |
| | catcccagcc | |
| | aggttcagtg gcagtgggtc tgggacagac ttcactctca ccatcagcag | |
| | cctagagcct | |
| | gaagattttg cattttatta ctgtcaacag cgcagcaact ggccgctcac | |
| | tttcggcgga | |
| | gggaccgagg tggagatcaa a | |
| 138. | EIVLTQSPAT LSLSPGERAT LSCRASQDIS TYLAWYQQRA | AA amino |
| | GQAPRLLIYG ASNRATGIPA | acid |
| | RFSGSGSGTD FTLTISSLEP EDFAFYYCQQ RSNWPLTFGG | sequence |
| | GTEVEIK | |
| 139. | caggatatta gcacctac | DNA |
| | | nucleotide |
| | | sequence |
| 140. | QDISTY | AA amino |
| | | acid |
| | | sequence |
| 141. | ggtgcttcc | DNA |
| | | nucleotide |
| | | sequence |
| 142. | GAS | AA amino |
| | | acid |
| | | sequence |
| 143. | caacagcgca gcaactggcc gctcact | DNA |
| | | nucleotide |
| | | sequence |
| 144. | QQRSNWPLT | AA amino |
| | | acid |
| | | sequence |
| 145. | caggtgcagc tacagcagtg gggcgcagga ctgttgaagc cttcggagac | DNA |
| | cctgtccctc | nucleotide |
| | acctgcgttg tccatggtgg gtccttcagt ggttactact ggaactggat | sequence |
| | ccgccagccc | |
| | ccagggaagg ggctggagtg gattggggaa atcaatcata gaggaaacac | |
| | caactacaac | |
| | ccgtccctca agagtcgagt caccgtatca gaagacacgt ccaagaacca | |
| | gttctccctg | |
| | aagctgagct ctttgaccgc cgcggacacg gctgtgtatt actgtgtgag | |
| | aggagaggat | |
| | tacgattttt ggagtgatta ttataatgac tactggggcc agggaaccct | |
| | ggtcaccgtc | |
| | tcctca | |
| 146. | QVQLQQWGAG LLKPSETLSL TCVVHGGSFS | AA amino |
| | GYYWNWIRQP PGKGLEWIGE INHRGNTNYN | acid |
| | PSLKSRVTVS EDTSKNQFSL KLSSLTAADT | sequence |
| | AVYYCVRGED YDFWSDYYND YWGQGTLVTV | |
| | SS | |
| 147. | ggtgggtcct tcagtggtta ctac | DNA |
| | | nucleotide |
| | | sequence |
| 148. | GGSFSGYY | AA amino |
| | | acid |
| | | sequence |
| 149. | atcaatcata gaggaaacac c | DNA |
| | | nucleotide |
| | | sequence |
| 150. | INHRGNT | AA amino |
| | | acid |
| | | sequence |
| 151. | gtgagaggag aggattacga tttttggagt gattattata atgactac | DNA |
| | | nucleotide |
| | | sequence |
| 152. | VRGEDYDFWS DYYNDY | AA amino |
| | | acid |
| | | sequence |
| 153. | gaaattgtgt tgacacagtc tccagccacc ctgtctttgt ctccagggga | DNA |
| | aagagccacc | nucleotide |
| | ctctcctgca gggccagtca gactattagc agctacttag cctggcacca | sequence |
| | acagaaacct | |
| | ggccaggctc ccaggctcct catctatgat gcatccaaaa gggccacggg | |
| | catcccagcc | |
| | aggttcagtg gcagtgggtc tgggacagac ttcactctca ccatcaccag | |
| | cctagagcct | |
| | gaagattttg cagtttatta ctgtcagcag cgtagcaact ggcctctcac | |
| | tttcggcgga | |
| | gggaccaagg tggagatcaa a | |
| 154. | EIVLTQSPAT LSLSPGERAT LSCRASQTIS SYLAWHQQKP | AA amino |
| | GQAPRLLIYD ASKRATGIPA | acid |
| | RFSGSGSGTD FTLTITSLEP EDFAVYYCQQ RSNWPLTFGG | sequence |
| | GTKVEIK | |
| 155. | cagactatta gcagctac | DNA |
| | | nucleotide |
| | | sequence |
| 156. | QTISSY | AA amino |
| | | acid |
| | | sequence |
| 157. | gatgcatcc | DNA |
| | | nucleotide |
| | | sequence |
| 158. | DAS | AA amino |
| | | acid |
| | | sequence |
| 159. | cagcagcgta gcaactggcc tctcact | DNA |
| | | nucleotide |
| | | sequence |
| 160. | QQRSNWPLT | AA amino |
| | | acid |
| | | sequence |
| 161. | caggtgcagc tacagcagtg gggcgcagga ctgttgccgc cttcggagac | DNA |
| | cctgtccctc | nucleotide |
| | atctgcgctg tctatggtgg gtccttcagt ggttactact ggagctggat | sequence |
| | ccgccagccc | |
| | ccagggaagg ggctggagtg gattggggaa atcaatcata gaggaagcac | |
| | caactacaac | |
| | ccgtccctca agagtcgagc caccatatca gttgacacgt ccaagaacca | |
| | gttctccctg | |
| | aagctgagct ctgtgaccgc cgcggacacg gctgtgtatt actgttcgag | |
| | aggcgaggat | |
| | tactatgata gtagtggtta ctcgtactac tttgactact ggggccaggg | |
| | aaccctggtc | |
| | accgtctcct ca | |
| 162. | QVQLQQWGAG LLPPSETLSL ICAVYGGSFS | AA amino |
| | GYYWSWIRQP PGKGLEWIGE INHRGSTNYN | acid |
| | PSLKSRATIS VDTSKNQFSL KLSSVTAADT | sequence |
| | AVYYCSRGED YYDSSGYSYY FDYWGQGTLV | |
| | TVSS | |
| 163. | ggtgggtcct tcagtggtta ctac | DNA |
| | | nucleotide |
| | | sequence |
| 164. | GGSFSGYY | AA amino |
| | | acid |
| | | sequence |
| 165. | atcaatcata gaggaagcac c | DNA |
| | | nucleotide |
| | | sequence |
| 166. | INHRGST | AA amino |
| | | acid |
| | | sequence |
| 167. | tcgagaggcg aggattacta tgatagtagt ggttactcgt actactttga | DNA |
| | ctac | nucleotide |
| | | sequence |
| 168. | SRGEDYYDSS GYSYYFDY | AA amino |
| | | acid |
| | | sequence |
| 169. | gaaattgtgt tgacacagtc tccagccacc ctgtctttgt ctccagggga | DNA |
| | aagagccacc | nucleotide |
| | ctctcctgca gggccagtca gagtgttagc agctacttag cctggtacca | sequence |
| | acagaaacct | |
| | ggccaggctc ccaggctcct catctatgat gcatccaaca gggccactgg | |
| | catcccagcc | |
| | aggttcagtg gcagtgggtc tgggacagac ttcactctca ccatcagcag | |
| | cctagagcct | |
| | gaagattttg cagtttatta ctgtcagcag cgtagcaact ggccgctcac | |
| | tttcggcgga | |
| | gggaccaagg tggagatcaa a | |
| 170. | EIVLTQSPAT LSLSPGERAT LSCRASQSVS | AA amino |
| | SYLAWYQQKP GQAPRLLIYD ASNRATGIPA | acid |
| | RFSGSGSGTD FTLTISSLEP EDFAVYYCQQ | sequence |
| | RSNWPLTFGG GTKVEIK | |
| 171. | cagagtgtta gcagctac | DNA |
| | | nucleotide |
| | | sequence |
| 172. | QSVSSY | AA amino |
| | | acid |
| | | sequence |
| 173. | gatgcatcc | DNA |
| | | nucleotide |
| | | sequence |
| 174. | DAS | AA amino |
| | | acid |
| | | sequence |
| 175. | cagcagcgta gcaactggcc gctcact | DNA |
| | | nucleotide |
| | | sequence |
| 176. | QQRSNWPLT | AA amino |
| | | acid |
| | | sequence |
| 177. | caggtgcagc tacagcagtg gggcgcagga ctgttgaggc cttcggagac | DNA |
| | cctgtccctc | nucleotide |
| | acctgcgctg tctatggtgg gtccttcagt ggttactact ggaattggat | sequence |
| | ccgccagtcc | |
| | ccagggacgg ggctggagtg gattggggaa atcaatcata gagggaacat | |
| | caacttcaac | |
| | ccgtccctca agagtcgagt caccatatca gaggacacgt ccaaaaacca | |
| | attctccctg | |
| | aggctgaact ctgtgaccgc cgcggacacg gctgtgtatt actgtgcgag | |
| | aggagaggat | |
| | tacgatattt ggagtggtta ttatagggag tactggggcc agggaaccct | |
| | ggtcaccgtc | |
| | tcctca | |
| 178. | QVQLQQWGAG LLRPSETLSL TCAVYGGSFS | AA amino |
| | GYYWNWIRQS PGTGLEWIGE INHRGNINFN | acid |
| | PSLKSRVTIS EDTSKNQFSL RLNSVTAADT | sequence |
| | AVYYCARGED YDIWSGYYRE YWGQGTLVTV | |
| | SS | |
| 179. | ggtgggtcct tcagtggtta ctac | DNA |
| | | nucleotide |
| | | sequence |
| 180. | GGSFSGYY | AA amino |
| | | acid |
| | | sequence |
| 181. | atcaatcata gagggaacat c | DNA |
| | | nucleotide |
| | | sequence |
| 182. | INHRGNI | AA amino |
| | | acid |
| | | sequence |
| 183. | gcgagaggag aggattacga tatttggagt ggttattata gggagtac | DNA |
| | | nucleotide |
| | | sequence |
| 184. | ARGEDYDIWS GYYREY | AA amino |
| | | acid |
| | | sequence |
| 185. | gaaattgtgt tgacacagtc tccagccacc ctgtctttgt ctccagggga | DNA |
| | aagagccact | nucleotide |
| | ctctcctgca gggccagtca gagtgttagc agctacttag cctggtacca | sequence |
| | gcagaaacct | |
| | ggccaggctc ccaggctcct catctatgat gcatccaaga gggccactgg | |
| | catcccagcc | |
| | aggttcagtg gcagtgggtc tgggacagac ttcactctca ccatcagcag | |
| | cctagagcct | |
| | gaagattttg ctgtttatta ctgtcagcag cgtagcaact ggcctctcgc | |
| | tttcggcgga | |
| | gggaccaagg tggagatcaa a | |
| 186. | EIVLTQSPAT LSLSPGERAT LSCRASQSVS | AA amino |
| | SYLAWYQQKP GQAPRLLIYD ASKRATGIPA | acid |
| | RFSGSGSGTD FTLTISSLEP EDFAVYYCQQ | sequence |
| | RSNWPLAFGG GTKVEIK | |
| 187. | cagagtgtta gcagctac | DNA |
| | | nucleotide |
| | | sequence |
| 188. | QSVSSY | AA amino |
| | | acid |
| | | sequence |
| 189. | gatgcatcc | DNA |
| | | nucleotide |
| | | sequence |
| 190. | DAS | AA amino |
| | | acid |
| | | sequence |
| 191. | cagcagcgta gcaactggcc tctcgct | DNA |
| | | nucleotide |
| | | sequence |
| 192. | QQRSNWPLA | AA amino |
| | | acid |
| | | sequence |
| 193. | caggtgcagc tacagcagtg gggcgcagga ctgttgaagc cttcggagac | DNA |
| | cctgtccctc | nucleotide |
| | acctgcgctg tctatggtgg gtccttcagt gagttctact ggaactggat | sequence |
| | ccgccagccc | |
| | ccagagaagg gcctggagtg gattggggaa atcaatcatc gtggaaacac | |
| | caactacaac | |
| | ccgtccctca agagtcgagt caccatatca gtagacatgt ccaagaacca | |
| | gttctccctg | |
| | cagctgaact ctgtgaccgt cgcggacacg gctctgtatt actgtgcgtt | |
| | tggctacgat | |
| | tttcggagtt cttatgagga cgtctggggc caagggacca cggtcaccgt | |
| | ctcctca | |
| 194. | QVQLQQWGAG LLKPSETLSL TCAVYGGSFS | AA amino |
| | EFYWNWIRQP PEKGLEWIGE INHRGNTNYN 60 | acid |
| | PSLKSRVTIS VDMSKNQFSL QLNSVTVADT | sequence |
| | ALYYCAFGYD FRSSYEDVWG QGTTVTVSS | |
| 195. | ggtgggtcct tcagtgagtt ctac | DNA |
| | | nucleotide |
| | | sequence |
| 196. | GGSFSEFY | AA amino |
| | | acid |
| | | sequence |
| 197. | atcaatcatc gtggaaacac c | DNA |
| | | nucleotide |
| | | sequence |
| 198. | INHRGNT | AA amino |
| | | acid |
| | | sequence |
| 199. | gcgtttggct acgattttcg gagttcttat gaggacgtc | DNA |
| | | nucleotide |
| | | sequence |
| 200. | AFGYDFRSSY EDV | AA amino |
| | | acid |
| | | sequence |
| 201. | gaaattgtgt tgacacagtc tccagccacc ctgtctttgt ctccagggga | DNA |
| | aagagccacc | nucleotide |
| | ctctcctgca gggccagtca ggatattagc acctacttag cctggcacca | sequence |
| | acagaaacct | |
| | ggccagcctc ccaggctcct catctatggt tcatccaaca gggccactgg | |
| | catcccagcc | |
| | aggttcagtg gcagtgggtc tgggacagac ttcactctca ccatcagcag | |
| | cctagagcct | |
| | gaagattttg cagtttatta ctgtcagcag cgtagcaact ggcctctcac | |
| | tttcggcgga | |
| | gggaccaagg tggagatcaa a | |
| 202. | EIVLTQSPAT LSLSPGERAT LSCRASQDIS TYLAWHQQKP | AA amino |
| | GQPPRLLIYG SSNRATGIPA | acid |
| | RFSGSGSGTD FTLTISSLEP EDFAVYYCQQ | sequence |
| | RSNWPLTFGG GTKVEIK | |
| 203. | caggatatta gcacctac | DNA |
| | | nucleotide |
| | | sequence |
| 204. | QDISTY | AA amino |
| | | acid |
| | | sequence |
| 205. | ggttcatcc | DNA |
| | | nucleotide |
| | | sequence |
| 206. | GSS | AA amino |
| | | acid |
| | | sequence |
| 207. | cagcagcgta gcaactggcc tctcact | DNA |
| | | nucleotide |
| | | sequence |
| 208. | QQRSNWPLT | AA amino |
| | | acid |
| | | sequence |
| 209. | gaggtgcagc tgttggagtc tgggggaggc ttggtacagc cgggggggtc | DNA |
| | cctgagactc | nucleotide |
| | tcctgtgcag cctctggatt caccttcaga agctatgcca tgagttgggt | sequence |
| | ccgccaggct | |
| | ccagggaagg ggctggagtg ggtctcagtt attagtggtg gtggtggtag | |
| | gacatactac | |
| | acagactccg tgaagggccg gttcaccatc tccagagaca attccaagag | |
| | catgctgtat | |
| | ctgcaaatga acagcctgag agccgaggac acggccattt attactgtgc | |
| | gaaagagagg | |
| | gtaactggaa tagaccacta ctactacggt gtggacgtct ggggccaagg | |
| | gaccacggtc | |
| | accgtctcct ca | |
| 210. | EVQLLESGGG LVQPGGSLRL SCAASGFTFR | AA amino |
| | SYAMSWWRQA PGKGLEWVSV ISGGGGRTYY | acid |
| | TDSVKGRFTI SRDNSKSMLY LQMNSLRAED | sequence |
| | TAIYYCAKER VTGIDHYYYG VDVWGQGTTV | |
| | TVSS | |
| 211. | ggattcacct tcagaagcta tgcc | DNA |
| | | nucleotide |
| | | sequence |
| 212. | GFTFRSYA | AA amino |
| | | acid |
| | | sequence |
| 213. | attagtggtg gtggtggtag gaca | DNA |
| | | nucleotide |
| | | sequence |
| 214. | ISGGGGRT | AA amino |
| | | acid |
| | | sequence |
| 215. | gcgaaagaga gggtaactgg aatagaccac tactactacg gtgtggacgt | DNA |
| | c | nucleotide |
| | | sequence |
| 216. | AKERVTGIDH YYYGVDV | AA amino |
| | | acid |
| | | sequence |
| 217. | gacatccaga tgacccagtc tccatcctcc ctgtctgcat ctgtaggaga | DNA |
| | cagagtcacc | nucleotide |
| | atcacttgcc gggcaagtca gagcattagt agctatttaa attggtatca | sequence |
| | gcagaaacca | |
| | gggaaagccc ctaagctcct gatctatgct acatccagtt tgcaaagtgg | |
| | ggtcccatca | |
| | cggttcagtg gcagtgcatc tggaacagat ttcactctcg ccatcagcag | |
| | tctgcaacct | |
| | gaagattttg caacttacta ctgtcaacag agttacacta cccccctcac | |
| | tttcggcgga | |
| | gggaccaagg tggagatcaa a | |
| 218. | DIQMTQSPSS LSASVGDRVT ITCRASQSIS | AA amino |
| | SYLNWYQQKP GKAPKLLIYA TSSLQSGVPS | acid |
| | RFSGSASGTD FTLAISSLQP EDFATYYCQQ | sequence |
| | SYTTPLTFGG GTKVEIK | |
| 219. | cagagcatta gtagctat | DNA |
| | | nucleotide |
| | | sequence |
| 220. | QSISSY | AA amino |
| | | acid |
| | | sequence |
| 221. | gctacatcc | DNA |
| | | nucleotide |
| | | sequence |
| 222. | ATS | AA amino |
| | | acid |
| | | sequence |
| 223. | caacagagtt acactacccc cctcact | DNA |
| | | nucleotide |
| | | sequence |
| 224. | QQSYTTPLT | AA amino |
| | | acid |
| | | sequence |
| 225. | gaggtgcagc tggtggagtc tgggggaggc ttggtacaac ctggagggtc | DNA |
| | cctgagactt | nucleotide |
| | tcctgtgcag cctctggatt tacattcagc agttatgaaa tgaactgggt | sequence |
| | ccgccaggct | |
| | ccagggaagg ggctggagtg ggtttcatat atcagtagta gtggtaatac | |
| | caaagactac | |
| | gcaggctctg tgaagggccg agtcaccatc tccagagaca acgccaagaa | |
| | cttactgtat | |
| | ctgcaaatga acagcctgag agccgaggac acggctgttt atcactgtgc | |
| | gagagatgga | |
| | gggcattacg atattttgac tggttccatg tcctactact actacgcttt | |
| | ggacgtctgg | |
| | ggccaaggga ccacggtcac cgtctcctca | |
| 226. | VQLVESGGG LVQPGGSLRL SCAASGFTFS | AA amino |
| | SYEMNWVRQA PGKGLEWVSY ISSSGNTKDY | acid |
| | AGSVKGRVTI SRDNAKNLLY LQMNSLRAED | sequence |
| | TAVYHCARDG GHYDILTGSM SYYYYALDVW | |
| | GQGTTVTVSS | |
| 227. | ggatttacat tcagcagtta tgaa | DNA |
| | | nucleotide |
| | | sequence |
| 228. | GFTFSSYE | AA amino |
| | | acid |
| | | sequence |
| 229. | atcagtagta gtggtaatac caaa | DNA |
| | | nucleotide |
| | | sequence |
| 230. | ISSSGNTK | AA amino |
| | | acid |
| | | sequence |
| 231. | gcgagagatg gagggcatta cgatattttg actggttcca tgtcctacta | DNA |
| | ctactacgct | nucleotide |
| | ttggacgtc | sequence |
| 232. | ARDGGHYDIL TGSMSYYYYA LDV | AA amino |
| | | acid |
| | | sequence |
| 233. | gacatccaga tgacccagtc tccatcctcc ctgtctgcat ctgtaggaga | DNA |
| | cagagtcacc | nucleotide |
| | atcacttgcc gggcaagtca gagcattagc agctatttaa attggtatca | sequence |
| | gcagaaacca | |
| | gggaaagccc ctaagctcct gatctatgct gcatccagtt tgcaaagtgg | |
| | ggtcccgtca | |
| | aggttcagtg gcagtggatc tgggacagat ttcactctca ccatcagcag | |
| | tctgcaacct | |
| | gaagattttg caacttacta ctgtcaacag agttacagta cccctccgat | |
| | caccttcggc | |
| | caagggacac gactggagat taaa | |
| 234. | DIQMTQSPSS LSASVGDRVT ITCRASQSIS | AA amino |
| | SYLNWYQQKP GKAPKLLIYA ASSLQSGVPS | acid |
| | RFSGSGSGTD FTLTISSLQP EDFATYYCQQ | sequence |
| | SYSTPPITFG QGTRLEIK | |
| 235. | cagagcatta gcagctat | DNA |
| | | nucleotide |
| | | sequence |
| 236 | QSISSY | AA amino |
| | | acid |
| | | sequence |
| 237. | gctgcatcc | DNA |
| | | nucleotide |
| | | sequence |
| 238. | AAS | AA amino |
| | | acid |
| | | sequence |
| 239. | caacagagtt acagtacccc tccgatcacc | DNA |
| | | nucleotide |
| | | sequence |
| 240. | QQSYSTPPIT | AA amino |
| | | acid |
| | | sequence |
| 241. | gaggtgcagc tggtggagtc tgggggaggc ttggtacagc ctggggggtc | DNA |
| | cctgagactc | nucleotide |
| | tcctgtgcag cctctggatt cacctttaaa acctatgcca tgagctgggt | sequence |
| | ccgccaggct | |
| | ccagggaggg ggctggagtg ggtctcaggt attagtggta gtggtagtac | |
| | ctcatactac | |
| | gcagactccg tgaagggccg gttcaccatc tccagagaca attacaagaa | |
| | gacgctgtct | |
| | ctgcaaatga acagtctgag agccgaggac acggccgttt attactgtgc | |
| | gctggatata | |
| | atggcaacgg taggaggtct ctttaacaac tggggccagg gaaccctggt | |
| | caccgtctcc | |
| | tca | |
| 242. | EVQLVESGGG LVQPGGSLRL SCAASGFTFK | AA amino |
| | TYAMSWVRQA PGRGLEWVSG ISGSGSTSYY | acid |
| | ADSVKGRFTI SRDNYKKTLS LQMNSLRAED | sequence |
| | TAVYYCALDI MATVGGLFNN WGQGTLVTVS | |
| | S | |
| 243. | ggattcacct ttaaaaccta tgcc | DNA |
| | | nucleotide |
| | | sequence |
| 244. | GFTFKTYA | AA amino |
| | | acid |
| | | sequence |
| 245. | attagtggta gtggtagtac ctca | DNA |
| | | nucleotide |
| | | sequence |
| 246. | ISGSGSTS | AA amino |
| | | acid |
| | | sequence |
| 247. | gcgctggata taatggcaac ggtaggaggt ctctttaaca ac | DNA |
| | | nucleotide |
| | | sequence |
| 248. | ALDIMATVGG LFNN | AA amino |
| | | acid |
| | | sequence |
| 249. | aaattgtgt tgacgcagtc tccaggcacc ctgtctttgt ctccagggga | DNA |
| | aagagccacc | nucleotide |
| | ctctcctgca gggccagtca gagtgttagc agcagctact tagcctggta | sequence |
| | ccagcagaaa | |
| | cctggccagg ctcccaggct cctcatctat ggtgcatcca gcagggccac | |
| | tggcatccca | |
| | gacaggttca gtggcagtgg gtctgggaca gacttcactc tcaccatcag | |
| | cagactggag | |
| | cctgaagatt ttgcagtgta ttactgtcag cagtatggta gctcaccttg | |
| | gacgttcggc | |
| | caagggacca aggtggaaat caaa | |
| 250. | EIVLTQSPGT LSLSPGERAT LSCRASQSVS | AA amino |
| | SSYLAWYQQK PGQAPRLLIY GASSRATGIP | acid |
| | DRFSGSGSGT DETLTISRLE PEDFAVYYCQ | sequence |
| | QYGSSPWTFG QGTKVEIK | |
| 251. | cagagtgtta gcagcagcta c | DNA |
| | | nucleotide |
| | | sequence |
| 252. | QSVSSSY | AA amino |
| | | acid |
| | | sequence |
| 253. | ggtgcatcc | DNA |
| | | nucleotide |
| | | sequence |
| 254. | GAS | AA amino |
| | | acid |
| | | sequence |
| 255. | cagcagtatg gtagctcacc ttggacg | DNA |
| | | nucleotide |
| | | sequence |
| 256. | QQYGSSPWT | AA amino |
| | | acid |
| | | sequence |
| 257. | aggtgcagc tggtgcagtc tggggctgag gtgaagaagc ctgggtcctc | DNA |
| | ggtgaaggtc | nucleotide |
| | tcctgcaagg cttctggagg caccttcagc agacatacta tcagctgggt | sequence |
| | gcgacaggcc | |
| | cctggacaag ggcttgagtg gatgggaggg atcatcccta tctttggtac | |
| | agcaaactac | |
| | gcacacaagt tccagggcag agtcacgatt accacggacg aatccacgag | |
| | cacagcctac | |
| | atggagctga gcagcctgag atctgaggac acggccgtat attattgtgc | |
| | gagagcccct | |
| | tatacccgac aggggtactt cgatctctgg ggccgtggca ccctggtcac | |
| | cgtctcctca | |
| 258. | QVQLVQSGAE VKKPGSSVKV SCKASGGTFS | AA amino |
| | RHTISWVRQA PGQGLEWMGG IIPIFGTANY | acid |
| | AHKFQGRVTI TTDESTSTAY MELSSLRSED | sequence |
| | TAVYYCARAP YTRQGYFDLW GRGTLVTVSS | |
| 259. | ggaggcacct tcagcagaca tact | DNA |
| | | nucleotide |
| | | sequence |
| 260 | GGTFSRHT | AA amino |
| | | acid |
| | | sequence |
| 261. | atcatcccta tctttggtac agca | DNA |
| | | nucleotide |
| | | sequence |
| 262. | IIPIFGTA | AA amino |
| | | acid |
| | | sequence |
| 263. | gcgagagccc cttatacccg acaggggtac ttcgatctc | DNA |
| | | nucleotide |
| | | sequence |
| 264. | ARAPYTRQGY FDL | AA amino |
| | | acid |
| | | sequence |
| 265. | gacatcgtga tgacccagtc tccagactcc ctggctgtgt ctctgggcga | DNA |
| | gagggccacc | nucleotide |
| | atcaactgca agtccagcca gagtgtttta tacagctcca acaataagaa | sequence |
| | ctacttagct | |
| | tggtaccagc agaaaccagg acagcctcct aagctactca tttactgggc | |
| | atctacccgg | |
| | gaatccgggg tccctgaccg attcagtggc agcgggtctg ggacagattt | |
| | cactctcacc | |
| | atcagcagcc tgcaggctga agatgtggca gtttattact gtcagcaaga | |
| | ttatagtact | |
| | ccgtggacgt tcggccaagg gaccaaggtg gaaatcaaa | |
| 266. | DIVMTQSPDS LAVSLGERAT INCKSSQSVL YSSNNKNYLA | AA amino |
| | WYQQKPGQPP KLLIYWASTR | acid |
| | ESGVPDRFSG SGSGTDFTLT ISSLQAEDVA VYYCQQDYST | sequence |
| | PWTFGQGTKV EIK | |
| 267. | cagagtgttt tatacagctc caacaataag aactac | DNA |
| | | nucleotide |
| | | sequence |
| 268. | QSVLYSSNNK NY | AA amino |
| | | acid |
| | | sequence |
| 269. | tgggcatct | DNA |
| | | nucleotide |
| | | sequence |
| 270. | WAS | AA amino |
| | | acid |
| | | sequence |
| 271. | cagcaagatt atagtactcc gtggacg | DNA |
| | | nucleotide |
| | | sequence |
| 272. | QQDYSTPWT | AA amino |
| | | acid |
| | | sequence |
| 273. | caggtgcagc tggtgcagtc tggggctgag gtgaagaagc ctggggcctc | DNA |
| | agtgaaggtt | nucleotide |
| | tcctgcaagg catctggata caccttcacc aactactata tacactgggt | sequence |
| | gcgacaggcc | |
| | cctggacaag ggcttgactg gatgggaatt atcaaccctg gtggtggtaa | |
| | cacaaactac | |
| | gcacagaagt tcctgggcag agtcaccatg accagggaca cgtccacgac | |
| | cacagtctac | |
| | atggagctga gcagcctgag atctgaggac acggccatat attactgtgc | |
| | gagagaaaac | |
| | tggaactctt actttgacaa ctggggccag ggaaccctgg tcaccgtctc | |
| | ctca | |
| 274. | QVQLVQSGAE VKKPGASVKV SCKASGYTFT | AA amino |
| | NYYIHWVRQA PGQGLDWMGI INPGGGNTNY 60 | acid |
| | AQKFLGRVTM TRDTSTTTVY MELSSLRSED | sequence |
| | TAIYYCAREN WNSYFDNWGQ GTLVTVSS | |
| 275. | ggatacacct tcaccaacta ctat | DNA |
| | | nucleotide |
| | | sequence |
| 276. | GYTFTNYY | AA amino |
| | | acid |
| | | sequence |
| 277. | atcaaccctg gtggtggtaa caca | DNA |
| | | nucleotide |
| | | sequence |
| 278. | INPGGGNT | AA amino |
| | | acid |
| | | sequence |
| 279. | gcgagagaaa actggaactc ttactttgac aac | DNA |
| | | nucleotide |
| | | sequence |
| 280. | ARENWNSYFD N | AA amino |
| | | acid |
| | | sequence |
| 281. | gacatcgtga tgacccagtc tccagactcc ctggctgtgt ctctgggcga | DNA |
| | gagggccacc | nucleotide |
| | atcaactgca agtccagcca gagtgtttta tacagctcca acaataagaa | sequence |
| | cttcttagct | |
| | tggtaccagc agaaaccagg acagcctcct aagctgctca tttactgggc | |
| | atctacccgg | |
| | gaatccgggg tccctgaccg attcagtggc agcgggtctg ggacagattt | |
| | cactctcacc | |
| | atcagcagcc tgcaggctga agatgtggca ctttattact gtcagcaata | |
| | ttatggtgct | |
| | ccgtggacgt tcggccaagg gaccaaggtg gaaatcaaa | |
| 282. | DIVMTQSPDS LAVSLGERAT INCKSSQSVL YSSNNKNFLA | AA amino |
| | WYQQKPGQPP KLLIYWASTR | acid |
| | ESGVPDRFSG SGSGTDFTLT ISSLQAEDVA LYYCQQYYGA | sequence |
| | PWTFGQGTKV EIK | |
| 283. | cagagtgttt tatacagctc caacaataag aacttc | DNA |
| | | nucleotide |
| | | sequence |
| 284. | QSVLYSSNNK NF | AA amino |
| | | acid |
| | | sequence |
| 285. | tgggcatct | DNA |
| | | nucleotide |
| | | sequence |
| 286. | WAS | AA amino |
| | | acid |
| | | sequence |
| 287. | cagcaatatt atggtgctcc gtggacg | DNA |
| | | nucleotide |
| | | sequence |
| 288. | QQYYGAPWT | AA amino |
| | | acid |
| | | sequence |
| 289. | caggtccagc tggtgcagtc tggggctgag gtgaagaagc ctgggtcctc | DNA |
| | ggtgaaggtc | nucleotide |
| | tcctgcaagg cttctggagg caccttcagc agctatacta tcaactgggt | sequence |
| | gcgacaggcc | |
| | cctggacaag ggcttgagtg gatgggaggg atcatcccta tctttggtat | |
| | agcaaactac | |
| | gcacagaagt tccagggcag agtcacgatt accacggacg aatccacgaa | |
| | cacagcctac | |
| | atggagctga gcagcctgag atctgaggac acggccattt attactgtgc | |
| | gagagcgaga | |
| | tatggttcgg ggagttatga ctactggggc cagggaaccc tggtcaccgt | |
| | ctcctca | |
| 290. | QVQLVQSGAE VKKPGSSVKV SCKASGGTFS | AA amino |
| | SYTINWWRQA PGQGLEWMGG IIPIFGIANY | acid |
| | AQKFQGRVTI TTDESTNTAY MELSSLRSED | sequence |
| | TAIYYCARAR YGSGSYDYWG QGTLVTVSS | |
| 291. | ggaggcacct tcagcagcta tact | DNA |
| | | nucleotide |
| | | sequence |
| 292. | GGTFSSYT | AA amino |
| | | acid |
| | | sequence |
| 293. | atcatcccta tctttggtat agca | DNA |
| | | nucleotide |
| | | sequence |
| 294. | IIPIFGIA | AA amino |
| | | acid |
| | | sequence |
| 295. | gcgagagcga gatatggttc ggggagttat gactac | DNA |
| | | nucleotide |
| | | sequence |
| 296. | ARARYGSGSY DY | AA amino |
| | | acid |
| | | sequence |
| 297. | gacatcgtga tgacccagtc tccagactcc ctggctgtgt ctctgggcga | DNA |
| | gagggccacc | nucleotide |
| | atcaactgca agtccagcca gagtgtttta tacacctcca acaataagaa | sequence |
| | ctacttagct | |
| | tggtaccagc agaaaccagg acagcctcct aagctgctca tttactgggc | |
| | atctacccgg | |
| | gaatccgggg tccctgaccg attcagtggc agcgggtctg ggacagattt | |
| | cactctcacc | |
| | atcagcagcc tgcaggctga agatgtggca gtttattact gtcagcaata | |
| | ttataatact | |
| | ccatggacgt tcggccaagg gaccaaggtg gaaatcaaa | |
| 298. | DIVMTQSPDS LAVSLGERAT INCKSSQSVL YTSNNKNYLA | AA amino |
| | WYQQKPGQPP KLLIYWASTR | acid |
| | ESGVPDRFSG SGSGTDFTLT ISSLQAEDVA VYYCQQYYNT | Sequence |
| | PWTFGQGTKV EIK | |
| 299. | cagagtgttt tatacacctc caacaataag aactac | DNA |
| | | nucleotide |
| | | sequence |
| 300. | QSVLYTSNNK NY | AA amino |
| | | acid |
| | | sequence |
| 301. | tgggcatct | DNA |
| | | nucleotide |
| | | sequence |
| 302. | WAS | AA amino |
| | | acid |
| | | sequence |
| 303. | cagcaatatt ataatactcc atggacg | DNA |
| | | nucleotide |
| | | sequence |
| 304. | QQYYNTPWT | AA amino |
| | | acid |
| | | sequence |
| 305. | cagatcacct tgaaggagtc tggtcctacg ctggtgaaac ccacacagac | DNA |
| | cctcacgctg | nucleotide |
| | acctgcacct tctctgggtt ctcactcagc actaatggag tgggtgtggg | sequence |
| | ctggatccgt | |
| | cagcccccag gaaaggccct ggagtggctt ggaatcattt attggaatga | |
| | tgataagcgc | |
| | tacagcccat ctctgaggag cagactcacc atcaccaagg acacctccaa | |
| | aaaccaggtg | |
| | gtccttacaa tgaccaacat ggaccctgtg gacacagcca catattactg | |
| | tgcacacaga | |
| | ggcctcttcg gaggttggtt cgacccctgg ggccagggaa ccctggtcac | |
| | cgtctcctca | |
| 306. | QITLKESGPT LVKPTQTLTL TCTFSGFSLS TNGVGVGWIR | AA amino |
| | QPPGKALEWL GIIYWNDDKR | acid |
| | YSPSLRSRLT ITKDTSKNQV VLTMTNMDPV DTATYYCAHR | sequence |
| | GLFGGWFDPW GQGTLVTVSS | |
| 307. | gggttctcac tcagcactaa tggagtgggt | DNA |
| | | nucleotide |
| | | sequence |
| 308. | GFSLSTNGVG | AA amino |
| | | acid |
| | | sequence |
| 309. | atttattgga atgatgataa g | DNA |
| | | nucleotide |
| | | sequence |
| 310. | IYWNDDK | AA amino |
| | | acid |
| | | sequence |
| 311. | gcacacagag gcctcttcgg aggttggttc gacccc | DNA |
| | | nucleotide |
| | | sequence |
| 312. | AHRGLFGGWF DP | AA amino |
| | | acid |
| | | sequence |
| 313. | gacatccaga tgacccagtc tccatcctcc ctgtctgcat ctgtaggaga | DNA |
| | cagagtcacc | nucleotide |
| | atcacttgcc gggcaagtca gagcattagc aggtatttaa attggtatca | sequence |
| | gcagaaacca | |
| | gggaaagccc ctaacctcct gatctttgct gcatccagtt tgcaaagtgg | |
| | ggtcccatca | |
| | aggttcagtg gcagtggatc tgggacagat ttcactctca ccatcagcag | |
| | tctgcaacct | |
| | gaagattttg caacttactt ctgtcaacag agttacaata ccccgctcac | |
| | tttcggcgga | |
| | gggaccaagg tggagatcaa a | |
| 314. | DIQMTQSPSS LSASVGDRVT ITCRASQSIS | AA amino |
| | RYLNWYQQKP GKAPNLLIFA ASSLQSGVPS | acid |
| | RFSGSGSGTD FTLTISSLQP EDFATYFCQQ | sequence |
| | SYNTPLTFGG GTKVEIK | |
| 315. | cagagcatta gcaggtat | DNA |
| | | nucleotide |
| | | sequence |
| 316. | QSISRY | AA amino |
| | | acid |
| | | sequence |
| 317. | gctgcatcc | DNA |
| | | nucleotide |
| | | sequence |
| 318. | AAS | AA amino |
| | | acid |
| | | sequence |
| 319. | caacagagtt acaatacccc gctcact | DNA |
| | | nucleotide |
| | | sequence |
| 320. | QQSYNTPLT | AA amino |
| | | acid |
| | | sequence |
| 321. | gaggtgcagc tggtggagtc tgggggaggc ttggtacagc cgggggggtc | DNA |
| | cctgagactc | nucleotide |
| | tcctgtgcaa tctctggatt cacctttagg agttatgcca tgacctgggt | sequence |
| | ccgccaggct | |
| | ccagggaagg cgctggagtg ggtctcagtt attagtggta gcggtggtaa | |
| | cacatactac | |
| | gcagactccg tgaagggccg gttcaccgtc tccagagaca attccaggaa | |
| | cacgctgtat | |
| | ctgcaaatga acagcctgag agccgaggac acggccgtat atttctgttc | |
| | gaaagttgca | |
| | gcagctaata attactatta cgctttggac gtctggggcc aagggaccac | |
| | ggtcaccgtc | |
| | tcctca | |
| 322. | EVQLVESGGG LVQPGGSLRL SCAISGFTFR | AA amino |
| | SYAMTWVRQA PGKALEWVSV ISGSGGNTYY | acid |
| | ADSVKGRFTV SRDNSRNTLY LQMNSLRAED | sequence |
| | TAVYFCSKVA AANNYYYALD VWGQGTTVTV | |
| | SS | |
| 323. | ggattcacct ttaggagtta tgcc | DNA |
| | | nucleotide |
| | | sequence |
| 324. | GFTFRSYA | AA amino |
| | | acid |
| | | sequence |
| 325. | attagtggta gcggtggtaa caca | DNA |
| | | nucleotide |
| | | sequence |
| 326. | ISGSGGNT | AA amino |
| | | acid |
| | | sequence |
| 327. | tcgaaagttg cagcagctaa taattactat tacgctttgg acgtc | DNA |
| | | nucleotide |
| | | sequence |
| 328. | SKVAAANNYY YALDV | AA amino |
| | | acid |
| | | sequence |
| 329. | gatattgtga tgactcagtc tccactctcc ctgcccgtca cccctggaga | DNA |
| | gccggcctcc | nucleotide |
| | atctcctgca ggtctagtca gagcctcctg catagtaatg gatacaagta | sequence |
| | tttggattgg | |
| | tacctgcaga agccagggca gtctccacaa ctcctgatct atttggtttc | |
| | taatcgggcc | |
| | tccggggtcc ctgacaggtt cagtggcagt ggatcaggca cagattttac | |
| | actgaaaatc | |
| | agcagagtgg aggctgagga tgttggggtt tattattgca tgcaagctct | |
| | acaaactccg | |
| | tacacttttg gccaggggac caagctggag atcaaa | |
| 330. | DIVMTQSPLS LPVTPGEPAS ISCRSSQSLL | AA amino |
| | HSNGYKYLDW YLQKPGQSPQ LLIYLVSNRA | acid |
| | SGVPDRFSGS GSGTDFTLKI SRVEAEDVGV | sequence |
| | YYCMQALQTP YTFGQGTKLE IK | |
| 331. | cagagcctcc tgcatagtaa tggatacaag tat | DNA |
| | | nucleotide |
| | | sequence |
| 332. | QSLLHSNGYK Y | AA amino |
| | | acid |
| | | sequence |
| 333. | ttggtttct | DNA |
| | | nucleotide |
| | | sequence |
| 334. | LVS | AA amino |
| | | acid |
| | | sequence |
| 335. | atgcaagctc tacaaactcc gtacact | DNA |
| | | nucleotide |
| | | sequence |
| 336. | MQALQTPYT | AA amino |
| | | acid |
| | | sequence |
| 337. | caggtgcagc tggtggagtc tgggggaggc gtggtccagc ctgggaggtc | DNA |
| | cctgagactc | nucleotide |
| | tcctgtgtag cgtctggatt caccttcagt aactatggca tgcactgggt | sequence |
| | ccgccaggct | |
| | ccaggcaagg ggctggagtg ggtggcagtt atatggaatg atggaagtaa | |
| | taaatactat | |
| | gcagactccg tgaagggccg attcaccatc tccagagaca attccaagaa | |
| | cacgctgtat | |
| | ctccaagtga gcagcctgag agccgatgac acggctgtat attactgtgc | |
| | gagggacgga | |
| | gaggtcgaat atagcagctc gaattacaac tactacggtc tggatgtctg | |
| | gggccaaggg | |
| | accacggtca ccgtctcctc a | |
| 338. | QVQLVESGGG VVQPGRSLRL SCVASGFTFS | AA amino |
| | NYGMHWVRQA PGKGLEWVAV IWNDGSNKYY | acid |
| | ADSVKGRFTI SRDNSKNTLY LQVSSLRADD | sequence |
| | TAVYYCARDG EVEYSSSNYN YYGLDVWGQG | |
| | TTVTVSS | |
| 339. | ggattcacct tcagtaacta tggc | DNA |
| | | nucleotide |
| | | sequence |
| 340. | GFTFSNYG | AA amino |
| | | acid |
| | | sequence |
| 341. | atatggaatg atggaagtaa taaa | DNA |
| | | nucleotide |
| | | sequence |
| 342. | IWNDGSNK | AA amino |
| | | acid |
| | | sequence |
| 343. | gcgagggacg gagaggtcga atatagcagc tcgaattaca actactacgg | DNA |
| | tctggatgtc | nucleotide |
| | | sequence |
| 344. | ARDGEVEYSS SNYNYYGLDV | AA amino |
| | | acid |
| | | sequence |
| 345. | gacatccaga tgacccagtc tccatcctcc ctgtctgcat ctgtaggaga | DNA |
| | cagagtcacc | nucleotide |
| | atcacttgcc aggcgagtca ggacattagc aactatttaa attggtatca | sequence |
| | gcagaaacca | |
| | gggaaagccc ctaaactcct gatctacgat gcatccaatt tggaaacagg | |
| | ggtcccatca | |
| | aggttcagtg gaagtggatc tgggacagat tttactttca ccatcagcag | |
| | cctgcagcct | |
| | gaagatattg taacatatta ctgtcaacag tatgatgatc tcccgatcac | |
| | cttcggccaa | |
| | gggacacgac tcgagattaa a | |
| 346. | DIQMTQSPSS LSASVGDRVT ITCQASQDIS | AA amino |
| | NYLNWYQQKP GKAPKLLIYD ASNLETGVPS | acid |
| | RFSGSGSGTD FTFTISSLQP EDIVTYYCQQ | sequence |
| | YDDLPITFGQ GTRLEIK | |
| 347. | caggacatta gcaactat | DNA |
| | | nucleotide |
| | | sequence |
| 348. | QDISNY | AA amino |
| | | acid |
| | | sequence |
| 349. | gatgcatcc | DNA |
| | | nucleotide |
| | | sequence |
| 350. | DAS | AA amino |
| | | acid |
| | | sequence |
| 351. | caacagtatg atgatctccc gatcacc | DNA |
| | | nucleotide |
| | | sequence |
| 352. | QQYDDLPIT | AA amino |
| | | acid |
| | | sequence |
| 353. | gaggtgcagc tggtggagtc tgggggaggc ttggtacagc ctggggggtc | DNA |
| | cctgagactc | nucleotide |
| | tcctgtgcag cctctggatt ctcctttcat aattttgcca tgaactgggt | sequence |
| | ccgccaggct | |
| | ccagggaagg ggctggagtg ggtctcagtt attactggta gtggtactag | |
| | cacacactac | |
| | gcagactccg tgaagggccg gttcaccatc tccagagaca attccaagaa | |
| | aacgctatat | |
| | ctgcaaatga atagcctgag agccgaggac acggccgtat attactgtgc | |
| | gaaagatcgg | |
| | ggctatgatt atagtggttc ttactacaac tggttcgacc cctggggcca | |
| | gggaaccctg | |
| | gtcaccgtct cctca | |
| 354. | EVQLVESGGG LVQPGGSLRL SCAASGFSFH | AA amino |
| | NFAMNWVRQA PGKGLEWVSV ITGSGTSTHY | acid |
| | ADSVKGRFTI SRDNSKKTLY LQMNSLRAED | sequence |
| | TAVYYCAKDR GYDYSGSYYN WFDPWGQGTL | |
| | VTVSS | |
| 355. | ggattctcct ttcataattt tgcc | DNA |
| | | nucleotide |
| | | sequence |
| 356. | GFSFHNFA | AA amino |
| | | acid |
| | | sequence |
| 357. | attactggta gtggtactag caca | DNA |
| | | nucleotide |
| | | sequence |
| 358. | ITGSGTST | AA amino |
| | | acid |
| | | sequence |
| 359. | gcgaaagatc ggggctatga ttatagtggt tcttactaca actggttcga | DNA |
| | cccc | nucleotide |
| | | sequence |
| 360. | AKDRGYDYSG SYYNWFDP | AA amino |
| | | acid |
| | | sequence |
| 361. | gacatccaga tgacccagtc tccatcctcc ctgtctgcat ctgtaggaga | DNA |
| | cagaatcacc | nucleotide |
| | atcacttgcc gggcaagtca gagtattagc agctatttaa attggtatca | sequence |
| | gcagaaacca | |
| | gggaaagccc ctaaactcct gatctttgct gcatcaaatt tgcaaagtgg | |
| | ggtcccatca | |
| | aggttcagtg gcagtggatc tgggacagat ttcactctca ccatcagtag | |
| | tctgcaacct | |
| | gaagattttg caacttacta ctgtcaacag agttacagta ccccatcctt | |
| | attcactttc | |
| | ggccctggga ccaaagtgga tatcaaa | |
| 362. | DIQMTQSPSS LSASVGDRIT ITCRASQSIS SYLNWYQQKP | AA amino |
| | GKAPKLLIFA ASNLQSGVPS | acid |
| | RFSGSGSGTD FTLTISSLQP EDFATYYCQQ SYSTPSLFTF | sequence |
| | GPGTKVDIK | |
| 363. | cagagtatta gcagctat | DNA |
| | | nucleotide |
| | | sequence |
| 364. | QSISSY | AA amino |
| | | acid |
| | | sequence |
| 365. | gctgcatca | DNA |
| | | nucleotide |
| | | sequence |
| 366. | AAS | AA amino |
| | | acid |
| | | sequence |
| 367. | caacagagtt acagtacccc atccttattc act | DNA |
| | | nucleotide |
| | | sequence |
| 368. | QQSYSTPSLF T | AA amino |
| | | acid |
| | | sequence |
| 369. | gaggtgcagc tggtggagtc tgggggaggc ttggtacagc ctggagggtc | DNA |
| | cctgagactc | nucleotide |
| | tcctgtgcag tctctggatt caccttcagt agttacgaga tgaactgggt | sequence |
| | ccgccaggct | |
| | ccagggaagg ggctggaatg ggtttcacac attagtagta gtggaagtac | |
| | catatactac | |
| | gcagactctg tgaagggccg attcaccatg tccagagaca acgccaagaa | |
| | ctcactgtat | |
| | ctgcaaatga acagcctgag agccgaggac acggctgttt attactgtgc | |
| | gagagatggg | |
| | aatatctgga gtggttatta tgccgcctac tactictacg gtatggacgt | |
| | ctggggccaa | |
| | gggaccacgg tcaccgtctc ctca | |
| 370. | EVQLVESGGG LVQPGGSLRL SCAVSGFTFS | AA amino |
| | SYEMNWVRQA PGKGLEWVSH ISSSGSTIYY | acid |
| | ADSVKGRFTM SRDNAKNSLY LQMNSLRAED | sequence |
| | TAVYYCARDG NIWSGYYAAY YFYGMDVWGQ | |
| | GTTVTVSS | |
| 371. | ggattcacct tcagtagtta cgag | DNA |
| | | nucleotide |
| | | sequence |
| 372. | GFTFSSYE | AA amino |
| | | acid |
| | | sequence |
| 373. | attagtagta gtggaagtac cata | DNA |
| | | nucleotide |
| | | sequence |
| 374. | ISSSGSTI | AA amino |
| | | acid |
| | | sequence |
| 375. | gcgagagatg ggaatatctg gagtggttat tatgccgcct actacttcta | DNA |
| | cggtatggac | nucleotide |
| | gtc | sequence |
| 376. | ARDGNIWSGY YAAYYFYGMD V | AA amino |
| | | acid |
| | | sequence |
| 377. | gatattgtga tgacccagac tccactctcc tcacctgtca cccttggaca | DNA |
| | gccggcctcc | nucleotide |
| | atctcctgca ggtctagtca aagcctcgta cacagtgatg gaaaaaccta | sequence |
| | cttgagttgg | |
| | cttcagcaga ggccaggcca gcctccaaga ctcctaattt ataagatttc | |
| | taaccggttc | |
| | tctggggtcc cagacagaat cagtggcagt ggggcaggga cagatttcac | |
| | actgaaaatc | |
| | agcagggtgg aagctgagga tgtcggggtt tattactgca tgcaagctgt | |
| | acaatttcct | |
| | cggacgttcg gccaagggac caaggtggaa atcaaa | |
| 378. | DIVMTQTPLS SPVTLGQPAS ISCRSSQSLV | AA amino |
| | HSDGKTYLSW LQQRPGQPPR LLIYKISNRF | acid |
| | SGVPDRISGS GAGTDFTLKI SRVEAEDVGV | sequence |
| | YYCMQAVQFP RTFGQGTKVE IK | |
| 379. | caaagcctcg tacacagtga tggaaaaacc tac | DNA |
| | | nucleotide |
| | | sequence |
| 380. | QSLVHSDGKT Y | AA amino |
| | | acid |
| | | sequence |
| 381. | aagatttct | DNA |
| | | nucleotide |
| | | sequence |
| 382. | KIS | AA amino |
| | | acid |
| | | sequence |
| 383. | atgcaagctg tacaatttcc tcggacg | DNA |
| | | nucleotide |
| | | sequence |
| 384. | MQAVQFPRT | AA amino |
| | | acid |
| | | sequence |
| 385. | caggtgcagc tacagcagtg gggcgcagga ctgttgaacc cttcggagac | DNA |
| | cctgtccctc | nucleotide |
| | acctgcgctg tctatggtgg ggccttcagt gattactact ggaattggat | sequence |
| | ccgccagccc | |
| | ccagggaagg ggctggagtg gattggggaa atcaatcatc gcggaagcac | |
| | caactacaac | |
| | ccgtccctca agagtcgtgt caccatttca gttgacacgt ccaagaacca | |
| | gttctccctg | |
| | aggatgagct ctgtgaccgc cgcggacgcg gctgtgtatt actgtgcgag | |
| | aggagaggat | |
| | tacgatattt ggaatggtta ttatcaggaa aaatggggcc agggaaccct | |
| | ggtcaccgtc | |
| | tcctca | |
| 386. | QVQLQQWGAG LLNPSETLSL TCAVYGGAFS | AA amino |
| | DYYWNWIRQP PGKGLEWIGE INHRGSTNYN | acid |
| | PSLKSRVTIS VDTSKNQFSL RMSSVTAADA | sequence |
| | AVYYCARGED YDIWNGYYQE KWGQGTLVTV | |
| | SS | |
| 387. | ggtggggcct tcagtgatta ctac | DNA |
| | | nucleotide |
| | | sequence |
| 388. | GGAFSDYY | AA amino |
| | | acid |
| | | sequence |
| 389. | atcaatcatc gcggaagcac c | DNA |
| | | nucleotide |
| | | sequence |
| 390. | INHRGST | AA amino |
| | | acid |
| | | sequence |
| 391. | gcgagaggag aggattacga tatttggaat ggttattatc aggaaaaa | DNA |
| | | nucleotide |
| | | sequence |
| 392. | ARGEDYDIWN GYYQEK | AA amino |
| | | acid |
| | | sequence |
| 393. | gaaattgtgt tgacacagtc tccagccacc ctgtctttgt ctccagggga | DNA |
| | aagagccacc | nucleotide |
| | ctctcctgca gggccagtca gagtattagc acctacttag cctggtacca | sequence |
| | acagaagcct | |
| | ggccaggctc ccaggctcct catctatgat gcatccaaga gggccactgg | |
| | catcccagcc | |
| | aggttcagtg gcagtgggtc tgggacagac ttcactctca ccatcagcag | |
| | cctagagcct | |
| | gaagattttg tagtttatta ctgtcaccag cgtagcaact ggcctctcac | |
| | tttcggcgga | |
| | gggaccaagg tggagatcaa a | |
| 394. | EIVLTQSPAT LSLSPGERAT LSCRASQSIS TYLAWYQQKP | AA amino |
| | GQAPRLLIYD ASKRATGIPA | acid |
| | RFSGSGSGTD FTLTISSLEP EDFVVYYCHQ RSNWPLTFGG | sequence |
| | GTKVEIK | |
| 395. | cagagtatta gcacctac | DNA |
| | | nucleotide |
| | | sequence |
| 396. | QSISTY | AA amino |
| | | acid |
| | | sequence |
| 397. | gatgcatcc | DNA |
| | | nucleotide |
| | | sequence |
| 398. | DAS | AA amino |
| | | acid |
| | | sequence |
| 399. | caccagcgta gcaactggcc tctcact | DNA |
| | | nucleotide |
| | | sequence |
| 400. | HQRSNWPLT | AA amino |
| | | acid |
| | | sequence |
| 401. | caggtgcagc tgcaggagtc ggggccagga ctggtgaagc cttcggagac | DNA |
| | cctgtccctc | nucleotide |
| | acctgcactg tctctggtgg ttccttcagt agttactact ggagttggct | sequence |
| | ccggcagccc | |
| | ccaggaaagg ggctggagtg gattggatat atcttttaca gtgggagtac | |
| | cgactacaac | |
| | ccctccctca agagtcgagt caccatttca gtagacacgt ccaagaagca | |
| | gttctccctg | |
| | aagctgacct ctgtgaccgc tgcggacacg gccgtctatt actgtgcgcg | |
| | aacaataagt | |
| | acgtggtggt tcgccccctg gggccaggga accctggtca ccgtctcctc | |
| | a | |
| 402. | QVQLQESGPG LVKPSETLSL TCTVSGGSFS | AA amino |
| | SYYWSWLRQP PGKGLEWIGY IFYSGSTDYN | acid |
| | PSLKSRVTIS VDTSKKQFSL KLTSVTAADT | sequence |
| | AVYYCARTIS TWWFAPWGQG TLVTVSS | |
| 403. | ggtggttcct tcagtagtta ctac | DNA |
| | | nucleotide |
| | | sequence |
| 404. | GGSFSSYY | AA amino |
| | | acid |
| | | sequence |
| 405. | atcttttaca gtgggagtac c | DNA |
| | | nucleotide |
| | | sequence |
| 406. | IFYSGST | AA amino |
| | | acid |
| | | sequence |
| 407. | gcgcgaacaa taagtacgtg gtggttcgcc ccc | DNA |
| | | nucleotide |
| | | sequence |
| 408. | ARTISTWWFA P | AA amino |
| | | acid |
| | | sequence |
| 409. | gaaatagtga tgacacagtc tccagccacc ctgtctgtgt ctccaggggg | DNA |
| | aagagccacc | nucleotide |
| | ctctcctgca gggccagtca gagtgttagc aacaacgtag cctggtacca | sequence |
| | gcagaaacct | |
| | ggccaggctc ccaggctcct catctatggt gcatccacca gggccactgg | |
| | tatcccaggc | |
| | aggttcagtg gcagtgggtc tggaacagag ttcactctca ccatcagcag | |
| | cctgcagtct | |
| | gaagattttg cagtttattc ctgtcagcag tataataact ggctcacttt | |
| | cggcggaggg | |
| | accaaggtgg agatcaaa | |
| 410. | EIVMTQSPAT LSVSPGGRAT LSCRASQSVS | AA amino |
| | NNVAWYQQKP GQAPRLLIYG ASTRATGIPG | acid |
| | RFSGSGSGTE FTLTISSLQS EDFAVYSCQQ | sequence |
| | YNNWLTFGGG TKVEIK | |
| 411. | cagagtgtta gcaacaac | DNA |
| | | nucleotide |
| | | sequence |
| 412. | QSVSNN | AA amino |
| | | acid |
| | | sequence |
| 413. | ggtgcatcc | DNA |
| | | nucleotide |
| | | sequence |
| 414. | GAS | AA amino |
| | | acid |
| | | sequence |
| 415. | cagcagtata ataactggct cact | DNA |
| | | nucleotide |
| | | sequence |
| 416. | QQYNNWLT | AA amino |
| | | acid |
| | | sequence |
| 417. | caggtgcagc tggtggagtc tgggggaggc gtggtccagc ctgggaggtc | DNA |
| | cctgagactc | nucleotide |
| | tcctgtgtag cgtctggatt cactttcagt agttatggca tgcactgggt | sequence |
| | ccgccaggct | |
| | ccaggcaagg ggctggagtg ggtggcaatt atatggtatg atggaagtaa | |
| | taaatactat | |
| | gcagactccg tgaagggccg attcaccata tccagagaca attccaagaa | |
| | cacacagtat | |
| | ctgcaaatga acagcctgag agccgaggac acggctgtgt attactgtgc | |
| | gtcagtagct | |
| | acgtctgggg acttcgacta ctacggtatg gacgtctggg gccaagggac | |
| | cacggtcacc | |
| | gtctcctca | |
| 418. | QVQLVESGGG VVQPGRSLRL SCVASGFTFS | AA amino |
| | SYGMHWVRQA PGKGLEWVAI IWYDGSNKYY | acid |
| | ADSVKGRFTI SRDNSKNTQY LQMNSLRAED | sequence |
| | TAVYYCASVA TSGDFDYYGM DVWGQGTTVT | |
| | VSS | |
| 419. | ggattcactt tcagtagtta tggc | DNA |
| | | nucleotide |
| | | sequence |
| 420. | GFTFSSYG | AA amino |
| | | acid |
| | | sequence |
| 421. | atatggtatg atggaagtaa taaa | DNA |
| | | nucleotide |
| | | sequence |
| 422. | IWYDGSNK | AA amino |
| | | acid |
| | | sequence |
| 423. | gcgtcagtag ctacgtctgg ggacttcgac tactacggta tggacgtc | DNA |
| | | nucleotide |
| | | sequence |
| 424. | ASVATSGDFD YYGMDV | AA amino |
| | | acid |
| | | sequence |
| 425. | gaaattgtgt tgacacagtc tccagccacc ctgtctttgt ctccagggga | DNA |
| | aagaaccacc | nucleotide |
| | ctctcctgca gggccagtca gagaattagc acctacttag cctggtatca | sequence |
| | acagaaacct | |
| | ggccaggctc ccaggctcct catctatgat gcatccaaaa gggccactgg | |
| | catcccagcc | |
| | aggttcagtg gtagtgggtc tgggacaggc ttcactctca ccatcagcag | |
| | cctagagcct | |
| | gaagattttg cagtttatta ctgtcagcag cgtagtaact ggcctctcac | |
| | tttcggcgga | |
| | gggaccaagg tggagatcaa a | |
| 426. | EIVLTQSPAT LSLSPGERTT LSCRASQRIS TYLAWYQQKP | AA amino |
| | GQAPRLLIYD ASKRATGIPA | acid |
| | RFSGSGSGTG FTLTISSLEP EDFAVYYCQQ RSNWPLTFGG | sequence |
| | GTKVEIK | |
| 427. | cagagaatta gcacctac | DNA |
| | | nucleotide |
| | | sequence |
| 428. | QRISTY | AA amino |
| | | acid |
| | | sequence |
| 429. | gatgcatcc | DNA |
| | | nucleotide |
| | | sequence |
| 430. | DAS | AA amino |
| | | acid |
| | | sequence |
| 431. | cagcagcgta gtaactggcc tctcact | DNA |
| | | nucleotide |
| | | sequence |
| 432. | QQRSNWPLT | AA amino |
| | | acid |
| | | sequence |
| 433. | gaggtgcagc tggtgcagtc tggagcagag gtgagaaagc ccggggagtc | DNA |
| | tctgaagatc | nucleotide |
| | tcctgtaagg gttctggata cagctttact aactactgga tcgtctgggt | sequence |
| | gcgccagatg | |
| | cccgggaaag gcctggagtg gatggggatc atctatcctg gtgactctga | |
| | taccagatac | |
| | agcccgtcct tccaaggcca ggtcaccatc tcagccgaca agtccatcag | |
| | caccgcctac | |
| | ctgcagtgga gcagcctgaa ggcctcggac accgccatgt attactgtgc | |
| | gagacgggat | |
| | acgattttcc cttcctatcc cctctggggc cagggaaccc tggtcaccgt | |
| | ctcctca | |
| 434. | EVQLVQSGAE VRKPGESLKI SCKGSGYSFT | AA amino |
| | NYWIVWVRQM PGKGLEWMGI IYPGDSDTRY | acid |
| | SPSFQGQVTI SADKSISTAY LQWSSLKASD | sequence |
| | TAMYYCARRD TIFPSYPLWG QGTLVTVSS | |
| 435. | ggatacagct ttactaacta ctgg | DNA |
| | | nucleotide |
| | | sequence |
| 436. | GYSFTNYW | AA amino |
| | | acid |
| | | sequence |
| 437. | atctatcctg gtgactctga tacc | DNA |
| | | nucleotide |
| | | sequence |
| 438. | IYPGDSDT | AA amino |
| | | acid |
| | | sequence |
| 439. | gcgagacggg atacgatttt cccttcctat cccctc | DNA |
| | | nucleotide |
| | | sequence |
| 440. | ARRDTIFPSY PL | AA amino |
| | | acid |
| | | sequence |
| 441. | gatattgtga tgactcagtc tcctctctcc ctgcccgtca cccctggaga | DNA |
| | gccggcctcc | nucleotide |
| | atctcctgca ggtctagtca gagcctcctg aatagtaatg gatacaactt | sequence |
| | tttggattgg | |
| | tacctgcaga agccagggca gtctccacaa ctcctgatct atttggtttc | |
| | taatcgggcc | |
| | tccggggtcc ctgacaggtt cagtggcagt ggatcaggca cagattttac | |
| | actgaaaatc | |
| | agcagagtgg aggctgagga tattggggtt tattactgca tgcaagctct | |
| | ccaaactccg | |
| | atcaccttcg gccaagggac acgactggag attaaa | |
| 442. | DIVMTQSPLS LPVTPGEPAS ISCRSSQSLL | AA amino |
| | NSNGYNFLDW YLQKPGQSPQ LLIYLVSNRA | acid |
| | SGVPDRFSGS GSGTDFTLKI SRVEAEDIGV | sequence |
| | YYCMQALQTP ITFGQGTRLE IK | |
| 443. | cagagcctcc tgaatagtaa tggatacaac ttt | DNA |
| | | nucleotide |
| | | sequence |
| 444. | QSLLNSNGYN F | AA amino |
| | | acid |
| | | sequence |
| 445. | ttggtttct | DNA |
| | | nucleotide |
| | | sequence |
| 446. | LVS | AA amino |
| | | acid |
| | | sequence |
| 447. | atgcaagctc tccaaactcc gatcacc | DNA |
| | | nucleotide |
| | | sequence |
| 448. | MQALQTPIT | AA amino |
| | | acid |
| | | sequence |
| 449. | cagatcacct tgaaggagtc tggtcctacg ctggtgaaac ccacacagac | DNA |
| | cctcacgctg | nucleotide |
| | acctgcacct tctctgggtt ctcactcagc actaatggag tgggtgtggg | sequence |
| | ctggatccgt | |
| | cagcccccag gaaaggccct ggagtggctt acactcattt attggaatga | |
| | aaataagcac | |
| | tacagcccat ctctgaaaaa caggatcacc atcaccaagg acacctccaa | |
| | aaaccaggtg | |
| | gtccttacaa tgaccaactt ggaccctgtg gacacagcca cttattactg | |
| | tgtacacagg | |
| | ggatggttgg gagcaatctt tgcctactgg ggccagggaa ccctggtcac | |
| | cgtctcctca | |
| 450. | QITLKESGPT LVKPTQTLTL TCTFSGFSLS TNGVGVGWIR | AA amino |
| | QPPGKALEWL TLIYWNENKH | acid |
| | YSPSLKNRIT ITKDTSKNQV VLTMTNLDPV DTATYYCVHR | sequence |
| | GWLGAIFAYW GQGTLVTVSS | |
| 451. | gggttctcac tcagcactaa tggagtgggt | DNA |
| | | nucleotide |
| | | sequence |
| 452. | GFSLSTNGVG | AA amino |
| | | acid |
| | | sequence |
| 453. | atttattgga atgaaaataa g | DNA |
| | | nucleotide |
| | | sequence |
| 454. | IYWNENK | AA amino |
| | | acid |
| | | sequence |
| 455. | gtacacaggg gatggttggg agcaatcttt gcctac | DNA |
| | | nucleotide |
| | | sequence |
| 456. | VHRGWLGAIF AY | AA amino |
| | | acid |
| | | sequence |
| 457. | gaggtgcagc tggtggagtc tgggggaggc ttggtacagc ctggggggtc | DNA |
| | cctgagactc | nucleotide |
| | tcctgtgcag cctctggatt cacctttact agttatgcca tgacctgggt | sequence |
| | ccgccaggct | |
| | ccagggaagg ggctggagtg ggtctcagat attagtggta gtggtggtag | |
| | aacatattac | |
| | gcagactccg tgaagggccg gttcaccatc tccagagaca attccaagaa | |
| | tatgctgtat | |
| | ctgcaaatga acatcctgag agccgaagac acggccgtat atcattgtgc | |
| | gaagggaaca | |
| | ggccagcagg tggaccttta caactactac tatgctttgg acgtctgggg | |
| | ccaagggacc | |
| | acggtcaccg tctcctca | |
| 458. | EVQLVESGGG LVQPGGSLRL SCAASGFTFT | AA amino |
| | SYAMTWVRQA PGKGLEWVSD ISGSGGRTYY | acid |
| | ADSVKGRFTI SRDNSKNMLY LQMNILRAED | sequence |
| | TAVYHCAKGT GQQVDLYNYY YALDVWGQGT | |
| | TVTVSS | |
| 459. | ggattcacct ttactagtta tgcc | DNA |
| | | nucleotide |
| | | sequence |
| 460. | GFTFTSYA | AA amino |
| | | acid |
| | | sequence |
| 461. | attagtggta gtggtggtag aaca | DNA |
| | | nucleotide |
| | | sequence |
| 462. | ISGSGGRT | AA amino |
| | | acid |
| | | sequence |
| 463. | gcgaagggaa caggccagca ggtggacctt tacaactact actatgcttt | DNA |
| | ggacgtc | nucleotide |
| | | sequence |
| 464. | AKGTGQQVDL YNYYYALDV | AA amino |
| | | acid |
| | | sequence |
| 465. | caggtgcagc tggtggagtc tgggggaggc gtggtccagc ctgggaggtc | DNA |
| | cctgagactc | nucleotide |
| | tcctgtgcag cgtctggatt caccttcagt tactatggca tgcactgggt | sequence |
| | ccgccaggct | |
| | ccaggcaagg ggctggagtg ggtggcagtt atatggtatg atggaagtaa | |
| | taaacactat | |
| | gcagactccg tgaagggccg attcaccatc tccagagaca attccaagaa | |
| | cacgctgtat | |
| | ctgcaaatga acagcctgag agccgacgac acggctgtct attactgtgc | |
| | gagagataag | |
| | ggtataagtg gaattaaggg gggttcttac tactactact atgccatgga | |
| | cgtctggggc | |
| | caagggacca cggtcaccgt ctcctca | |
| 466. | QVQLVESGGG VVQPGRSLRL SCAASGFTFS | AA amino |
| | YYGMHWVRQA PGKGLEWVAV IWYDGSNKHY | acid |
| | ADSVKGRFTI SRDNSKNTLY LQMNSLRADD | sequence |
| | TAVYYCARDK GISGIKGGSY YYYYAMDVWG | |
| | QGTTVTVSS | |
| 467. | ggattcacct tcagttacta tggc | DNA |
| | | nucleotide |
| | | sequence |
| 468. | GFTFSYYG | AA amino |
| | | acid |
| | | sequence |
| 469. | atatggtatg atggaagtaa taaa | DNA |
| | | nucleotide |
| | | sequence |
| 470. | IWYDGSNK | AA amino |
| | | acid |
| | | sequence |
| 471. | gcgagagata agggtataag tggaattaag gggggttctt actactacta | DNA |
| | ctatgccatg | nucleotide |
| | gacgtc | sequence |
| 472. | ARDKGISGIK GGSYYYYYAM DV | AA amino |
| | | acid |
| | | sequence |
| 473. | gaggtgcagc tggtggagtc tgggggaggc ttggtaaagc ctggggggtc | DNA |
| | ccttagactc | nucleotide |
| | tcctgtgcag cctctggatt cactttcagt aacgcctgga tgacctgggt | sequence |
| | ccgccaggct | |
| | ccagggaagg ggctggagtg ggttggccgt attaaaaaca aaattgatgg | |
| | tcggacaaca | |
| | gactacgctg cacccgtgaa aggcagattc accatctcaa gagatgattc | |
| | aaaaaacacg | |
| | gtttatctgc aaatgaacag cctgaaaacc gaggacacag ccgtttatta | |
| | ctgttccacg | |
| | gtggactaca attggtactt cgatttctgg ggccgtggca ccctggtcac | |
| | tgtctcctca | |
| 474. | EVQLVESGGG LVKPGGSLRL SCAASGFTFS | AA amino |
| | NAWMTWVRQA PGKGLEWVGR IKNKIDGGTT | acid |
| | DYAAPVKGRF TISRDDSKNT VYLQMNSLKT | sequence |
| | EDTAVYYCST VDYNWYFDFW GRGTLVTVSS | |
| 475. | ggattcactt tcagtaacgc ctgg | DNA |
| | | nucleotide |
| | | sequence |
| 476. | GFTFSNAW | AA amino |
| | | acid |
| | | sequence |
| 477. | attaaaaaca aaattgatgg tgggacaaca | DNA |
| | | nucleotide |
| | | sequence |
| 478. | IKNKIDGGTT | AA amino |
| | | acid |
| | | sequence |
| 479. | tccacggtgg actacaattg gtacttcgat ttc | DNA |
| | | nucleotide |
| | | sequence |
| 480. | STVDYNWYFD F | AA amino |
| | | acid |
| | | sequence |
| 481. | caggtgcagc tggtggagtc tgggggaggc gtggtccagc ctgggaggtc | DNA |
| | cctgagactc | nucleotide |
| | tcctgtgcag cgtctggatt caccttcagt ttctttggca tgcactgggt | sequence |
| | ccgccaggct | |
| | ccaggcaagg ggctggagtg ggtggcactt atatggtatg atggaactaa | |
| | tgaaaactat | |
| | gcagactccg tgaagggccg attcaccatc tccagagaca attccaagtc | |
| | cacgctgtat | |
| | ctgcaaatga acagtctgag agccgaggac acggctgttt actactgtgc | |
| | gagagatagg | |
| | ggagtggcga catttacgag ggggaattac tactacaact acggtatgga | |
| | cgtctggggc | |
| | caagggacca cggtcaccgt ctcctca | |
| 482. | QVQLVESGGG VVQPGRSLRL SCAASGFTFS | AA amino |
| | FFGMHWVRQA PGKGLEWVAL IWYDGTNENY | acid |
| | ADSVKGRFTI SRDNSKSTLY LQMNSLRAED | sequence |
| | TAVYYCARDR GVATFTRGNY YYNYGMDVWG | |
| | QGTTVTVSS | |
| 483. | ggattcacct tcagtttctt tggc | DNA |
| | | nucleotide |
| | | sequence |
| 484. | GFTFSFFG | AA amino |
| | | acid |
| | | sequence |
| 485. | atatggtatg atggaactaa tgaa | DNA |
| | | nucleotide |
| | | sequence |
| 486. | IWYDGTNE | AA amino |
| | | acid |
| | | sequence |
| 487. | gcgagagata ggggagtggc gacatttacg agggggaatt actactacaa | DNA |
| | ctacggtatg | nucleotide |
| | gacgtc | sequence |
| 488. | ARDRGVATFT RGNYYYNYGM DV | AA amino |
| | | acid |
| | | sequence |
| 489. | caggtgcagc tggtggagtc tgggggaggc gtggtccagc ctgggaggtc | DNA |
| | cctgagactc | nucleotide |
| | tcctgtgcag cgtctggatt caccttcagt ttctatggca tgcactgggt | sequence |
| | ccgccaggct | |
| | ccaggcaagg ggctggaggg ggtggcagtt atatggtatg atggaagtaa | |
| | taaatactat | |
| | gcagactccg tgaagggccg attcaccata tccagagaca attccaagaa | |
| | catgctgtat | |
| | ctacaaatga ccagcctgag agccgaggac acggctgtgt attactgtgc | |
| | gagagattcg | |
| | ggtaaaactg gaactgggat aactgggtac tcctactact acggtatgga | |
| | cgtctggggc | |
| | caagggacca cggtcaccgt ctcctca | |
| 490. | QVQLVESGGG VVQPGRSLRL SCAASGFTFS | AA amino |
| | FYGMHWWRQA PGKGLEGVAV IWYDGSNKYY | acid |
| | ADSVKGRFTI SRDNSKNMLY LQMTSLRAED | sequence |
| | TAVYYCARDS GKTGTGITGY SYYYGMDVWG | |
| | QGTTVTVSS | |
| 491. | ggattcacct tcagtttcta tggc | DNA |
| | | nucleotide |
| | | sequence |
| 492. | GFTFSFYG | AA amino |
| | | acid |
| | | sequence |
| 493. | atatggtatg atggaagtaa taaa | DNA |
| | | nucleotide |
| | | sequence |
| 494. | IWYDGSNK | AA amino |
| | | acid |
| | | sequence |
| 495. | gcgagagatt cgggtaaaac tggaactggg ataactgggt actcctacta | DNA |
| | ctacggtatg | nucleotide |
| | gacgtc | sequence |
| 496. | ARDSGKTGTG ITGYSYYYGM DV | AA amino |
| | | acid |
| | | sequence |
| 497. | cagctgcagc tgcaggagtc gggcccagga ctggtgaagc cttcggagac | DNA |
| | cctgtccctc | nucleotide |
| | acctgcactg tctctggtgg ctccatcatc actaatagtt attactgggg | sequence |
| | ctggatccgc | |
| | cagcccccag ggaagggtct ggagtggatt ggtagtatct attatagtgg | |
| | gaggacctac | |
| | tacaacccgt ccctcgagag tcgagtcacc atatccgtgg acacgtccaa | |
| | gaaccagttc | |
| | tccctgaagt tgacctctgt gaccgccgca gacacggcta tatattactg | |
| | tgcgagggaa | |
| | ggggatccgt cgctcgaccc ctggggccag ggaaccctgg tcaccgtctc | |
| | ctca | |
| 498. | QLQLQESGPG LVKPSETLSL TCTVSGGSII TNSYYWGWIR | AA amino |
| | QPPGKGLEWI GSIYYSGRTY | acid |
| | YNPSLESRVT ISVDTSKNQF SLKLTSVTAA DTAIYYCARE | sequence |
| | GDPSLDPWGQ GTLVTVSS | |
| 499. | ggtggctcca tcatcactaa tagttattac | DNA |
| | | nucleotide |
| | | sequence |
| 500. | GGSIITNSYY | AA amino |
| | | acid |
| | | sequence |
| 501. | atctattata gtgggaggac c | DNA |
| | | nucleotide |
| | | sequence |
| 502. | IYYSGRT | AA amino |
| | | acid |
| | | sequence |
| 503. | gcgagggaag gggatccgtc gctcgacccc | DNA |
| | | nucleotide |
| | | sequence |
| 504. | AREGDPSLDP | AA amino |
| | | acid |
| | | sequence |
| 505. | gaggtgcagc tggtggagtc tgggggagac ttggtacagc ctggggggtc | DNA |
| | cctgagactc | nucleotide |
| | tcctgtgcag cctctggatt cacctttagc acctatgcca tgaactgggt | sequence |
| | ccgccaggct | |
| | ccagggaagg ggctggagtg ggtctcacat attagtggta gtggtggtaa | |
| | ttcatactcc | |
| | gcagactccg tgaagggccg gttcaccatc tccagagaca attccaagaa | |
| | cacgctatat | |
| | ctgcaaatga acagcctgcg agccgaggac acggccatat attactgttc | |
| | gctggatata | |
| | atggctacag taggcggtct ctttgcctac tggggccagg gaaccctggt | |
| | caccgtctcc | |
| | tca | |
| 506. | EVQLVESGGD LVQPGGSLRL SCAASGFTFS | AA amino |
| | TYAMNWVRQA PGKGLEWVSH ISGSGGNSYS | acid |
| | ADSVKGRFTI SRDNSKNTLY LQMNSLRAED | sequence |
| | TAIYYCSLDI MATVGGLFAY WGQGTLVTVS | |
| | S | |
| 507. | ggattcacct ttagcaccta tgcc | DNA |
| | | nucleotide |
| | | sequence |
| 508. | GFTFSTYA | AA amino |
| | | acid |
| | | sequence |
| 509. | attagtggta gtggtggtaa ttca | DNA |
| | | nucleotide |
| | | sequence |
| 510. | ISGSGGNS | AA amino |
| | | acid |
| | | sequence |
| 511. | tcgctggata taatggctac agtaggcggt ctctttgcct ac | DNA |
| | | nucleotide |
| | | sequence |
| 512. | SLDIMATVGG LFAY | AA amino |
| | | acid |
| | | sequence |
| 513. | caggtgcagc tggtggagtc tgggggaggc gtggtccagc ctgggaggtc | DNA |
| | cctgagactc | nucleotide |
| | tcctgtgtag cgtctggatt catcttcagt ttctatggca tgcactgggt | sequence |
| | ccgccaggct | |
| | ccagacaagg ggctggagtg ggtggcagtt atatggtatg atggaagtaa | |
| | tgaatactat | |
| | gcagactccg tgaagggccg attcaccatc tccagagaca attccaagaa | |
| | cacgctgtat | |
| | ctgcaaatga acagcctgag agccgaggac acggctgtgt attactgtgg | |
| | gagagatcaa | |
| | ggtatttcgt attacgatat tttgactggt aattataact attactacgg | |
| | tgtggacgtc | |
| | tggggccaag ggaccacggt caccgtctcc tca | |
| 514. | QVQLVESGGG VVQPGRSLRL SCVASGFIFS | AA amino |
| | FYGMHWVRQA PDKGLEWVAV IWYDGSNEYY | acid |
| | ADSVKGRFTI SRDNSKNTLY LQMNSLRAED | sequence |
| | TAVYYCGRDQ GISYYDILTG NYNYYYGVDV | |
| | WGQGTTVTVS S | |
| 515. | ggattcatct tcagtttcta tggc | DNA |
| | | nucleotide |
| | | sequence |
| 516. | GFIFSFYG | AA amino |
| | | acid |
| | | sequence |
| 517. | atatggtatg atggaagtaa tgaa | DNA |
| | | nucleotide |
| | | sequence |
| 518. | IWYDGSNE | AA amino |
| | | acid |
| | | sequence |
| 519. | gggagagatc aaggtatttc gtattacgat attttgactg gtaattataa | DNA |
| | ctattactac | nucleotide |
| | ggtgtggacg tc | sequence |
| 520. | GRDQGISYYD ILTGNYNYYY GVDV | AA amino |
| | | acid |
| | | sequence |
| 521. | gacatccaga tgacccagtc tccatcctcc ctgtctgcat ctgtaggaga | DNA |
| | cagagtcacc | nucleotide |
| | atcacttgcc gggcaagtca gagcattagc agctatttaa attggtatca | sequence |
| | gcagaaacca | |
| | gggaaagccc ctaagctcct gatctatgct gcatccagtt tgcaaagtgg | |
| | ggtcccgtca | |
| | aggttcagtg gcagtggatc tgggacagat ttcactctca ccatcagcag | |
| | tctgcaacct | |
| | gaagattttg caacttacta ctgtcaacag agttacagta cccctccgat | |
| | caccttcggc | |
| | caagggacac gactggagat taaa | |
| 522. | DIQMTQSPSS LSASVGDRVT ITCRASQSIS | AA amino |
| | SYLNWYQQKP GKAPKLLIYA ASSLQSGVPS | acid |
| | RFSGSGSGTD FTLTISSLQP EDFATYYCQQ | sequence |
| | SYSTPPITFG QGTRLEIK | |
| 523. | cagagcatta gcagctat | DNA |
| | | nucleotide |
| | | sequence |
| 524. | QSISSY | AA amino |
| | | acid |
| | | sequence |
| 525. | gctgcatcc | DNA |
| | | nucleotide |
| | | sequence |
| 526. | AAS | AA amino |
| | | acid |
| | | sequence |
| 527. | caacagagtt acagtacccc tccgatcacc | DNA |
| | | nucleotide |
| | | sequence |
| 528. | QQSYSTPPIT | AA amino |
| | | acid |
| | | sequence |
| 529. | gaaattgtgt tgacgcagtc tccaggcacc ctgtctttgt ctccagggga | DNA |
| | aagagccacc | nucleotide |
| | ctctcctgca gggccagtca gagtgttagc agcagctact tagcctggta | sequence |
| | ccagcagaaa | |
| | cctggccagg ctcccaggct cctcatctat ggtgcatcca gcagggccac | |
| | tggcatccca | |
| | gacaggttca gtggcagtgg gtctgggaca gacttcactc tcaccatcag | |
| | cagactggag | |
| | cctgaagatt ttgcagtgta ttactgtcag cagtatggta gctcaccttg | |
| | gacgttcggc | |
| | caagggacca aggtggaaat caaa | |
| 530. | EIVLTQSPGT LSLSPGERAT LSCRASQSVS | AA amino |
| | SSYLAWYQQK PGQAPRLLIY GASSRATGIP | acid |
| | DRFSGSGSGT DFTLTISRLE PEDFAVYYCQ | sequence |
| | QYGSSPWTFG QGTKVEIK | |
| 531. | cagagtgtta gcagcagcta c | DNA |
| | | nucleotide |
| | | sequence |
| 532. | QSVSSSY | AA amino |
| | | acid |
| | | sequence |
| 533. | ggtgcatcc | DNA |
| | | nucleotide |
| | | sequence |
| 534. | GAS | AA amino |
| | | acid |
| | | sequence |
| 535. | cagcagtatg gtagctcacc ttggacg | DNA |
| | | nucleotide |
| | | sequence |
| 536. | QQYGSSPWT | AA amino |
| | | acid |
| | | sequence |
| 537. | caggtgcagc tacagcagtg gggcgcagga ctgttgaagc cttcggagac | DNA |
| | cctgtccctc | nucleotide |
| | acctgcgctg tctatggtgg gtccttcagt ggttactact ggaactggat | sequence |
| | ccgccagccc | |
| | ccagggaagg ggctggagtg ggttggggaa atcagtcata gaggaagcac | |
| | caactacaac | |
| | ccgtccctca agagtcgagt caccatatca ctggacacgt ccaagaacca | |
| | gttctccctg | |
| | aagctgacct ctgtgaccgc cgcggacacg gctgtgtatt actgttcgag | |
| | agacgaggaa | |
| | ctggaattcc gtttctttga ctactggggc cagggaaccc tggtcaccgt | |
| | ctcctca | |
| 538. | QVQLQQWGAG LLKPSETLSL TCAVYGGSFS | AA amino |
| | GYYWNWIRQP PGKGLEWVGE ISHRGSTNYN | acid |
| | PSLKSRVTIS LDTSKNQFSL KLTSVTAADT | sequence |
| | AVYYCSRDEE LEFRFFDYWG QGTLVTVSS | |
| 539. | ggtgggtcct tcagtggtta ctac | DNA |
| | | nucleotide |
| | | sequence |
| 540. | GGSFSGYY | AA amino |
| | | acid |
| | | sequence |
| 541. | atcagtcata gaggaagcac c | DNA |
| | | nucleotide |
| | | sequence |
| 542. | ISHRGST | AA amino |
| | | acid |
| | | sequence |
| 543. | tcgagagacg aggaactgga attccgtttc tttgactac | DNA |
| | | nucleotide |
| | | sequence |
| 544. | SRDEELEFRF FDY | AA amino |
| | | acid |
| | | sequence |
| 545. | gaaattgtgt tgacacagtc tccagccacc ctgtctttgt ctccagggga | DNA |
| | aagagccacc | nucleotide |
| | ctctcctgca gggccagtca gagtgttagc agctatttag cctggtacca | sequence |
| | acaaaaacct | |
| | ggccaggctc ccaggctcct cgtctatggt gcatccaaca gggccactgg | |
| | catcccagcc | |
| | aggttcagtg gcagtgggtc tgggacagac ttcactctca ccatcagcag | |
| | cctagagcct | |
| | gaagattttg cattttatta ctgtcagcag cgtagcaact ggccgctcac | |
| | tttcggcgga | |
| | gggaccaagg tggagatcaa a | |
| 546. | EIVLTQSPAT LSLSPGERAT LSCRASQSVS | AA amino |
| | SYLAWYQQKP GQAPRLLVYG ASNRATGIPA | acid |
| | RFSGSGSGTD FTLTISSLEP EDFAFYYCQQ | Sequence |
| | RSNWPLTFGG GTKVEIK | |
| 547. | cagagtgtta gcagctat | DNA |
| | | nucleotide |
| | | sequence |
| 548. | QSVSSY | AA amino |
| | | acid |
| | | sequence |
| 549. | ggtgcatcc | DNA |
| | | nucleotide |
| | | sequence |
| 550. | GAS | AA amino |
| | | acid |
| | | sequence |
| 551. | cagcagcgta gcaactggcc gctcact | DNA |
| | | nucleotide |
| | | sequence |
| 552. | QQRSNWPLT | AA amino |
| | | acid |
| | | sequence |
| 553. | gaggtgcagc tggtggagtc tgggggaggc ttggtacagc ctggggggtc | DNA |
| | cctgagactc | nucleotide |
| | tcctgtgtag cctctggatt cacctttagc ctctatgcca tgacctgggt | sequence |
| | ccgccaggtt | |
| | ccagggaagg ggctggaatg ggtctcaact attagtggta gtggtggtgg | |
| | cacatactac | |
| | acagactccg ttaagggccg gttcaccatc tccagagaca attccaagaa | |
| | cacactgtat | |
| | ctgcaaatga acagcctgag agccgacgac acggccgttt tttactgtac | |
| | gaaagagagt | |
| | acaactggaa cttactccta cttctacggt atggacgtct ggggccaagg | |
| | gaccacggtc | |
| | accgtctcct ca | |
| 554. | EVQLVESGGG LVQPGGSLRL SCVASGFTFS | AA amino |
| | LYAMTWVRQV PGKGLEWVST ISGSGGGTYY | acid |
| | TDSVKGRFTI SRDNSKNTLY LQMNSLRADD | sequence |
| | TAVFYCTKES TTGTYSYFYG MDVWGQGTTV | |
| | TVSS | |
| 555. | ggattcacct ttagcctcta tgcc | DNA |
| | | nucleotide |
| | | sequence |
| 556. | GFTFSLYA | AA amino |
| | | acid |
| | | sequence |
| 557. | attagtggta gtggtggtgg caca | DNA |
| | | nucleotide |
| | | sequence |
| 558. | ISGSGGGT | AA amino |
| | | acid |
| | | sequence |
| 559. | acgaaagaga gtacaactgg aacttactcc tacttctacg gtatggacgt | DNA |
| | c | nucleotide |
| | | sequence |
| 560. | TKESTIGTYS YFYGMDV | AA amino |
| | | acid |
| | | sequence |
| 561. | gacatccaga tgacccagtc tccatcctcc ctgtctgcat ctgtaggaga | DNA |
| | cagagtcacc | nucleotide |
| | atcacttgcc gggcaagtca gaccattagc agctatttaa attggtatca | sequence |
| | gcagaaacca | |
| | gggaaagccc ctaagctcct gatctatgct gcatccagtt tgcaaagtgg | |
| | ggtcccatca | |
| | aggttcagtg gcagtggatc tgggacagat ttcactctca ccctcagcgg | |
| | tctccaacct | |
| | gaagattttg caacttacta ctgtcaacag agttacagta ccccgctcac | |
| | tttcggcgga | |
| | gggaccaagg tggagatcaa a | |
| 562. | DIQMTQSPSS LSASVGDRVT ITCRASQTIS | AA amino |
| | SYLNWYQQKP GKAPKLLIYA ASSLQSGVPS | acid |
| | RFSGSGSGTD FTLTLSGLQP EDFATYYCQQ | sequence |
| | SYSTPLTFGG GTKVEIK | |
| 563. | cagaccatta gcagctat | DNA |
| | | nucleotide |
| | | sequence |
| 564. | QTISSY | AA amino |
| | | acid |
| | | sequence |
| 565. | gctgcatcc | DNA |
| | | nucleotide |
| | | sequence |
| 566. | AAS | AA amino |
| | | acid |
| | | sequence |
| 567. | caacagagtt acagtacccc gctcact | DNA |
| | | nucleotide |
| | | sequence |
| 568. | QQSYSTPLT | AA amino |
| | | acid |
| | | sequence |
| 569. | ASTKGPSVFP LAPCSRSTSE STAALGCLVK | AA amino |
| | DYFPEPVTVS WNSGALTSGV HTFPAVLQSS | acid |
| | GLYSLSSVVT VPSSSLGTKT YTCNVDHKPS | sequence |
| | NTKVDKRVES KYGPPCPPCP APEFLGGPSV | |
| | FLFPPKPKDT LMISRTPEVT CVVVDVSQED | |
| | PEVQFNWYVD GVEVHNAKTK PREEQFNSTY | |
| | RVVSVLTVLH QDWLNGKEYK CKVSNKGLPS | |
| | SIEKTISKAK GQPREPQVYT LPPSQEEMTK | |
| | NQVSLTCLVK GFYPSDIAVE WESNGQPENN | |
| | YKTTPPVLDS DGSFFLYSRL TVDKSRWQEG | |
| | NVFSCSVMHE ALHNHYTQKS LSLSLGK | |
| 570. | ASTKGPSVFP LAPCSRSTSE STAALGQLVK | AA amino |
| | DYFPEPVTVS WNSGALTSGV HTFPAVLQSS | acid |
| | GLYSLSSVVT VPSSSLGTKT YTCNVDHKPS | sequence |
| | NTKVDKRVES KYGPPCPPCP APPVAGPSVF | |
| | LFPPKPKDTL MISRTPEVTC VVVDVSQEDP | |
| | EVQFNWYVDG VEVHNAKTKP REEQFNSTYR | |
| | VVSVLTVLHQ DWLNGKEYKC KVSNKGLPSS | |
| | IEKTISKAKG QPREPQVYTL PPSQEEMTKN | |
| | QVSLTCLVKG FYPSDIAVEW ESNGQPENNY | |
| | KTTPPVLDSD GSFFLYSRLT VDKSRWQEGN | |
| | VFSCSVMHEA LHNHYTQKSL SLSLGK | |
| 571. | ASTKGPSVFP LAPCSRSTSE STAALGQLVK | AA amino |
| | DYFPEPVTVS WNSGALTSGV HTFPAVLQSS | acid |
| | GLYSLSSVVT VPSSSLGTKT YTCNVDHKPS | sequence |
| | NTKVDKRVES KYGPPCPPCP APPVAGPSVF | |
| | LFPPKPKDTL MISRTPEVTC VVVDVSQEDP | |
| | EVQFNWYVDG VEVHNAKTKP REEQFNSTYR | |
| | VVSVLTVLHQ DWLNGKEYKQ KVSNKGLPSS | |
| | IEKTISKAKG QPREPQVYTL PPSQEEMTKN | |
| | QVSLTCLVKG FYPSDIAVEW ESNGQPENNY | |
| | KTTPPVLDSD GSFFLYSRLT VDKSRWQEGN | |
| | VFSCSVMHEA LHNRFTQKSL SLSPGK | |
| 572. | ASTKGPSVFP LAPCSRSTSE STAALGQLVK | AA amino |
| | DYFPEPVTVS WNSGALTSGV HTFPAVLQSS | acid |
| | GLYSLSSVVT VPSSSLGTKT YTCNVDHKPS | sequence |
| | NTKVDKRVES KYGPPCPPCP APGGGGPSVF | |
| | LFPPKPKDTL MISRTPEVTC VVVDVSQEDP | |
| | EVQFNWYVDG VEVHNAKTKP REEQFNSTYR | |
| | VVSVLTVLHQ DWLNGKEYKC KVSNKGLPSS | |
| | IEKTISKAKG QPREPQVYTL PPSQEEMTKN | |
| | QVSLTCLVKG FYPSDIAVEW ESNGQPENNY | |
| | KTTPPVLDSD GSFFLYSRLT VDKSRWQEGN | |
| | VFSCSVMHEA LHNHYTQKSL SLSLGK | |
| 573. | ASTKGPSVFP LAPCSRSTSE STAALGQLVK | AA amino |
| | DYFPEPVTVS WNSGALTSGV HTFPAVLQSS | acid |
| | GLYSLSSVVT VPSSSLGTKT YTCNVDHKPS | sequence |
| | NTKVDKRVES KYGPPCPPCP APGGGGPSVF | |
| | LFPPKPKDTL MISRTPEVTC VVVDVSQEDP | |
| | EVQFNWYVDG VEVHNAKTKP REEQFNSTYR | |
| | VVSVLTVLHQ DWLNGKEYKC KVSNKGLPSS | |
| | IEKTISKAKG QPREPQVYTL PPSQEEMTKN | |
| | QVSLTCLVKG FYPSDIAVEW ESNGQPENNY | |
| | KTTPPVLDSD GSFFLYSRLT VDKSRWQEGN | |
| | VFSCSVMHEA LHNRFTQKSL SLSPGK | |
| 574. | VPVVWAQEGA PAQLPCSPTI PLQDLSLLRR | AA amino |
| | AGVTWQHQPD SGPPAAAPGH PLAPGPHPAA | acid |
| | PSSWGPRPRR YTVLSVGPGG LRSGRLPLQP | sequence |
| | RVQLDERGRQ RGDFSLWLRP ARRADAGEYR | |
| | AAVHLRDRAL SCRLRLRLGQ ASMTASPPGS | |
| | LRASDWVILN CSFSRPDRPA SVHWFRNRGQ | |
| | GRVPVRESPH HHLAESFLFL PQVSPMDSGP | |
| | WGCILTYRDG FNVSIMYNLT VLGLEPPTPL | |
| | TVYAGAGSRV GLPCRLPAGV GTRSFLTAKW | |
| | TPPGGGPDLL VTGDNGDFTL RLEDVSQAQA | |
| | GTYTCHIHLQ EQQLNATVTL AIITVTPKSF | |
| | GSPGSLGKLL CEVTPVSGQE RFVWSSLDTP | |
| | SQRSFSGPWL EAQEAQLLSQ PWQCQLYQGE | |
| | RLLGAAVYFT ELSSPGAQRS GRAPGALPAG | |
| | HLEQKLISEE DLGGEQKLIS EEDLHHHHHH | |
| 575. | VPVVWAQEGA PAQLPCSPTI PLQDLSLLRR | AA amino |
| | AGVTWQHQPD SGPPAAAPGH PLAPGPHPAA | acid |
| | PSSWGPRPRR YTVLSVGPGG LRSGRLPLQP | sequence |
| | RVQLDERGRQ RGDFSLWLRP ARRADAGEYR | |
| | AAVHLRDRAL SCRLRLRLGQ ASMTASPPGS | |
| | LRASDWVILN CSFSRPDRPA SVHWFRNRGQ | |
| | GRVPVRESPH HHLAESFLFL PQVSPMDSGP | |
| | WGCILTYRDG FNVSIMYNLT VLGLEPPTPL | |
| | TVYAGAGSRV GLPCRLPAGV GTRSFLTAKW | |
| | TPPGGGPDLL VTGDNGDFTL RLEDVSQAQA | |
| | GTYTCHIHLQ EQQLNATVTL AIITVTPKSF | |
| | GSPGSLGKLL CEVTPVSGQE RFVWSSLDTP | |
| | SQRSFSGPWL EAQEAQLLSQ PWQCQLYQGE | |
| | RLLGAAVYFT ELSSPGAQRS GRAPGALPAG | |
| | HLEPRGPTIK PCPPCKCPAP NLLGGPSVFI | |
| | FPPKIKDVLM ISLSPIVTCV VVDVSEDDPD | |
| | VQISWFVNNV EVHTAQTQTH REDYNSTLRV | |
| | VSALPIQHQD WMSGKEFKCK VNNKDLPAPI | |
| | ERTISKPKGS VRAPQVYVLP PPEEEMTKKQ | |
| | VTLTCMVTDF MPEDIYVEWT NNGKTELNYK | |
| | NTEPVLDSDG SYFMYSKLRV EKKNWVERNS | |
| | YSCSVVHEGL HNHHTTKSFS RTPGK | |
| 576. | APVKPPQPGA EISVVWAQEG APAQLPCSPT | AA amino |
| | IPLQDLSLLR RAGVTWQHQP DSGPPAPAPG | acid |
| | HPPVPGHRPA APYSWGPRPR RYTVLSVGPG | sequence |
| | GLRSGRLPLQ PRVQLDERGR QRGDFSLWLR | |
| | PARRADAGEY RATVHLRDRA LSCRLRLRVG | |
| | QASMTASPPG SLRTSDWVIL NCSFSRPDRP | |
| | ASVHWFRSRG QGRVPVQGSP HHHLAESFLF | |
| | LPHVGPMDSG LWGCILTYRD GFNVSIMYNL | |
| | TVLGLEPATP LTVYAGAGSR VELPCRLPPA | |
| | VGTQSFLTAK WAPPGGGPDL LVAGDNGDFT | |
| | LRLEDVSQAQ AGTYICHIRL QGQQLNATVT | |
| | LAIITVTPKS FGSPGSLGKL LCEVTPASGQ | |
| | EHFVWSPLNT PSQRSFSGPW LEAQEAQLLS | |
| | QPWQCQLHQG ERLLGAAVYF TELSSPGAQR | |
| | SGRAPGALRA GHLPLFLILG VLFLLLLVTG | |
| | AFGFHLWRRQ WRPRRFSALE QGIHPPQAQS | |
| | KIEELEQEPE LEPEPELERE LGPEPEPGPE | |
| | PEPEQL | |
| 577. | QVQLVESGGG VVQPGRSLRL SCVASGFTFS | AA amino |
| | SYGMHWRQA PGKGLEWWVAI IWYDGSNKYY | acid |
| | ADSVKGRFTI SRDNSKNTQY LQMNSLRAED | sequence |
| | TAVYYCASVA TSGDFDYYGM DVWGQGTTVT | |
| | VSSASTKGPS VFPLAPCSRS TSESTAALGQ | |
| | LVKDYFPEPV TVSWNSGALT SGVHTFPAVL | |
| | QSSGLYSLSS VVTVPSSSLG TKTYTQNVDH | |
| | KPSNTKVDKR VESKYGPPCP PCPAPPVAGP | |
| | SVFLFPPKPK DTLMISRTPE VTCVVVDVSQ | |
| | EDPEVQFNWY VDGVEVHNAK TKPREEQFNS | |
| | TYRVVSVLTV LHQDWLNGKE YKCKVSNKGL | |
| | PSSIEKTISK AKGQPREPQV YTLPPSQEEM | |
| | TKNQVSLTCL VKGFYPSDIA VEWESNGQPE | |
| | NNYKTTPPVL DSDGSFFLYS RLTVDKSRWQ | |
| | EGNVFSCSVM HEALHNHYTQ KSLSLSLGK | |
| 578. | EIVLTQSPAT LSLSPGERTT LSCRASQRIS | AA amino |
| | TYLAWYQQKP GQAPRLLIYD ASKRATGIPA | acid |
| | RFSGSGSGTG FTLTISSLEP EDFAVYYCQQ | sequence |
| | RSNWPLTFGG GTKVEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSEN | |
| | RGEC | |
| 579. | QVQLVESGGG VVQPGRSLRL SCVASGFTFS | AA amino |
| | SYGMHWVRQA PGKGLEWVAI IWYDGSNKYY | acid |
| | ADSVKGRFTI SRDNSKNTQY LQMNSLRAED | sequence |
| | TAVYYCASVA TSGDFDYYGM DVWGQGTTVT | |
| | VSSASTKGPS VFPLAPCSRS TSESTAALGC | |
| | LVKDYFPEPV TVSWNSGALT SGVHTFPAVL | |
| | QSSGLYSLSS VVTVPSSSLG TKTYTCNVDH | |
| | KPSNTKVDKR VESKYGPPCP PCPAPEFLGG | |
| | PSVFLFPPKP KDTLMISRTP EVTCVVVDVS | |
| | QEDPEVQFNW YVDGVEVHNA KTKPREEQFN | |
| | STYRVVSVLT VLHQDWLNGK EYKCKVSNKG | |
| | LPSSIEKTIS KAKGQPREPQ VYTLPPSQEE | |
| | MTKNQVSLTC LVKGFYPSDI AVEWESNGQP | |
| | ENNYKTTPPV LDSDGSFFLY SRLTVDKSRW | |
| | QEGNVFSCSV MHEALHNHYT QKSLSLSLGK | |
| 580. | QVQLQQWGAG LLKPSETLSL TCAVYGGSFS | AA amino |
| | GYYWNWIRQP PGKGLEWVGE ISHRGSTNYN | acid |
| | PSLKSRVTIS LDTSKNQFSL KLTSVTAADT | sequence |
| | AVYYCSRDEE LEFRFFDYWG QGTLVTVSSA | |
| | STKGPSVFPL APCSRSTSES TAALGQLVKD | |
| | YFPEPVTVSW NSGALTSGVH TFPAVLQSSG | |
| | LYSLSSVVTV PSSSLGTKTY TCNVDHKPSN | |
| | TKVDKRVESK YGPPCPPCPA PPVAGPSVFL | |
| | FPPKPKDTLM ISRTPEVTCV VVDVSQEDPE | |
| | VQFNWYVDGV EVHNAKTKPR EEQFNSTYRV | |
| | VSVLTVLHQD WLNGKEYKCK VSNKGLPSSI | |
| | EKTISKAKGQ PREPQVYTLP PSQEEMTKNQ | |
| | VSLTCLVKGF YPSDIAVEWE SNGQPENNYK | |
| | TTPPVLDSDG SFFLYSRLTV DKSRWQEGNV | |
| | FSCSVMHEAL HNHYTQKSLS LSLGK | |
| 581. | EIVLTQSPAT LSLSPGERAT LSCRASQSVS | AA amino |
| | SYLAWYQQKP GQAPRLLVYG ASNRATGIPA | acid |
| | RFSGSGSGTD FTLTISSLEP EDFAFYYCQQ | sequence |
| | RSNWPLTFGG GTKVEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSFN | |
| | RGEC | |
| 582. | MWEAQFLGLL FLQPLWVAPV KPLQPGAEVP | AA amino |
| | VVWAQEGAPA QLPCSPTIPL QDLSLLRRAG | acid |
| | VTWQHQPDSG PPAAAPGHPL APGPHPAAPS | sequence |
| | SWGPRPRRYT VLSVGPGGLR SGRLPLQPRV | |
| | QLDERGRQRG DFSLWLRPAR RADAGEYRAA | |
| | VHLRDRALSC RLRLRLGQAS MTASPPGSLR | |
| | ASDWVILNCS FSRPDRPASV HWFRNRGQGR | |
| | VPVRESPHHH LAESFLFLPQ VSPMDSGPWG | |
| | CILTYRDGEN VSIMYNLTVL GLEPPTPLTV | |
| | YAGAGSRVGL PCRLPAGVGT RSFLTAKWTP | |
| | PGGGPDLLVT GDNGDFTLRL EDVSQAQAGT | |
| | YTCHIHLQEQ QLNATVTLAI ITVTPKSFGS | |
| | PGSLGKLLCE VTPVSGQERF VWSSLDTPSQ | |
| | RSFSGPWLEA QEAQLLSQPW QCQLYQGERL | |
| | LGAAVYFTEL SSPGAQRSGR APGALPAGHL | |
| | LLFLILGVLS LLLLVTGAFG FHLWRRQWRP | |
| | RRFSALEQGI HPPQAQSKIE ELEQEPEPEP | |
| | EPEPEPEPEP EPEQL | |
| 583. | ENPVVHFFKN IVTPR | AA amino |
| | | acid |
| | | sequence |
| 584. | agcagctctg ccctcat | DNA |
| | | nucleotide |
| | | sequence |
| 585. | gctctggctg gtcttcagta tg | DNA |
| | | nucleotide |
| | | sequence |
| 586. | ttgccgtatg gttggtttga ac | DNA |
| | | nucleotide |
| | | sequence |
| 587. | LQPGAEVPVV WAQEGAPAQL PCSPTIPLQD | AA amino |
| | LSLLRRAGVT WQHQPDSGPP AAAPGHPLAP | acid |
| | GPHPAAPSSW GPRPRRYTVL SVGPGGLRSG | sequence |
| | RLPLQPRVQL DERGRQRGDF SLWLRPARRA | |
| | DAGEYRAAVH LRDRALSCRL RLRLGQASMT | |
| | ASPPGSLRAS DWVILNCSFS RPDRPASVHW | |
| | FRNRGQGRVP VRESPHHHLA ESFLFLPQVS | |
| | PMDSGPWGCI LTYRDGFNVS IMYNLTVLGL | |
| | EPPTPLTVYA GAGSRVGLPC RLPAGVGTRS | |
| | FLTAKWTPPG GGPDLLVTGD NGDFTLRLED | |
| | VSQAQAGTYT CHIHLQEQQL NATVTLAIIT | |
| | VTPKSFGSPG SLGKLLCEVT PVSGQERFVW | |
| | SSLDTPSQRS FSGPWLEAQE AQLLSQPWQC | |
| | QLYQGERLLG AAVYFTELSS PGAQRSGRAP | |
| | GALPAGHLIE GRMDPKSCDK THTCPPCPAP | |
| | ELLGGPSVFL FPPKPKDTLM ISRTPEVTCV | |
| | VVDVSHEDPE VKFNWYVDGV EVHNAKTKPR | |
| | EEQYNSTYRV VSVLTVLHQD WLNGKEYKCK | |
| | VSNKALPAPI EKTISKAKGQ PREPQVYTLP | |
| | PSRDELTKNQ VSLTCLVKGF YPSDIAVEWE | |
| | SNGQPENNYK TTPPVLDSDG SFFLYSKLTV | |
| | DKSRWQQGNV FSCSVMHEAL HNHYTQKSLS | |
| | LSPGK | |
| 588. | VPVVWAQEGA PAQLPCSPTI PLQDLSLLRR | AA amino |
| | AGVTWQHQPD SGPPAAAPGH PLAPGPHPAA | acid |
| | PSSWGPRPRR YTVLSVGPGG LRSGRLPLQP | sequence |
| | RVQLDERGRQ RGDFSLWLRP ARRADAGEYR | |
| | AAVHLRDRAL SCRLRLRLGQ ASMTASPPGS | |
| | LRASDWVILN CSFSRPDRPA SVHWFRNRGQ | |
| | GRVPVRESPH HHLAESFLFL PQVSPMDSGP | |
| | WGCILTYRDG FNVSIMYNLT VLGLEPPTPL | |
| | TVYAGAGSRV GLPCRLPAGV GTRSFLTAKW | |
| | TPPGGGPDLL VTGDNGDFTL RLEDVSQAQA | |
| | GTYTCHIHLQ EQQLNATVTL AIITVTPKSF | |
| | GSPGSLGKLL CEVTPVSGQE RFVWSSLDTP | |
| | SQRSFSGPWL EAQEAQLLSQ PWQCQLYQGE | |
| | RLLGAAVYFT ELSSPGAQRS GRAPGALPAG | |
| | HL | |
| 589. | LRRAGVTWQH QPDSGPPAAA PGHPLAPGPH | AA amino |
| | PAAPSSWGPR PRRY | acid |
| | | sequence |

What is claimed is:

1. A composition comprising:
(i) a radiolabeled antibody conjugate comprising an antibody or antigen-binding fragment thereof that binds lymphocyte activation gene-3 (LAG3), a chelating moiety, and a positron emitter; and
(ii) LAG3 expressing T cells.

2. The composition of claim 1, wherein the antibody or antigen-binding fragment thereof comprises three heavy chain complementarity determining regions (HCDRs) and three light chain complementarity determining regions (LCDRs) within the heavy chain variable region (HCVR)/light chain variable region (LCVR) amino acid sequence pair, wherein the HCVR/LCVR amino acid sequence pair is selected from the group consisting of SEQ ID NOs: 2/10, 18/26, 34/42, 50/58, 66/74, 82/90, 98/106, 114/122, 130/138, 146/154, 162/170, 178/186, 194/202, 210/218, 226/234, 242/250, 258/266, 274/282, 290/298, 306/314, 322/330, 338/346, 354/362, 370/378, 386/394, 402/410, 418/426, 434/442, 450/522, 458/522, 466/522, 474/522, 482/522, 490/522, 498/530, 506/530, 514/530, 538/546, and 554/562.

3. The composition of claim 2, wherein the antibody or antigen-binding fragment thereof comprises a set of three HCDRs and three LCDRs, wherein the set is of CDRs is selected from the group consisting of SEQ ID NOs: 4/6/8/12/14/16, 20/22/24/28/30/32, 36/38/40/44/46/48, 52/54/56/60/62/64, 68/70/72/76/78/80, 84/86/88/92/94/96, 100/102/104/108/110/112, 132/134/136/140/142/144, 148/150/152/156/158/160, 164/166/168/172/174/176, 180/182/184/188/190/192, 196/198/200/204/206/208, 212/214/216/220/222/224, 228/230/232/236/238/240, 244/246/248/252/254/256, 260/262/264/268/270/272, 276/278/280/284/286/288, 292/294/296/300/302/304, 324/326/328/332/334/336, 340/342/344/348/350/352, 356/358/360/364/366/368, 372/374/376/380/382/384, 388/390/392/396/398/400, 404/406/408/412/414/416, 420/422/424/428/430/432, 436/438/440/444/446/448, 460/462/464/524/526/528, 468/470/472/524/526/528, 476/478/480/524/526/528, 484/486/488/524/526/528, 492/494/496/524/526/528, 500/502/504/532/534/536, 508/510/512/532/534/536, 516/518/520/532/534/536, 540/542/544/548/550/552, and 556/558/560/564/566/568, respectively.

4. The composition of claim 1, wherein the antibody or antigen-binding fragment thereof comprises three HCDRs in an HCVR amino acid sequence of SEQ ID NO: 418; and three LCDRs in an LCVR amino acid sequence of SEQ ID NO: 426.

5. The composition of claim 4, wherein the antibody or antigen-binding fragment thereof comprises an HCDR1 amino acid sequence comprising SEQ ID NO: 420; an HCDR2 amino acid sequence comprising SEQ ID NO: 422; an HCDR3 amino acid sequence comprising SEQ ID NO: 424; an LCDR1 amino acid sequence comprising SEQ ID NO: 428; an LCDR2 amino acid sequence comprising SEQ ID NO: 430; and an LCDR3 amino acid sequence comprising SEQ ID NO: 432.

6. The composition of claim 1, wherein the chelating moiety comprises desferrioxamine.

7. The composition of claim 1, wherein the positron emitter is ⁸⁹Zr.

8. A method for making a radiolabeled anti-LAG3 binding protein, comprising:
loading a positron emitter onto a conjugate comprising an anti-LAG3 binding protein modified with a chelating agent, thereby forming the radiolabeled anti-LAG3 binding protein,
wherein the anti-LAG3 binding protein comprises three HCDRs within an HCVR amino acid sequence of SEQ ID NO: 418, and three LCDRs within an LCVR amino acid sequence of SEQ ID NO: 426.

9. The method of claim 8, wherein the method further comprises:
forming the conjugate by reacting the anti-LAG3 binding protein with the chelating agent, wherein the chelating agent comprises a chelating moiety and a reactive moiety for conjugation to the anti-LAG3 binding protein, and
wherein the step of forming the conjugate occurs prior to loading the positron emitter.

10. The method of claim 9, wherein the reactive moiety is selected from the group consisting of an isothiocyanatobenzyl group, an n-hydroxysuccinimide ester, a 2,3,5,6 tetrafluorophenol ester, and an n-succinimidyl-S-acetylthioacetate moiety.

11. The method of claim 9, wherein the chelating agent has the following formula:

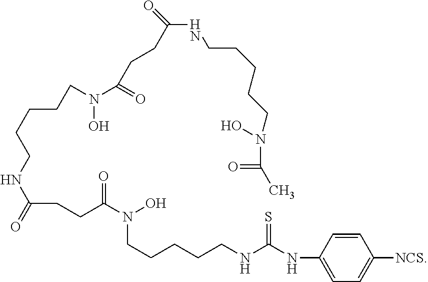

12. The method of claim 9, wherein the chelating moiety of the chelating agent comprises desferrioxamine.

13. The method of claim 9, wherein the chelating agent is p-isothiocyanatobenzyl-desferrioxamine.

14. The method of claim 8, wherein the positron emitter is ⁸⁹Zr.

15. The method of claim 8, wherein the positron emitter is an oxalic acid or other salt of ⁸⁹Zr.

16. The method of claim 8, wherein the chelating agent-to-anti-LAG3-binding protein ratio is from one to four.

17. The method of claim 8, wherein the chelating agent-to-anti-LAG3-binding protein ratio is from one to two.

18. The method of claim 8, wherein the chelating agent-to-anti-LAG3-binding protein ratio is 1.48.

19. The method of claim 8, wherein the conjugate has a formula: 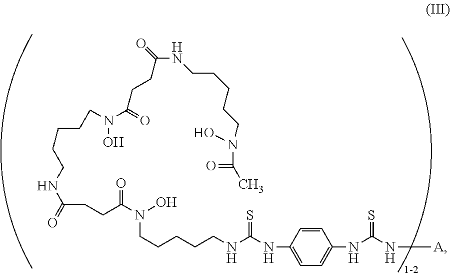
or a salt thereof, wherein -A is the anti-LAG3 binding protein.

20. The method of claim 8, wherein the radiolabeled anti-LAG3 binding protein is: 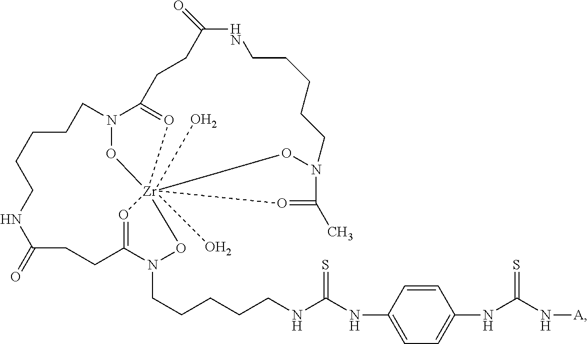
wherein A is the anti-LAG3 binding protein and Zr is the positron emitter ⁸⁹Zr.

21. The method of claim 8, wherein the step of loading the positron emitter comprises incubating the conjugate with the positron emitter.

22. The method of claim 8, wherein the anti-LAG3 binding protein has one or more properties selected from the group consisting of:
(a) binds monomeric human LAG3 with a binding dissociation equilibrium constant (K_{D}) of less than 8 nM as measured in a surface plasmon resonance assay at 37 °C;
(b) binds monomeric human LAG3 with a K_{D} less than 10 nM in a surface plasmon resonance assay at 25 °C;
(c) binds dimeric human LAG3 with a K_{D} of less than 1 nM as measured in a surface plasmon resonance assay at 37 °C; and
(d) binds dimeric human LAG3 with a K_{D} less than 1.1 nM in a surface plasmon resonance assay at 25 °C.

23. The method of claim 8, wherein the anti-LAG3 binding protein comprises the HCDR1 amino acid sequence of SEQ ID NO: 420, the HCDR2 amino acid sequence of SEQ ID NO: 422, the HCDR3 amino acid sequence of SEQ ID NO: 424, the LCDR1 amino acid sequence of SEQ ID NO: 428, the LCDR2 amino acid sequence of SEQ ID NO: 430, and the LCDR3 amino acid sequence of SEQ ID NO: 432.

24. The method of claim 8, wherein the anti-LAG3 binding protein comprises the HCVR amino acid sequence of SEQ ID NO: 418, and the LCVR amino acid sequence of SEQ ID NO: 426.


SEQUENCE LISTING (589 sequences)

SEQ ID NO: 1 - DNA, 372 bp, synthetic construct
  misc_feature 1..372: note = synthetic
  source 1..372: mol_type = other DNA
gaggtgcagctgttggagtctgggggaggcttggtacagcctggggggtccctgagactctcctgtgtggcctctggattcacctttagcacctatgccatgagttgggtccgccaggctccagggatggggctggagtgggtctcaagtattagtggtagtggtcgtaacacatactatgcagactccgtgaagggccggttcaccatctccagagacaattccaagaacacgctgtttcttcaaatgaacagcctgagagccgaggacacggccgtttattactgtgcgaaagagtccgtaactggaacttcgtcctactactacggtgtggacgtctggggccaagggaccacggtcaccgtctcctcg

SEQ ID NO: 2 - protein, 124 aa, synthetic construct
  REGION 1..124: note = synthetic
EVQLLESGGGLVQPGGSLRLSCVASGFTFSTYAMSWVRQAPGMGLEWVSSISGSGRNTYYADSVKGRFTISRDNSKNTLFLQMNSLRAEDTAVYYCAKESVTGTSSYYYGVDVWGQGTTVTVSS

SEQ ID NO: 3 - DNA, 24 bp, synthetic construct
  misc_feature 1..24: note = synthetic
  source 1..24: mol_type = other DNA
ggattcacctttagcacctatgcc

SEQ ID NO: 4 - protein, 8 aa, synthetic construct
  REGION 1..8: note = synthetic
GFTFSTYA

SEQ ID NO: 5 - DNA, 24 bp, synthetic construct
  misc_feature 1..24: note = synthetic
  source 1..24: mol_type = other DNA
attagtggtagtggtcgtaacaca

SEQ ID NO: 6 - protein, 8 aa, synthetic construct
  REGION 1..8: note = synthetic
ISGSGRNT

SEQ ID NO: 7 - DNA, 51 bp, synthetic construct
  misc_feature 1..51: note = synthetic
  source 1..51: mol_type = other DNA
gcgaaagagtccgtaactggaacttcgtcctactactacggtgtggacgtc

SEQ ID NO: 8 - protein, 17 aa, synthetic construct
  REGION 1..17: note = synthetic
AKESVTGTSSYYYGVDV

SEQ ID NO: 9 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagcattagcagttatttaaattggtatcatcagaaaccagggaaagccccaaagctcctgatctatgctgcatccagtttgcaaaatggggtcccatcaaggttcagtggcagtggatctgggacagatttcactctcaccatcagcagtctgcaacctgaagattttgcatcttactactgtcaacagagttacagaaccccgctcactttcggcggagggaccaaggtggagatcaaa

SEQ ID NO: 10 - protein, 107 aa, synthetic construct
  REGION 1..107: note = synthetic
DIQMTQSPSSLSASVGDRVTITCRASQSISSYLNWYHQKPGKAPKLLIYAASSLQNGVPSRFSGSGSGTDFTLTISSLQPEDFASYYCQQSYRTPLTFGGGTKVEIK

SEQ ID NO: 11 - DNA, 18 bp, synthetic construct
  misc_feature 1..18: note = synthetic
  source 1..18: mol_type = other DNA
cagagcattagcagttat

SEQ ID NO: 12 - protein, 6 aa, synthetic construct
  REGION 1..6: note = synthetic
QSISSY

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacagagttacagaaccccgctcact

SEQ ID NO: 16 - protein, 9 aa, synthetic construct
  REGION 1..9: note = synthetic
QQSYRTPLT

SEQ ID NO: 17 - DNA, 387 bp, synthetic construct
  misc_feature 1..387: note = synthetic
  source 1..387: mol_type = other DNA
caggtgcagctggaggagtctgggggaggcgtggtccagcctgggaggtccctgagactctcctgtgcagcgtctggattcaccttcagttggtatggcatgcactgggtccgccaggctccaggcaaggggctggagtgggtggcacttatatggtatgatggaactaataaaaagtatggagactccgtgaagggccgattcaccatttccagagacaattccaagaacacggtgtatctgcaaatgaacagcctgagagccgaggacacggctgtgtattactgtgcgagagattgtggacatagtggcaacgatcgggggacttactattactactacggtatggacgtctggggccaagggaccacggtcaccgtctcctca

SEQ ID NO: 18 - protein, 129 aa, synthetic construct
  REGION 1..129: note = synthetic
QVQLEESGGGVVQPGRSLRLSCAASGFTFSWYGMHWVRQAPGKGLEWVALIWYDGTNKKYGDSVKGRFTISRDNSKNTVYLQMNSLRAEDTAVYYCARDCGHSGNDRGTYYYYYGMDVWGQGTTVTVSS

SEQ ID NO: 19 - DNA, 24 bp, synthetic construct
  misc_feature 1..24: note = synthetic
  source 1..24: mol_type = other DNA
ggattcaccttcagttggtatggc

SEQ ID NO: 20 - protein, 8 aa, synthetic construct
  REGION 1..8: note = synthetic
GFTFSWYG

SEQ ID NO: 21 - DNA, 24 bp, synthetic construct
  misc_feature 1..24: note = synthetic
  source 1..24: mol_type = other DNA
atatggtatgatggaactaataaa

SEQ ID NO: 22 - protein, 8 aa, synthetic construct
  REGION 1..8: note = synthetic
IWYDGTNK

SEQ ID NO: 23 - DNA, 66 bp, synthetic construct
  misc_feature 1..66: note = synthetic
  source 1..66: mol_type = other DNA
gcgagagattgtggacatagtggcaacgatcgggggacttactattactactacggtatggacgtc

SEQ ID NO: 24 - protein, 22 aa, synthetic construct
  REGION 1..22: note = synthetic
ARDCGHSGNDRGTYYYYYGMDV

SEQ ID NO: 25 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 26 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 27 - DNA, 18 bp, synthetic construct
  misc_feature 1..18: note = synthetic
  source 1..18: mol_type = other DNA
cagagcattagcagctat

SEQ ID NO: 28 - protein, 6 aa, synthetic construct
  REGION 1..6: note = synthetic
QSISSY

SEQ ID NO: 29-30 - skipped (2 entries)

SEQ ID NO: 31 - DNA, 30 bp, synthetic construct
  misc_feature 1..30: note = synthetic
  source 1..30: mol_type = other DNA
caacagagttacagtacccctccgatcacc

SEQ ID NO: 32 - protein, 10 aa, synthetic construct
  REGION 1..10: note = synthetic
QQSYSTPPIT

SEQ ID NO: 33 - DNA, 357 bp, synthetic construct
  misc_feature 1..357: note = synthetic
  source 1..357: mol_type = other DNA
caggtgcagctacagcagtggggcgcaggactgttgaagccttcggagaccctgtccctcacctgcgctgtctatggtgggtccttcagtggttactactggaactggatccgccagcccccagggaaggggctggagtgggttggggaaatcagtcatagaggaaccaccaactacaacccgtccctcaagagtcgagtcaccatatcactggacacgtccaagaaccagttctccctgaaactgacctctgtgaccgccgcggacacggctgtgtattactgttcgagagacgaggaactggaattccgtttctttgactactggggccagggaaccctggtcaccgtctcctca

SEQ ID NO: 34 - protein, 119 aa, synthetic construct
  REGION 1..119: note = synthetic
QVQLQQWGAGLLKPSETLSLTCAVYGGSFSGYYWNWIRQPPGKGLEWVGEISHRGTTNYNPSLKSRVTISLDTSKNQFSLKLTSVTAADTAVYYCSRDEELEFRFFDYWGQGTLVTVSS

SEQ ID NO: 35 - DNA, 24 bp, synthetic construct
  misc_feature 1..24: note = synthetic
  source 1..24: mol_type = other DNA
ggtgggtccttcagtggttactac

SEQ ID NO: 36 - protein, 8 aa, synthetic construct
  REGION 1..8: note = synthetic
GGSFSGYY

SEQ ID NO: 37 - DNA, 21 bp, synthetic construct
  misc_feature 1..21: note = synthetic
  source 1..21: mol_type = other DNA
atcagtcatagaggaaccacc

SEQ ID NO: 38 - protein, 7 aa, synthetic construct
  REGION 1..7: note = synthetic
ISHRGTT

SEQ ID NO: 39 - DNA, 39 bp, synthetic construct
  misc_feature 1..39: note = synthetic
  source 1..39: mol_type = other DNA
tcgagagacgaggaactggaattccgtttctttgactac

SEQ ID NO: 40 - protein, 13 aa, synthetic construct
  REGION 1..13: note = synthetic
SRDEELEFRFFDY

SEQ ID NO: 41 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtgttagcagctatttagcctggtaccaacaaaaacctggccaggctcccaggctcctcgtctatggtgcatccaacagggccactggcatcccagccaggttcagtggcagtgggtctgggacagacttcactctcaccatcagcagcctagagcctgaagattttgcattttattactgtcagcagcgtagcaactggccgctcactttcggcggagggaccaaggtggagatcaaa

SEQ ID NO: 42 - protein, 107 aa, synthetic construct
  REGION 1..107: note = synthetic
EIVLTQSPATLSLSPGERATLSCRASQSVSSYLAWYQQKPGQAPRLLVYGASNRATGIPARFSGSGSGTDFTLTISSLEPEDFAFYYCQQRSNWPLTFGGGTKVEIK

SEQ ID NO: 43 - DNA, 18 bp, synthetic construct
  misc_feature 1..18: note = synthetic
  source 1..18: mol_type = other DNA
cagagtgttagcagctat

SEQ ID NO: 44 - protein, 6 aa, synthetic construct
  REGION 1..6: note = synthetic
QSVSSY

SEQ ID NO: 45-46 - skipped (2 entries)

SEQ ID NO: 47 - DNA, 27 bp, synthetic construct
  misc_feature 1..27: note = synthetic
  source 1..27: mol_type = other DNA
cagcagcgtagcaactggccgctcact

SEQ ID NO: 48 - protein, 9 aa, synthetic construct
  REGION 1..9: note = synthetic
QQRSNWPLT

SEQ ID NO: 49 - DNA, 357 bp, synthetic construct
  misc_feature 1..357: note = synthetic
  source 1..357: mol_type = other DNA
cagctgcagctgcaggagtcgggcccaggactggtgaagccttcggagaccctgtccctcacctgcactgtctctggtgactccatcatcagtaatagttattactggggctggatccgccagcccccagggaaggggctggagtggattggcaatttcttttatactggggccacctactacaacccgtccctcaagagtcgagtcaccatatccgctgacacgtccaagaatcagttctccctgaagctgagctctgtgaccgccgcagacacggctctgtattattgtgcgagttataataggaattaccggttcgacccctggggccagggaaccctggtcaccgtctcctca

SEQ ID NO: 50 - protein, 119 aa, synthetic construct
  REGION 1..119: note = synthetic
QLQLQESGPGLVKPSETLSLTCTVSGDSIISNSYYWGWIRQPPGKGLEWIGNFFYTGATYYNPSLKSRVTISADTSKNQFSLKLSSVTAADTALYYCASYNRNYRFDPWGQGTLVTVSS

SEQ ID NO: 51 - DNA, 30 bp, synthetic construct
  misc_feature 1..30: note = synthetic
  source 1..30: mol_type = other DNA
ggtgactccatcatcagtaatagttattac

SEQ ID NO: 52 - protein, 10 aa, synthetic construct
  REGION 1..10: note = synthetic
GDSIISNSYY

SEQ ID NO: 53 - DNA, 21 bp, synthetic construct
  misc_feature 1..21: note = synthetic
  source 1..21: mol_type = other DNA
ttcttttatactggggccacc

SEQ ID NO: 54 - protein, 7 aa, synthetic construct
  REGION 1..7: note = synthetic
FFYTGAT

SEQ ID NO: 55 - DNA, 33 bp, synthetic construct
  misc_feature 1..33: note = synthetic
  source 1..33: mol_type = other DNA
gcgagttataataggaattaccggttcgacccc

SEQ ID NO: 56 - protein, 11 aa, synthetic construct
  REGION 1..11: note = synthetic
ASYNRNYRFDP

SEQ ID NO: 57 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagagttacagtacccctccgatcaccttcggccaagggacacgactggagattaaa

SEQ ID NO: 58 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYFCQQSYSTPPITFGQGTRLEIK

SEQ ID NO: 59 - DNA, 18 bp, synthetic construct
  misc_feature 1..18: note = synthetic
  source 1..18: mol_type = other DNA
cagagcattagcagctat

SEQ ID NO: 60 - protein, 6 aa, synthetic construct
  REGION 1..6: note = synthetic
QSISSY

SEQ ID NO: 61-62 - skipped (2 entries)

SEQ ID NO: 63 - DNA, 30 bp, synthetic construct; identical to SEQ ID NO: 31
  misc_feature 1..30: note = synthetic
  source 1..30: mol_type = other DNA

SEQ ID NO: 64 - protein, 10 aa, synthetic construct
  REGION 1..10: note = synthetic
QQSYSTPPIT

SEQ ID NO: 65 - DNA, 381 bp, synthetic construct
  misc_feature 1..381: note = synthetic
  source 1..381: mol_type = other DNA
caggtgcagctacagcagtggggcgcaggactgttgaagccttcggagaccctgtccctcacctgcgctgtctatggtgggtccttcagtacttactactggagctggatccgccagcccccagggaaggggctggagtggattggagagatcaatcatagtggaaacgccgactacaacccgtccctcaagagtcgagtctccatatcagtggacacgtccaagaaccagttctccctgaggctgagctctgtgaccgccgcggacacggctatttattactgtgcgagagcgggctattgtagtagtcccacctgctattcctactactacttcggtatggacgtctggggccaagggaccacggtcaccgtctcctca

SEQ ID NO: 66 - protein, 127 aa, synthetic construct
  REGION 1..127: note = synthetic
QVQLQQWGAGLLKPSETLSLTCAVYGGSFSTYYWSWIRQPPGKGLEWIGEINHSGNADYNPSLKSRVSISVDTSKNQFSLRLSSVTAADTAIYYCARAGYCSSPTCYSYYYFGMDVWGQGTTVTVSS

SEQ ID NO: 67 - DNA, 24 bp, synthetic construct
  misc_feature 1..24: note = synthetic
  source 1..24: mol_type = other DNA
ggtgggtccttcagtacttactac

SEQ ID NO: 68 - protein, 8 aa, synthetic construct
  REGION 1..8: note = synthetic
GGSFSTYY

SEQ ID NO: 69 - DNA, 21 bp, synthetic construct
  misc_feature 1..21: note = synthetic
  source 1..21: mol_type = other DNA
atcaatcatagtggaaacgcc

SEQ ID NO: 70 - protein, 7 aa, synthetic construct
  REGION 1..7: note = synthetic
INHSGNA

SEQ ID NO: 71 - DNA, 63 bp, synthetic construct
  misc_feature 1..63: note = synthetic
  source 1..63: mol_type = other DNA
gcgagagcgggctattgtagtagtcccacctgctattcctactactacttcggtatggacgtc

SEQ ID NO: 72 - protein, 21 aa, synthetic construct
  REGION 1..21: note = synthetic
ARAGYCSSPTCYSYYYFGMDV

SEQ ID NO: 73 - DNA, 324 bp, synthetic construct
  misc_feature 1..324: note = synthetic
  source 1..324: mol_type = other DNA
gaaattgtgttgacgcagtctccaggcaccctgtctttgtctctaggggaaagagccaccctctcctgcagggccagtcagagtgttatcagcagcttcttagcctggtaccagcagaaacctggccaggctcccaggctcctcatctatggtgcatccagcagggccactggcttcccagacaggttcagtggcagtgggtctgggacagacttcactctcaccatccgcagactggagcctgaagattttgcagtgtattactgtcagcagtatggtaactcaccttggacgttcggccaagggaccaaggtggagatcaaa

SEQ ID NO: 74 - protein, 108 aa, synthetic construct
  REGION 1..108: note = synthetic
EIVLTQSPGTLSLSLGERATLSCRASQSVISSFLAWYQQKPGQAPRLLIYGASSRATGFPDRFSGSGSGTDFTLTIRRLEPEDFAVYYCQQYGNSPWTFGQGTKVEIK

SEQ ID NO: 75 - DNA, 21 bp, synthetic construct
  misc_feature 1..21: note = synthetic
  source 1..21: mol_type = other DNA
cagagtgttatcagcagcttc

SEQ ID NO: 76 - protein, 7 aa, synthetic construct
  REGION 1..7: note = synthetic
QSVISSF

SEQ ID NO: 77-78 - skipped (2 entries)

SEQ ID NO: 79 - DNA, 27 bp, synthetic construct
  misc_feature 1..27: note = synthetic
  source 1..27: mol_type = other DNA
cagcagtatggtaactcaccttggacg

SEQ ID NO: 80 - protein, 9 aa, synthetic construct
  REGION 1..9: note = synthetic
QQYGNSPWT

SEQ ID NO: 81 - DNA, 387 bp, synthetic construct
  misc_feature 1..387: note = synthetic
  source 1..387: mol_type = other DNA
caggtcaccttgaaggagtctggtcctgtgctggtgaaacccacagagaccctcacgctgacctgcaccgtctctgggttctcactcagcaatgctgggatgggtgtgagctgggtccgtcagccccctgggaaggccctggagtggcttgcacacattttttcgaatgacgagaagtcctacagcacatctctgaggaccagactcaccatctccaaggacacctccaaaagccaggtggtccttaccgtgaccaacttggaccctgtggacacagccacatatttctgtgcacggataccagagtttaccagctcgtcgtgggctctctactacttctacggtatggacgtctggggccaagggaccacggtcaccgtctcctca

SEQ ID NO: 82 - protein, 129 aa, synthetic construct
  REGION 1..129: note = synthetic
QVTLKESGPVLVKPTETLTLTCTVSGFSLSNAGMGVSWVRQPPGKALEWLAHIFSNDEKSYSTSLRTRLTISKDTSKSQVVLTVTNLDPVDTATYFCARIPEFTSSSWALYYFYGMDVWGQGTTVTVSS

SEQ ID NO: 83 - DNA, 30 bp, synthetic construct
  misc_feature 1..30: note = synthetic
  source 1..30: mol_type = other DNA
gggttctcactcagcaatgctgggatgggt

SEQ ID NO: 84 - protein, 10 aa, synthetic construct
  REGION 1..10: note = synthetic
GFSLSNAGMG

SEQ ID NO: 85 - DNA, 21 bp, synthetic construct
  misc_feature 1..21: note = synthetic
  source 1..21: mol_type = other DNA
attttttcgaatgacgagaag

SEQ ID NO: 86 - protein, 7 aa, synthetic construct
  REGION 1..7: note = synthetic
IFSNDEK

SEQ ID NO: 87 - DNA, 63 bp, synthetic construct
  misc_feature 1..63: note = synthetic
  source 1..63: mol_type = other DNA
gcacggataccagagtttaccagctcgtcgtgggctctctactacttctacggtatggacgtc

SEQ ID NO: 88 - protein, 21 aa, synthetic construct
  REGION 1..21: note = synthetic
ARIPEFTSSSWALYYFYGMDV

SEQ ID NO: 89 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagcgccaccctctcctgcagggccagtcagagtattaccagcacctacttcgcctggtaccagcagaaacctggccaggctcccaggctcctcatctatgctacatccagcagggccactggcgtcccagacaggttcagtggcagtgggtctgggacggacttcactctcaccatcagcagactggagcctgatgattttgcagtgtattactgtcagcaatatggtaggtcaccttggacgttcggccaagggaccaaggtggaagtcaaa

SEQ ID NO: 90 - protein, 108 aa, synthetic construct
  REGION 1..108: note = synthetic
EIVLTQSPGTLSLSPGESATLSCRASQSITSTYFAWYQQKPGQAPRLLIYATSSRATGVPDRFSGSGSGTDFTLTISRLEPDDFAVYYCQQYGRSPWTFGQGTKVEVK

SEQ ID NO: 91 - DNA, 21 bp, synthetic construct
  misc_feature 1..21: note = synthetic
  source 1..21: mol_type = other DNA
cagagtattaccagcacctac

SEQ ID NO: 92 - protein, 7 aa, synthetic construct
  REGION 1..7: note = synthetic
QSITSTY

SEQ ID NO: 93-94 - skipped (2 entries)

SEQ ID NO: 95 - DNA, 27 bp, synthetic construct
  misc_feature 1..27: note = synthetic
  source 1..27: mol_type = other DNA
cagcaatatggtaggtcaccttggacg

SEQ ID NO: 96 - protein, 9 aa, synthetic construct
  REGION 1..9: note = synthetic
QQYGRSPWT

SEQ ID NO: 97 - DNA, 363 bp, synthetic construct
  misc_feature 1..363: note = synthetic
  source 1..363: mol_type = other DNA
caggttcagctggtgcagtctggagctgaggtgaagaagcctggggcctcagtgaaggtctcctgcaaggcttctggttacacctttaccagttatggtatcagctgggtgcgacaggcccctggacaagggcttgagtggatgggatggatcagcgcttacaatgataacacaaactatgcacagaagctccagggcagagtcaccatgaccgcagacacatccacgaatacagcctacatggagctaaggagcctgagatctgacgacacggccatttattactgtgtgcgatggaattggggttccgtctactggtacttcgatctctggggccgtggcaccctggtcactgtctcctca

SEQ ID NO: 98 - protein, 121 aa, synthetic construct
  REGION 1..121: note = synthetic
QVQLVQSGAEVKKPGASVKVSCKASGYTFTSYGISWVRQAPGQGLEWMGWISAYNDNTNYAQKLQGRVTMTADTSTNTAYMELRSLRSDDTAIYYCVRWNWGSVYWYFDLWGRGTLVTVSS

SEQ ID NO: 99 - DNA, 24 bp, synthetic construct
  misc_feature 1..24: note = synthetic
  source 1..24: mol_type = other DNA
ggttacacctttaccagttatggt

SEQ ID NO: 100 - protein, 8 aa, synthetic construct
  REGION 1..8: note = synthetic
GYTFTSYG

SEQ ID NO: 101 - DNA, 24 bp, synthetic construct
  misc_feature 1..24: note = synthetic
  source 1..24: mol_type = other DNA
atcagcgcttacaatgataacaca

SEQ ID NO: 102 - protein, 8 aa, synthetic construct
  REGION 1..8: note = synthetic
ISAYNDNT

SEQ ID NO: 103 - DNA, 42 bp, synthetic construct
  misc_feature 1..42: note = synthetic
  source 1..42: mol_type = other DNA
gtgcgatggaattggggttccgtctactggtacttcgatctc

SEQ ID NO: 104 - protein, 14 aa, synthetic construct
  REGION 1..14: note = synthetic
VRWNWGSVYWYFDL

SEQ ID NO: 105 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagagccaccctctcctgcagggccagtcagattattagcagcagctactttgcctggtaccagcagaaacctggccaggctcccaggctcctcatctatggtgcgtccagcagggccactggcatcccagacaggttcagtggcagtgtgtctgggacagacttcactctcaccatcagcagactggagcctgaagattttgcaatgtatttctgtcagcagtatggtaactcaccttggacgttcggccaagggaccaaggtggaaatcaaa

SEQ ID NO: 106 - protein, 108 aa, synthetic construct
  REGION 1..108: note = synthetic
EIVLTQSPGTLSLSPGERATLSCRASQIISSSYFAWYQQKPGQAPRLLIYGASSRATGIPDRFSGSVSGTDFTLTISRLEPEDFAMYFCQQYGNSPWTFGQGTKVEIK

SEQ ID NO: 107 - DNA, 21 bp, synthetic construct
  misc_feature 1..21: note = synthetic
  source 1..21: mol_type = other DNA
cagattattagcagcagctac

SEQ ID NO: 108 - protein, 7 aa, synthetic construct
  REGION 1..7: note = synthetic
QIISSSY

SEQ ID NO: 109-110 - skipped (2 entries)

SEQ ID NO: 111 - DNA, 27 bp, synthetic construct
  misc_feature 1..27: note = synthetic
  source 1..27: mol_type = other DNA
cagcagtatggtaactcaccttggacg

SEQ ID NO: 112 - protein, 9 aa, synthetic construct
  REGION 1..9: note = synthetic
QQYGNSPWT

SEQ ID NO: 113 - DNA, 351 bp, synthetic construct
  misc_feature 1..351: note = synthetic
  source 1..351: mol_type = other DNA
cagatcaccttgaaggagtctggtcctacgctggtgaaacccacacagaccctcacgctgacttgcaccttctctgggttctcactcaacactcatagagtgggtgtaggctggatccggcagcccccaggaaaggccctggagtggcttgcactcatttatgggaatgatgttaagaactacagcccatctctggagaccaggctcaccatcgccaaggacacctccaaaaaccaggtggtccttacaatgaccaacatggaccctgtggacacagccacatatttctgttcgtacataacgggggaaggaatgtactggggccagggaaccctggtcaccgtctcctca

SEQ ID NO: 114 - protein, 117 aa, synthetic construct
  REGION 1..117: note = synthetic
QITLKESGPTLVKPTQTLTLTCTFSGFSLNTHRVGVGWIRQPPGKALEWLALIYGNDVKNYSPSLETRLTIAKDTSKNQVVLTMTNMDPVDTATYFCSYITGEGMYWGQGTLVTVSS

SEQ ID NO: 115 - DNA, 30 bp, synthetic construct
  misc_feature 1..30: note = synthetic
  source 1..30: mol_type = other DNA
gggttctcactcaacactcatagagtgggt

SEQ ID NO: 116 - protein, 10 aa, synthetic construct
  REGION 1..10: note = synthetic
GFSLNTHRVG

SEQ ID NO: 117 - DNA, 21 bp, synthetic construct
  misc_feature 1..21: note = synthetic
  source 1..21: mol_type = other DNA
atttatgggaatgatgttaag

SEQ ID NO: 118 - protein, 7 aa, synthetic construct
  REGION 1..7: note = synthetic
IYGNDVK

SEQ ID NO: 119 - DNA, 27 bp, synthetic construct
  misc_feature 1..27: note = synthetic
  source 1..27: mol_type = other DNA
tcgtacataacgggggaaggaatgtac

SEQ ID NO: 120 - protein, 9 aa, synthetic construct
  REGION 1..9: note = synthetic
SYITGEGMY

SEQ ID NO: 121 - DNA, 333 bp, synthetic construct
  misc_feature 1..333: note = synthetic
  source 1..333: mol_type = other DNA
gatgttgtgatgactcagtctccactctccctgtccgtcacccttggacagccggcctccatttcctgtaggtctagtcaaaacctcatgtacagtgatggaaacacctacttgaattggtttcaccagaggccaggccaatctccaaggcgtctaatttataaggtttctaaccgggactctggggtcccagacagattcagcggcagtgggtcaggcactgatttcacactgaaaatcagcagggtggaggctgaggatgttggggtttattactgcatgcaaggtacacactggtacacatttggccaggggaccaagctggagatcaaa

SEQ ID NO: 122 - protein, 111 aa, synthetic construct
  REGION 1..111: note = synthetic
DVVMTQSPLSLSVTLGQPASISCRSSQNLMYSDGNTYLNWFHQRPGQSPRRLIYKVSNRDSGVPDRFSGSGSGTDFTLKISRVEAEDVGVYYCMQGTHWYTFGQGTKLEIK

SEQ ID NO: 123 - DNA, 33 bp, synthetic construct
  misc_feature 1..33: note = synthetic
  source 1..33: mol_type = other DNA
caaaacctcatgtacagtgatggaaacacctac

SEQ ID NO: 124 - protein, 11 aa, synthetic construct
  REGION 1..11: note = synthetic
QNLMYSDGNTY

SEQ ID NO: 125-126 - skipped (2 entries)

SEQ ID NO: 127 - DNA, 24 bp, synthetic construct
  misc_feature 1..24: note = synthetic
  source 1..24: mol_type = other DNA
atgcaaggtacacactggtacaca

SEQ ID NO: 128 - protein, 8 aa, synthetic construct
  REGION 1..8: note = synthetic
MQGTHWYT

SEQ ID NO: 129 - DNA, 357 bp, synthetic construct
  misc_feature 1..357: note = synthetic
  source 1..357: mol_type = other DNA
caggtgcagctgcagcagtggggcgcaggactattgaagccttcggagaccctgtccctcacctgcgctgtctatggtgggtctttcagtggttattactggagctggatccgccagcccccagggaagggtctggaatggattggggaaatcaatcatagaggaaacaccaactacaacccgtccctcaagagtcgagtcaccatatcactcgacacgtccaagaaacagttctccctgaacctgagttctgtgaccgccgcggacacggctatgtattactgtacgagagacgaagaacaggaactacgtttccttgactactggggccagggaaccctggtcaccgtctcctca

SEQ ID NO: 130 - protein, 119 aa, synthetic construct
  REGION 1..119: note = synthetic
QVQLQQWGAGLLKPSETLSLTCAVYGGSFSGYYWSWIRQPPGKGLEWIGEINHRGNTNYNPSLKSRVTISLDTSKKQFSLNLSSVTAADTAMYYCTRDEEQELRFLDYWGQGTLVTVSS

SEQ ID NO: 131 - DNA, 24 bp, synthetic construct
  misc_feature 1..24: note = synthetic
  source 1..24: mol_type = other DNA
ggtgggtctttcagtggttattac

SEQ ID NO: 132 - protein, 8 aa, synthetic construct
  REGION 1..8: note = synthetic
GGSFSGYY

SEQ ID NO: 133 - DNA, 21 bp, synthetic construct
  misc_feature 1..21: note = synthetic
  source 1..21: mol_type = other DNA
atcaatcatagaggaaacacc

SEQ ID NO: 134 - protein, 7 aa, synthetic construct
  REGION 1..7: note = synthetic
INHRGNT

SEQ ID NO: 135 - DNA, 39 bp, synthetic construct
  misc_feature 1..39: note = synthetic
  source 1..39: mol_type = other DNA
acgagagacgaagaacaggaactacgtttccttgactac

SEQ ID NO: 136 - protein, 13 aa, synthetic construct
  REGION 1..13: note = synthetic
TRDEEQELRFLDY

SEQ ID NO: 137 - DNA, 321 bp, synthetic construct
  misc_feature 1..321: note = synthetic
  source 1..321: mol_type = other DNA
gagattgtgttgacacagtctccagccaccctgtctttgtctccaggggaaagagccaccctctcctgcagggccagtcaggatattagcacctacttagcctggtaccaacagagagctggccaggctcccaggctcctcatctatggtgcttccaacagggccactggcatcccagccaggttcagtggcagtgggtctgggacagacttcactctcaccatcagcagcctagagcctgaagattttgcattttattactgtcaacagcgcagcaactggccgctcactttcggcggagggaccgaggtggagatcaaa

SEQ ID NO: 138 - protein, 107 aa, synthetic construct
  REGION 1..107: note = synthetic
EIVLTQSPATLSLSPGERATLSCRASQDISTYLAWYQQRAGQAPRLLIYGASNRATGIPARFSGSGSGTDFTLTISSLEPEDFAFYYCQQRSNWPLTFGGGTEVEIK

SEQ ID NO: 139 - DNA, 18 bp, synthetic construct
  misc_feature 1..18: note = synthetic
  source 1..18: mol_type = other DNA
caggatattagcacctac

SEQ ID NO: 140 - protein, 6 aa, synthetic construct
  REGION 1..6: note = synthetic
QDISTY

SEQ ID NO: 141-142 - skipped (2 entries)

SEQ ID NO: 143 - DNA, 27 bp, synthetic construct
  misc_feature 1..27: note = synthetic
  source 1..27: mol_type = other DNA
caacagcgcagcaactggccgctcact

SEQ ID NO: 144 - protein, 9 aa, synthetic construct
  REGION 1..9: note = synthetic
QQRSNWPLT

SEQ ID NO: 145 - DNA, 366 bp, synthetic construct
  misc_feature 1..366: note = synthetic
  source 1..366: mol_type = other DNA
caggtgcagctacagcagtggggcgcaggactgttgaagccttcggagaccctgtccctcacctgcgttgtccatggtgggtccttcagtggttactactggaactggatccgccagcccccagggaaggggctggagtggattggggaaatcaatcatagaggaaacaccaactacaacccgtccctcaagagtcgagtcaccgtatcagaagacacgtccaagaaccagttctccctgaagctgagctctttgaccgccgcggacacggctgtgtattactgtgtgagaggagaggattacgatttttggagtgattattataatgactactggggccagggaaccctggtcaccgtctcctca

SEQ ID NO: 146 - protein, 122 aa, synthetic construct
  REGION 1..122: note = synthetic
QVQLQQWGAGLLKPSETLSLTCVVHGGSFSGYYWNWIRQPPGKGLEWIGEINHRGNTNYNPSLKSRVTVSEDTSKNQFSLKLSSLTAADTAVYYCVRGEDYDFWSDYYNDYWGQGTLVTVSS

SEQ ID NO: 147 - DNA, 24 bp, synthetic construct
  misc_feature 1..24: note = synthetic
  source 1..24: mol_type = other DNA
ggtgggtccttcagtggttactac

SEQ ID NO: 148 - protein, 8 aa, synthetic construct
  REGION 1..8: note = synthetic
GGSFSGYY

SEQ ID NO: 149 - DNA, 21 bp, synthetic construct
  misc_feature 1..21: note = synthetic
  source 1..21: mol_type = other DNA
atcaatcatagaggaaacacc

SEQ ID NO: 150 - protein, 7 aa, synthetic construct
  REGION 1..7: note = synthetic
INHRGNT

SEQ ID NO: 151 - DNA, 48 bp, synthetic construct
  misc_feature 1..48: note = synthetic
  source 1..48: mol_type = other DNA
gtgagaggagaggattacgatttttggagtgattattataatgactac

SEQ ID NO: 152 - protein, 16 aa, synthetic construct
  REGION 1..16: note = synthetic
VRGEDYDFWSDYYNDY

SEQ ID NO: 153 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagactattagcagctacttagcctggcaccaacagaaacctggccaggctcccaggctcctcatctatgatgcatccaaaagggccacgggcatcccagccaggttcagtggcagtgggtctgggacagacttcactctcaccatcaccagcctagagcctgaagattttgcagtttattactgtcagcagcgtagcaactggcctctcactttcggcggagggaccaaggtggagatcaaa

SEQ ID NO: 154 - protein, 107 aa, synthetic construct
  REGION 1..107: note = synthetic
EIVLTQSPATLSLSPGERATLSCRASQTISSYLAWHQQKPGQAPRLLIYDASKRATGIPARFSGSGSGTDFTLTITSLEPEDFAVYYCQQRSNWPLTFGGGTKVEIK

SEQ ID NO: 155 - DNA, 18 bp, synthetic construct
  misc_feature 1..18: note = synthetic
  source 1..18: mol_type = other DNA
cagactattagcagctac

SEQ ID NO: 156 - protein, 6 aa, synthetic construct
  REGION 1..6: note = synthetic
QTISSY

SEQ ID NO: 157-158 - skipped (2 entries)

SEQ ID NO: 159 - DNA, 27 bp, synthetic construct
  misc_feature 1..27: note = synthetic
  source 1..27: mol_type = other DNA
cagcagcgtagcaactggcctctcact

SEQ ID NO: 160 - protein, 9 aa, synthetic construct
  REGION 1..9: note = synthetic
QQRSNWPLT

SEQ ID NO: 161 - DNA, 372 bp, synthetic construct
  misc_feature 1..372: note = synthetic
  source 1..372: mol_type = other DNA
caggtgcagctacagcagtggggcgcaggactgttgccgccttcggagaccctgtccctcatctgcgctgtctatggtgggtccttcagtggttactactggagctggatccgccagcccccagggaaggggctggagtggattggggaaatcaatcatagaggaagcaccaactacaacccgtccctcaagagtcgagccaccatatcagttgacacgtccaagaaccagttctccctgaagctgagctctgtgaccgccgcggacacggctgtgtattactgttcgagaggcgaggattactatgatagtagtggttactcgtactactttgactactggggccagggaaccctggtcaccgtctcctca

SEQ ID NO: 162 - protein, 124 aa, synthetic construct
  REGION 1..124: note = synthetic
QVQLQQWGAGLLPPSETLSLICAVYGGSFSGYYWSWIRQPPGKGLEWIGEINHRGSTNYNPSLKSRATISVDTSKNQFSLKLSSVTAADTAVYYCSRGEDYYDSSGYSYYFDYWGQGTLVTVSS

SEQ ID NO: 163 - DNA, 24 bp, synthetic construct
  misc_feature 1..24: note = synthetic
  source 1..24: mol_type = other DNA
ggtgggtccttcagtggttactac

SEQ ID NO: 164 - protein, 8 aa, synthetic construct
  REGION 1..8: note = synthetic
GGSFSGYY

SEQ ID NO: 165 - DNA, 21 bp, synthetic construct
  misc_feature 1..21: note = synthetic
  source 1..21: mol_type = other DNA
atcaatcatagaggaagcacc

SEQ ID NO: 166 - protein, 7 aa, synthetic construct
  REGION 1..7: note = synthetic
INHRGST

SEQ ID NO: 167 - DNA, 54 bp, synthetic construct
  misc_feature 1..54: note = synthetic
  source 1..54: mol_type = other DNA
tcgagaggcgaggattactatgatagtagtggttactcgtactactttgactac

SEQ ID NO: 168 - protein, 18 aa, synthetic construct
  REGION 1..18: note = synthetic
SRGEDYYDSSGYSYYFDY

SEQ ID NO: 169 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggagatcaaa

SEQ ID NO: 170 - protein, 107 aa, synthetic construct
  REGION 1..107: note = synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 171 - DNA, 18 bp, synthetic construct
  misc_feature 1..18: note = synthetic
  source 1..18: mol_type = other DNA
cagagtgttagcagctac

SEQ ID NO: 172 - protein, 6 aa, synthetic construct
  REGION 1..6: note = synthetic
QSVSSY

SEQ ID NO: 173-174 - skipped (2 entries)

SEQ ID NO: 175 - DNA, 27 bp, synthetic construct
  misc_feature 1..27: note = synthetic
  source 1..27: mol_type = other DNA
cagcagcgtagcaactggccgctcact

SEQ ID NO: 176 - protein, 9 aa, synthetic construct
  REGION 1..9: note = synthetic
QQRSNWPLT

SEQ ID NO: 177 - DNA, 366 bp, synthetic construct
  misc_feature 1..366: note = synthetic
  source 1..366: mol_type = other DNA
caggtgcagctacagcagtggggcgcaggactgttgaggccttcggagaccctgtccctcacctgcgctgtctatggtgggtccttcagtggttactactggaattggatccgccagtccccagggacggggctggagtggattggggaaatcaatcatagagggaacatcaacttcaacccgtccctcaagagtcgagtcaccatatcagaggacacgtccaaaaaccaattctccctgaggctgaactctgtgaccgccgcggacacggctgtgtattactgtgcgagaggagaggattacgatatttggagtggttattatagggagtactggggccagggaaccctggtcaccgtctcctca

SEQ ID NO: 178 - protein, 122 aa, synthetic construct
  REGION 1..122: note = synthetic
QVQLQQWGAGLLRPSETLSLTCAVYGGSFSGYYWNWIRQSPGTGLEWIGEINHRGNINFNPSLKSRVTISEDTSKNQFSLRLNSVTAADTAVYYCARGEDYDIWSGYYREYWGQGTLVTVSS

SEQ ID NO: 179 - DNA, 24 bp, synthetic construct
  misc_feature 1..24: note = synthetic
  source 1..24: mol_type = other DNA
ggtgggtccttcagtggttactac

SEQ ID NO: 180 - protein, 8 aa, synthetic construct
  REGION 1..8: note = synthetic
GGSFSGYY

SEQ ID NO: 181 - DNA, 21 bp, synthetic construct
  misc_feature 1..21: note = synthetic
  source 1..21: mol_type = other DNA
atcaatcatagagggaacatc

SEQ ID NO: 182 - protein, 7 aa, synthetic construct
  REGION 1..7: note = synthetic
INHRGNI

SEQ ID NO: 183 - DNA, 48 bp, synthetic construct
  misc_feature 1..48: note = synthetic
  source 1..48: mol_type = other DNA
gcgagaggagaggattacgatatttggagtggttattatagggagtac

SEQ ID NO: 184 - protein, 16 aa, synthetic construct
  REGION 1..16: note = synthetic
ARGEDYDIWSGYYREY

SEQ ID NO: 185 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccactctctcctgcagggccagtcagagtgttagcagctacttagcctggtaccagcagaaacctggccaggctcccaggctcctcatctatgatgcatccaagagggccactggcatcccagccaggttcagtggcagtgggtctgggacagacttcactctcaccatcagcagcctagagcctgaagattttgctgtttattactgtcagcagcgtagcaactggcctctcgctttcggcggagggaccaaggtggagatcaaa

SEQ ID NO: 186 - protein, 107 aa, synthetic construct
  REGION 1..107: note = synthetic
EIVLTQSPATLSLSPGERATLSCRASQSVSSYLAWYQQKPGQAPRLLIYDASKRATGIPARFSGSGSGTDFTLTISSLEPEDFAVYYCQQRSNWPLAFGGGTKVEIK

SEQ ID NO: 187 - DNA, 18 bp, synthetic construct
  misc_feature 1..18: note = synthetic
  source 1..18: mol_type = other DNA
cagagtgttagcagctac

SEQ ID NO: 188 - protein, 6 aa, synthetic construct
  REGION 1..6: note = synthetic
QSVSSY

SEQ ID NO: 189-190 - skipped (2 entries)

SEQ ID NO: 191 - DNA, 27 bp, synthetic construct
  misc_feature 1..27: note = synthetic
  source 1..27: mol_type = other DNA
cagcagcgtagcaactggcctctcgct

SEQ ID NO: 192 - protein, 9 aa, synthetic construct
  REGION 1..9: note = synthetic
QQRSNWPLA

SEQ ID NO: 193 - DNA, 357 bp, synthetic construct
  misc_feature 1..357: note = synthetic
  source 1..357: mol_type = other DNA
caggtgcagctacagcagtggggcgcaggactgttgaagccttcggagaccctgtccctcacctgcgctgtctatggtgggtccttcagtgagttctactggaactggatccgccagcccccagagaagggcctggagtggattggggaaatcaatcatcgtggaaacaccaactacaacccgtccctcaagagtcgagtcaccatatcagtagacatgtccaagaaccagttctccctgcagctgaactctgtgaccgtcgcggacacggctctgtattactgtgcgtttggctacgattttcggagttcttatgaggacgtctggggccaagggaccacggtcaccgtctcctca

SEQ ID NO: 194 - protein, 119 aa, synthetic construct
  REGION 1..119: note = synthetic
QVQLQQWGAGLLKPSETLSLTCAVYGGSFSEFYWNWIRQPPEKGLEWIGEINHRGNTNYNPSLKSRVTISVDMSKNQFSLQLNSVTVADTALYYCAFGYDFRSSYEDVWGQGTTVTVSS

SEQ ID NO: 195 - DNA, 24 bp, synthetic construct
  misc_feature 1..24: note = synthetic
  source 1..24: mol_type = other DNA
ggtgggtccttcagtgagttctac

SEQ ID NO: 196 - protein, 8 aa, synthetic construct
  REGION 1..8: note = synthetic
GGSFSEFY

SEQ ID NO: 197 - DNA, 21 bp, synthetic construct
  misc_feature 1..21: note = synthetic
  source 1..21: mol_type = other DNA
atcaatcatcgtggaaacacc

SEQ ID NO: 198 - protein, 7 aa, synthetic construct
  REGION 1..7: note = synthetic
INHRGNT

SEQ ID NO: 199 - DNA, 39 bp, synthetic construct
  misc_feature 1..39: note = synthetic
  source 1..39: mol_type = other DNA
gcgtttggctacgattttcggagttcttatgaggacgtc

SEQ ID NO: 200 - protein, 13 aa, synthetic construct
  REGION 1..13: note = synthetic
AFGYDFRSSYEDV

SEQ ID NO: 201 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcaggatattagcacctacttagcctggcaccaacagaaacctggccagcctcccaggctcctcatctatggttcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaa

SEQ ID NO: 202 - protein, 107 aa, synthetic construct
  REGION 1..107: note = synthetic
EIVLTQSPATLSLSPGERATLSCRASQDISTYLAWHQQKPGQPPRLLIYGSSNRATGIPARFSGSGSGTDFTLTISSLEPEDFAVYYCQQRSNWPLTFGGGTKVEIK

SEQ ID NO: 203 - DNA, 18 bp, synthetic construct
  misc_feature 1..18: note = synthetic
  source 1..18: mol_type = other DNA
caggatattagcacctac

SEQ ID NO: 204 - protein, 6 aa, synthetic construct
  REGION 1..6: note = synthetic
QDISTY

SEQ ID NO: 205-206 - skipped (2 entries)

SEQ ID NO: 207 - DNA, 27 bp, synthetic construct
  misc_feature 1..27: note = synthetic
  source 1..27: mol_type = other DNA
cagcagcgtagcaactggcctctcact

SEQ ID NO: 208 - protein, 9 aa, synthetic construct
  REGION 1..9: note = synthetic
QQRSNWPLT

SEQ ID NO: 209 - DNA, 372 bp, synthetic construct
  misc_feature 1..372: note = synthetic
  source 1..372: mol_type = other DNA
gaggtgcagctgttggagtctgggggaggcttggtacagccgggggggtccctgagactctcctgtgcagcctctggattcaccttcagaagctatgccatgagttgggtccgccaggctccagggaaggggctggagtgggtctcagttattagtggtggtggtggtaggacatactacacagactccgtgaagggccggttcaccatctccagagacaattccaagagcatgctgtatctgcaaatgaacagcctgagagccgaggacacggccatttattactgtgcgaaagagagggtaactggaatagaccactactactacggtgtggacgtctggggccaagggaccacggtcaccgtctcctca

SEQ ID NO: 210 - protein, 124 aa, synthetic construct
  REGION 1..124: note = synthetic
EVQLLESGGGLVQPGGSLRLSCAASGFTFRSYAMSWVRQAPGKGLEWVSVISGGGGRTYYTDSVKGRFTISRDNSKSMLYLQMNSLRAEDTAIYYCAKERVTGIDHYYYGVDVWGQGTTVTVSS

SEQ ID NO: 211 - DNA, 24 bp, synthetic construct
  misc_feature 1..24: note = synthetic
  source 1..24: mol_type = other DNA
ggattcaccttcagaagctatgcc

SEQ ID NO: 212 - protein, 8 aa, synthetic construct
  REGION 1..8: note = synthetic
GFTFRSYA

SEQ ID NO: 213 - DNA, 24 bp, synthetic construct
  misc_feature 1..24: note = synthetic
  source 1..24: mol_type = other DNA
attagtggtggtggtggtaggaca

SEQ ID NO: 214 - protein, 8 aa, synthetic construct
  REGION 1..8: note = synthetic
ISGGGGRT

SEQ ID NO: 215 - DNA, 51 bp, synthetic construct
  misc_feature 1..51: note = synthetic
  source 1..51: mol_type = other DNA
gcgaaagagagggtaactggaatagaccactactactacggtgtggacgtc

SEQ ID NO: 216 - protein, 17 aa, synthetic construct
  REGION 1..17: note = synthetic
AKERVTGIDHYYYGVDV

SEQ ID NO: 217 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagcattagtagctatttaaattggtatcagcagaaaccagggaaagcccctaagctcctgatctatgctacatccagtttgcaaagtggggtcccatcacggttcagtggcagtgcatctggaacagatttcactctcgccatcagcagtctgcaacctgaagattttgcaacttactactgtcaacagagttacactacccccctcactttcggcggagggaccaaggtggagatcaaa

SEQ ID NO: 218 - protein, 107 aa, synthetic construct
  REGION 1..107: note = synthetic
DIQMTQSPSSLSASVGDRVTITCRASQSISSYLNWYQQKPGKAPKLLIYATSSLQSGVPSRFSGSASGTDFTLAISSLQPEDFATYYCQQSYTTPLTFGGGTKVEIK

SEQ ID NO: 219 - DNA, 18 bp, synthetic construct
  misc_feature 1..18: note = synthetic
  source 1..18: mol_type = other DNA
cagagcattagtagctat

SEQ ID NO: 220 - protein, 6 aa, synthetic construct
  REGION 1..6: note = synthetic
QSISSY

SEQ ID NO: 221-222 - skipped (2 entries)

SEQ ID NO: 223 - DNA, 27 bp, synthetic construct
  misc_feature 1..27: note = synthetic
  source 1..27: mol_type = other DNA
caacagagttacactacccccctcact

SEQ ID NO: 224 - protein, 9 aa, synthetic construct
  REGION 1..9: note = synthetic
QQSYTTPLT

SEQ ID NO: 225 - DNA, 390 bp, synthetic construct
  misc_feature 1..390: note = synthetic
  source 1..390: mol_type = other DNA
gaggtgcagctggtggagtctgggggaggcttggtacaacctggagggtccctgagactttcctgtgcagcctctggatttacattcagcagttatgaaatgaactgggtccgccaggctccagggaaggggctggagtgggtttcatatatcagtagtagtggtaataccaaagactacgcaggctctgtgaagggccgagtcaccatctccagagacaacgccaagaacttactgtatctgcaaatgaacagcctgagagccgaggacacggctgtttatcactgtgcgagagatggagggcattacgatattttgactggttccatgtcctactactactacgctttggacgtctggggccaagggaccacggtcaccgtctcctca

SEQ ID NO: 226 - protein, 130 aa, synthetic construct
  REGION 1..130: note = synthetic
EVQLVESGGGLVQPGGSLRLSCAASGFTFSSYEMNWVRQAPGKGLEWVSYISSSGNTKDYAGSVKGRVTISRDNAKNLLYLQMNSLRAEDTAVYHCARDGGHYDILTGSMSYYYYALDVWGQGTTVTVSS

SEQ ID NO: 227 - DNA, 24 bp, synthetic construct
  misc_feature 1..24: note = synthetic
  source 1..24: mol_type = other DNA
ggatttacattcagcagttatgaa

SEQ ID NO: 228 - protein, 8 aa, synthetic construct
  REGION 1..8: note = synthetic
GFTFSSYE

SEQ ID NO: 229 - DNA, 24 bp, synthetic construct
  misc_feature 1..24: note = synthetic
  source 1..24: mol_type = other DNA
atcagtagtagtggtaataccaaa

SEQ ID NO: 230 - protein, 8 aa, synthetic construct
  REGION 1..8: note = synthetic
ISSSGNTK

SEQ ID NO: 231 - DNA, 69 bp, synthetic construct
  misc_feature 1..69: note = synthetic
  source 1..69: mol_type = other DNA
gcgagagatggagggcattacgatattttgactggttccatgtcctactactactacgctttggacgtc

SEQ ID NO: 232 - protein, 23 aa, synthetic construct
  REGION 1..23: note = synthetic
ARDGGHYDILTGSMSYYYYALDV

SEQ ID NO: 233 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 234 - protein, 108 aa, synthetic construct; identical to SEQ ID NO: 26
  REGION 1..108: note = synthetic

SEQ ID NO: 235 - DNA, 18 bp, synthetic construct
  misc_feature 1..18: note = synthetic
  source 1..18: mol_type = other DNA
cagagcattagcagctat

SEQ ID NO: 236 - protein, 6 aa, synthetic construct
  REGION 1..6: note = synthetic
QSISSY

SEQ ID NO: 237-238 - skipped (2 entries)

SEQ ID NO: 239 - DNA, 30 bp, synthetic construct; identical to SEQ ID NO: 31
  misc_feature 1..30: note = synthetic
  source 1..30: mol_type = other DNA

SEQ ID NO: 240 - protein, 10 aa, synthetic construct
  REGION 1..10: note = synthetic
QQSYSTPPIT

SEQ ID NO: 241 - DNA, 363 bp, synthetic construct
  misc_feature 1..363: note = synthetic
  source 1..363: mol_type = other DNA
gaggtgcagctggtggagtctgggggaggcttggtacagcctggggggtccctgagactctcctgtgcagcctctggattcacctttaaaacctatgccatgagctgggtccgccaggctccagggagggggctggagtgggtctcaggtattagtggtagtggtagtacctcatactacgcagactccgtgaagggccggttcaccatctccagagacaattacaagaagacgctgtctctgcaaatgaacagtctgagagccgaggacacggccgtttattactgtgcgctggatataatggcaacggtaggaggtctctttaacaactggggccagggaaccctggtcaccgtctcctca

SEQ ID NO: 242 - protein, 121 aa, synthetic construct
  REGION 1..121: note = synthetic
EVQLVESGGGLVQPGGSLRLSCAASGFTFKTYAMSWVRQAPGRGLEWVSGISGSGSTSYYADSVKGRFTISRDNYKKTLSLQMNSLRAEDTAVYYCALDIMATVGGLFNNWGQGTLVTVSS

SEQ ID NO: 243 - DNA, 24 bp, synthetic construct
  misc_feature 1..24: note = synthetic
  source 1..24: mol_type = other DNA
ggattcacctttaaaacctatgcc

SEQ ID NO: 244 - protein, 8 aa, synthetic construct
  REGION 1..8: note = synthetic
GFTFKTYA

SEQ ID NO: 245 - DNA, 24 bp, synthetic construct
  misc_feature 1..24: note = synthetic
  source 1..24: mol_type = other DNA
attagtggtagtggtagtacctca

SEQ ID NO: 246 - protein, 8 aa, synthetic construct
  REGION 1..8: note = synthetic
ISGSGSTS

SEQ ID NO: 247 - DNA, 42 bp, synthetic construct
  misc_feature 1..42: note = synthetic
  source 1..42: mol_type = other DNA
gcgctggatataatggcaacggtaggaggtctctttaacaac

SEQ ID NO: 248 - protein, 14 aa, synthetic construct
  REGION 1..14: note = synthetic
ALDIMATVGGLFNN

SEQ ID NO: 249 - DNA, 324 bp, synthetic construct
  misc_feature 1..324: note = synthetic
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 250 - protein, 108 aa, synthetic construct
  REGION 1..108: note = synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 251 - DNA, 21 bp, synthetic construct
  misc_feature 1..21: note = synthetic
  source 1..21: mol_type = other DNA
cagagtgttagcagcagctac

SEQ ID NO: 252 - protein, 7 aa, synthetic construct
  REGION 1..7: note = synthetic
QSVSSSY

SEQ ID NO: 253-254 - skipped (2 entries)

SEQ ID NO: 255 - DNA, 27 bp, synthetic construct
  misc_feature 1..27: note = synthetic
  source 1..27: mol_type = other DNA
cagcagtatggtagctcaccttggacg

SEQ ID NO: 256 - protein, 9 aa, synthetic construct
  REGION 1..9: note = synthetic
QQYGSSPWT

SEQ ID NO: 257 - DNA, 360 bp, synthetic construct
  misc_feature 1..360: note = synthetic
  source 1..360: mol_type = other DNA
caggtgcagctggtgcagtctggggctgaggtgaagaagcctgggtcctcggtgaaggtctcctgcaaggcttctggaggcaccttcagcagacatactatcagctgggtgcgacaggcccctggacaagggcttgagtggatgggagggatcatccctatctttggtacagcaaactacgcacacaagttccagggcagagtcacgattaccacggacgaatccacgagcacagcctacatggagctgagcagcctgagatctgaggacacggccgtatattattgtgcgagagccccttatacccgacaggggtacttcgatctctggggccgtggcaccctggtcaccgtctcctca

SEQ ID NO: 258 - protein, 120 aa, synthetic construct
  REGION 1..120: note = synthetic
QVQLVQSGAEVKKPGSSVKVSCKASGGTFSRHTISWVRQAPGQGLEWMGGIIPIFGTANYAHKFQGRVTITTDESTSTAYMELSSLRSEDTAVYYCARAPYTRQGYFDLWGRGTLVTVSS

SEQ ID NO: 259 - DNA, 24 bp, synthetic construct
  misc_feature 1..24: note = synthetic
  source 1..24: mol_type = other DNA
ggaggcaccttcagcagacatact

SEQ ID NO: 260 - protein, 8 aa, synthetic construct
  REGION 1..8: note = synthetic
GGTFSRHT

SEQ ID NO: 261 - DNA, 24 bp, synthetic construct
  misc_feature 1..24: note = synthetic
  source 1..24: mol_type = other DNA
atcatccctatctttggtacagca

SEQ ID NO: 262 - protein, 8 aa, synthetic construct
  REGION 1..8: note = synthetic
IIPIFGTA

SEQ ID NO: 263 - DNA, 39 bp, synthetic construct
  misc_feature 1..39: note = synthetic
  source 1..39: mol_type = other DNA
gcgagagccccttatacccgacaggggtacttcgatctc

SEQ ID NO: 264 - protein, 13 aa, synthetic construct
  REGION 1..13: note = synthetic
ARAPYTRQGYFDL

SEQ ID NO: 265 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctactcatttactgggcatctacccgggaatccggggtccctgaccgattcagtggcagcgggtctgggacagatttcactctcaccatcagcagcctgcaggctgaagatgtggcagtttattactgtcagcaagattatagtactccgtggacgttcggccaagggaccaaggtggaaatcaaa

SEQ ID NO: 266 - protein, 113 aa, synthetic construct
  REGION 1..113: note = synthetic
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQDYSTPWTFGQGTKVEIK

SEQ ID NO: 267 - DNA, 36 bp, synthetic construct
  misc_feature 1..36: note = synthetic
  source 1..36: mol_type = other DNA
cagagtgttttatacagctccaacaataagaactac

SEQ ID NO: 268 - protein, 12 aa, synthetic construct
  REGION 1..12: note = synthetic
QSVLYSSNNKNY

SEQ ID NO: 269-270 - skipped (2 entries)

SEQ ID NO: 271 - DNA, 27 bp, synthetic construct
  misc_feature 1..27: note = synthetic
  source 1..27: mol_type = other DNA
cagcaagattatagtactccgtggacg

SEQ ID NO: 272 - protein, 9 aa, synthetic construct
  REGION 1..9: note = synthetic
QQDYSTPWT

SEQ ID NO: 273 - DNA, 354 bp, synthetic construct
  misc_feature 1..354: note = synthetic
  source 1..354: mol_type = other DNA
caggtgcagctggtgcagtctggggctgaggtgaagaagcctggggcctcagtgaaggtttcctgcaaggcatctggatacaccttcaccaactactatatacactgggtgcgacaggcccctggacaagggcttgactggatgggaattatcaaccctggtggtggtaacacaaactacgcacagaagttcctgggcagagtcaccatgaccagggacacgtccacgaccacagtctacatggagctgagcagcctgagatctgaggacacggccatatattactgtgcgagagaaaactggaactcttactttgacaactggggccagggaaccctggtcaccgtctcctca

SEQ ID NO: 274 - protein, 118 aa, synthetic construct
  REGION 1..118: note = synthetic
QVQLVQSGAEVKKPGASVKVSCKASGYTFTNYYIHWVRQAPGQGLDWMGIINPGGGNTNYAQKFLGRVTMTRDTSTTTVYMELSSLRSEDTAIYYCARENWNSYFDNWGQGTLVTVSS

SEQ ID NO: 275 - DNA, 24 bp, synthetic construct
  misc_feature 1..24: note = synthetic
  source 1..24: mol_type = other DNA
ggatacaccttcaccaactactat

SEQ ID NO: 276 - protein, 8 aa, synthetic construct
  REGION 1..8: note = synthetic
GYTFTNYY

SEQ ID NO: 277 - DNA, 24 bp, synthetic construct
  misc_feature 1..24: note = synthetic
  source 1..24: mol_type = other DNA
atcaaccctggtggtggtaacaca

SEQ ID NO: 278 - protein, 8 aa, synthetic construct
  REGION 1..8: note = synthetic
INPGGGNT

SEQ ID NO: 279 - DNA, 33 bp, synthetic construct
  misc_feature 1..33: note = synthetic
  source 1..33: mol_type = other DNA
gcgagagaaaactggaactcttactttgacaac

SEQ ID NO: 280 - protein, 11 aa, synthetic construct
  REGION 1..11: note = synthetic
ARENWNSYFDN

SEQ ID NO: 281 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacagctccaacaataagaacttcttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcactttattactgtcagcaatattatggtgctccgtggacgttcggccaagggaccaaggtggaaatcaaa

SEQ ID NO: 282 - protein, 113 aa, synthetic construct
  REGION 1..113: note = synthetic
DIVMTQSPDSLAVSLGERATINCKSSQSVLYSSNNKNFLAWYQQKPGQPPKLLIYWASTRESGVPDRFSGSGSGTDFTLTISSLQAEDVALYYCQQYYGAPWTFGQGTKVEIK

SEQ ID NO: 283 - DNA, 36 bp, synthetic construct
  misc_feature 1..36: note = synthetic
  source 1..36: mol_type = other DNA
cagagtgttttatacagctccaacaataagaacttc

SEQ ID NO: 284 - protein, 12 aa, synthetic construct
  REGION 1..12: note = synthetic
QSVLYSSNNKNF

SEQ ID NO: 285-286 - skipped (2 entries)

SEQ ID NO: 287 - DNA, 27 bp, synthetic construct
  misc_feature 1..27: note = synthetic
  source 1..27: mol_type = other DNA
cagcaatattatggtgctccgtggacg

SEQ ID NO: 288 - protein, 9 aa, synthetic construct
  REGION 1..9: note = synthetic
QQYYGAPWT

SEQ ID NO: 289 - DNA, 357 bp, synthetic construct
  misc_feature 1..357: note = synthetic
  source 1..357: mol_type = other DNA
caggtccagctggtgcagtctggggctgaggtgaagaagcctgggtcctcggtgaaggtctcctgcaaggcttctggaggcaccttcagcagctatactatcaactgggtgcgacaggcccctggacaagggcttgagtggatgggagggatcatccctatctttggtatagcaaactacgcacagaagttccagggcagagtcacgattaccacggacgaatccacgaacacagcctacatggagctgagcagcctgagatctgaggacacggccatttattactgtgcgagagcgagatatggttcggggagttatgactactggggccagggaaccctggtcaccgtctcctca

SEQ ID NO: 290 - protein, 119 aa, synthetic construct
  REGION 1..119: note = synthetic
QVQLVQSGAEVKKPGSSVKVSCKASGGTFSSYTINWVRQAPGQGLEWMGGIIPIFGIANYAQKFQGRVTITTDESTNTAYMELSSLRSEDTAIYYCARARYGSGSYDYWGQGTLVTVSS

SEQ ID NO: 291 - DNA, 24 bp, synthetic construct
  misc_feature 1..24: note = synthetic
  source 1..24: mol_type = other DNA
ggaggcaccttcagcagctatact

SEQ ID NO: 292 - protein, 8 aa, synthetic construct
  REGION 1..8: note = synthetic
GGTFSSYT

SEQ ID NO: 293 - DNA, 24 bp, synthetic construct
  misc_feature 1..24: note = synthetic
  source 1..24: mol_type = other DNA
atcatccctatctttggtatagca

SEQ ID NO: 294 - protein, 8 aa, synthetic construct
  REGION 1..8: note = synthetic
IIPIFGIA

SEQ ID NO: 295 - DNA, 36 bp, synthetic construct
  misc_feature 1..36: note = synthetic
  source 1..36: mol_type = other DNA
gcgagagcgagatatggttcggggagttatgactac

SEQ ID NO: 296 - protein, 12 aa, synthetic construct
  REGION 1..12: note = synthetic
ARARYGSGSYDY

SEQ ID NO: 297 - DNA, 339 bp, synthetic construct
  misc_feature 1..339: note = synthetic
  source 1..339: mol_type = other DNA
gacatcgtgatgacccagtctccagactccctggctgtgtctctgggcgagagggccaccatcaactgcaagtccagccagagtgttttatacacctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattataatactccatggacgttcggccaagggaccaaggtggaaatcaaa

SEQ ID NO: 298 - protein, 113 aa, synthetic construct
  REGION 1..113: note = synthetic
DIVMTQSPDSLAVSLGERATINCKSSQSVLYTSNNKNYLAWYQQKPGQPPKLLIYWASTRESGVPDRFSGSGSGTDFTLTISSLQAEDVAVYYCQQYYNTPWTFGQGTKVEIK

SEQ ID NO: 299 - DNA, 36 bp, synthetic construct
  misc_feature 1..36: note = synthetic
  source 1..36: mol_type = other DNA
cagagtgttttatacacctccaacaataagaactac

SEQ ID NO: 300 - protein, 12 aa, synthetic construct
  REGION 1..12: note = synthetic
QSVLYTSNNKNY

SEQ ID NO: 301-302 - skipped (2 entries)

SEQ ID NO: 303 - DNA, 27 bp, synthetic construct
  misc_feature 1..27: note = synthetic
  source 1..27: mol_type = other DNA
cagcaatattataatactccatggacg

SEQ ID NO: 304 - protein, 9 aa, synthetic construct
  REGION 1..9: note = synthetic
QQYYNTPWT

SEQ ID NO: 305 - DNA, 360 bp, synthetic construct
  misc_feature 1..360: note = synthetic
  source 1..360: mol_type = other DNA
cagatcaccttgaaggagtctggtcctacgctggtgaaacccacacagaccctcacgctgacctgcaccttctctgggttctcactcagcactaatggagtgggtgtgggctggatccgtcagcccccaggaaaggccctggagtggcttggaatcatttattggaatgatgataagcgctacagcccatctctgaggagcagactcaccatcaccaaggacacctccaaaaaccaggtggtccttacaatgaccaacatggaccctgtggacacagccacatattactgtgcacacagaggcctcttcggaggttggttcgacccctggggccagggaaccctggtcaccgtctcctca

SEQ ID NO: 306 - protein, 120 aa, synthetic construct
  REGION 1..120: note = synthetic
QITLKESGPTLVKPTQTLTLTCTFSGFSLSTNGVGVGWIRQPPGKALEWLGIIYWNDDKRYSPSLRSRLTITKDTSKNQVVLTMTNMDPVDTATYYCAHRGLFGGWFDPWGQGTLVTVSS

SEQ ID NO: 307 - DNA, 30 bp, synthetic construct
  misc_feature 1..30: note = synthetic
  source 1..30: mol_type = other DNA
gggttctcactcagcactaatggagtgggt

SEQ ID NO: 308 - protein, 10 aa, synthetic construct
  REGION 1..10: note = synthetic
GFSLSTNGVG

SEQ ID NO: 309 - DNA, 21 bp, synthetic construct
  misc_feature 1..21: note = synthetic
  source 1..21: mol_type = other DNA
atttattggaatgatgataag

SEQ ID NO: 310 - protein, 7 aa, synthetic construct
  REGION 1..7: note = synthetic
IYWNDDK

SEQ ID NO: 311 - DNA, 36 bp, synthetic construct
  misc_feature 1..36: note = synthetic
  source 1..36: mol_type = other DNA
gcacacagaggcctcttcggaggttggttcgacccc

SEQ ID NO: 312 - protein, 12 aa, synthetic construct
  REGION 1..12: note = synthetic
AHRGLFGGWFDP

SEQ ID NO: 313 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagagcattagcaggtatttaaattggtatcagcagaaaccagggaaagcccctaacctcctgatctttgctgcatccagtttgcaaagtggggtcccatcaaggttcagtggcagtggatctgggacagatttcactctcaccatcagcagtctgcaacctgaagattttgcaacttacttctgtcaacagagttacaataccccgctcactttcggcggagggaccaaggtggagatcaaa

SEQ ID NO: 314 - protein, 107 aa, synthetic construct
  REGION 1..107: note = synthetic
DIQMTQSPSSLSASVGDRVTITCRASQSISRYLNWYQQKPGKAPNLLIFAASSLQSGVPSRFSGSGSGTDFTLTISSLQPEDFATYFCQQSYNTPLTFGGGTKVEIK

SEQ ID NO: 315 - DNA, 18 bp, synthetic construct
  misc_feature 1..18: note = synthetic
  source 1..18: mol_type = other DNA
cagagcattagcaggtat

SEQ ID NO: 316 - protein, 6 aa, synthetic construct
  REGION 1..6: note = synthetic
QSISRY

SEQ ID NO: 317-318 - skipped (2 entries)

SEQ ID NO: 319 - DNA, 27 bp, synthetic construct
  misc_feature 1..27: note = synthetic
  source 1..27: mol_type = other DNA
caacagagttacaataccccgctcact

SEQ ID NO: 320 - protein, 9 aa, synthetic construct
  REGION 1..9: note = synthetic
QQSYNTPLT

SEQ ID NO: 321 - DNA, 366 bp, synthetic construct
  misc_feature 1..366: note = synthetic
  source 1..366: mol_type = other DNA
gaggtgcagctggtggagtctgggggaggcttggtacagccgggggggtccctgagactctcctgtgcaatctctggattcacctttaggagttatgccatgacctgggtccgccaggctccagggaaggcgctggagtgggtctcagttattagtggtagcggtggtaacacatactacgcagactccgtgaagggccggttcaccgtctccagagacaattccaggaacacgctgtatctgcaaatgaacagcctgagagccgaggacacggccgtatatttctgttcgaaagttgcagcagctaataattactattacgctttggacgtctggggccaagggaccacggtcaccgtctcctca

SEQ ID NO: 322 - protein, 122 aa, synthetic construct
  REGION 1..122: note = synthetic
EVQLVESGGGLVQPGGSLRLSCAISGFTFRSYAMTWVRQAPGKALEWVSVISGSGGNTYYADSVKGRFTVSRDNSRNTLYLQMNSLRAEDTAVYFCSKVAAANNYYYALDVWGQGTTVTVSS

SEQ ID NO: 323 - DNA, 24 bp, synthetic construct
  misc_feature 1..24: note = synthetic
  source 1..24: mol_type = other DNA
ggattcacctttaggagttatgcc

SEQ ID NO: 324 - protein, 8 aa, synthetic construct
  REGION 1..8: note = synthetic
GFTFRSYA

SEQ ID NO: 325 - DNA, 24 bp, synthetic construct
  misc_feature 1..24: note = synthetic
  source 1..24: mol_type = other DNA
attagtggtagcggtggtaacaca

SEQ ID NO: 326 - protein, 8 aa, synthetic construct
  REGION 1..8: note = synthetic
ISGSGGNT

SEQ ID NO: 327 - DNA, 45 bp, synthetic construct
  misc_feature 1..45: note = synthetic
  source 1..45: mol_type = other DNA
tcgaaagttgcagcagctaataattactattacgctttggacgtc

SEQ ID NO: 328 - protein, 15 aa, synthetic construct
  REGION 1..15: note = synthetic
SKVAAANNYYYALDV

SEQ ID NO: 329 - DNA, 336 bp, synthetic construct
  misc_feature 1..336: note = synthetic
  source 1..336: mol_type = other DNA
gatattgtgatgactcagtctccactctccctgcccgtcacccctggagagccggcctccatctcctgcaggtctagtcagagcctcctgcatagtaatggatacaagtatttggattggtacctgcagaagccagggcagtctccacaactcctgatctatttggtttctaatcgggcctccggggtccctgacaggttcagtggcagtggatcaggcacagattttacactgaaaatcagcagagtggaggctgaggatgttggggtttattattgcatgcaagctctacaaactccgtacacttttggccaggggaccaagctggagatcaaa

SEQ ID NO: 330 - protein, 112 aa, synthetic construct
  REGION 1..112: note = synthetic
DIVMTQSPLSLPVTPGEPASISCRSSQSLLHSNGYKYLDWYLQKPGQSPQLLIYLVSNRASGVPDRFSGSGSGTDFTLKISRVEAEDVGVYYCMQALQTPYTFGQGTKLEIK

SEQ ID NO: 331 - DNA, 33 bp, synthetic construct
  misc_feature 1..33: note = synthetic
  source 1..33: mol_type = other DNA
cagagcctcctgcatagtaatggatacaagtat

SEQ ID NO: 332 - protein, 11 aa, synthetic construct
  REGION 1..11: note = synthetic
QSLLHSNGYKY

SEQ ID NO: 333-334 - skipped (2 entries)

SEQ ID NO: 335 - DNA, 27 bp, synthetic construct
  misc_feature 1..27: note = synthetic
  source 1..27: mol_type = other DNA
atgcaagctctacaaactccgtacact

SEQ ID NO: 336 - protein, 9 aa, synthetic construct
  REGION 1..9: note = synthetic
MQALQTPYT

SEQ ID NO: 337 - DNA, 381 bp, synthetic construct
  misc_feature 1..381: note = synthetic
  source 1..381: mol_type = other DNA
caggtgcagctggtggagtctgggggaggcgtggtccagcctgggaggtccctgagactctcctgtgtagcgtctggattcaccttcagtaactatggcatgcactgggtccgccaggctccaggcaaggggctggagtgggtggcagttatatggaatgatggaagtaataaatactatgcagactccgtgaagggccgattcaccatctccagagacaattccaagaacacgctgtatctccaagtgagcagcctgagagccgatgacacggctgtatattactgtgcgagggacggagaggtcgaatatagcagctcgaattacaactactacggtctggatgtctggggccaagggaccacggtcaccgtctcctca

SEQ ID NO: 338 - protein, 127 aa, synthetic construct
  REGION 1..127: note = synthetic
QVQLVESGGGVVQPGRSLRLSCVASGFTFSNYGMHWVRQAPGKGLEWVAVIWNDGSNKYYADSVKGRFTISRDNSKNTLYLQVSSLRADDTAVYYCARDGEVEYSSSNYNYYGLDVWGQGTTVTVSS

SEQ ID NO: 339 - DNA, 24 bp, synthetic construct
  misc_feature 1..24: note = synthetic
  source 1..24: mol_type = other DNA
ggattcaccttcagtaactatggc

SEQ ID NO: 340 - protein, 8 aa, synthetic construct
  REGION 1..8: note = synthetic
GFTFSNYG

SEQ ID NO: 341 - DNA, 24 bp, synthetic construct
  misc_feature 1..24: note = synthetic
  source 1..24: mol_type = other DNA
atatggaatgatggaagtaataaa

SEQ ID NO: 342 - protein, 8 aa, synthetic construct
  REGION 1..8: note = synthetic
IWNDGSNK

SEQ ID NO: 343 - DNA, 60 bp, synthetic construct
  misc_feature 1..60: note = synthetic
  source 1..60: mol_type = other DNA
gcgagggacggagaggtcgaatatagcagctcgaattacaactactacggtctggatgtc

SEQ ID NO: 344 - protein, 20 aa, synthetic construct
  REGION 1..20: note = synthetic
ARDGEVEYSSSNYNYYGLDV

SEQ ID NO: 345 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaaactcctgatctacgatgcatccaatttggaaacaggggtcccatcaaggttcagtggaagtggatctgggacagattttactttcaccatcagcagcctgcagcctgaagatattgtaacatattactgtcaacagtatgatgatctcccgatcaccttcggccaagggacacgactggagattaaa

SEQ ID NO: 346 - protein, 107 aa, synthetic construct
  REGION 1..107: note = synthetic
DIQMTQSPSSLSASVGDRVTITCQASQDISNYLNWYQQKPGKAPKLLIYDASNLETGVPSRFSGSGSGTDFTFTISSLQPEDIVTYYCQQYDDLPITFGQGTRLEIK

SEQ ID NO: 347 - DNA, 18 bp, synthetic construct
  misc_feature 1..18: note = synthetic
  source 1..18: mol_type = other DNA
caggacattagcaactat

SEQ ID NO: 348 - protein, 6 aa, synthetic construct
  REGION 1..6: note = synthetic
QDISNY

SEQ ID NO: 349-350 - skipped (2 entries)

SEQ ID NO: 351 - DNA, 27 bp, synthetic construct
  misc_feature 1..27: note = synthetic
  source 1..27: mol_type = other DNA
caacagtatgatgatctcccgatcacc

SEQ ID NO: 352 - protein, 9 aa, synthetic construct
  REGION 1..9: note = synthetic
QQYDDLPIT

SEQ ID NO: 353 - DNA, 375 bp, synthetic construct
  misc_feature 1..375: note = synthetic
  source 1..375: mol_type = other DNA
gaggtgcagctggtggagtctgggggaggcttggtacagcctggggggtccctgagactctcctgtgcagcctctggattctcctttcataattttgccatgaactgggtccgccaggctccagggaaggggctggagtgggtctcagttattactggtagtggtactagcacacactacgcagactccgtgaagggccggttcaccatctccagagacaattccaagaaaacgctatatctgcaaatgaatagcctgagagccgaggacacggccgtatattactgtgcgaaagatcggggctatgattatagtggttcttactacaactggttcgacccctggggccagggaaccctggtcaccgtctcctca

SEQ ID NO: 354 - protein, 125 aa, synthetic construct
  REGION 1..125: note = synthetic
EVQLVESGGGLVQPGGSLRLSCAASGFSFHNFAMNWVRQAPGKGLEWVSVITGSGTSTHYADSVKGRFTISRDNSKKTLYLQMNSLRAEDTAVYYCAKDRGYDYSGSYYNWFDPWGQGTLVTVSS

SEQ ID NO: 355 - DNA, 24 bp, synthetic construct
  misc_feature 1..24: note = synthetic
  source 1..24: mol_type = other DNA
ggattctcctttcataattttgcc

SEQ ID NO: 356 - protein, 8 aa, synthetic construct
  REGION 1..8: note = synthetic
GFSFHNFA

SEQ ID NO: 357 - DNA, 24 bp, synthetic construct
  misc_feature 1..24: note = synthetic
  source 1..24: mol_type = other DNA
attactggtagtggtactagcaca

SEQ ID NO: 358 - protein, 8 aa, synthetic construct
  REGION 1..8: note = synthetic
ITGSGTST

SEQ ID NO: 359 - DNA, 54 bp, synthetic construct
  misc_feature 1..54: note = synthetic
  source 1..54: mol_type = other DNA
gcgaaagatcggggctatgattatagtggttcttactacaactggttcgacccc

SEQ ID NO: 360 - protein, 18 aa, synthetic construct
  REGION 1..18: note = synthetic
AKDRGYDYSGSYYNWFDP

SEQ ID NO: 361 - DNA, 327 bp, synthetic construct
  misc_feature 1..327: note = synthetic
  source 1..327: mol_type = other DNA
gacatccagatgacccagtctccatcctccctgtctgcatctgtaggagacagaatcaccatcacttgccgggcaagtcagagtattagcagctatttaaattggtatcagcagaaaccagggaaagcccctaaactcctgatctttgctgcatcaaatttgcaaagtggggtcccatcaaggttcagtggcagtggatctgggacagatttcactctcaccatcagtagtctgcaacctgaagattttgcaacttactactgtcaacagagttacagtaccccatccttattcactttcggccctgggaccaaagtggatatcaaa

SEQ ID NO: 362 - protein, 109 aa, synthetic construct
  REGION 1..109: note = synthetic
DIQMTQSPSSLSASVGDRITITCRASQSISSYLNWYQQKPGKAPKLLIFAASNLQSGVPSRFSGSGSGTDFTLTISSLQPEDFATYYCQQSYSTPSLFTFGPGTKVDIK

SEQ ID NO: 363 - DNA, 18 bp, synthetic construct
  misc_feature 1..18: note = synthetic
  source 1..18: mol_type = other DNA
cagagtattagcagctat

SEQ ID NO: 364 - protein, 6 aa, synthetic construct
  REGION 1..6: note = synthetic
QSISSY

SEQ ID NO: 365-366 - skipped (2 entries)

SEQ ID NO: 367 - DNA, 33 bp, synthetic construct
  misc_feature 1..33: note = synthetic
  source 1..33: mol_type = other DNA
caacagagttacagtaccccatccttattcact

SEQ ID NO: 368 - protein, 11 aa, synthetic construct
  REGION 1..11: note = synthetic
QQSYSTPSLFT

SEQ ID NO: 369 - DNA, 384 bp, synthetic construct
  misc_feature 1..384: note = synthetic
  source 1..384: mol_type = other DNA
gaggtgcagctggtggagtctgggggaggcttggtacagcctggagggtccctgagactctcctgtgcagtctctggattcaccttcagtagttacgagatgaactgggtccgccaggctccagggaaggggctggaatgggtttcacacattagtagtagtggaagtaccatatactacgcagactctgtgaagggccgattcaccatgtccagagacaacgccaagaactcactgtatctgcaaatgaacagcctgagagccgaggacacggctgtttattactgtgcgagagatgggaatatctggagtggttattatgccgcctactacttctacggtatggacgtctggggccaagggaccacggtcaccgtctcctca

SEQ ID NO: 370 - protein, 128 aa, synthetic construct
  REGION 1..128: note = synthetic
EVQLVESGGGLVQPGGSLRLSCAVSGFTFSSYEMNWVRQAPGKGLEWVSHISSSGSTIYYADSVKGRFTMSRDNAKNSLYLQMNSLRAEDTAVYYCARDGNIWSGYYAAYYFYGMDVWGQGTTVTVSS

SEQ ID NO: 371 - DNA, 24 bp, synthetic construct
  misc_feature 1..24: note = synthetic
  source 1..24: mol_type = other DNA
ggattcaccttcagtagttacgag

SEQ ID NO: 372 - protein, 8 aa, synthetic construct
  REGION 1..8: note = synthetic
GFTFSSYE

SEQ ID NO: 373 - DNA, 24 bp, synthetic construct
  misc_feature 1..24: note = synthetic
  source 1..24: mol_type = other DNA
attagtagtagtggaagtaccata

SEQ ID NO: 374 - protein, 8 aa, synthetic construct
  REGION 1..8: note = synthetic
ISSSGSTI

SEQ ID NO: 375 - DNA, 63 bp, synthetic construct
  misc_feature 1..63: note = synthetic
  source 1..63: mol_type = other DNA
gcgagagatgggaatatctggagtggttattatgccgcctactacttctacggtatggacgtc

SEQ ID NO: 376 - protein, 21 aa, synthetic construct
  REGION 1..21: note = synthetic
ARDGNIWSGYYAAYYFYGMDV

SEQ ID NO: 377 - DNA, 336 bp, synthetic construct
  misc_feature 1..336: note = synthetic
  source 1..336: mol_type = other DNA
gatattgtgatgacccagactccactctcctcacctgtcacccttggacagccggcctccatctcctgcaggtctagtcaaagcctcgtacacagtgatggaaaaacctacttgagttggcttcagcagaggccaggccagcctccaagactcctaatttataagatttctaaccggttctctggggtcccagacagaatcagtggcagtggggcagggacagatttcacactgaaaatcagcagggtggaagctgaggatgtcggggtttattactgcatgcaagctgtacaatttcctcggacgttcggccaagggaccaaggtggaaatcaaa

SEQ ID NO: 378 - protein, 112 aa, synthetic construct
  REGION 1..112: note = synthetic
DIVMTQTPLSSPVTLGQPASISCRSSQSLVHSDGKTYLSWLQQRPGQPPRLLIYKISNRFSGVPDRISGSGAGTDFTLKISRVEAEDVGVYYCMQAVQFPRTFGQGTKVEIK

SEQ ID NO: 379 - DNA, 33 bp, synthetic construct
  misc_feature 1..33: note = synthetic
  source 1..33: mol_type = other DNA
caaagcctcgtacacagtgatggaaaaacctac

SEQ ID NO: 380 - protein, 11 aa, synthetic construct
  REGION 1..11: note = synthetic
QSLVHSDGKTY

SEQ ID NO: 381-382 - skipped (2 entries)

SEQ ID NO: 383 - DNA, 27 bp, synthetic construct
  misc_feature 1..27: note = synthetic
  source 1..27: mol_type = other DNA
atgcaagctgtacaatttcctcggacg

SEQ ID NO: 384 - protein, 9 aa, synthetic construct
  REGION 1..9: note = synthetic
MQAVQFPRT

SEQ ID NO: 385 - DNA, 366 bp, synthetic construct
  misc_feature 1..366: note = synthetic
  source 1..366: mol_type = other DNA
caggtgcagctacagcagtggggcgcaggactgttgaacccttcggagaccctgtccctcacctgcgctgtctatggtggggccttcagtgattactactggaattggatccgccagcccccagggaaggggctggagtggattggggaaatcaatcatcgcggaagcaccaactacaacccgtccctcaagagtcgtgtcaccatttcagttgacacgtccaagaaccagttctccctgaggatgagctctgtgaccgccgcggacgcggctgtgtattactgtgcgagaggagaggattacgatatttggaatggttattatcaggaaaaatggggccagggaaccctggtcaccgtctcctca

SEQ ID NO: 386 - protein, 122 aa, synthetic construct
  REGION 1..122: note = synthetic
QVQLQQWGAGLLNPSETLSLTCAVYGGAFSDYYWNWIRQPPGKGLEWIGEINHRGSTNYNPSLKSRVTISVDTSKNQFSLRMSSVTAADAAVYYCARGEDYDIWNGYYQEKWGQGTLVTVSS

SEQ ID NO: 387 - DNA, 24 bp, synthetic construct
  misc_feature 1..24: note = synthetic
  source 1..24: mol_type = other DNA
ggtggggccttcagtgattactac

SEQ ID NO: 388 - protein, 8 aa, synthetic construct
  REGION 1..8: note = synthetic
GGAFSDYY

SEQ ID NO: 389 - DNA, 21 bp, synthetic construct
  misc_feature 1..21: note = synthetic
  source 1..21: mol_type = other DNA
atcaatcatcgcggaagcacc

SEQ ID NO: 390 - protein, 7 aa, synthetic construct
  REGION 1..7: note = synthetic
INHRGST

SEQ ID NO: 391 - DNA, 48 bp, synthetic construct
  misc_feature 1..48: note = synthetic
  source 1..48: mol_type = other DNA
gcgagaggagaggattacgatatttggaatggttattatcaggaaaaa

SEQ ID NO: 392 - protein, 16 aa, synthetic construct
  REGION 1..16: note = synthetic
ARGEDYDIWNGYYQEK

SEQ ID NO: 393 - DNA, 321 bp, synthetic construct
  misc_feature 1..321: note = synthetic
  source 1..321: mol_type = other DNA
gaaattgtgttgacacagtctccagccaccctgtctttgtctccaggggaaagagccaccctctcctgcagggccagtcagagtattagcacctacttagcctggtaccaacagaagcctggccaggctcccaggctcctcatctatgatgcatccaagagggccactggcatcccagccaggttcagtggcagtgggtctgggacagacttcactctcaccatcagcagcctagagcctgaagattttgtagtttattactgtcaccagcgtagcaactggcctctcactttcggcggagggaccaaggtggagatcaaa

SEQ ID NO: 394 - protein, 107 aa, synthetic construct
  REGION 1..107: note = synthetic
EIVLTQSPATLSLSPGERATLSCRASQSISTYLAWYQQKPGQAPRLLIYDASKRATGIPARFSGSGSGTDFTLTISSLEPEDFVVYYCHQRSNWPLTFGGGTKVEIK

SEQ ID NO: 395 - DNA, 18 bp, synthetic construct
  misc_feature 1..18: note = synthetic
  source 1..18: mol_type = other DNA
cagagtattagcacctac

SEQ ID NO: 396 - protein, 6 aa, synthetic construct
  REGION 1..6: note = synthetic
QSISTY

SEQ ID NO: 397-398 - skipped (2 entries)

SEQ ID NO: 399 - DNA, 27 bp, synthetic construct
  misc_feature 1..27: note = synthetic
  source 1..27: mol_type = other DNA
caccagcgtagcaactggcctctcact

SEQ ID NO: 400 - protein, 9 aa, synthetic construct
  REGION 1..9: note = synthetic
HQRSNWPLT

SEQ ID NO: 401 - DNA, 351 bp, synthetic construct
  misc_feature 1..351: note = synthetic
  source 1..351: mol_type = other DNA
caggtgcagctgcaggagtcggggccaggactggtgaagccttcggagaccctgtccctcacctgcactgtctctggtggttccttcagtagttactactggagttggctccggcagcccccaggaaaggggctggagtggattggatatatcttttacagtgggagtaccgactacaacccctccctcaagagtcgagtcaccatttcagtagacacgtccaagaagcagttctccctgaagctgacctctgtgaccgctgcggacacggccgtctattactgtgcgcgaacaataagtacgtggtggttcgccccctggggccagggaaccctggtcaccgtctcctca

SEQ ID NO: 402 - protein, 117 aa, synthetic construct
  REGION 1..117: note = synthetic
QVQLQESGPGLVKPSETLSLTCTVSGGSFSSYYWSWLRQPPGKGLEWIGYIFYSGSTDYNPSLKSRVTISVDTSKKQFSLKLTSVTAADTAVYYCARTISTWWFAPWGQGTLVTVSS

SEQ ID NO: 403 - DNA, 24 bp, synthetic construct
  misc_feature 1..24: note = synthetic
  source 1..24: mol_type = other DNA
ggtggttccttcagtagttactac

SEQ ID NO: 404 - protein, 8 aa, synthetic construct
  REGION 1..8: note = synthetic
GGSFSSYY

SEQ ID NO: 405 - DNA, 21 bp, synthetic construct
  misc_feature 1..21: note = synthetic
  source 1..21: mol_type = other DNA
atcttttacagtgggagtacc

SEQ ID NO: 406 - protein, 7 aa, synthetic construct
  REGION 1..7: note = synthetic
IFYSGST

SEQ ID NO: 407 - DNA, 33 bp, synthetic construct
  misc_feature 1..33: note = synthetic
  source 1..33: mol_type = other DNA
gcgcgaacaataagtacgtggtggttcgccccc

SEQ ID NO: 408 - protein, 11 aa, synthetic construct
  REGION 1..11: note = synthetic
ARTISTWWFAP

SEQ ID NO: 409 - DNA, 318 bp, synthetic construct
  misc_feature 1..318: note = synthetic
  source 1..318: mol_type = other DNA
gaaatagtgatgacacagtctccagccaccctgtctgtgtctccagggggaagagccaccctctcctgcagggccagtcagagtgttagcaacaacgtagcctggtaccagcagaaacctggccaggctcccaggctcctcatctatggtgcatccaccagggccactggtatcccaggcaggttcagtggcagtgggtctggaacagagttcactctcaccatcagcagcctgcagtctgaagattttgcagtttattcctgtcagcagtataataactggctcactttcggcggagggaccaaggtggagatcaaa

SEQ ID NO: 410 - protein, 106 aa, synthetic construct
  REGION 1..106: note = synthetic
EIVMTQSPATLSVSPGGRATLSCRASQSVSNNVAWYQQKPGQAPRLLIYGASTRATGIPGRFSGSGSGTEFTLTISSLQSEDFAVYSCQQYNNWLTFGGGTKVEIK

SEQ ID NO: 411 - DNA, 18 bp, synthetic construct
  misc_feature 1..18: note = synthetic
  source 1..18: mol_type = other DNA
cagagtgttagcaacaac

SEQ ID NO: 412 - protein, 6 aa, synthetic construct
  REGION 1..6: note = synthetic
QSVSNN

SEQ ID NO: 413-414 - skipped (2 entries)

SEQ ID NO: 415 - DNA, 24 bp, synthetic construct
  misc_feature 1..24: note = synthetic
  source 1..24: mol_type = other DNA
cagcagtataataactggctcact

SEQ ID NO: 416 - protein, 8 aa, synthetic construct
  REGION 1..8: note = synthetic
QQYNNWLT

SEQ ID NO: 417 - DNA, 369 bp, synthetic construct
  misc_feature 1..369: note = synthetic
  source 1..369: mol_type = other DNA
caggtgcagctggtggagtctgggggaggcgtggtccagcctgggaggtccctgagactctcctgtgtagcgtctggattcactttcagtagttatggcatgcactgggtccgccaggctccaggcaaggggctggagtgggtggcaattatatggtatgatggaagtaataaatactatgcagactccgtgaagggccgattcaccatatccagagacaattccaagaacacacagtatctgcaaatgaacagcctgagagccgaggacacggctgtgtattactgtgcgtcagtagctacgtctggggacttcgactactacggtatggacgtctggggccaagggaccacggtcaccgtctcctca

SEQ ID NO: 418 - protein, 123 aa, synthetic construct
  REGION 1..123: note = synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSS

SEQ ID NO: 419 - DNA, 24 bp, synthetic construct
  misc_feature 1..24: note = synthetic
  source 1..24: mol_type = other DNA
ggattcactttcagtagttatggc

SEQ ID NO: 420 - protein, 8 aa, synthetic construct
  REGION 1..8: note = synthetic
GFTFSSYG

SEQ ID NO: 421 - DNA, 24 bp, synthetic construct
  misc_feature 1..24: note = synthetic
  source 1..24: mol_type = other DNA
atatggtatgatggaagtaataaa

SEQ ID NO: 422 - protein, 8 aa, synthetic construct
  REGION 1..8: note = synthetic
IWYDGSNK

SEQ ID NO: 423 - DNA, 48 bp, synthetic construct
  misc_feature 1..48: note = synthetic
  source 1..48: mol_type = other DNA
gcgtcagtagctacgtctggggacttcgactactacggtatggacgtc

SEQ ID NO: 424 - protein, 16 aa, synthetic construct
  REGION 1..16: note = synthetic
ASVATSGDFDYYGMDV

SEQ ID NO: 425 - DNA, 321 bp, synthetic construct
  misc_feature 1..321: note = synthetic
  source 1..321: mol_type = other DNA
gaaattgtgttgacacagtctccagccaccctgtctttgtctccaggggaaagaaccaccctctcctgcagggccagtcagagaattagcacctacttagcctggtatcaacagaaacctggccaggctcccaggctcctcatctatgatgcatccaaaagggccactggcatcccagccaggttcagtggtagtgggtctgggacaggcttcactctcaccatcagcagcctagagcctgaagattttgcagtttattactgtcagcagcgtagtaactggcctctcactttcggcggagggaccaaggtggagatcaaa

SEQ ID NO: 426 - protein, 107 aa, synthetic construct
  REGION 1..107: note = synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIK

SEQ ID NO: 427 - DNA, 18 bp, synthetic construct
  misc_feature 1..18: note = synthetic
  source 1..18: mol_type = other DNA
cagagaattagcacctac

SEQ ID NO: 428 - protein, 6 aa, synthetic construct
  REGION 1..6: note = synthetic
QRISTY

SEQ ID NO: 429-430 - skipped (2 entries)

SEQ ID NO: 431 - DNA, 27 bp, synthetic construct
  misc_feature 1..27: note = synthetic
  source 1..27: mol_type = other DNA
cagcagcgtagtaactggcctctcact

SEQ ID NO: 432 - protein, 9 aa, synthetic construct
  REGION 1..9: note = synthetic
QQRSNWPLT

SEQ ID NO: 433 - DNA, 357 bp, synthetic construct
  misc_feature 1..357: note = synthetic
  source 1..357: mol_type = other DNA
gaggtgcagctggtgcagtctggagcagaggtgagaaagcccggggagtctctgaagatctcctgtaagggttctggatacagctttactaactactggatcgtctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacgggatacgattttcccttcctatcccctctggggccagggaaccctggtcaccgtctcctca

SEQ ID NO: 434 - protein, 119 aa, synthetic construct
  REGION 1..119: note = synthetic
EVQLVQSGAEVRKPGESLKISCKGSGYSFTNYWIVWVRQMPGKGLEWMGIIYPGDSDTRYSPSFQGQVTISADKSISTAYLQWSSLKASDTAMYYCARRDTIFPSYPLWGQGTLVTVSS

SEQ ID NO: 435 - DNA, 24 bp, synthetic construct
  misc_feature 1..24: note = synthetic
  source 1..24: mol_type = other DNA
ggatacagctttactaactactgg

SEQ ID NO: 436 - protein, 8 aa, synthetic construct
  REGION 1..8: note = synthetic
GYSFTNYW

SEQ ID NO: 437 - DNA, 24 bp, synthetic construct
  misc_feature 1..24: note = synthetic
  source 1..24: mol_type = other DNA
atctatcctggtgactctgatacc

SEQ ID NO: 438 - protein, 8 aa, synthetic construct
  REGION 1..8: note = synthetic
IYPGDSDT

SEQ ID NO: 439 - DNA, 36 bp, synthetic construct
  misc_feature 1..36: note = synthetic
  source 1..36: mol_type = other DNA
gcgagacgggatacgattttcccttcctatcccctc

SEQ ID NO: 440 - protein, 12 aa, synthetic construct
  REGION 1..12: note = synthetic
ARRDTIFPSYPL

SEQ ID NO: 441 - DNA, 336 bp, synthetic construct
  misc_feature 1..336: note = synthetic
  source 1..336: mol_type = other DNA
gatattgtgatgactcagtctcctctctccctgcccgtcacccctggagagccggcctccatctcctgcaggtctagtcagagcctcctgaatagtaatggatacaactttttggattggtacctgcagaagccagggcagtctccacaactcctgatctatttggtttctaatcgggcctccggggtccctgacaggttcagtggcagtggatcaggcacagattttacactgaaaatcagcagagtggaggctgaggatattggggtttattactgcatgcaagctctccaaactccgatcaccttcggccaagggacacgactggagattaaa

SEQ ID NO: 442 - protein, 112 aa, synthetic construct
  REGION 1..112: note = synthetic
DIVMTQSPLSLPVTPGEPASISCRSSQSLLNSNGYNFLDWYLQKPGQSPQLLIYLVSNRASGVPDRFSGSGSGTDFTLKISRVEAEDIGVYYCMQALQTPITFGQGTRLEIK

SEQ ID NO: 443 - DNA, 33 bp, synthetic construct
  misc_feature 1..33: note = synthetic
  source 1..33: mol_type = other DNA
cagagcctcctgaatagtaatggatacaacttt

SEQ ID NO: 444 - protein, 11 aa, synthetic construct
  REGION 1..11: note = synthetic
QSLLNSNGYNF

SEQ ID NO: 445-446 - skipped (2 entries)

SEQ ID NO: 447 - DNA, 27 bp, synthetic construct
  misc_feature 1..27: note = synthetic
  source 1..27: mol_type = other DNA
atgcaagctctccaaactccgatcacc

SEQ ID NO: 448 - protein, 9 aa, synthetic construct
  REGION 1..9: note = synthetic
MQALQTPIT

SEQ ID NO: 449 - DNA, 360 bp, synthetic construct
  misc_feature 1..360: note = synthetic
  source 1..360: mol_type = other DNA
cagatcaccttgaaggagtctggtcctacgctggtgaaacccacacagaccctcacgctgacctgcaccttctctgggttctcactcagcactaatggagtgggtgtgggctggatccgtcagcccccaggaaaggccctggagtggcttacactcatttattggaatgaaaataagcactacagcccatctctgaaaaacaggatcaccatcaccaaggacacctccaaaaaccaggtggtccttacaatgaccaacttggaccctgtggacacagccacttattactgtgtacacaggggatggttgggagcaatctttgcctactggggccagggaaccctggtcaccgtctcctca

SEQ ID NO: 450 - protein, 120 aa, synthetic construct
  REGION 1..120: note = synthetic
QITLKESGPTLVKPTQTLTLTCTFSGFSLSTNGVGVGWIRQPPGKALEWLTLIYWNENKHYSPSLKNRITITKDTSKNQVVLTMTNLDPVDTATYYCVHRGWLGAIFAYWGQGTLVTVSS

SEQ ID NO: 451 - DNA, 30 bp, synthetic construct; identical to SEQ ID NO: 307
  misc_feature 1..30: note = synthetic
  source 1..30: mol_type = other DNA

SEQ ID NO: 452 - protein, 10 aa, synthetic construct
  REGION 1..10: note = synthetic
GFSLSTNGVG

SEQ ID NO: 453 - DNA, 21 bp, synthetic construct
  misc_feature 1..21: note = synthetic
  source 1..21: mol_type = other DNA
atttattggaatgaaaataag

SEQ ID NO: 454 - protein, 7 aa, synthetic construct
  REGION 1..7: note = synthetic
IYWNENK

SEQ ID NO: 455 - DNA, 36 bp, synthetic construct
  misc_feature 1..36: note = synthetic
  source 1..36: mol_type = other DNA
gtacacaggggatggttgggagcaatctttgcctac

SEQ ID NO: 456 - protein, 12 aa, synthetic construct
  REGION 1..12: note = synthetic
VHRGWLGAIFAY

SEQ ID NO: 457 - DNA, 378 bp, synthetic construct
  misc_feature 1..378: note = synthetic
  source 1..378: mol_type = other DNA
gaggtgcagctggtggagtctgggggaggcttggtacagcctggggggtccctgagactctcctgtgcagcctctggattcacctttactagttatgccatgacctgggtccgccaggctccagggaaggggctggagtgggtctcagatattagtggtagtggtggtagaacatattacgcagactccgtgaagggccggttcaccatctccagagacaattccaagaatatgctgtatctgcaaatgaacatcctgagagccgaagacacggccgtatatcattgtgcgaagggaacaggccagcaggtggacctttacaactactactatgctttggacgtctggggccaagggaccacggtcaccgtctcctca

SEQ ID NO: 458 - protein, 126 aa, synthetic construct
  REGION 1..126: note = synthetic
EVQLVESGGGLVQPGGSLRLSCAASGFTFTSYAMTWVRQAPGKGLEWVSDISGSGGRTYYADSVKGRFTISRDNSKNMLYLQMNILRAEDTAVYHCAKGTGQQVDLYNYYYALDVWGQGTTVTVSS

SEQ ID NO: 459 - DNA, 24 bp, synthetic construct
  misc_feature 1..24: note = synthetic
  source 1..24: mol_type = other DNA
ggattcacctttactagttatgcc

SEQ ID NO: 460 - protein, 8 aa, synthetic construct
  REGION 1..8: note = synthetic
GFTFTSYA

SEQ ID NO: 461 - DNA, 24 bp, synthetic construct
  misc_feature 1..24: note = synthetic
  source 1..24: mol_type = other DNA
attagtggtagtggtggtagaaca

SEQ ID NO: 462 - protein, 8 aa, synthetic construct
  REGION 1..8: note = synthetic
ISGSGGRT

SEQ ID NO: 463 - DNA, 57 bp, synthetic construct
  misc_feature 1..57: note = synthetic
  source 1..57: mol_type = other DNA
gcgaagggaacaggccagcaggtggacctttacaactactactatgctttggacgtc

SEQ ID NO: 464 - protein, 19 aa, synthetic construct
  REGION 1..19: note = synthetic
AKGTGQQVDLYNYYYALDV

SEQ ID NO: 465 - DNA, 387 bp, synthetic construct
  misc_feature 1..387: note = synthetic
  source 1..387: mol_type = other DNA
caggtgcagctggtggagtctgggggaggcgtggtccagcctgggaggtccctgagactctcctgtgcagcgtctggattcaccttcagttactatggcatgcactgggtccgccaggctccaggcaaggggctggagtgggtggcagttatatggtatgatggaagtaataaacactatgcagactccgtgaagggccgattcaccatctccagagacaattccaagaacacgctgtatctgcaaatgaacagcctgagagccgacgacacggctgtctattactgtgcgagagataagggtataagtggaattaaggggggttcttactactactactatgccatggacgtctggggccaagggaccacggtcaccgtctcctca

SEQ ID NO: 466 - protein, 129 aa, synthetic construct
  REGION 1..129: note = synthetic
QVQLVESGGGVVQPGRSLRLSCAASGFTFSYYGMHWVRQAPGKGLEWVAVIWYDGSNKHYADSVKGRFTISRDNSKNTLYLQMNSLRADDTAVYYCARDKGISGIKGGSYYYYYAMDVWGQGTTVTVSS

SEQ ID NO: 467 - DNA, 24 bp, synthetic construct
  misc_feature 1..24: note = synthetic
  source 1..24: mol_type = other DNA
ggattcaccttcagttactatggc

SEQ ID NO: 468 - protein, 8 aa, synthetic construct
  REGION 1..8: note = synthetic
GFTFSYYG

SEQ ID NO: 469 - DNA, 24 bp, synthetic construct
  misc_feature 1..24: note = synthetic
  source 1..24: mol_type = other DNA
atatggtatgatggaagtaataaa

SEQ ID NO: 470 - protein, 8 aa, synthetic construct
  REGION 1..8: note = synthetic
IWYDGSNK

SEQ ID NO: 471 - DNA, 66 bp, synthetic construct
  misc_feature 1..66: note = synthetic
  source 1..66: mol_type = other DNA
gcgagagataagggtataagtggaattaaggggggttcttactactactactatgccatggacgtc

SEQ ID NO: 472 - protein, 22 aa, synthetic construct
  REGION 1..22: note = synthetic
ARDKGISGIKGGSYYYYYAMDV

SEQ ID NO: 473 - DNA, 360 bp, synthetic construct
  misc_feature 1..360: note = synthetic
  source 1..360: mol_type = other DNA
gaggtgcagctggtggagtctgggggaggcttggtaaagcctggggggtcccttagactctcctgtgcagcctctggattcactttcagtaacgcctggatgacctgggtccgccaggctccagggaaggggctggagtgggttggccgtattaaaaacaaaattgatggtgggacaacagactacgctgcacccgtgaaaggcagattcaccatctcaagagatgattcaaaaaacacggtttatctgcaaatgaacagcctgaaaaccgaggacacagccgtttattactgttccacggtggactacaattggtacttcgatttctggggccgtggcaccctggtcactgtctcctca

SEQ ID NO: 474 - protein, 120 aa, synthetic construct
  REGION 1..120: note = synthetic
EVQLVESGGGLVKPGGSLRLSCAASGFTFSNAWMTWVRQAPGKGLEWVGRIKNKIDGGTTDYAAPVKGRFTISRDDSKNTVYLQMNSLKTEDTAVYYCSTVDYNWYFDFWGRGTLVTVSS

SEQ ID NO: 475 - DNA, 24 bp, synthetic construct
  misc_feature 1..24: note = synthetic
  source 1..24: mol_type = other DNA
ggattcactttcagtaacgcctgg

SEQ ID NO: 476 - protein, 8 aa, synthetic construct
  REGION 1..8: note = synthetic
GFTFSNAW

SEQ ID NO: 477 - DNA, 30 bp, synthetic construct
  misc_feature 1..30: note = synthetic
  source 1..30: mol_type = other DNA
attaaaaacaaaattgatggtgggacaaca

SEQ ID NO: 478 - protein, 10 aa, synthetic construct
  REGION 1..10: note = synthetic
IKNKIDGGTT

SEQ ID NO: 479 - DNA, 33 bp, synthetic construct
  misc_feature 1..33: note = synthetic
  source 1..33: mol_type = other DNA
tccacggtggactacaattggtacttcgatttc

SEQ ID NO: 480 - protein, 11 aa, synthetic construct
  REGION 1..11: note = synthetic
STVDYNWYFDF

SEQ ID NO: 481 - DNA, 387 bp, synthetic construct
  misc_feature 1..387: note = synthetic
  source 1..387: mol_type = other DNA
caggtgcagctggtggagtctgggggaggcgtggtccagcctgggaggtccctgagactctcctgtgcagcgtctggattcaccttcagtttctttggcatgcactgggtccgccaggctccaggcaaggggctggagtgggtggcacttatatggtatgatggaactaatgaaaactatgcagactccgtgaagggccgattcaccatctccagagacaattccaagtccacgctgtatctgcaaatgaacagtctgagagccgaggacacggctgtttactactgtgcgagagataggggagtggcgacatttacgagggggaattactactacaactacggtatggacgtctggggccaagggaccacggtcaccgtctcctca

SEQ ID NO: 482 - protein, 129 aa, synthetic construct
  REGION 1..129: note = synthetic
QVQLVESGGGVVQPGRSLRLSCAASGFTFSFFGMHWVRQAPGKGLEWVALIWYDGTNENYADSVKGRFTISRDNSKSTLYLQMNSLRAEDTAVYYCARDRGVATFTRGNYYYNYGMDVWGQGTTVTVSS

SEQ ID NO: 483 - DNA, 24 bp, synthetic construct
  misc_feature 1..24: note = synthetic
  source 1..24: mol_type = other DNA
ggattcaccttcagtttctttggc

SEQ ID NO: 484 - protein, 8 aa, synthetic construct
  REGION 1..8: note = synthetic
GFTFSFFG

SEQ ID NO: 485 - DNA, 24 bp, synthetic construct
  misc_feature 1..24: note = synthetic
  source 1..24: mol_type = other DNA
atatggtatgatggaactaatgaa

SEQ ID NO: 486 - protein, 8 aa, synthetic construct
  REGION 1..8: note = synthetic
IWYDGTNE

SEQ ID NO: 487 - DNA, 66 bp, synthetic construct
  misc_feature 1..66: note = synthetic
  source 1..66: mol_type = other DNA
gcgagagataggggagtggcgacatttacgagggggaattactactacaactacggtatggacgtc

SEQ ID NO: 488 - protein, 22 aa, synthetic construct
  REGION 1..22: note = synthetic
ARDRGVATFTRGNYYYNYGMDV

SEQ ID NO: 489 - DNA, 387 bp, synthetic construct
  misc_feature 1..387: note = synthetic
  source 1..387: mol_type = other DNA
caggtgcagctggtggagtctgggggaggcgtggtccagcctgggaggtccctgagactctcctgtgcagcgtctggattcaccttcagtttctatggcatgcactgggtccgccaggctccaggcaaggggctggagggggtggcagttatatggtatgatggaagtaataaatactatgcagactccgtgaagggccgattcaccatatccagagacaattccaagaacatgctgtatctacaaatgaccagcctgagagccgaggacacggctgtgtattactgtgcgagagattcgggtaaaactggaactgggataactgggtactcctactactacggtatggacgtctggggccaagggaccacggtcaccgtctcctca

SEQ ID NO: 490 - protein, 129 aa, synthetic construct
  REGION 1..129: note = synthetic
QVQLVESGGGVVQPGRSLRLSCAASGFTFSFYGMHWVRQAPGKGLEGVAVIWYDGSNKYYADSVKGRFTISRDNSKNMLYLQMTSLRAEDTAVYYCARDSGKTGTGITGYSYYYGMDVWGQGTTVTVSS

SEQ ID NO: 491 - DNA, 24 bp, synthetic construct
  misc_feature 1..24: note = synthetic
  source 1..24: mol_type = other DNA
ggattcaccttcagtttctatggc

SEQ ID NO: 492 - protein, 8 aa, synthetic construct
  REGION 1..8: note = synthetic
GFTFSFYG

SEQ ID NO: 493 - DNA, 24 bp, synthetic construct
  misc_feature 1..24: note = synthetic
  source 1..24: mol_type = other DNA
atatggtatgatggaagtaataaa

SEQ ID NO: 494 - protein, 8 aa, synthetic construct
  REGION 1..8: note = synthetic
IWYDGSNK

SEQ ID NO: 495 - DNA, 66 bp, synthetic construct
  misc_feature 1..66: note = synthetic
  source 1..66: mol_type = other DNA
gcgagagattcgggtaaaactggaactgggataactgggtactcctactactacggtatggacgtc

SEQ ID NO: 496 - protein, 22 aa, synthetic construct
  REGION 1..22: note = synthetic
ARDSGKTGTGITGYSYYYGMDV

SEQ ID NO: 497 - DNA, 354 bp, synthetic construct
  misc_feature 1..354: note = synthetic
  source 1..354: mol_type = other DNA
cagctgcagctgcaggagtcgggcccaggactggtgaagccttcggagaccctgtccctcacctgcactgtctctggtggctccatcatcactaatagttattactggggctggatccgccagcccccagggaagggtctggagtggattggtagtatctattatagtgggaggacctactacaacccgtccctcgagagtcgagtcaccatatccgtggacacgtccaagaaccagttctccctgaagttgacctctgtgaccgccgcagacacggctatatattactgtgcgagggaaggggatccgtcgctcgacccctggggccagggaaccctggtcaccgtctcctca

SEQ ID NO: 498 - protein, 118 aa, synthetic construct
  REGION 1..118: note = synthetic
QLQLQESGPGLVKPSETLSLTCTVSGGSIITNSYYWGWIRQPPGKGLEWIGSIYYSGRTYYNPSLESRVTISVDTSKNQFSLKLTSVTAADTAIYYCAREGDPSLDPWGQGTLVTVSS

SEQ ID NO: 499 - DNA, 30 bp, synthetic construct
  misc_feature 1..30: note = synthetic
  source 1..30: mol_type = other DNA
ggtggctccatcatcactaatagttattac

SEQ ID NO: 500 - protein, 10 aa, synthetic construct
  REGION 1..10: note = synthetic
GGSIITNSYY

SEQ ID NO: 501 - DNA, 21 bp, synthetic construct
  misc_feature 1..21: note = synthetic
  source 1..21: mol_type = other DNA
atctattatagtgggaggacc

SEQ ID NO: 502 - protein, 7 aa, synthetic construct
  REGION 1..7: note = synthetic
IYYSGRT

SEQ ID NO: 503 - DNA, 30 bp, synthetic construct
  misc_feature 1..30: note = synthetic
  source 1..30: mol_type = other DNA
gcgagggaaggggatccgtcgctcgacccc

SEQ ID NO: 504 - protein, 10 aa, synthetic construct
  REGION 1..10: note = synthetic
AREGDPSLDP

SEQ ID NO: 505 - DNA, 363 bp, synthetic construct
  misc_feature 1..363: note = synthetic
  source 1..363: mol_type = other DNA
gaggtgcagctggtggagtctgggggagacttggtacagcctggggggtccctgagactctcctgtgcagcctctggattcacctttagcacctatgccatgaactgggtccgccaggctccagggaaggggctggagtgggtctcacatattagtggtagtggtggtaattcatactccgcagactccgtgaagggccggttcaccatctccagagacaattccaagaacacgctatatctgcaaatgaacagcctgcgagccgaggacacggccatatattactgttcgctggatataatggctacagtaggcggtctctttgcctactggggccagggaaccctggtcaccgtctcctca

SEQ ID NO: 506 - protein, 121 aa, synthetic construct
  REGION 1..121: note = synthetic
EVQLVESGGDLVQPGGSLRLSCAASGFTFSTYAMNWVRQAPGKGLEWVSHISGSGGNSYSADSVKGRFTISRDNSKNTLYLQMNSLRAEDTAIYYCSLDIMATVGGLFAYWGQGTLVTVSS

SEQ ID NO: 507 - DNA, 24 bp, synthetic construct
  misc_feature 1..24: note = synthetic
  source 1..24: mol_type = other DNA
ggattcacctttagcacctatgcc

SEQ ID NO: 508 - protein, 8 aa, synthetic construct
  REGION 1..8: note = synthetic
GFTFSTYA

SEQ ID NO: 509 - DNA, 24 bp, synthetic construct
  misc_feature 1..24: note = synthetic
  source 1..24: mol_type = other DNA
attagtggtagtggtggtaattca

SEQ ID NO: 510 - protein, 8 aa, synthetic construct
  REGION 1..8: note = synthetic
ISGSGGNS

SEQ ID NO: 511 - DNA, 42 bp, synthetic construct
  misc_feature 1..42: note = synthetic
  source 1..42: mol_type = other DNA
tcgctggatataatggctacagtaggcggtctctttgcctac

SEQ ID NO: 512 - protein, 14 aa, synthetic construct
  REGION 1..14: note = synthetic
SLDIMATVGGLFAY

SEQ ID NO: 513 - DNA, 393 bp, synthetic construct
  misc_feature 1..393: note = synthetic
  source 1..393: mol_type = other DNA
caggtgcagctggtggagtctgggggaggcgtggtccagcctgggaggtccctgagactctcctgtgtagcgtctggattcatcttcagtttctatggcatgcactgggtccgccaggctccagacaaggggctggagtgggtggcagttatatggtatgatggaagtaatgaatactatgcagactccgtgaagggccgattcaccatctccagagacaattccaagaacacgctgtatctgcaaatgaacagcctgagagccgaggacacggctgtgtattactgtgggagagatcaaggtatttcgtattacgatattttgactggtaattataactattactacggtgtggacgtctggggccaagggaccacggtcaccgtctcctca

SEQ ID NO: 514 - protein, 131 aa, synthetic construct
  REGION 1..131: note = synthetic
QVQLVESGGGVVQPGRSLRLSCVASGFIFSFYGMHWVRQAPDKGLEWVAVIWYDGSNEYYADSVKGRFTISRDNSKNTLYLQMNSLRAEDTAVYYCGRDQGISYYDILTGNYNYYYGVDVWGQGTTVTVSS

SEQ ID NO: 515 - DNA, 24 bp, synthetic construct
  misc_feature 1..24: note = synthetic
  source 1..24: mol_type = other DNA
ggattcatcttcagtttctatggc

SEQ ID NO: 516 - protein, 8 aa, synthetic construct
  REGION 1..8: note = synthetic
GFIFSFYG

SEQ ID NO: 517 - DNA, 24 bp, synthetic construct
  misc_feature 1..24: note = synthetic
  source 1..24: mol_type = other DNA
atatggtatgatggaagtaatgaa

SEQ ID NO: 518 - protein, 8 aa, synthetic construct
  REGION 1..8: note = synthetic
IWYDGSNE

SEQ ID NO: 519 - DNA, 72 bp, synthetic construct
  misc_feature 1..72: note = synthetic
  source 1..72: mol_type = other DNA
gggagagatcaaggtatttcgtattacgatattttgactggtaattataactattactacggtgtggacgtc

SEQ ID NO: 520 - protein, 24 aa, synthetic construct
  REGION 1..24: note = synthetic
GRDQGISYYDILTGNYNYYYGVDV

SEQ ID NO: 521 - DNA, 324 bp, synthetic construct; identical to SEQ ID NO: 233
  misc_feature 1..324: note = synthetic
  source 1..324: mol_type = other DNA

SEQ ID NO: 522 - protein, 108 aa, synthetic construct; identical to SEQ ID NO: 26
  REGION 1..108: note = synthetic

SEQ ID NO: 523 - DNA, 18 bp, synthetic construct
  misc_feature 1..18: note = synthetic
  source 1..18: mol_type = other DNA
cagagcattagcagctat

SEQ ID NO: 524 - protein, 6 aa, synthetic construct
  REGION 1..6: note = synthetic
QSISSY

SEQ ID NO: 525-526 - skipped (2 entries)

SEQ ID NO: 527 - DNA, 30 bp, synthetic construct; identical to SEQ ID NO: 31
  misc_feature 1..30: note = synthetic
  source 1..30: mol_type = other DNA

SEQ ID NO: 528 - protein, 10 aa, synthetic construct
  REGION 1..10: note = synthetic
QQSYSTPPIT

SEQ ID NO: 529 - DNA, 324 bp, synthetic construct; identical to SEQ ID NO: 249
  misc_feature 1..324: note = synthetic
  source 1..324: mol_type = other DNA

SEQ ID NO: 530 - protein, 108 aa, synthetic construct; identical to SEQ ID NO: 250
  REGION 1..108: note = synthetic

SEQ ID NO: 531 - DNA, 21 bp, synthetic construct
  misc_feature 1..21: note = synthetic
  source 1..21: mol_type = other DNA
cagagtgttagcagcagctac

SEQ ID NO: 532 - protein, 7 aa, synthetic construct
  REGION 1..7: note = synthetic
QSVSSSY

SEQ ID NO: 533-534 - skipped (2 entries)

SEQ ID NO: 535 - DNA, 27 bp, synthetic construct
  misc_feature 1..27: note = synthetic
  source 1..27: mol_type = other DNA
cagcagtatggtagctcaccttggacg

SEQ ID NO: 536 - protein, 9 aa, synthetic construct
  REGION 1..9: note = synthetic
QQYGSSPWT

SEQ ID NO: 537 - DNA, 357 bp, synthetic construct
  misc_feature 1..357: note = synthetic
  source 1..357: mol_type = other DNA
caggtgcagctacagcagtggggcgcaggactgttgaagccttcggagaccctgtccctcacctgcgctgtctatggtgggtccttcagtggttactactggaactggatccgccagcccccagggaaggggctggagtgggttggggaaatcagtcatagaggaagcaccaactacaacccgtccctcaagagtcgagtcaccatatcactggacacgtccaagaaccagttctccctgaagctgacctctgtgaccgccgcggacacggctgtgtattactgttcgagagacgaggaactggaattccgtttctttgactactggggccagggaaccctggtcaccgtctcctca

SEQ ID NO: 538 - protein, 119 aa, synthetic construct
  REGION 1..119: note = synthetic
QVQLQQWGAGLLKPSETLSLTCAVYGGSFSGYYWNWIRQPPGKGLEWVGEISHRGSTNYNPSLKSRVTISLDTSKNQFSLKLTSVTAADTAVYYCSRDEELEFRFFDYWGQGTLVTVSS

SEQ ID NO: 539 - DNA, 24 bp, synthetic construct
  misc_feature 1..24: note = synthetic
  source 1..24: mol_type = other DNA
ggtgggtccttcagtggttactac

SEQ ID NO: 540 - protein, 8 aa, synthetic construct
  REGION 1..8: note = synthetic
GGSFSGYY

SEQ ID NO: 541 - DNA, 21 bp, synthetic construct
  misc_feature 1..21: note = synthetic
  source 1..21: mol_type = other DNA
atcagtcatagaggaagcacc

SEQ ID NO: 542 - protein, 7 aa, synthetic construct
  REGION 1..7: note = synthetic
ISHRGST

SEQ ID NO: 543 - DNA, 39 bp, synthetic construct; identical to SEQ ID NO: 39
  misc_feature 1..39: note = synthetic
  source 1..39: mol_type = other DNA

SEQ ID NO: 544 - protein, 13 aa, synthetic construct
  REGION 1..13: note = synthetic
SRDEELEFRFFDY

SEQ ID NO: 545 - DNA, 321 bp, synthetic construct; identical to SEQ ID NO: 41
  misc_feature 1..321: note = synthetic
  source 1..321: mol_type = other DNA

SEQ ID NO: 546 - protein, 107 aa, synthetic construct; identical to SEQ ID NO: 42
  REGION 1..107: note = synthetic

SEQ ID NO: 547 - DNA, 18 bp, synthetic construct
  misc_feature 1..18: note = synthetic
  source 1..18: mol_type = other DNA
cagagtgttagcagctat

SEQ ID NO: 548 - protein, 6 aa, synthetic construct
  REGION 1..6: note = synthetic
QSVSSY

SEQ ID NO: 549-550 - skipped (2 entries)

SEQ ID NO: 551 - DNA, 27 bp, synthetic construct
  misc_feature 1..27: note = synthetic
  source 1..27: mol_type = other DNA
cagcagcgtagcaactggccgctcact

SEQ ID NO: 552 - protein, 9 aa, synthetic construct
  REGION 1..9: note = synthetic
QQRSNWPLT

SEQ ID NO: 553 - DNA, 372 bp, synthetic construct
  misc_feature 1..372: note = synthetic
  source 1..372: mol_type = other DNA
gaggtgcagctggtggagtctgggggaggcttggtacagcctggggggtccctgagactctcctgtgtagcctctggattcacctttagcctctatgccatgacctgggtccgccaggttccagggaaggggctggaatgggtctcaactattagtggtagtggtggtggcacatactacacagactccgttaagggccggttcaccatctccagagacaattccaagaacacactgtatctgcaaatgaacagcctgagagccgacgacacggccgttttttactgtacgaaagagagtacaactggaacttactcctacttctacggtatggacgtctggggccaagggaccacggtcaccgtctcctca

SEQ ID NO: 554 - protein, 124 aa, synthetic construct
  REGION 1..124: note = synthetic
EVQLVESGGGLVQPGGSLRLSCVASGFTFSLYAMTWVRQVPGKGLEWVSTISGSGGGTYYTDSVKGRFTISRDNSKNTLYLQMNSLRADDTAVFYCTKESTTGTYSYFYGMDVWGQGTTVTVSS

SEQ ID NO: 555 - DNA, 24 bp, synthetic construct
  misc_feature 1..24: note = synthetic
  source 1..24: mol_type = other DNA
ggattcacctttagcctctatgcc

SEQ ID NO: 556 - protein, 8 aa, synthetic construct
  REGION 1..8: note = synthetic
GFTFSLYA

SEQ ID NO: 557 - DNA, 24 bp, synthetic construct
  misc_feature 1..24: note = synthetic
  source 1..24: mol_type = other DNA
attagtggtagtggtggtggcaca

SEQ ID NO: 558 - protein, 8 aa, synthetic construct
  REGION 1..8: note = synthetic
ISGSGGGT

SEQ ID NO: 559 - DNA, 51 bp, synthetic construct
  misc_feature 1..51: note = synthetic
  source 1..51: mol_type = other DNA
acgaaagagagtacaactggaacttactcctacttctacggtatggacgtc

SEQ ID NO: 560 - protein, 17 aa, synthetic construct
  REGION 1..17: note = synthetic
TKESTTGTYSYFYGMDV

SEQ ID NO: 561 - DNA, 321 bp, synthetic construct
  misc_feature 1..321: note = synthetic
  source 1..321: mol_type = other DNA
gacatccagatgacccagtctccatcctccctgtctgcatctgtaggagacagagtcaccatcacttgccgggcaagtcagaccattagcagctatttaaattggtatcagcagaaaccagggaaagcccctaagctcctgatctatgctgcatccagtttgcaaagtggggtcccatcaaggttcagtggcagtggatctgggacagatttcactctcaccctcagcggtctccaacctgaagattttgcaacttactactgtcaacagagttacagtaccccgctcactttcggcggagggaccaaggtggagatcaaa

SEQ ID NO: 562 - protein, 107 aa, synthetic construct
  REGION 1..107: note = synthetic
DIQMTQSPSSLSASVGDRVTITCRASQTISSYLNWYQQKPGKAPKLLIYAASSLQSGVPSRFSGSGSGTDFTLTLSGLQPEDFATYYCQQSYSTPLTFGGGTKVEIK

SEQ ID NO: 563 - DNA, 18 bp, synthetic construct
  misc_feature 1..18: note = synthetic
  source 1..18: mol_type = other DNA
cagaccattagcagctat

SEQ ID NO: 564 - protein, 6 aa, synthetic construct
  REGION 1..6: note = synthetic
QTISSY

SEQ ID NO: 565-566 - skipped (2 entries)

SEQ ID NO: 567 - DNA, 27 bp, synthetic construct
  misc_feature 1..27: note = synthetic
  source 1..27: mol_type = other DNA
caacagagttacagtaccccgctcact

SEQ ID NO: 568 - protein, 9 aa, synthetic construct
  REGION 1..9: note = synthetic
QQSYSTPLT

SEQ ID NO: 569 - protein, 327 aa, synthetic construct
  REGION 1..327: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 570 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 571 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 572 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 573 - protein, 326 aa, synthetic construct
  REGION 1..326: note = synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPGGGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 574 - protein, 450 aa, synthetic construct
  REGION 1..450: note = aa 1-478: Human Lag3 (aa 29 through 450 of NP_002277.4) aa 451-478: myc-myc-hexahistidine tag
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLEQKLISEEDLGGEQKLISEEDLHHHHHH

SEQ ID NO: 575 - protein, 655 aa, synthetic construct
  REGION 1..655: note = aa 1-683: Human Lag3 (aa 29 through 450 of NP_002277.4) aa 451-683: mIgG2aFc
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 576 - protein, 516 aa, synthetic construct
  REGION 1..516: note = aa 1-533 (aa 18 through 533 of cynomolgus XP_005570011.1 modified to replace amino acid at position 74 with a P based on Rhesus macaque XP_001108923.1
APVKPPQPGAEISVVWAQEGAPAQLPCSPTIPLQDLSLLRRAGVTWQHQPDSGPPAPAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 577 - protein, 449 aa, synthetic construct
  REGION 1..449: note = synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 578 - protein, 214 aa, synthetic construct
  REGION 1..214: note = synthetic
EIVLTQSPATLSLSPGERTTLSCRASQRISTYLAWYQQKPGQAPRLLIYDASKRATGIPARFSGSGSGTGFTLTISSLEPEDFAVYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 579 - protein, 450 aa, synthetic construct
  REGION 1..450: note = synthetic
QVQLVESGGGVVQPGRSLRLSCVASGFTFSSYGMHWVRQAPGKGLEWVAIIWYDGSNKYYADSVKGRFTISRDNSKNTQYLQMNSLRAEDTAVYYCASVATSGDFDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 580 - protein, 445 aa, synthetic construct
  REGION 1..445: note = synthetic
QVQLQQWGAGLLKPSETLSLTCAVYGGSFSGYYWNWIRQPPGKGLEWVGEISHRGSTNYNPSLKSRVTISLDTSKNQFSLKLTSVTAADTAVYYCSRDEELEFRFFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 581 - protein, 214 aa, synthetic construct
  REGION 1..214: note = synthetic
EIVLTQSPATLSLSPGERATLSCRASQSVSSYLAWYQQKPGQAPRLLVYGASNRATGIPARFSGSGSGTDFTLTISSLEPEDFAFYYCQQRSNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 582 - protein, 525 aa, synthetic construct
  REGION 1..525: note = synthetic
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 583 - protein, 15 aa, synthetic construct
  REGION 1..15: note = synthetic
ENPVVHFFKNIVTPR

SEQ ID NO: 584 - DNA, 17 bp, synthetic construct
  misc_feature 1..17: note = synthetic
  source 1..17: mol_type = other DNA
agcagctctgccctcat

SEQ ID NO: 585 - DNA, 22 bp, synthetic construct
  misc_feature 1..22: note = synthetic
  source 1..22: mol_type = other DNA
gctctggctggtcttcagtatg

SEQ ID NO: 586 - DNA, 22 bp, synthetic construct
  misc_feature 1..22: note = synthetic
  source 1..22: mol_type = other DNA
ttgccgtatggttggtttgaac

SEQ ID NO: 587 - protein, 665 aa, synthetic construct
  REGION 1..665: note = hLAG3.Fc
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 588 - protein, 422 aa, synthetic construct
  REGION 1..422: note = hLAG3 extracellular domain P18627
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 589 - protein, 44 aa, synthetic construct
  REGION 1..44: note = hLAG3 epitope
LRRAGVTWQHQPDSGPPAAAPGHPLAPGPHPAAPSSWGPRPRRY